(19) United States
(12) Patent Application Publication
(10) Pub. No.: US 2010/0022766 A1
(43) Pub. Date: Jan. 28, 2010

(54) BIOSYNTHETIC GENE CLUSTER FOR THE PRODUCTION OF A COMPLEX POLYKETIDE

(75) Inventor: MIN HE, CONGERS, NY (US)

Correspondence Address: WYETH LLC;PATENT LAW GROUP, 5 GIRALDA FARMS, MADISON, NJ, 07940, US

(73) Assignee: WYETH, MADISON, NJ (US)

(21) Appl. No.: 12/508,735

(22) Filed: Jul. 24, 2009

Related U.S. Application Data
(60) Provisional application No. 61/083,631, filed on Jul. 25, 2008.

(51) Int. Cl. C07D 498/08; C12N 9/14; C07H 21/04; C12N 15/63; C12P 17/18; C12N 5/00

(52) U.S. Cl. 540/456; 435/195; 536/23.2; 435/320.1; 435/119; 435/325

(57) ABSTRACT

A polyketide synthase complex composed of polyketide synthase with 15 total modules, a non-ribosomal peptide synthetase with 1 module, and a cytochrome P450 hydroxylase is described. Also provided are novel Streptomyces species and methods of modified Streptomyces species. Further described are novel compounds, C-36-ketomeridamycin, C9-deoxomeridamycin, and C9-deoxoprolylmeridamcyin and uses thereof.

### CROSS REFERENCE TO RELATED APPLICATIONS

This application claims priority from U.S. Provisional Application No. 61/083,631, filed Jul. 25, 2008, which is incorporated by reference in its entirety.

### BACKGROUND OF THE INVENTION

The present invention relates to the cloning and sequencing of the biosynthetic gene cluster that encodes a Type I polyketide synthase (PKS) and a non-ribosomal peptide synthase responsible for the production of meridamycin. The present invention also relates to methods for genetically manipulating the meridamycin biosynthetic pathway to produce derivatives of meridamycin.

Polyketides represent a large group of natural products that are derived from successive condensations of simple carboxylates, such as acetate, propionate or butyrate. Naturally occurring polyketides possess a broad range of biological activities, including antibiotics such as tetracyclines and erythromycin, anticancer agents such as daunomycin and bryostatin, immunosuppressants such as FK506 and rapamycin, and veterinary products such as monensin and avermectin. Polyketides are produced in most groups of organisms and are especially abundant in a class of mycelial bacteria, the actinomycetes, which produce various types of polyketides.

The enzymes responsible for the biosynthesis of polyketides are called polyketide synthases (PKSs). Two general classes of PKSs exist. One class, known as Type I PKSs, is represented by the PKSs for the synthesis of macrolide polyketides such as erythromycin and rapamycin. This type of PKSs has a modular enzymatic structure, in which a module is defined as a set of enzymatic domains that are necessary to catalyze the recognition and incorporation of a specific 2-carbon extending unit (usually a malonyl-CoA, a methyl malonyl-CoA or a propionyl-CoA) into the growing polyketide chain. A minimal type I PKS module contains three enzymatic domains: (1) a ketosynthase domain (KS) which is responsible for catalyzing the Claisen condensation reaction between a starter unit or a growing polyketide chain and an extender unit; (2) an acyltransferase domain (AT) which selectively binds a specific extender unit from the intracellular pools of the various CoA carboxylates and then transfers it to the acyl carrier center; (3) an acyl carrier protein domain (ACP) which contains a serine residue that has been post-translationally modified with a 4-phosphopantethein group and serves as the acceptor for the extender unit or a growing polyketide chain. In addition to the KS, AT, and ACP domains, a type I PKS module can also have one, two or three of the following domains: a ketoreductase domain (KR) which reduces the β-ketone to the hydroxyl function, a dehydratase domain (DH) which eliminates water from the α,β carbon centers to generate a double bond between them, and a enoylreductase domain (ER) which further reduces the double bond generated by DH domain to yield the β-methylene group.

A co-linear relationship exists between the primary organization of the Type I PKS and the structure of the polyketide backbone. For examples, the number of modules in the PKS determines the number of the two-carbon units in the carbon backbone of the final polyketide product, the presence of a specific AT domain determines which extender (malonate, methylmalonate or ethylmalonate, etc.) is incorporated into the growing polyketide chain, and the presence of the reduction domains (KR, DH and ER) in a module determines the extent of reduction of the β-carbon formed at the give condensation.

The second class of PKSs, called Type II PKSs, is responsible for the synthesis of aromatic polyketides such as daunorubicin and tetracenomycin. Type II PKSs have a single set of enzymatic activities (KS, AT, ACP, KR etc.) that reside in individual proteins and are used iteratively to generate polyketides with poly-cyclic ring structure. There is no clear correlation between the type II PKS enzymatic organization and the final polyketide structure.

The genes encoding PKSs and the necessary tailoring enzymes to make a polyketide compound have been shown in all cases to be clustered together on the chromosome of the producing microorganism, and thus are collectively called “PKS biosynthetic gene cluster”. Tremendous research work has been done in academic and industrial fields aimed at generating novel polyketide compounds with potential therapeutic applications through genetic manipulation of PKS biosynthetic gene clusters. There is a continuing need in the art to determine the genes encoding novel PKS complexes.

### SUMMARY OF THE INVENTION

The present invention provides a biosynthetic gene cluster encoding a polyketide synthase complex for producing a polyketide compound. The invention further provides a meridamycin synthase complex comprising three polyketide synthases, each comprises at least one module, a non-ribosomal peptide synthase, which in one embodiment comprises 4 catalytic domains, and, in one embodiment, a cytochrome P450 hydroxylase. In one embodiment, the polyketide synthases comprise 15 modules in total.

In one embodiment, the modules of the polyketide synthase comprise a ketosynthase domain, an acyltransferase domain, and an acyl carrier protein.

In another embodiment, the modules further comprise a ketoreductase domain, a dehydratase domain and an enoylreductase domain.

The present invention also provides isolated nucleic acids which comprise open reading frames comprised within the polyketide synthase and encode polypeptides required for synthesis of a polyketide compound. The corresponding amino acid sequences are also provided.

The present invention also provides nucleic acid sequences which are complementary to, and/or hybridize under stringent conditions to the nucleic acids comprising the polyketide synthase.

Further provided by the present invention is a method of producing a polyketide compound produced by the polyketide synthase. In one embodiment, the polyketide compound is meridamycin.

The present invention also provides a method of modifying the polyketide synthase of the invention to produce modified polyketide compounds, and the modified polyketide compounds thereof.

In one embodiment, the modification comprises addition, removal, or substitution of at least one amino acid, wherein such modification results in alterations of i) the ring size, ii) the reduction extent of a β-keto group on the ring, iii) a side chain at an α-carbon, or iv) the starting unit of the polyketide compound.

In another embodiment, the modified polyketide compound is a keto-derivative of meridamycin.

The present invention further provides a method for preventing neurodegeneration by contacting neuronal cells with an effective amount of a polyketide compound produced by the polyketide synthase of SEQ ID NO: 1, which sequence may contain appropriate modifications.

A method for promoting neuroregeneration by contacting neuronal cells with an effective amount of a polyketide compound produced by the polyketide synthase having a nucleic acid sequence comprising SEQ ID NO: 1, which sequence may contain appropriate modifications.

In one aspect, the present invention relates macrolides and other chemical compounds produced by a novel actinomycete strain, as well as pharmaceutical compositions containing such compounds.

In particular, the invention relates to meridamycin compounds, including meridamycin and derivatives thereof of formula:

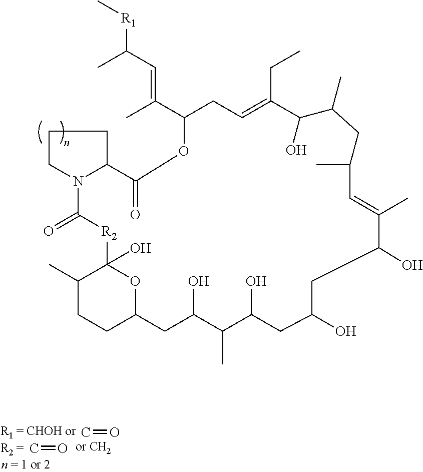

a salt thereof, or mixtures thereof. Such compounds can be used to prepare compositions further comprising one or more pharmaceutically acceptable carriers, excipients, or diluents. Also provided are methods for treating a mammal comprising administering to the mammal a compound or composition of the invention, particularly for treatment of a neurological disorder.

The invention further relates to methods of producing the compounds in an actinomycete strain, such as by growth in cell culture of the actinomycete strain LL-BB0005. Cell culture of the actinomycete strain LL-BB0005, for example, has been found to produce compounds having formulas (I), which can be isolated from the fermentation broth.

Other aspects and advantages of the invention will be readily apparent from the following detailed description of the invention.

### BRIEF DESCRIPTION OF THE DRAWINGS

FIG. 1 is a schematic representation of the genetic organization of the meridamycin biosynthetic gene cluster.

FIG. 2A is a schematic representation of the wild-type genomic DNA of a MerP gene.

FIG. 2B is a schematic representation of the MerP::Apr mutant construct.

FIG. 3A shows a schematic representation of the production of meridamycin.

FIG. 3B shows a schematic representation of the production of C36-keto-meridamycin.

FIG. 4 is a proton NMR spectrum of the compound of formula (III), wherein n=2, in CD₃OD at 400 MHz.

FIG. 5 is a carbon NMR spectrum of the compound of formula (III), wherein n=2, in CD₃OD at 100 MHz.

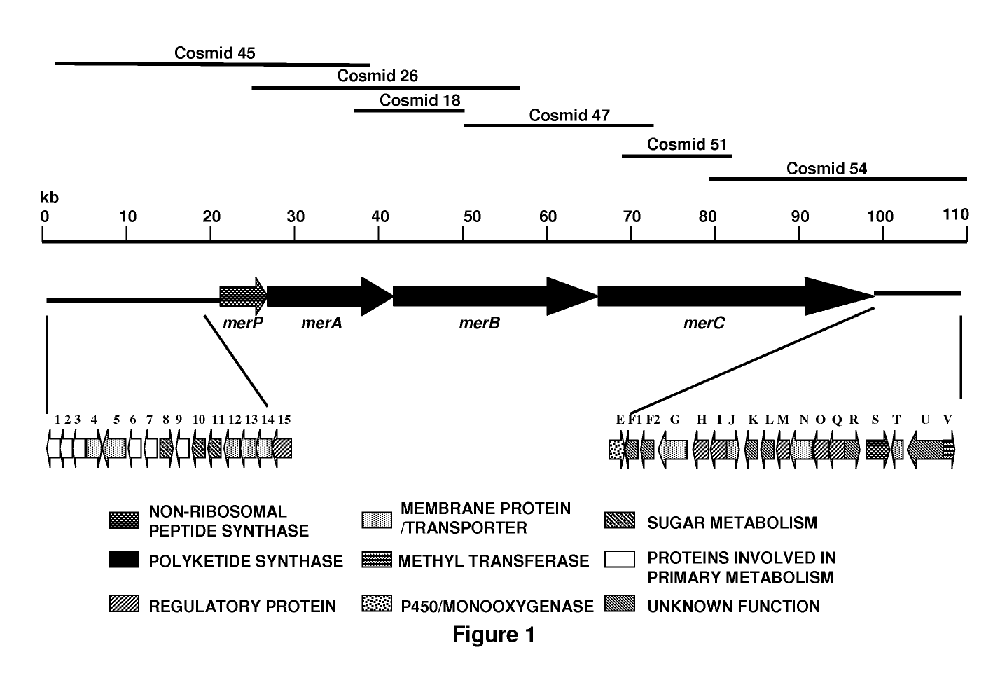

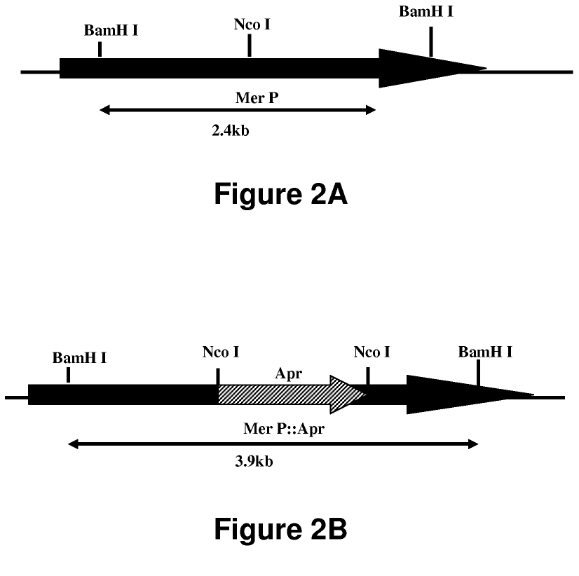

### DETAILED DESCRIPTION OF THE INVENTION

The present invention provides an isolated biosynthetic gene cluster for a polyketide compound. Suitably, the biosynthetic gene cluster is a meridamycin biosynthetic nucleic acid sequence substantially isolated from cellular materials, i.e., an Actinomycete species, with which it is naturally found.

In one embodiment, the biosynthetic gene cluster nucleic acid sequence encodes three polyketide synthases which comprise 15 modules in total, and a non-ribosomal peptide synthase, which comprises 4 catalytic domains. In one embodiment, the modules of the polyketide synthase comprise a ketosynthase domain, an acyltransferase domain, and an acyl carrier protein. In another embodiment, the modules further comprise a ketoreductase domain, a dehydratase domain and an enoylreductase domain.

The present invention further provides nucleic acids of genes and/or open reading frames encoding these polypeptides and enzymes, such as polyketide synthases (PKS), non-ribososomal peptide synthases (NRPS), of an isolated meridamycin biosynthetic cluster.

The present invention also provides nucleic acids which comprise open reading frames comprised within the biosynthetic gene cluster and encode polypeptides and enzymes required for synthesis of a polyketide compound. The corresponding amino acid sequences are also provided.

In one embodiment, the present invention provides for the use of recombinant technology to produce one or more of the polypeptides and/or enzymes of the meridamycin biosynthetic pathway using the sequences provided herein.

In one embodiment, the invention provides a method of generating mutant Streptomyces strains, generated by modification of one or more of the genes of the biosynthetic gene cluster.

The present invention advantageously permits specific changes to be made to individual modules of the meridamycin biosynthetic gene cluster, either by site directed mutagenesis or replacement, to genetically modify the polyketide core. Additionally, the modules can be used to modify other biosynthetic gene clusters that direct the synthesis of other useful peptides through module swapping.

In another embodiment, the present invention provides methods of modifying one or more of the genes and/or open reading frames of the meridamycin biosynthetic gene cluster. Such modifications can be used to generate macrolide compounds, e.g., meridamycin, 36-ketomeridamycin, and 9-deoxomeridamycin.

The present invention further provides nucleic acids, genes and/or open reading frames encoding the proteins for the synthesis of meridamycin and meridamycin derivatives.

### I. DEFINITIONS

The following definitions and those provided elsewhere in the specification are provided for the full understanding of terms and abbreviations used in this specification.

Meridamycin is a macrolide polyketide that has been shown to have strong FKBP12 binding activity and significant neuroprotective activity in vitro, having the structure (I):

### Error! Objects Cannot be Created from Editing Field Codes. (I)

It is produced by terrestrial actinomycetes Wyeth culture LL-BB0005, deposited under the terms of the Budapest Treaty with the Agricultural Research Service Culture Collection (NRRL) on May 18, 2004 (Accession No. NRLL 30748). Meridamycin functions as an immunophilin which binds to FK-binding proteins.

The abbreviations in the specification correspond to units of measure, techniques, properties or compounds as follows: “hr” means hour(s), “μL” means microliter(s), “nM” means nanomolar, “μM” means micromolar, “M” means molar, “mmole” means millimole(s), “kb” means kilobase, “bp” means base pair(s), and “polymerase chain reaction” is abbreviated PCR; “non-ribosomal peptide synthetase” is abbreviated NRPS; dopamine is abbreviated “DA”; polyketide synthase is abbreviated “PKS”.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

“Amplification” of DNA as used herein denotes the use of any amplification method for increasing the concentration of a particular DNA sequence within a mixture of DNA sequences. For example, polymerase chain reaction (PCR) (see, for example, Saiki et al., Science 1988, 239:487) or the ligase chain reaction.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

A coding sequence is “under the control of” or “operatively associated with” expression control sequences in a cell when RNA polymerase transcribes the coding sequence into RNA, particularly mRNA, which is then trans-RNA spliced (if it contains introns) and translated into the protein encoded by the coding sequence.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. For example, the present invention includes chimeric DNA molecules that comprise a DNA sequence and a heterologous DNA sequence that is not part of the DNA sequence. In this context, the heterologous DNA sequence refers to a DNA sequence that is not naturally located within the biosynthetic gene cluster sequence. Alternatively, the heterologous DNA sequence can be naturally located within the biosynthetic gene cluster at a location where it does not natively occur. For example, a sequence encoding a functional enzyme or domain may be natively located within the NRPS sequence, but deleted from this site and inserted elsewhere in the biosynthetic gene cluster sequence. A heterologous expression regulatory element is an element operatively associated with a different gene than the one it is operatively associated with in nature. In the context of the present invention, a gene encoding a protein of interest is heterologous to the vector DNA in which it is inserted for cloning or expression, and it is heterologous to a host cell containing such a vector, in which it is expressed.

The term “expression control sequence” refers to a promoter, any enhancer element, or suppression elements (e.g., an origin of replication) that combine to regulate the transcription of a coding sequence. The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cells genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

As used herein, the term “amino acid equivalent” refers to compounds which depart from the structure of the naturally occurring amino acids, but which have substantially the structure of an amino acid, such that they can be substituted within a peptide that retains biological activity. Thus, for example, amino acid equivalents can include amino acids having side chain modifications and/or substitutions, and also include related organic acids, amides or the like. The term “amino acid” is intended to include amino acid equivalents. The term “residues” refers both to amino acids and amino acid equivalents.

As sometimes used herein, the terms “homologous” and “homology” refer to the relationship between proteins that possess a “common evolutionary origin,” including proteins from superfamilies (e.g., the immunoglobulin superfamily) and homologous proteins from different species (e.g., myosin light chain, etc.) as described, for example, in Reeck et al., Cell 50:667, 1987, the disclosure of which is herein incorporated by reference. Such proteins (and their encoding genes) have sequence homology, as reflected by their sequence similarity, whether in terms of percent similarity or the presence of specific residues or motifs at conserved positions.

Accordingly, the term “sequence similarity” refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90% or 95% of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90% are similar. Preferably, the amino acids are functionally identical. Preferably, the similar or homologous sequences are identified by alignment using, for example, the GCG (Genetics Computer Group, Program Manual for the GCG Package, Version 10, Madison, Wis.) pileup program, or any of the programs described above (BLAST, FASTA, etc.).

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length, and may be up to about 700 amino acids. Examples of suitable fragments are described herein.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., Molecular Cloning: A Laboratory Manual, Second Edition (1989) Cold Spring Harbor Laboratory Press, Cold Spring Harbor, N.Y. (herein “Sambrook et al., 1989”), the disclosure of which is herein incorporated by reference. The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tm, e.g., 40% formamide, with 5× or 6×SCC. High stringency hybridization conditions correspond to the highest Tm, e.g., 50% formamide, 5× or 6×SCC. SCC is a 0.15M NaCl, 0.015M Na citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tm of 55° C., and utilizes conditions as set forth above. In a preferred embodiment, the Tm is 60° C.; in a more preferred embodiment, the Tm is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligoncucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence.

The term “variant” may also be used to indicate a modified or altered gene, DNA sequence, enzyme, cell, etc., i.e., any kind of mutant. Two specific types of variants are “sequence conservative variants”, a polynucleotide sequence where a change of one or more nucleotides in a given codon position results in no alteration in the amino acid encoded at that position, and “function conservative variants”, where a given amino acid residue in a protein or enzyme has been changed without altering the overall conformation and function of the polypeptide. Amino acids with similar properties are well known in the art. Amino acids other than those indicated as conserved may differ in a protein or enzyme so that the percent protein or amino acid sequence similarity between any two proteins of similar function may vary and may be, for example, from about 70% to about 99% as determined according to an alignment scheme such as by the Clustal Method, wherein similarity is based on the algorithms available in MEGALIGN. A “function conservative variant” also includes a polypeptide or enzyme which has at least about 60% amino acid identity as determined by BLAST or FASTA alignments, preferably at least about 75%, more preferably at least about 85%, and most preferably at least about 90%, and which has the same or substantially similar properties or functions as the native or parent protein or enzyme to which it is compared.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994), the disclosures of which are herein incorporated by reference.

### II. BIOSYNTHETIC GENE CLUSTER

In one aspect, the invention provides an isolated meridamycin biosynthetic gene cluster. The invention discloses the isolation of a group of cosmids identified as pMH45, pMH18, pMH26, pMH47, pMH51 and pMH54, which contain the genetic information for the biosynthesis of meridamycin. SEQ ID NO:1 provides the nucleic acid sequence of the isolated meridamycin biosynthetic gene cluster. Also included in the present invention are the strands complementary to the nucleic acid sequences of Table 1, as wells as natural variants and engineered modifications of the sequences of the biosynthetic gene cluster and their complementary strands. Such modifications include, for example, labels which are known in the art, methylation, and substitution of one or more of the naturally occurring nucleotides with a degenerate nucleotide. These modifications may be to increase expression, yield, and/or to improve purification in the selected expression systems, or for another desired purpose.

Further, the invention encompasses functional fragments of the nucleic acid sequences of SEQ ID NO:1 and its reverse complement. Examples of suitable fragments are provided in Table 1 with reference to the nucleic acid sequences of SEQ ID NO:1. Table 1 further identifies the length of the polypeptides encoded by the coding sequences and references the relevant sequence identification number and function for each.

Notably, some of the coding sequences are located on the sense strain of SEQ ID NO:1, including, e.g., ORF4, ORF8, ORF 21, MerA, MerB, MerC, MerE, ORF F-2, MerJ, ORFr, MerS, and ORFV. In addition, some of the coding sequences are located on the strand which is the reverse complement of SEQ ID NO:1, including e.g., ORF1-3, ORF5-7, ORF9-15, MerM-MerQ, MerT, and MerU. For convenience, separate SEQ ID NO:s are provided for those coding sequences located on the reverse strand of SEQ ID NO:1. Other suitable nucleic acid fragments include nucleic acid sequences encoding the amino acids of Table 1 [SEQ ID NO:31-67] and nucleic acid sequences encoding the amino acid sequences of the modules and catalytic domains provided in Table 2, i.e., the specified fragments of SEQ ID NO:47, 48 and 49. Still other suitable fragments will be readily apparent to one of skill in the art.

Thus, the present invention provides an isolated nucleic acid sequence of a coding region from the meridamycin biosynthetic gene cluster. These include, e.g., any of ORF1-15, ORF F1-1, ORF F-2, ORFK, ORFR, ORFV, MerP, MerA, MerB, MerC, MerE, any of MerG-J, Mer M-O, MerQ, or Mer S-U. In one embodiment, the isolated nucleic acid sequence contains a single open reading frame or gene. In another embodiment, the isolated nucleic acid sequence contains one or more open reading frames or genes. For example, a selected host cell may contain the sequences spanning of MerP and MerA-MerC, optionally also in combination with MerE, nucleotides 26284-99586 of SEQ ID NO:1. Alternatively, a selected host cell may contain the isolated sequences of one or more of these coding regions, e.g., MerP [nt 21592-26311 of SEQ ID NO:1], MerA [nt 26284-43422 of SEQ ID NO:1], MerB [nt 43480-64788 of SEQ ID NO:1] and MerC [nt 64785-98351 of SEQ ID NO:1]. Alternatively, a vector host cell may contain any combination of sequences encoding MerP [SEQ ID NO:46], MerA [SEQ ID NO:47], MerB [SEQ ID NO:48], MerC [SEQ ID NO:49] and/or MerE [SEQ ID NO:50].

Also included are modifications of the fragments of the biosynthetic gene cluster and their complementary strands. Such modifications include, for example, labels which are known in the art, methylation, and substitution of one or more of the naturally occurring nucleotides with a degenerate nucleotide. These modifications may be to increase expression, yield, and/or to improve purification in the selected expression systems, or for another desired purpose.

TABLE 1
Summary of ORFs in Meridamycin Biosynthetic Gene Cluster

| | | Position (bp) | No. of Amino | |
|---|---|---|---|---|
| | | With reference to | Acids/ | |
| Orf | SEQ ID NO. | SEQ ID NO: 1 | SEQ ID NO: | Function |
| Orf 1 | SEQ ID NO: 6 | 1108 . . . 221 | 295 | Purine synthase |
| | | | SEQ ID NO: 31 | |
| Orf 2 | SEQ ID NO: 7 | 2830 . . . 1265 | 521 | Purine synthase |
| | | | SEQ ID NO: 32 | |
| Orf 3 | SEQ ID NO: 8 | 3483 . . . 2827 | 218 | Purine synthase |
| | | with RBS | SEQ ID NO: 33 | |
| Orf 4 | | 3885 . . . 4727 | 280 | Ion transport protein |
| | | | SEQ ID NO: 34 | |
| Orf 5 | SEQ ID NO: 9 | 6643 . . . 4790 | 617 | Membrane protein |
| | | | SEQ ID NO: 35 | |
| Orf 6 | SEQ ID NO: | 7762 . . . 6878 | 294 | Succinyl-CoA |
| | 10 | | SEQ ID NO: 36 | synthetase (α chain) |
| Orf 7 | SEQ ID NO: | 8902 . . . 7784 | 372 | Succinyl-CoA |
| | 11 | | SEQ ID NO: 37 | synthetase (β chain) |
| Orf 8 | | 9320 . . . 10960 | 546 | β-1,4-endo glucanase |
| | | | SEQ ID NO: 38 | |
| Orf 9 | SEQ ID NO: | 12377 . . . 11037 | 446 | Argininosuccinate |
| | 12 | | SEQ ID NO: 39 | synthase |
| Orf 10 | SEQ ID NO: | 14809 . . . 12677 | 710 | Mycodextranase |
| | 13 | | SEQ ID NO: 40 | |
| Orf 11 | SEQ ID NO: | 16517 . . . 14919 | 532 | α-1,4-glucosidase |
| | 14 | | SEQ ID NO: 41 | |
| Orf 12 | SEQ ID NO: | 17423 . . . 16548 | 291 | Sugar transporter (ABC |
| | 15 | | SEQ ID NO: 42 | type permease, inner |
| | | | | portion)) |
| Orf 13 | SEQ ID NO: | 18442 . . . 17420 | 340 | Sugar transporter (ABC |
| | 16 | | SEQ ID NO: 43 | type permease, inner |
| | | | | portion) |
| Orf 14 | SEQ ID NO: | 19811 . . . 18381 | 476] | Sugar transporter |
| | 17 | | SEQ ID NO: 44 | (extracellular sugar |
| | | | | binding portion) |
| Orf 15 | SEQ ID NO: | 20919 . . . 19942 | 325 | LacI family |
| | 18 | | SEQ ID NO: 45 | transcription regulator |
| MerP | | 21592 . . . 26311 | 1572 | NRPS for incorporating |
| | | | SEQ ID NO: 46 | pipecolic acid (single |
| | | | | module with 4 |
| | | | | domains: CATC) |
| MerA | | 26284-43422 | 5712 | Type I PKS |
| | | | SEQ ID NO: 47 | (4 modules) |
| MerB | | 43480 . . . 64788 | 7102 | Type I PKS |
| | | | SEQ ID NO: 48 | (4 modules) |
| MerC | | 64785 . . . 98351 | 11,188 | Type I PKS |
| | | | SEQ ID NO: 49 | (7 modules) |
| MerE | | 98392 . . . 99585 | 397 | Cytochrome P450 |
| | | | SEQ ID NO: 50 | hydroxylase |
| ORF | SEQ ID NO: | 100253 . . . 99735 | 172 | |
| F-1 | 19 | | SEQ ID NO: 51 | |
| ORF | | 100527 . . . 101036 | 169 | |
| F-2 | | | SEQ ID NO: 52 | |
| MerG | SEQ ID NO: | 102697 . . . 101213 | 494 | Drug efflux |
| | 20 | | SEQ ID NO: 53 | transporter |
| Mer H | SEQ ID NO: | 103295 . . . 102816 | 159 | Drug resistance |
| | 21 | | SEQ ID NO: 54 | regulatory protein |
| Mer I | SEQ ID NO: | 104321 . . . 103377 | 314 | Regulatory |
| | 22 | | SEQ ID NO: 55 | protein |
| Mer J | | 104276 . . . 105271 | 331 | Membrane |
| | | | SEQ ID NO: 56 | protein |
| ORF K | SEQ ID NO: | 106205 . . . 105381 | 274 | |
| | 23 | | SEQ ID NO: 57 | |
| Mer L | SEQ ID NO: | 107367 . . . 106318 | 349 | |
| | 24 | | SEQ ID NO: 58 | |
| Mer M | SEQ ID NO: | 107844 . . . 107437 | 135 | Resistance related |
| | 25 | | SEQ ID NO: 59 | regulator |
| Mer N | SEQ ID NO: | 109422 . . . 107929 | 497 | Putative drug efflux |
| | 26 | | SEQ ID NO: 60 | transporter |
| Mer O | SEQ ID NO: | 110060 . . . 109419) | 213 | Drug resistant |
| | 27 | | SEQ ID NO: 61 | related regulator |
| Mer Q | SEQ ID NO: | 111196 . . . 110150 | 348 | LysR family |
| | 28 | | SEQ ID NO: 62 | regulator |
| ORF R | | 111061 . . . 111717 | 218 | |
| | | | SEQ ID NO: 63 | |
| Mer S | | 111846 . . . 113225 | 459 | FAD-dependent |
| | | | SEQ ID NO: 64 | monooxygenase |
| Mer T | SEQ ID NO: | 113682 . . . 113275 | 135 | Putative short |
| | 29 | | SEQ ID NO: 65 | membrane protein |
| | | | | with unknown |
| | | | | function |
| Mer U | SEQ ID NO: | 116365 . . . 113915 | 816 | |
| | 30 | | SEQ ID NO: 66 | |
| ORF V | | 116453 . . . 116854, | 134 | (quinone) methyl |
| | | (incomplete) | SEQ ID NO: 67 | transferase |

As indicated in Table 1, the MerP, MerA, MerB, MerC and MerE genes are those responsible for producing the core of the meridamycin molecule. MerP encodes a non-ribosomal peptide synthetase. Each of MerA, MerB and MerC encodes a type I polyketide synthetase (PKS), each composed of multiple modules. Each module provides a catalytic domain, e.g., a ketosynthase reduction domain (KR), an acyltransferase reduction domain (AT), a dehydratase reduction domain (DH) or an enoylreductase (ER) reduction domain. For example, MerA contains 4 modules, MerB contains 4 modules and MerC contains 7.0 modules. See Table 2.

TABLE 2
Module and catalytic domain organization in meridamycin PKSs:

| | | | | Catalytic domain (start aa#- |
|---|---|---|---|---|
| | | Start | End | end aa#), |
| | | position | position | with reference to SEQ ID NO: of |
| Protein | Module | (aa #) | (aa #) | referenced Mer Gene |
| MerA | Loading | 1 | 1050 | KS(21-442), AT(580-879), ACP |
| SEQ | module | | | (970-1040) |
| ID NO: | 1 | 1051 | 2510 | KS (1060-1484), AT (1589-1877), |
| 47 | | | | KR (2147-2322), |
| | | | | ACP (2411-2496) |
| | 2 | 2511 | 4183 | KS (2523-2943), AT (3041-3330), |
| | | | | DH (3385-3548), KR3823-4002), |
| | | | | ACP (4091-4176) |
| | 3 | 4184 | 5172 | KS (4195-4621), AT (4719-4989), |
| | | | | KR (5299-5472), |
| | | | | ACP (5553-5629) |
| MerB | 4 | 1 | 1717 | KS (33-455), AT (556-857), |
| SEQ | | | | DH (914-1078), KR (1355-1537), |
| | | | | ACP (1623-1708) |
| ID NO: | 5 | 1718 | 3263 | KS (1728-2155), AT (2266-2555), |
| 48 | | | | KR (2896-3072), |
| | | | | ACP (3168-3253) |
| | 6 | 3264 | 5293 | KS (3276-3704), AT (3806-4096), |
| | | | | DH (4154-4320), ER (4633-4939), |
| | | | | KR (4940-5126), |
| | | | | ACP (5211-5296) |
| | 7 | 5294 | 7102 | KS (5317-5744), AT (5841-6129), |
| | | | | DH (6188-6354), KR (6664-6848), |
| | | | | ACP (6935-7020) |
| MerC | 8 | 1 | 1496 | KS (49-476), AT (540-714), |
| SEQ | | | | KR (1141-1317), |
| ID | | | | ACP (1409-1487) |
| NO: 49 | 9 | 1497 | 2942 | KS (1507-1929), AT (2024-2294), |
| | | | | KR (2598-2774), |
| | | | | ACP (2848-2933) |
| | 10 | 2943 | 4470 | KS (2953-3371), AT (3475-3765), |
| | | | | KR (4104-4280), |
| | | | | ACP (4376-4461) |
| | 11 | 4471 | 5930 | KS ((4481-4909), AT (5004-5274), |
| | | | | KR (5578-5751), |
| | | | | ACP (5837-5918) |
| | 12 | 5931 | 7386 | KS (5941-6368), AT (6458-6728), |
| | | | | KR (7038-7211), |
| | | | | ACP (7292-7376) |
| | 13 | 7387 | 9487 | KS (7396-7823) AT (7917-8190), |
| | | | | DH (8303-8477), ER (8822-9124), |
| | | | | KR (9124-9312), |
| | | | | ACP (9404-9489) |
| | 14 | 9510 | 11134 | KS (9510-9935), |
| | | | | AT (10051-10340), |
| | | | | ACP (11049-11134) |

After production of the core modules (e.g., by MerP, MerA, MerB and MerC), a polyketide core can then be modified by additional enzymes that are herein termed “tailoring enzymes”. These enzymes alter the side chains of the polyketide core without altering the number of the carbon atoms present within the polyketide core. Such tailoring enzymes may include, but are not limited to, hydroxylation and methylation. An example of one such tailoring enzyme, a cytochrome P450-like hydroxylase, is encoded by MerE.

Other functional polypeptides and enzymes including, e.g., a purine synthase, succinyl-CoA synthetase, a glucanase, arginonosuccinate synthase, mycodextranase, glucosidase, sugar transporter, regulatory proteins, drug efflux transporters, and membrane proteins, have been identified.

In one embodiment, a host cell is provided which contains the genes encoding at least the polyketide core. The host cell may be a modified streptomyces and/or actinomycete strain. Alternatively, the host cell may be of type that does not natively carry these biosynthetic genes. In one embodiment, the host cell contains one or more of the other genes of the biosynthetic gene cluster, e.g., merE, (any one from merG-U), ORF1-15, ORFF-1, ORFF-2, or ORFK, ORFR or ORFV.

In one embodiment, the invention provides a mutant gene in which the function of one or more of the catalytic domains (e.g., modules) within the gene region is eliminated. This mutation can be accomplished by deletion, a frame shift mutation, or other methods known in the art. Desirably, the function of each of these modules is retained, where the function of the selected gene is retained.

In another embodiment, the invention provides novel amino acid sequences, including, inter alia, polypeptides, and enzymes of the meridamycin biosynthetic synthase complex provided in Table 1 and 2 [SEQ ID NO:31-67 and fragments thereof, e.g., those in Table 2]. The amino acid sequences of the invention may be expressed from the nucleic acid sequences of the invention, e.g., from SEQ ID NO:1 or fragments thereof such as are identified herein, or from other nucleic acid sequences encoding these amino acids.

In still another embodiment, these amino acid sequences, or fragments thereof, may be produced synthetically using techniques known to those of skill in the art, including, e.g., by chemical synthesis. For example, peptides can also be synthesized by the well-known solid phase peptide synthesis methods (see e.g., Merrifield, J. Am. Chem. Soc., 85:2149 (1962); Stewart and Young, Solid Phase Peptide Synthesis (Freeman, San Francisco, 1969) pp. 27-62, the disclosure of which is herein incorporated by reference).

Suitable production techniques are well known to those of skill in the art. See, e.g., Sambrook et al, Molecular Cloning: A Laboratory Manual, Cold Spring Harbor Press (Cold Spring Harbor, N.Y.) supra. The sequences of any of the amino acid sequences provided herein can be readily generated using a variety of techniques. These and other suitable production methods are within the knowledge of those of skill in the art and are not a limitation of the present invention.

In one particular embodiment, the amino acid sequences of the invention are produced by expression of one or more of the ORFs or genes in a selected host cell. Typically, a vector is designed to carry the nucleic acid sequences encoding one or more ORFs or genes into a desired host cell.

The terms “vector”, “cloning vector” and “expression vector” refer to the vehicle by which DNA can be introduced into a host cell, resulting in expression of the introduced sequence. In one embodiment, vectors comprise a promoter and one or more control elements (e.g., enhancer elements) that are heterologous to the introduced DNA but are recognized and used by the host cell. In another embodiment, the sequence that is introduced into the vector retains its natural promoter that may be recognized and expressed by the host cell (Bormann et al., J. Bacteriol 1996; 178:1216-1218). In one embodiment, the vector compatible with the present invention is an intergeneric shuttle vector that permits conjugation between e.g., Streptomyces and E. coli. In another embodiment, the vector is a cosmid.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

An “intergeneric vector” is a vector that permits intergeneric conjugation, i.e., utilizes a system of passing DNA from E. coli to actinomycetes directly (Keiser, T. et al., Practical Streptomyces Genetics (2000) John Innes Foundation, John Innes Centre (England) the disclosure of which is herein incorporated by reference). Intergeneric conjugation has fewer manipulations than transformation.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA (which may be circular), usually of bacterial origin, that can readily accept additional (foreign) DNA and which can readily be introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes.

Vector constructs may be produced using conventional molecular biology and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); F. M. Ausubel et al. (eds.), Current Protocols in Molecular Biology, John Wiley & Sons, Inc. (1994), supra.

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal).

The host cell itself may be selected from any biological organism, including prokaryotic (e.g., bacterial) cells, plant cells, and eukaryotic cells, including, insect cells, yeast cells and mammalian cells. Representative examples of appropriate host cells include bacterial cells, such as, E. coli, Streptomyces and Bacillus subtilis cells; fungal cells, such as yeast cells and Aspergillus cells; and insect cells such as Drosophila S2 and Spodoptera Sf9 cells.

The term “expression system” means a host cell and compatible vector under suitable conditions, e.g. for the expression of a protein coded for by foreign DNA carried by the vector and introduced to the host cell. A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids, BAC vector and phagemids. The expression systems may contain control regions that regulate as well as engender expression. Generally, any system or vector that is able to maintain, propagate or express a polynucleotide to produce an enzyme in a host may be used. The appropriate nucleotide sequence may be inserted into an expression system by any of a variety of well-known and routine techniques, such as, for example, those set forth in Sambrook et al., Molecular Cloning: A Laboratory Manual, 2nd Ed., Cold Spring Harbor Laboratory Press, Cold Spring Harbor, N.Y. (1989), supra. Appropriate secretion signals may be incorporated into the desired enzyme to allow secretion of the translated protein into the lumen of the endoplasmic reticulum, the periplasmic space or the extracellular environment. These signals may be endogenous to the enzyme or they may be heterologous signals.

Thus, the determination of the biosynthetic pathway of meridamycin by the inventors permits, in one embodiment, one of ordinary skill in the art to clone and express the pathway, and thus, a polyketide, in a heterologous organism. The invention also permits portions of isolated nucleic acid sequences of the biosynthetic gene cluster (e.g., one or more ORFs or genes) to be expressed in a heterologous host cell, i.e., another Streptomycete strain, a non-Streptomycete and/or a non-Actinomycete. Although the examples illustrate use of a bacterial strain, any organism or expression system can be used, as described herein. The choice of organism is dependent upon the needs of the skilled artisan. For example, a strain that is amenable to genetic manipulation may be used in order to facilitate modification and production of meridamycin.

### III. METHOD OF MODIFYING UNITS WITHIN BIOSYNTHETIC GENE CLUSTER

In one aspect, the present invention provides methods of modifying one or more of the genes, open reading frames, modules or catalytic domains of the meridamycin biosynthetic gene cluster. Alterations can be for the purpose of improving expression in a selected expression system. Other alterations can be to extinguish, modify, or enhance function of a selected domain.

In one embodiment, the nucleic acid sequences of such altered units can be provided to a heterologous host cell (i.e., another streptomycete strain, a non-Streptomycete and/or a non-Actinomycete) via a suitable vector in a selected host cell and used to express a product. Examples of suitable vectors, expression systems and host cells are described herein. In another embodiment, the invention provides a method of generating mutant Streptomyces strains, generated by modification of one or more of the genes of the biosynthetic gene cluster. Such a mutant Actinomycete strain which contains the biosynthetic gene cluster in which the function of one or more of the genes is partially or entirely altered or destroyed according to the present invention, can be used to generate macrolide compounds, e.g., meridamycin, 36-ketomeridamycin, or 9-deoxomeridamycin.

Where production of a macrolide compound is desired, a host cell expresses the functions necessary to produce the polyketide core, i.e., MerP, MerA, MerB and MerC. However, ancillary functions may be altered or extinguished. For example, after production of the core modules, a polyketide core can then modified by additional enzymes which are herein termed “tailoring enzymes”. These enzymes alter the side chains of the polyketide core without altering the number of the carbon atoms present within the polyketide core. Such tailoring enzymes may include, but are not limited to, hydroxylation and methylation. In one embodiment, the function of the tailoring enzyme Cytochrome P450 hydroxylase (SEQ ID NO:51) can be destroyed.

In another example, one or more of the 4 modules, or the catalytic domains thereof, of the non-ribosomal peptide synthase which composes part of the biosynthetic gene cluster is modified. In another embodiment, one or more of the three polyketide synthases, which comprise 15 modules in total is modified. In another embodiment, one of the modules of the polyketide synthase, e.g., a ketosynthase domain, an acyltransferase domain, and an acyl carrier protein is modified. In still another embodiment, another of the modules, e.g., a ketoreductase domain, a dehydratase domain or an enoylreductase domain is altered. Other suitable mutations, including mutations to genes other than tailoring enzymes, can be readily made by one of skill in the art.

The present invention contemplates any method of altering any of the nucleic acid sequences encoding the proteins of the present invention. More specifically, the invention contemplates any method that inserts amino acids, deletes amino acids or replaces amino acids in the proteins of the invention. Additionally, a whole domain in a module may be replaced, such as the KR, DH or ER domains, which alters the reduction extent of the β-keto group on the polyketide ring. The modifications may be performed at the nucleic acid level. These modifications are performed by standard techniques and are well known within the art. For example, in one embodiment of the invention, the gene encoding the NRPS of the biosynthetic cluster, which is responsible for incorporation of a pipcoleic acid in to the meridamycin macrolide core, is inactivated.

Given the information in the present specification regarding the co-linear relationship between the primary organization of the meridamycin polyketide synthases and the structure of the meridamycin polyketide core structure, one of skill in the art can readily to introduce specific changes in one or more of the individual PKS modules by manipulating the genes encoding these modules, therefore modify certain portions of the polyketide backbone of meridamycin that cannot be easily accessed by chemical modification.

In one embodiment, the invention provides changes of the reduction extent of the β-keto group on the polyketide ring by inactivation, deletion or insertion of a selected reduction domains, e.g., KR, DH, or ER, in selected modules. For example, the hydroxyl function at C36 of meridamycin is derived from a keto group by the action of the KR domain of Module 1 in meridamycin polyketide synthetase A (MerA). By eliminating this KR domain from MerA, the keto group would be restored at C36 position. This has been successfully done, as described in detail in Example 4 (see below).

In another embodiment, the invention provides a meridamycin having a polyketide ring size modified by deletion or addition of one or more PKS modules. The number of the two-carbon units in the polyketide ring (the size of the ring) is determined by the number of modules present in the PKS. Therefore, the size of the polyketide ring can be increased or decreased by two carbon unit through addition or deletion of a module into the corresponding PKS. This can be achieved through inserting a DNA fragment which encodes such a module into selected PKS gene (merA, merB or merC) in a way that maintains the integrity of the whole open reading frame.

In yet another embodiment, the invention provides a meridamycin polyketide ring having one or more side chains modified by site-directed mutagenesis or replacement of AT domains. As mentioned before, the composition of the side chain at the α-carbon of a macrolide polyketide is determined by the specificity of the AT domain present in the corresponding module. For example, an ethyl group is present at C28 of meridamycin because the AT domain in module 4 has the specificity of recognizing ethylmalonyl CoA and incorporate it into the polyketide ring during the 4th cycle of condensation. If this AT domain is replaced by another AT domain which specifically recognizes methylmalonyl CoA, a methyl group, instead of ethyl group, will be present at C28. Alternatively, if this AT domain is replaced by another AT domain which specifically recognizes malonyl CoA, a hydrogen will be present at C28. All these changes can be achieved either by introducing point mutations into the DNA fragment encoding a specific AT domain through site-directed mutagenesis, or by replacing the DNA fragment encoding the AT domain with another DNA fragment which encodes another AT domain with different substrate specificity.

In yet a further embodiment, the invention provides for meridamycin having a starting unit altered by replacement of the loading module. C36 and C37 in meridamycin are incorporated by the loading module of mer PKS from malonyl-CoA. Sequencing analysis of the mer PKS gene cluster revealed a loading module comprising a KSQ-AT-ACP tridomain, suggesting a type of chain initiation as found in the biosynthetic gene clusters of tylosin, pikromycin/methymycin, spinosyn and monensin. Previous studies have demonstrated that this type of loading module has a strict substrate specificity, in contrast to the relaxed specificity of the AT-ACP didomain loading modules found in erythromycin and avermectin PKSs. Therefore, a mutated meridamycin producing strain can be generated, in which the mer PKS loading module is replaced with one of broad substrate specificity. Such a mutated meridamycin may provide more than one meridamycin analog, dependent on the various substrates added to the culture.

The present invention also contemplates a method for using an intergeneric conjugation vector, described infra in the examples, to manipulate, modify, or isolate a protein involved in the synthesis of a specific product. For example, the vector may be used to alter an enzyme which is involved in incorporation of the pipecolic acid residue into the polyketide core, so that a proline residue is incorporated instead. The effect of this modification on peptide function may then be evaluated for biological efficacy. In the above example, modifications to the enzyme may include, but are not limited to, removal of amino acids and/or sequences that specifically recognize pipecolic acid and/or incorporation of amino acids and/or sequences that specifically recognize proline.

Therefore, in general terms, an intergeneric vector may be used to alter a gene sequence by insertion of nucleic acid sequences, deletion of nucleic acid sequences, or alteration of specific bases within a nucleic acid sequence to alter the sequence of a protein of interest; thereby producing a modified protein of interest. Preferably, the protein of interest is involved in the synthesis of a compound of interest. The method of modifying a protein comprises (i) transfecting a first bacterial cell with the vector, (ii) culturing the first bacterial cell under conditions that allow for replication of the vector, (iii) conjugating the first bacterial cell with a second bacterial cell under conditions that allow for the direct transfer of the vector from the first bacterial cell to the second bacterial cell, and (iv) isolating the second bacterial cell transformed with the vector. In a preferred embodiment, the first cell is a Gram-negative bacterial cell and the second cell is a Gram-positive cell.

In one embodiment, based on the fact that the genes encoding the PKSs for the production of the meridamycin core structure are linked together on the chromosome of LL-BB0005, those skilled in the art will be able to transfer these genes into the chromosome of another bacterium that has been optimized for the high yield production of macrolide compound, e.g., rapamycin. This can be done in two steps: first, by deleting the native rapamycin PKS genes from the rapamycin high producer; followed by integration of the meridamycin PKS genes into the chromosome of the mutated rapamycin high producer.

The role of the proteins encoded by a mutant gene generated according to the present invention and/or MerA-V, or ORF1-ORF15 is evaluated using any methods known in the art. For example, specific modifications to a protein sequence may be produced to alter the final product. Other non-limiting examples of studies that may be conducted with these proteins include (i) evaluation of the biological activity of a protein and (ii) manipulation of a synthetic pathway to alter the final product from bacteria. More detailed discussion of these proposed uses follows.

Genetic manipulations and expression of the proteins discussed herein may be conducted by any method known in the art. For example, the effect of point mutations may be evaluated. The mutations may be produced by any method known in the art. In one specific method the manipulations and protein expression may be conducted using a vector that comprises at least one Gram-negative and at least one Gram-positive origin of replication. The origins of replication allow for replication of the nucleic acid encoded by the vector, in either a Gram-negative or a Gram-positive cell line. In one embodiment, the vector comprises one Gram-negative and one Gram-positive origin of replication. Additionally, the vector comprises a multiple cloning site that allows for the insertion of a heterologous nucleic acid that may be replicated and transcribed by a host cell.

The most evolved mechanism of transfer of nucleic acids is conjugation. As used herein, the term “conjugation” refers to the direct transfer of nucleic acid from one prokaryotic cell to another via direct contact of cells. The origin of transfer is determined by a vector, so that the donor cells retain and the recipient cells obtain copies of the vector. Transmissibility by conjugation is controlled by a set of genes in the tra region, which also has the ability to mobilize the transfer of chromosomes when the origin of transfer is integrated into them (Pansegrau et al., J. Mol. Biol., 239:623-663, 1994; Fong and Stanisich, J. Bact., 175:448-456, 1993).

Upon production of the nucleic acid encoding the modified protein, the protein can be expressed in a host cell. Then the host cell can be cultured under conditions that permit production of a product of the altered pathway.

Once the product is isolated, the activity of the product may be assessed using any method known in the art. The activity can be compared to the product of the non-modified biosynthetic pathway and to products produced by other modifications. Correlations may be drawn between specific alterations and activity. For example, it may be determined that an active residue at a specific position may increase activity. These types of correlations will allow one of ordinary skill to determine the most preferred product structure for specified activity.

Evaluation of the mechanism of a protein and role the protein plays in the synthesis of a compound has traditionally been determined using sequence homology techniques. Intergeneric shuttle vectors described previously, e.g., pNWA200 (see US Published Patent Application No. 2003-0219872 A1 (Ser. No. 10/402,842 filed Mar. 28, 2003), the disclosure of which is herein incorporated by reference) may be used to assess the biological activity of an unknown protein. The vector may be used to disrupt a protein, either by partial or complete removal of the gene encoding the protein, or by disruption of that gene. Evaluation of the products produced when the altered protein is present is useful in determining the function of the protein.

### IV. MUTANT ACTINOMYCETE STRAINS

In one embodiment, the present invention provides a mutant Streptomcyes strain produced by modification of one or more of the biosynthetic genes of the invention.

The invention further provides a mutant strain MH1104-1, produced according to the present invention, which has been deposited under the terms of the Budapest Treaty with the Agricultural Research Service Culture Collection (NRRL) on Mar. 14, 2005 (Accession No. NRRL B-30829).

Fermentation conditions to culture the Streptomyces species described herein can be performed in flasks. Alternatively, production of higher volumes can be performed in fermentors under similar conditions.

Media useful for the cultivation of Streptomyces species and the production of the macrolide compounds include assimilable carbon sources such as, for example, dextrose, sucrose, glycerol, molasses, starch galactose, fructose, corn starch, malt extract and combinations thereof; an assimilable source of nitrogen such as, for example, ammonium chloride, ammonium sulfate, ammonium nitrate, sodium nitrate, amino acids, protein hydrolysates, corn steep liquor, casamino acid, yeast extract, peptone, tryptone and combinations thereof; and inorganic anions and cations such as, for example, potassium, sodium, sulfate, calcium, magnesium, chloride. Trace elements such as, for example, zinc, cobalt, iron, boron, molybdenum, and copper are supplied as impurities of other constituents of the media. Aeration in tanks and bottles is supplied by forcing sterile air through or onto the surface of the fermenting medium. A mechanical impeller provides further agitation in tanks. An antifoam agent such as polypropylene glycol can be added as needed.

In one embodiment, a fermentation production medium is prepared by combining dextrose in a weight percentage of about 1% to about 2%; about 1% to about 3% of a soy source, about 0.25% to about 1% of yeast, about 0.1% of a calcium source, about 5% to about 10%, and preferably 6% to 8% maltodextrin, and, optionally, proline, from 0 to 0.5%. Optionally, other components may be included. Suitably, the media is adjusted to a pH in the range of about 6.5 to 7.5, and preferably about 6.8 to 7. Typically, the culture is allowed to ferment with suitable agitation and aeration. Alternatively, other suitable fermentation media may be prepared by one of skill in the art substituting other appropriate carbon source or other components and/or purchased commercially. See, generally, e.g., Sigma Aldrich (St. Louis, Mo.); G. J. Tortora et al., Microbiology: An Introduction Media Update (Benjamin Cummings Publishing Co; Oct. 1, 2001); Maintaining Cultures for Biotechnology and Industry, eds. J. C. Hunter-Cevera and A. Bet (Academic Press, Jan. 25, 1996), the disclosure of which is herein incorporated by reference.

After about 5 to 10 days, and preferably about 7 days of fermentation, the cells from the culture are pelleted by centrifugation. In one embodiment, the cells are extracted with a suitable solvent, e.g., ethyl acetate. The extract is concentrated in vacuo and resuspended in a minimum volume of a suitable solvent, e.g., methanol. The solution is loaded onto a reverse phase silica column and eluted with 20%-100% methanol in water. The fractions eluting from 60% methanol to 100% methanol are concentrated in vacuo. The meridamycin and/or meridamcyin analog(s) containing fractions are separated by suitable means, e.g., chromatographic methods.

In another embodiment, the supernatant is mixed with a suitable resin and allowed to rest from about 8 to 16 hours. Thereafter, the resin is washed with a suitable solvent, e.g., methanol, and the filtrate collected. To the cell pellet, an ethyl acetate-methanol mixture is added. This is repeatedly shaken and centrifuged, and the supernatant collected. The cell supernatant and the broth methanol filtrate are combined and concentrated in vacuo. Crude extract is adsorbed onto silica, and fractionated by vacuum liquid chromatography (VLC). The compound is eluted with a suitable solvent, e.g., methanol in dichloromethane. This extract is concentrated, adsorbed onto silica and loaded onto a flash silica column. The compound is eluted with a suitable solvent, concentrated and further purified by column chromatography.

Enzymes of the present invention can be recovered and purified from cell cultures by well-known methods including ammonium sulfate or ethanol precipitation, acid extraction, anion or cation exchange chromatography, phosphocellulose chromatography, hydrophobic interaction chromatography, high performance liquid chromatography, hydroxylapatite chromatography and Tectin chromatography. Most preferably, affinity chromatography is employed for purification. Well-known techniques for refolding proteins may be employed to regenerate active conformation when the enzyme is denatured during isolation and or purification.

The presence of a compound produced by the organism in the crude or semi-purified material can be confirmed by conventional methods, e.g., liquid chromatography mass spectrometric (LCMS) analysis of fractions. These fractions may be pooled and further purified by chromatographic methods, and optionally concentrated, e.g., in vacuo.

The resulting purified compounds are free of cells and cellular materials, by-products, reagents, and other foreign material as necessary to permit handling and formulating of the compound for laboratory and/or clinical purposes. It is preferable that purity of the compounds used in the present invention have a purity of greater than about 80% by weight; more preferably at least about 90% by weight, even more preferably greater than about 95% by weight; yet even more preferably at least about 99% by weight. In one embodiment, the invention provides compositions containing the compounds of the invention, regardless of how such compounds are produced.

In yet another embodiment, the invention provides a novel compound produced by modification of a gene in the biosynthetic gene cluster. The compound may be generated by a mutant Streptomyces species generated as described herein, or by recombinant production of a modified gene in the biosynthetic gene cluster as described herein.

### V. POLYKETIDE COMPOUNDS

In another aspect, the invention provides novel meridamycin compounds. These compounds include, 36-ketomeridamycin, C9-deoxomeridamycin, and C9-deoxoprolylmeridamycin.

In one embodiment, the invention provides a C36-ketomeridamycin compound of formula (II), or a pharmaceutically acceptable salt thereof.

### Error! Objects Cannot be Created from Editing Field Codes. (II)

In another embodiment, the invention provides a 9-deoxomeridamycin compound characterized by the structure (III):

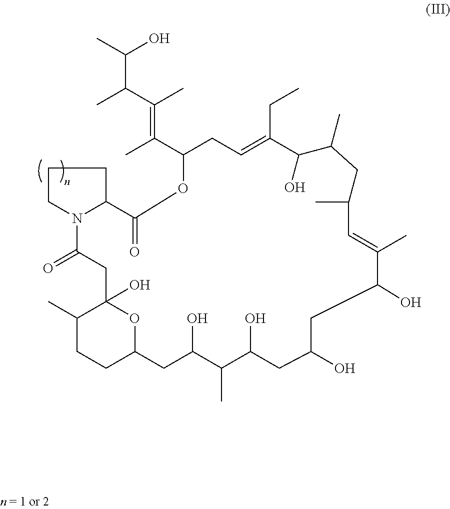

The terms “pharmaceutically acceptable salts” and “pharmaceutically acceptable salt” refer to salts derived from organic and inorganic acids such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids.

While shown without respect to stereochemistry in formula (II) or (III), the compounds of formula (II) and (III) can contain one or more chiral centers. Reference to “compound of formula (II) or (III)” is understood to include any compound of the implicated structural formula including all stereoisomers thereof.

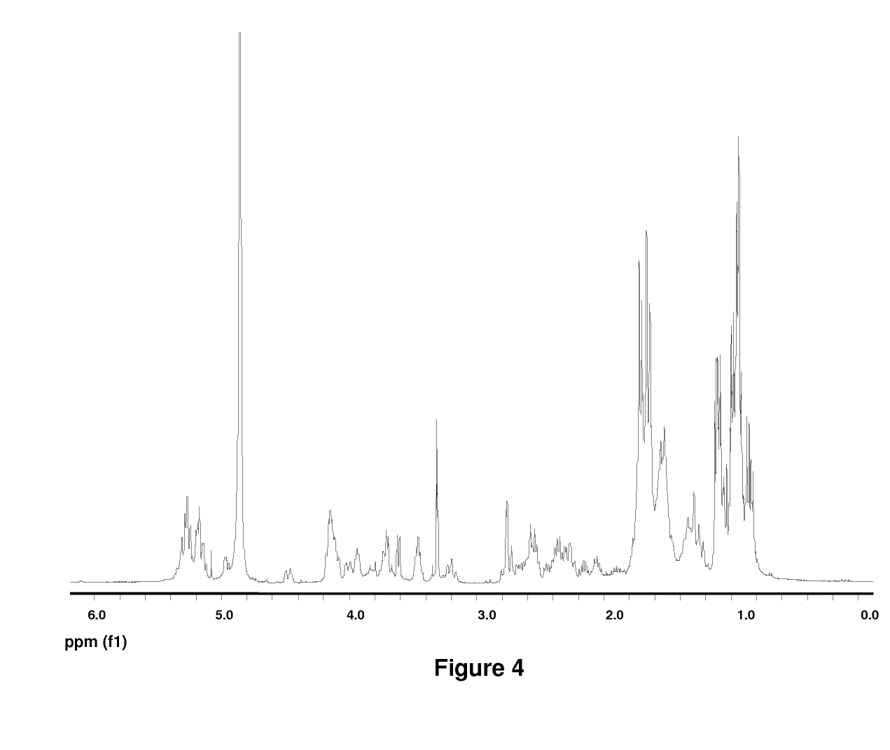

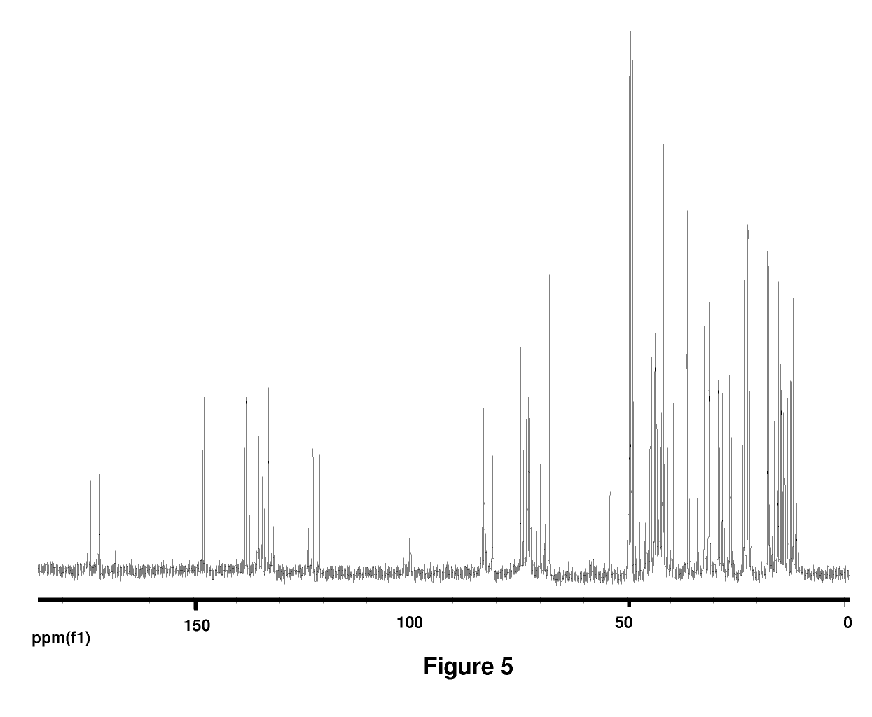

The physicochemical characteristics of the compound of formula (III), wherein n=2, are as follows:
Apparent molecular formula: C₄₅H₇₇NO₁₁
Molecular weight: Positive Ion Electrospray MS m/z=808.1 (M+H)⁺; Negative Ion Electrospray MS m/z=806.5 (M−H)⁻; High Resolution Fourier Transform MS m/z=830.53683 (M+Na)⁺
Ultraviolet Absorption Spectrum: λₘₐₓ nm (acetonitrile/water)=210 nm, end absorption
Proton Magnetic Resonance Spectrum: (400 MHz, CD₃OD): See FIG. 4
Carbon Magnetic Resonance Spectrum: (100 MHz, CD₃OD): See FIG. 5

The production of the neuroprotective compounds (II) or (III) of the invention are not limited to a particular organism, for example, actinomycete species designated LL-BB0005. In fact, it is desired and intended to include the use of naturally-occurring mutants of this organism, as well as induced mutants produced according to the present invention, or alternatively, from BB0005 by various mutagenic means known to those skilled in the art, for example, exposure to nitrogen mustard, X-ray radiation, ultraviolet radiation, N′-methyl-N′-nitro-N-nitrosoguanidine, or actinophages. It is also desired and intended to include inter- and intraspecific genetic recombinants produced by genetic techniques known to those skilled in the art such as, for example, conjugation, transduction and genetic engineering techniques. In one particularly desirable embodiment, the organism used for production of compound (III) is the mutant designated M507 of actinomycete LL-BB0005.

The culture designated actinomycete LL-BB0005, was deposited under the terms of the Budapest Treaty with the Agricultural Research Service Culture Collection (NRRL), 1815 North University Avenue, Peoria, Ill. 61604, on May 18, 2004 and assigned Accession No. NRRL 30748.

The invention also provides a mutant strain of actinomycete LL-BB0005, designated M507. This organism was deposited under the terms of the Budapest Treaty with Agricultural Research Service Culture Collection (NRRL), 1815 North University Avenue, Peoria, Ill. 61604, on Jan. 24, 2005 and assigned Accession No. NRRL 30815. This mutant strain has been found, when cultured under appropriate conditions, to generate higher yields of the compounds of formula (III) of the invention than its parent strain. For example, M507 can generate about 3-fold greater yields of the compound of formula (III wherein n=2) than the parent strain when metyrapone is added during the fermentation process. The mutant strain M507 can generate 3-fold greater yields of meridamycin than the parent strain as well as generate significantly lower amounts of undesired products. The mutant strain M507 sporulates which makes it amenable to genetic manipulation.

The invention further provides mutants, recombinants, and modified forms of the actinomycete strain of the invention, which are characterized by the ability to produce a compound of formula (III).

Fermentation of culture actinomycete strains for production of compound (III) can be performed in flasks. Alternatively, production of higher volumes can be performed in fermentors under similar conditions.

Media useful for the cultivation of actinomycete strain LL-BB0005 and mutants thereof including the M507 mutant, and the production of the compound include assimilable carbon sources such as, for example, dextrose, sucrose, glycerol, molasses, starch; an assimilable source of nitrogen such as, for example, ammonium chloride, amino acids, protein hydrolysates, corn steep liquor; and inorganic anions and cations such as, for example, potassium, sodium, sulfate, calcium, magnesium, chloride. Trace elements such as, for example, zinc, cobalt, iron, boron, molybdenum, and copper are supplied as impurities of other constituents of the media. Aeration in tanks and bottles is supplied by forcing sterile air through or onto the surface of the fermenting medium. A mechanical impeller provides further agitation in tanks. An antifoam agent such as polypropylene glycol can be added as needed.

The compound III (wherein n=2) is produced under standard fermentation conditions by the parent strain LL-BB0005 in very small amounts detectable by LCMS after partial purification. Without adding metyrapone to the fermentation, LL-BB0005 and M507 produce compound III (n=2) at the level of 1-2 mg/L. To increase the titer of compound II wherein n=2, one can add metyrapone to either the parent strain or the mutant M507. When metyrapone is added, M507 produces compound III (n=2) at the level of 15-20 mg/L. Metyrapone is a known P450 inhibitor which prevents the final oxidative step in the production of meridamycin resulting in the production of compound III wherein n=2.

Typically, for production of a compound of the invention (e.g., compound III), the actinomycete strain LL-BB0005 is cultured in a suitable media for several days, e.g., 2-4, preferably at a temperature in the range of about 25° C. to about 30° C., and preferably, about 28° C. Typically, after a total of about 2 to 5 days incubation, 2-methyl-1,2-di-3-pyridyle-1-propanone (metyrapone) is added and fermentation continued for about 3 to 6 days.

Following culture of the actinomycete under suitable conditions to produce a compound of the invention, the compound is isolated and purified using methods known to those of skill in the art. For example, the culture can be centrifuged to separate the broth and cell pellet which contain the compounds of the invention. Typically, the cell pellet is extracted and the extract concentrated. The broth is then treated to obtain any compound which was excreted by the cells, or released during centrifugation. The semi-crude material is then further purified, e.g., by chromatographic methods.

The resulting purified compounds are free of cells and cellular materials, by-products, reagents, and other foreign material as necessary to permit handling and formulating of the compound for laboratory and/or clinical purposes. It is preferable that purity of the compounds used in the present invention have a purity of greater than 80% by weight; more preferably at least 90% by weight, even more preferably greater than 95% by weight; yet even more preferably at least 99% by weight. In one embodiment, the invention provides compositions containing the compounds of the invention, regardless of how such compounds are produced.

### VI. USE OF POLYKETIDE COMPOUNDS

In one aspect, the invention provides the use of compounds produced by the novel strains described herein and the novel compounds of the invention in pharmaceutical compositions and methods for a variety of neurological disorders. Thus, a meridamycin compound produced by a mutant or other novel host cell described herein, 36-ketomeridamycin, or 9-deoxomeridamycin, or 9-deoxoprolylmeridamycin can be so used.

The term “preventing neurodegeneration” refers to preventing neuronal cell death by apoptosis, or any other mechanism, resulting from a pathological condition including but not limited to a neurodegenerative disease, ischemia, trauma, and any condition resulting from an excess of an excitatory amino acid such as glutamate.

The term “promoting neuroregeneration” refers to inducing in a neuronal cell events which include but are not limited to neurite outgrowth or long term potentiation. Neuroprotective agents are useful for the treatment of e.g., neurodegenerative diseases such as Alzheimer's and Parkinson's diseases, neuronal damage following ischemia or trauma, and any other pathological condition in which neuronal damage is implicated. Other compounds derived from meridamycin (described in commonly owned International Patent Application No. PCT/US2005/06246, formerly provisional patent application 60/549,430, filed Mar. 2, 2004) have been shown to demonstrate neuroprotective effects (see also, commonly owned international application PCT/US2005/005895 and U.S. patent application Ser. No. 11/065,934 (formerly U.S. Provisional Application No. 60/549,480, filed Mar. 2, 2004), as does the meridamycin of the present invention.

Although not intending to be limited in its therapeutic applications, it is desirable to use a 36-ketomeridamycin or other macrolide compounds described herein for treatment of conditions of the central nervous system, neurological disorders, and disorders of the peripheral nervous system. Conditions affecting the central nervous system include, but are not limited to, epilepsy, stroke, cerebral ischemia, cerebral palsy, multiple sclerosis, Alper's disease, Parkinson's disease, Alzheimer's disease, Huntington's disease, amyotrophic lateral sclerosis (ALS), dementia with Lewy bodies, Rhett syndrome, neuropathic pain, spinal cord trauma, or traumatic brain injury.

Neurological disorders according to the invention include, but are not limited to, various peripheral neuropathic and neurological disorders related to neurodegeneration including, but not limited to: trigeminal neuralgia, glossopharyngeal neuralgia, Bell's palsy, myasthenia gravis, muscular dystrophy, amyotrophic lateral sclerosis, progressive muscular atrophy, progressive bulbar inherited muscular atrophy, herniated, ruptured or prolapsed vertebral disk syndromes, cervical spondylosis, plexus disorders, thoracic outlet destruction syndromes, peripheral neuropathies such as those caused by lead, acrylamides, gamma-diketones (glue-sniffer's neuropathy), carbon disulfide, dapsone, ticks, porphyria, Gullain-Barre syndrome, dimentia, Alzheimer's disease, Parkinson's disease, and Huntington's chorea.

Specific situations in which neurotrophic therapy is indicated to be warranted include, but are not limited to, central nervous system disorders, Alzheimer's disease, aging, Parkinson's disease, Huntington's disease, multiple sclerosis, amyotrophic lateral sclerosis, traumatic brain injury, spinal cord injury, epilepsy, inflammatory disorders, rheumatoid arthritis, autoimmune diseases, respiratory distress, emphysema, psoriasis, adult respiratory distress syndrome, central nervous system trauma, and stroke.

The compounds of this invention are also useful in preventing, treating or inhibiting senile dementias, dementia with Lewy bodies, mild cognitive impairment, Alzheimer's disease, cognitive decline, associated neurodegenerative disorders, as well as providing neuroprotection or cognition enhancement.

The term “subject” or “patient,” as used herein, refers to a mammal, which may be a human or a non-human animal.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

The terms “effective amount” and “therapeutically effective amount,” as used herein, refer to the amount of a compound that, when administered to a patient, is effective to at least partially ameliorate a condition from which the patient is suspected to suffer.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual subject being treated. Effective administration of the macrolide compounds of this invention may be given at monthly, weekly, or daily, or other suitable intervals. For example, a parenteral dose may be delivered on a weekly basis at a dose of about 10 mg to about 1000 mg, about 50 mg to about 500 mg, or about 100 mg to about 250 mg per week. A suitable oral dose may be greater than about 0.1 mg/day. Preferably, administration will be greater than about 10 mg/day, more specifically greater than about 50 mg/day in a single dose or in two or more divided doses. The oral dose generally will not exceed about 1,000 mg/day and more specifically will not exceed about 600 mg/day. The projected daily dosages are expected to vary with route of administration.

Such doses may be administered in any manner useful in directing the active compounds herein to the recipient's bloodstream, including orally, via implants, parenterally (including intravenous, intraperitoneal and subcutaneous injections), rectally, intranasally, vaginally, and transdermally.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The macrolide compounds can also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

The invention further provides products, including packaging, containing the compounds formulated for delivery. In another aspect, the invention provides kits including, e.g., needles, syringes, and other packaging, for delivery of the compound of the invention. Optionally, such a kit may include directions for administration of the drug, diluent, and or a carrier for mixing of a solid form of a compound of the invention.

The reagents used in the preparation of the compounds of this invention can be either commercially obtained or can be prepared by standard procedures described in the literature.

The preparation of representative examples of this invention are described in the following examples.

### EXAMPLES

The invention is also described by means of particular examples. However, the use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled. All references cited herein are incorporated by reference in their entirety.

### Example 1

### Cloning and Isolation of the Meridamycin Biosynthetic Gene Cluster

### Methods

A. Generation of DNA Probes.

Two pairs of degenerate PCR primers were used to amplify DNA fragments from the genomic DNA of Streptomyces sp. LL-BB0005 by PCR. The first pair of primers were designed based on the conserved amino acid motifs in type I PKS ACP and KS domains. The forward primer (ACP sense) had the sequence 5′-GA(GC) CT(GC) GG(GC) (TC)T(GC) GAC TC(CG) CT(AC)-3′ (SEQ ID NO: 2), and the reverse primer (KS antisense) had the sequence 5′-(GC)GA (GC)GA (AG)CA (GC)GC (GC)GT GTC (GC)AC-3′ (SEQ ID NO: 3). The second pair of primers were designed based on the highly conserved core motifs of the adenylation domain of non-ribosomal peptide synthetases. The forward primer (A3 motif) had the sequence 5′-AC(GC) TC(GC) GGC (TA)C(GC) ACC GGC CIG CC(GC) AAG-3′ (SEQ ID NO: 4), and the reverse primer (A8 motif) had the sequence 5′-AGC TC(GC) A(TC)GC CG(GC) (TA)(GA)G CC(GC) CG(GC) A(TC)C TT(GC) ACC TG-3′ (SEQ ID NO: 5). Each 50 μL PCR mixture contained: approximately 0.1 μg Streptomyces sp. LL-BB0005 genomic DNA, 1.6 μM of each primer, 8% DMSO, 1×Pfu reaction buffer (Stratagene, La Jolla, Calif.), 200 μM of each dNTP, and 2.5 unit of Pfu Turbo DNA polymerase.

The PCR reaction was performed on the Whatman Biometra TGRADIENT thermocycler system with the following condition: 1 cycle of initial denaturation (96° C., 4 min), 34 cycles of denaturation (96° C., 1 min)/annealing (gradient from 45° C. to 65° C., 1 min)/extension (72° C., 1 min), and 1 cycle of a final extension (72° C., 5 min). The about 0.7 kb DNA fragment obtained with the ACP/KS primers and the 0.7˜0.8 kb mixed DNA fragments obtained with the A3/A8 primers were cloned into pCR4Blunt-TOPO vector following the manufacture's instruction. Several clones of each cloning were subjected to DNA sequencing analysis using the M13 Reverse and Forward primers.

B. Isolation of the Meridamycin Biosynthetic Gene Cluster.

A cosmid library of size-fractionated genomic DNA of Streptomyces sp. LL-BB0005 was constructed using vector pWEB (Epicentre, Madison Wis.), following the manufacture's instruction. About 800 cosmid clones were screened with the above-mentioned type I PKS gene probe by colony hybridization. Cosmids from 56 positive clones were extracted, digested with BamH I, and then hybridized with the above-mentioned pipecolate acid-incorporating enzyme gene probe after electrophoresis. Cosmid 45 was identified to contain an approximately 2.5 kb DNA fragment which encodes a pipecolate-specific peptide synthetase. The insert of Cosmid 45 was completely sequenced by custom sequencing (MWG Biotech, High Point, N.C.) and was used to identify several other cosmids through restriction mapping, chromosomal walking and end-sequencing of the cosmid inserts.

C. Results.

One DNA fragment from the PCR using ACP/KS primers was identified to encode a type I PKS, and another DNA fragment from the PCR using A3/A8 primers was identified to encode a non-ribosomal peptide synthetase homologous to the pipecolate-incorporating enzymes for rapamycin biosynthesis (RapP) and FK506 biosynthesis (FKBP). These two fragments were purified and later used to screen the Streptomcyes sp. LL-BB0005 cosmids library.

Cosmid 45 was sequenced and used to identify other cosmids, resulting in the set of overlapping inserts. Inserts of these cosmids were completely sequenced and assembled, giving a contiguous DNA stretch of 116,856 nt which includes the meridamycin biosynthesis cluster. The complete nucleotide sequence of this DNA assembly is depicted in SEQ ID NO: 1.

### Example 2

### Computational Sequence Analysis of the Meridamycin Biosynthetic Gene Cluster

A. Methods.

DNA sequence analysis was done using Lasergene (DNASTAR, Madison, Wis.) and Vector NTI (InforMax, Frederick, Md.). A correlation between the open reading frames that have been identified in this gene cluster and their proposed function are summarized in Table 1.

B. Results.

A biosynthetic pathway for the production of meridamycin has been proposed based on the sequence analysis of the cloned gene cluster.

### Example 3

### Genetic Disruption of the merP Gene

To confirm the cloned gene cluster is responsible for the production of meridamycin, a disruption experiment was conducted to inactivate the gene encoding the NRPS which is responsible for the incorporation of a pipecolic acid into the meridamycin macrolide core.

A. Methods and Results.

A 2450 bp BamH I fragment from Cosmid 45, which spans the internal part of merP gene, was cloned into pUC19 to give pMH100. About a 1.5 kb Nco I fragment containing apramycin resistant gene from pUC120 was cloned into a Nco I site located in the middle of the 2450 bp BamH I fragment. The resulting about 3.9 kb BamH I insert was then excised and cloned into the BamH I site of a Streptomyces/E. coli conjugation shuttle vector pNWA200 to give pBWA27. Conjugation between E. coli ET12567(Z8002pUB307) harboring pBWA27 and Streptomcyes sp. LL-BB0005 was performed according to the following: Briefly, equal volume of donor cells and the spore suspension of LL-BB0005 were mixed and plated on pre-dried R6 agar medium. The plates were incubated at 37° C. for 20 hours before being overlaid with 1 mL of ddH₂O containing 0.5 gmb/mL apramycin and 0.5 gmb/mL nalidixic acid on each of them. The plates were then incubated at 30° C. for 5 to 7 days. Apramycin resistant exconjugants were isolated and then grown under non-selective condition. Apramycin resistant/kanamycin sensitive colonies were identified and the double crossover mutation was confirmed by Southern hybridization analysis of their genomic DNA.

B. LC/MS Analysis of Metabolites

Wild type LL-BB0005 and three individual Pmerp::apr mutants were grown in a seed medium (dextrose 10 g/L, soluble starch 20 g/L, yeast extract 5 g/L, NZ-amine A 5 g/L, calcium carbonate 1 g/L, pH 7.3) for 3 days at 28° C. before inoculated into the fermentation medium (dextrose 30 g/L, soy flour 15 g/L, sodium chloride 2 g/L, calcium carbonate Ig/L, pH 6.8-7) and grew at 28° C. for 5 days. 1 mL broth samples were taken at day 4 and day 5 and extracted with equal volume of ethyl acetate. The extracts were then dried down to dryness and then re-suspended in 100 μL methanol for liquid chromatography/mass spectrometry (LC/MS) analysis.

### Example 4

### Generation of a Keto-Derivative of Meridamycin by Inactivating the KR1 Domain in Module I Keto-Reductase of Meridamycin Polyketide Synthetase A

This example describes the generation of a mutated LL-BB0005 strain in which the DNA encoding KR domain in Module 1 of meridamycin polyketide synthase has been deleted, thereby resulting in the production of a novel meridamcyin analogue, C-36 keto-meridamycin.

A. Generation of C36-keto-meridamycin.

A DNA fragment of about 4158 basepair (bp) encoding the majority of Module I of Mer A was cut from Cosmid 45 through digestion with restriction enzyme EcoR I and Not I and cloned into a vector pUC19 at Hinc II site. The resulting construct was then digested by restriction enzyme Nco I to delete a 1291 bp DNA fragment that encodes the KR domain. The remainder of the construct was then religated into a circular plasmid. The insert of this plasmid was excised by digestion with Hind III and Xba I, and then cloned into a Streptomyces-E. coli conjugation shuttle vector pKC1139. The resulting construct was named pMH1102. pMH1102 was then introduced into LL-BB0005 strain through conjugation between LL-BB0005 and E. coli ET12567/pMH1102. Double cross-over between pMH1102 and the chromosomal DNA of LL-BB0005 resulted in a complete deletion of a 1291 bp DNA fragment that encodes the KR domain of the module 1 of Mer A. This mutated strain was named MH1102, deposited under the terms of the Budapest Treaty with the Agricultural Research Service Culture Collection (NRRL) on May 3, 2004 (Accession No. NRRL 30743).

B. Chemical Detection of C36-keto-meridamycin by LC/MS.

For LC/MS analysis, fermentation broth supernatants were extracted with equal volume of ethyl acetate and concentrated 10×. Extracts were then fractionated on the LC/MS using a linear gradient of 5% to 95% acetonitrile in water on a YMC-ODS 4.6×150 mm Su column. Fractions were collected every minute into a 96 well plate. The plate was concentrated by speed vacuum for the high-resolution and accurate mass measurement (HRMS). HRMS was conducted using a Bruker (Billerica, Mass.) APEXII FTICR mass spectrometer equipped with an actively shielded 7.1 Tesla superconducting magnet (Magnex Scientific Ltd., UK), an external Bruker APOLLO ESI source, and a Synrad 50 W CO2 CW laser. Typically, 5 μl sample was loaded into NanoESI tip (New Objective, Woburn, Mass.) and a high voltage about 800 V was applied between the NanoESI tip and the capillary. Data reported here are based on internal calibration using HP tuning mix.

C. Production

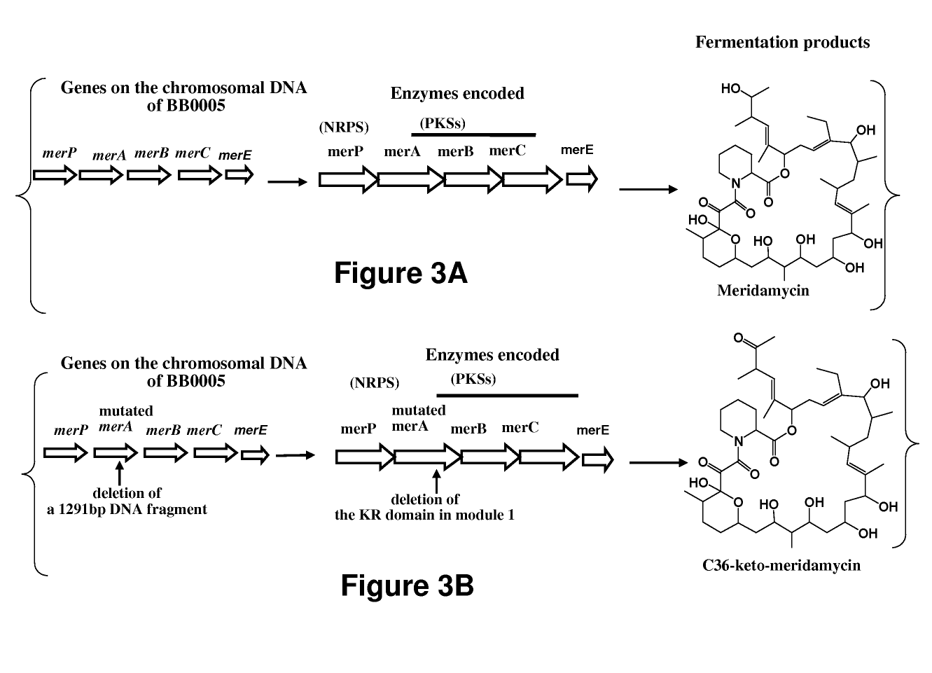

Fermentation of this MH1102 strain gave the production of C36-keto-meridamycin. The schematic representation of the experiment is shown in FIG. 3B.

D. Detection

Sodium adduct molecular ion was detected in the positive ESI detection mode with average m/z=842.50245 (842.50435, 842.50409, 842.50187, 842.50156, 842.50190, 842.50192, 842.50149), and this agrees with the calculated value ([M+Na]¹⁺, calculated: 842.50250, Δ=−0.05 mmu, see Table 3). The measured isotopic distribution of the sodium adduct molecular ions also agrees very well with the simulated one. There is no indication of the presence of meridamycin ions from the positive mode ESI FTMS mass spectra. Deprotonated molecular ion was also detected in the negative ESI detection mode with m/z=818.50531, and this agrees very well with the calculated value ([M−H]¹⁻, calculated: 818.50600, Δ=−0.69 mmu, see Table 3). The measured isotopic distribution of the deprotonated molecular ions also agrees very well with the simulated one. There is no indication of the presence of meridamycin ions in the negative mode ESI FTMS mass spectra either.

TABLE 3
Accurate mass measurement by FTMS

| | ESI | Experimental | | Theoretical | Δ | Ion |
|---|---|---|---|---|---|---|
| Sample ID | mode | Mass | Elemental Formula | Mass | (mDa) | Assignment |
| LL- | Positive | 842.50245 | C45H73NO12Na¹⁺ | 842.50250 | −0.05 | [M + Na]¹⁺ |
| BB0005 | | | | | | |
| (ΔKR1) | | | | | | |
| LL- | Negative | 818.50531 | C45H72NO12¹⁻ | 818.50600 | −0.69 | [M − H]¹⁻ |
| BB0005 | | | | | | |
| (ΔKR1) | | | | | | |

### Example 5

### Generation and Yield Improvement of Meridamycin Analogues Through Manipulation of the NRPS Gene and/or the P450 Hydroxylase Gene

The pipecolyl moiety in the meridamycin macrolactam ring is incorporated by the NRPS MerP (SEQ ID: 46) encoded by MerP gene (nt 21592-26311 of SEQ ID NO:1). The amino acid sequence of the adenylation domain of merP shows significant homology with the adenylation domains from other NRPSs that recognize pipecolic acid, and with those that recognize proline. Accordingly, a meridamycin analogue, prolylmeridamycin (see co-owned international Patent Application PCT/US2005/005895 and U.S. patent application Ser. No. 11/065,934, formerly provisional patent application 60/549,480, filed Mar. 2, 2004, herein incorporated by reference), was also produced by the wild type LL-BB0005 at a very low level. The yield of this compound will be significantly improved by those of ordinary skill in the art, using well-known techniques, by replacing the NRPS gene with another gene which encodes a NRPS that exhibits much higher preference to proline than pipecolic acid. Similarly, the merP gene could also be replaced with any other NRPS gene that recognizes a specific amino acid other than pipecolic acid, thus giving more novel meridamycin analogues with different amino acid residues within the macrolactam ring.

The wild type LL-BB0005 strain also produces another analogue, C9-deoxomeridamycin, at a very low level. This compound resulted from omitting the last step in the biosynthesis of meridamycin: the hydroxylation of C9 by the P450 hydroxylase MerE (SEQ ID: 51) encoded by the merE gene (nt 98393-99586 of SEQ ID NO:1). The yield of this compound thus will be significantly improved through genetic knock-out of merE gene, either through insertion of an antibiotic resistant gene into merE or through deletion of merE gene, by those of ordinary skill in the art, using well-known techniques.

Further, more meridamycin analogues will also be generated by combining the two types of genetic modifications described above, thereby resulting in another set of meridamycin analogues that have pipecolyl moiety replaced with another amino acid residue in the C9-deoxyl macrolactam ring.

### Example 6

### Increasing the Yield of Meridamycin and/or its Analogues Through Genetic Manipulation of the Regulatory Genes

At least six genes in the cloned DNA assembly (SEQ ID NO:1) are predicted to be pathway specific regulatory genes. The protein (SEQ ID NO:45) encoded by Orf15 (SEQ ID NO:18) belongs to the Lac I family of bacterial regulatory proteins. Both Mer I (SEQ ID NO:55) and MerQ (SEQ ID NO:62) belong to the LysR family of prokaryotic transcriptional regulatory proteins. MerH (SEQ ID NO:54) shares high sequence similarity with the MarR group of repressors that appeared to be involved in the multiple antibiotic resistance, a non-specific resistance system. MerM (SEQ ID NO:59) appears to be a member of the MerR family regulatory proteins that have been found to be involved in the resistance to certain small molecules. MerO (SEQ ID NO:61) belongs to the tetR family of bacterial regulatory proteins.

It is possible for those skilled in the art to generate a mutated strain with improved production of meridamycin, and/or its analogues, through manipulation of these regulatory genes. This can be achieved in several ways. For example, targeted disruption or deletion (i.e., knock-out) of each individual regulatory gene would identify its protein product as an activator or repressor of meridamycin production. This can be done either through insertion of an antibiotic resistant gene into each regulatory gene, or by deletion of the regulatory gene. If the investigated gene encodes a pathway repressor, knock-out of this gene would directly increase the yield of meridamycin and/or its analogue(s). If the gene encodes an activator, the yield of meridamycin and/or its analogue(s) might be improved through introducing extra copies of this activator gene into the wild-type producing strain. This can be achieved either through insertion of the activator gene into the chromosomal DNA, or through transfecting the activator gene in a plasmid which can replicate inside a meridamycin producing strain. In either case, the activator gene should be placed under the control of an appropriate promoter to ensure its expression.

### Example 7

### Neuroprotective Effects of Meridamycin and Assay

Neuroprotective effects of compounds produced by actinomycetes (LL-C31037 having NRRL Accession number 30721) are described in commonly-owned International Patent Application No. PCT/US2005/005895 and U.S. patent application Ser. No. 11/065,934 (formerly U.S. provisional patent application 60/549,480, filed Mar. 4, 2004), which is herein incorporated by reference in its entirety.

A. Isolation of Mesecephalic Neurons.

Ventral mesencephalic cultures were prepared from E15 rat embryos and maintained for 7 divisions before experimentation according to the method of Pong et al., J Neurochem. 69: 986-994, 1997.

B. Drug Treatment and Assay.

Cultures were pre-treated with designated drugs: immunophilin ligands meridamycin, rapamycin and FK-506 (1, 10, 100 and 1000 nM), cyclophilin ligand cyclosporine (CsA) at the same concentrations, and glial-derived neurotrophic factor (GDNF-control-1 and 10 ng/ml) for 1 hr or 24 hr. Cultures were then exposed to 10 μM 1-methyl-4-phenylpyridinium (MPP+) for 1 hr, in the presence of drug. After the 1 hr exposure, media was changed 3×, and fresh drug was added for an additional 24 hr or 48 hr. At the end of the 24 hr or 48 hr recovery period, high-affinity ³H-DA uptake was determined as percent of untreated controls (Prochiantz et al., Nature 293: 570-572, 1981).

C. Results

GDNF and FK506 enhanced DA uptake in normal mesencephalic dopanergic neuron cultures. Uptake was reduced by the addition of 10 mM MPP+ in addition to treatment. Pre-treatment with GDNF, FK506, CsA and meridamycin provided partial, but significant protection against MPP toxicity.

Increased neuroprotection was seen following increases in post-treatment and recovery time.

### Example 8

### Generation of a Mutated Strain of BB0005 by Inactivating the merE Gene Which Encodes a P450 Monooxygenase

This example describes the generation of a BB0005 strain in which the DNA encoding a P450 Monooxygenase has been deleted. The resulted mutant, designated MH1104-1, produces a compound of formula (III). This organism was deposited under the terms of the Budapest Treaty with Agricultural Research Service Culture Collection (NRRL), 1815 North University Avenue, Peoria, Ill. 61604, on Mar. 14, 2005, and assigned accession number NRRL B-30829.

A. Generation of MH1104-1.

Two DNA fragments were amplified from cosmid 54, which contains the 3′ end of the biosynthetic gene cluster, including a 3′ portion of merC, full-length merD, merE, F1, F2, G, and H-V, which was isolated from BB0005.

The first fragment (˜1450 bp) was amplified using forward primer 5′-TGCAAGCTTCTCGCGTCTGGTGCTGGTG-3′ [SEQ ID NO:68] and reverse primer 5′-ATCTTCGCCCTTGTCCCGCAGTC-3′ [SEQ ID NO:69], with a Hind III restriction site introduced at the 5′. The second fragment (˜1440 bp) was amplified using forward primer 5′-ATCGCTCTGCGGCTGGCGGTG-3′ [SEQ ID NO:70] and reverse primer 5′-TGCTCTAGAGCCACGAAGACGCCGGAAC-3′ [SEQ ID NO:71], with a Xba I restriction site introduced at the 3′. These two fragments were then ligated into pUC18 through Hind III and Xba I site. A EcoR V restriction site was generated at the join site of the two DNA fragments, which was used to insert a ˜800 bp DNA fragment encoding the apromycin resistance gene. The insert of the final construct was excised by digestion with Hind III and Xba I, and then cloned into a Streptomyces-E. coli conjugation shuttle vector pNWA200. The resulting construct was named pMH1104. pMH1104 was then introduced into BB0005 strain through conjugation between BB0005 and E. coli ET12567/pMH1104. Double cross-over between pMH1104 and the chromosomal DNA of BB0005 resulted in merE gene, which encodes a P450 Monooxygenase.

This mutated BB0005 strain was named MH1104-1, deposited under the terms of the Budapest Treaty with the Agricultural Research Service Culture Collection (NRRL) on Mar. 14, 2005 (Accession No. NRRL B-30829).

B. Production.

Fermentation of this MH1104-1 strain produced C9-deoxo-meridamycin at the titer of ˜30 mg/L, which was an increase as compared with the titer of ˜1 mg/L C9-deoxo-meridamycin produced by BB0005.

### Example 9

### The Compound C9-deoxomeridamycin

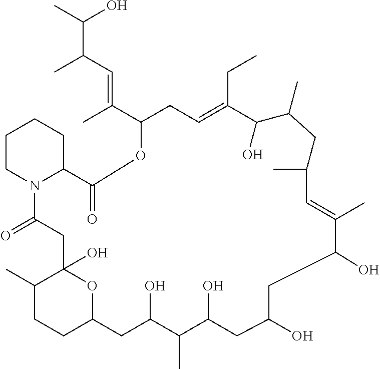

A. Production of Compound

A 50 μL aliquot of spore suspension of strain M507 was inoculated into 5 mL seed medium (dextrose 10 g/L, soluble starch 20 g/L, yeast extract 5 g/L, NZ-amine A 5 g/L, calcium carbonate 1 g/L, pH 7.3) and incubated for 3 days at 28° C. on a rotary shaker with an agitation rate of 200 rpm. This seed culture was then transferred into five 250-mL Erlenmeyer flasks, each containing 25 mL fresh seed medium (1 mL aliquot of seed culture per flask). The second stage seed cultures were incubated for 2 days under the same conditions and then were used to inoculate eighty 250-mL Erlenmeyer flasks each containing 25 mL fermentation medium (dextrose 30 g/L, soy flour 15 g/L, sodium chloride 2 g/L, calcium carbonate 1 g/L, pH 6.8-7) (1 mL aliquot of second stage seed culture to each flask). After shaking at 200 rpm for one day at 28° C., metyrapone (2-methyl-1,2-di-3-pyridyl-1-propanone) was added to the fermentation to a final concentration of 2 mM. The fermentation was subsequently continued for another four days under the same conditions.

Upon harvesting, the whole broth was centrifuged to separate the broth and cell pellet. The cell pellet was extracted with ethyl acetate (3×600 ml) and the ethyl acetate extract was concentrated in vacuo. The broth was stirred with 300 ml Diaion HP20 resin and poured into a column. The column was washed with 1 L water and then eluted using a step gradient (1:3, 1:1, 3:1, 1:0 MeOH:H₂O; 500 ml each). The 75% and 100% MeOH in H₂O fractions were combined, concentrated in vacuo, and combined with the EtOAc extract of the cells. This material was dissolved in methylene chloride/methanol, loaded onto 50 ml silica gel (ICN silica gel, 32-63 μm, 60A), and concentrated in vacuo. This material was then processed using Si VLC (400 ml ICN silica gel) eluting with a step gradient (1 L each of 100:0, 95:5, 90:10, 80:20 CH₂Cl₂:MeOH) collecting 500 ml per fraction. Fractions 4-6 were combined, concentrated in vacuo, redissolved in 45:45:10 hexanes:EtOAc:isopropanol, and loaded onto a flash Si column (ICN silica gel; 5.0 cm×8.5 in; 45:45:10 hex:EtOAc:iPrOH). The column was eluted with 2 L 45:45:10 hex:EtOAc:iPrOH and 40 ml fractions were collected. Fractions 17-40 were combined and concentrated. This semi-crude material was chromatographed by reversed phase (RP) high performance liquid chromatography (HPLC) (YMC ODS-A 30×250 mm S-5 column; 65% to 85% MeOH in H₂O over 50 minutes, then 85% to 100% MeOH in H₂O over 20 minutes, flow rate of 12 ml/min). The title compound eluted from 44 to 52 min as determined by liquid chromatography mass spectrometric (LCMS) analysis (t_{R}=48 min). These fractions were pooled and subjected to further purification by RPHPLC (YMC ODS-A 10×250 mm S-5 column; 40% to 70% acetonitrile in H₂O over 30 min, flow rate of 2.5 ml/min) to yield 16.4 mg of the title compound (t_{R}=25 min).

B. Characterization of C9-deoxomeridamycin

The compound prepared as described in Part A is characterized by having an apparent molecular formula: C₄₅H₇₇NO₁₁

Molecular weight: Positive Ion Electrospray MS m/z=808.1 (M+H)⁺; Negative Ion Electrospray MS m/z=806.5 (M−H)⁻; High Resolution Fourier Transform MS m/z=830.53683 (M+Na)⁺

Ultraviolet Absorption Spectrum: λₘₐₓ nm (acetonitrile/water)=210 nm, end absorption

A proton magnetic resonance spectrum: (400 MHz, CD₃OD) of FIG. 4.

A carbon magnetic resonance spectrum (100 MHz, CD₃OD) of FIG. 5

### Example 10

### The Compound of Formula (III), n=1

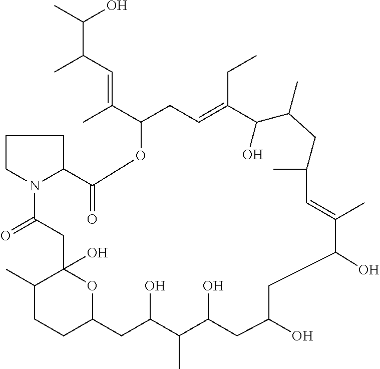

The 5-membered ring can be obtained through biosynthetic regulation including precursor feeding and inhibition of pipecolate biosynthesis in a manner analogous to that for the production of prolylrapamycin [Russo, R. J.; Howell, S. R.; Sehgal, S, N. U.S. Pat. No. 5,441,977, 1995; Nishida, H.; Sakakibara, T.; Aoki, F.; Saito, T.; Ichikawa, K.; Inagaki, T.; Kojima, Y.; Yamauchi, Y.; Huang, L. H.; Guadliana, M. A.; Kaneko, T.; Kojima, N. J. Antibiot. 1995, 48 (7), 657-666; Kojima, I.; Demain, A. L. J. Ind. Microbiolo. Biotechnol. 1998, 20, 309-316] and prolylimmunomycin. Nielsen, J. B.; Hsu, M. J.; Byrne, K. M.; Kaplan, L. Biochemistry 1991, 30, 5789-5796. Based on literature precedent for rapamycin, the 5-membered ring could be produced by fermentation of the actinomycete strain BB0005-MH1104-2 (Accession No. NRRL 30820) with the addition of proline and a known inhibitor of pipecolate biosynthesis such as nipecotic acid [Graziani, E. I.; Ritacco, F. V.; Summers, M. Y.; Zabriskie, M.; Yu, K.; Bernan, V. S.; Greenstein, M.; Carter, G. T. Org. Lett. 2003, 5, 2385-238], thiaproline (L-thiazolidine-4-carboxylic acid), or thiazolidine-2-carboxylic acid (T2CA).

The procedure previously outlined for the isolation of the compound in Example 9 can be used for the purification of the 5-membered ring.

### Example 11

### Neuroregenerative Properties of Compound of Formula III (n=2) in Neuronal Cell Culture

Dissociated cortical neuron cultures were prepared as previously described [Pong et al, “Attenuation of staurosporine-induced apoptosis, oxidative stress, and mitochondrial dysfunction by synthetic superoxide dismutase and catalase mimetics, in cultured cortical neurons”, Exp Neurol. 2001 September; 171(1):84-97.] Briefly, embryonic day 15 rat fetuses were collected and dissected in ice-cold PBS. Dissected cortices were pooled together and transferred to an enzymatic dissociation medium containing papain. After 30 min, the tissue was mechanically triturated with a fire-polished glass Pasteur pipette. Single-cell suspensions in complete media were seeded on poly-L-ornithine and laminin coated 96-well plates. Twenty-four hours later, cultures were treated with various concentrations of compound of formula III for 72 hours. The cultures were then fixed and stained with an anti-tubulin antibody (TUJ-1) and a fluorescent-tagged secondary antibody. Neurite outgrowth was determined by using the Enhanced Neurite Outgrowth (ENO) algorithm with the Cellomics ArrayScan and expressed as average neurite length or neurite length per cell

The compound prepared as described in Example 9 was active in the cortical neuron assay with an EC₅₀ of less than 1 μM.

### Example 12

### Neuroregenerative Properties of Compound (III) in Neuronal Cell Culture

Dissociated cortical neuron cultures were prepared as previously described [Pong et al., Exp Neurol. 2001 September; 171(1):84-97 (2001)]. Briefly, embryonic day 15 rat fetuses were collected and dissected in ice-cold PBS. Dissected cortices were pooled together and transferred to an enzymatic dissociation medium containing papain. After 30 min, the tissue was mechanically triturated with a fire-polished glass Pasteur pipette. Single-cell suspensions in complete media were seeded on poly-L-ornithine and laminin coated 96-well plates. 24 hours later, cultures were treated with various concentrations of compound of formula (III) for 72 hours. The cultures were then fixed and stained with a neurofilament primary antibody and a peroxidase-tagged secondary antibody. A peroxidase substrate (K-Blue Max) was added and the calorimetric change was measure on a calorimetric plate reader.

TABLE 4
NEUROFILAMENT CONTENT IN CULTURED
CORTICAL NEURONS

| | NEUROFILAMENT CONTENT |
|---|---|
| TREATMENT | (FOLD-INCREASE ABOVE CONTROL) |
| 10 nM Compound | 1.9 |
| 100 nM Compound | 2.19 |
| 1 μM Compound | 2.24 |
| 10 μM Compound | 2.29 |

### Example 13

### Neuroregenerative Properties of Compound (III) in Cultured Cortical Neurons

Dissociated cortical neuron cultures were prepared as previously described (Pong et al., cited above, 2001). Briefly, embryonic day 15 rat fetuses were collected and dissected in ice-cold PBS. Dissected cortices were pooled together and transferred to an enzymatic dissociation medium containing papain. After 30 minutes, the tissue was mechanically triturated with a fire-polished glass Pasteur pipette. Single-cell suspensions in complete media were seeded on poly-L-ornithine and laminin coated 96-well plates. After 24 hours, cultures were treated with various concentrations of the compound of formula III for 72 hours. The cultures were then fixed and stained with an anti-tubulin primary antibody (TU-1) and a fluorescent-tagged secondary antibody. Neurite outgrowth was determined by using the Enhanced Neurite Outgrowth (ENO) algorithm with the Cellomics ArrayScan and expressed as total neurite length per cell.

TABLE 5
TOTAL NEURITE LENGTH IN CULTURED
CORTICAL NEURONS

| | TOTAL NEURITE LENGTH |
|---|---|
| TREATMENT | (% ABOVE CONTROL) |
| 10 nM Compound | 10% |
| 100 nM Compound | 54% |
| 1 μM Compound | 86% |
| 10 μM Compound | 121% |

### Example 14

### Neuroregenerative Properties of Compound (III) in Cultured Dorsal Root Ganglia

Dissociated dorsal root ganglia cultures were prepared as previously described [A. Wood et al., “Stimulation of neurite outgrowth by immunophilin ligands: quantitative analysis by Cellomics Array scan” Society for Neuroscience (2004), abstract 104.31. Briefly, postnatal day 3-5 rat pups were euthanized. The spinal columns were removed and individual dorsal root ganglia (DRG) were dissected out. Dissected DRG were pooled together and transferred to an enzymatic dissociation medium containing papain. After 60 minutes, the tissue was mechanically triturated with a fire-polished glass Pasteur pipette. Single-cell suspensions in complete media were seeded on poly-L-ornithine and laminin coated 96-well plates. After 24 hours, cultures were treated with various concentrations of the compound of formula III for 72 hours. The cultures were then fixed and stained with an anti-tubulin primary antibody (TUJ-1) and a fluorescent-tagged secondary antibody. Neurite outgrowth was determined by using the Enhanced Neurite Outgrowth (ENO) algorithm with the Cellomics ArrayScan and expressed as total neurite length per cell.

TABLE 6
TOTAL NEURITE LENGTH IN CULTURED
DORSAL ROOT GANGLIA

| | TOTAL NEURITE LENGTH |
|---|---|
| TREATMENT | (% ABOVE CONTROL) |
| 10 nM Compound | 17% |
| 100 nM Compound | 24% |
| 1 μM Compound | 36% |
| 10 μM Compound | 64% |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description. Patents, patent applications, publications, procedures, and the like are cited throughout this application, the disclosures of which are incorporated herein by reference in their entireties.

What is claimed is:

1. A meridamycin synthase complex comprising three polyketide synthases each of which comprises modules, a non-ribosomal peptide synthetase with 1 module, and a cytochrome P450 hydroxylase.

2. The meridamycin synthase complex according to claim 1, wherein the polyketide synthase comprises the amino acid sequence of SEQ ID NO:47, the amino acid sequence of SEQ ID NO:48 and the amino acid sequence of SEQ ID NO:49.

3. The meridamycin synthase complex according to claim 2, wherein the amino acid sequence of SEQ ID NO:47 is encoded by a MerA gene having the nucleotide sequence of nt 26284 to 43422 of SEQ ID NO: 1.

4. The meridamycin synthase complex according to claim 2, wherein the amino acid sequence of SEQ ID NO:48 is encoded by a MerB gene having the nucleotide sequence of 63480 to 64788 of SEQ ID NO:1.

5. The meridamycin synthase complex according to claim 2, wherein the amino acid sequence of SEQ ID NO:49 is encoded by a MerC gene having the nucleotide sequence of nt 64785 to 98351 of SEQ ID NO:1.

6. The meridamycin synthase complex according to claim 1, wherein the cytochrome P450 hydroxylase has the amino acid sequence of SEQ ID NO:50.

7. The meridamycin synthase complex according to claim 6, wherein the amino acid sequence of SEQ ID NO:50 is encoded by a MerE gene having the nucleotide sequence of 98392 to 99585 of SEQ ID NO:1.

8. The meridamycin synthase complex according to claim 1, wherein the non-ribosomal peptide synthase has the amino acid sequence of SEQ ID NO:46.

9. The meridamycin synthase complex according to claim 8, wherein the amino acid sequence of SEQ ID NO:46 is encoded by a MerP gene having the nucleotide sequence of 21592 to 26311 of SEQ ID NO:1.

10. The meridamycin synthase complex according to claim 1, wherein the polyketide synthases comprises 15 modules in total.

11. The meridamycin synthase complex according to claim 10, wherein each of the modules comprises an (i) ketosynthase domain, (ii) acyltransferase domain, and (iii) acyl carrier protein domain and/or at least one of a (i) ketoreductase domain, (ii) dehydratase domain, and (iii) enoylreductase domain.

12. The meridamycin synthase complex according to claim 1 comprising a module encoded by a MerA gene, said module having an amino acid sequence selected from the group consisting of amino acids 1 to 1050, amino acids 2511 to 4184, and amino acids 4184 to 5172 of SEQ ID NO:47.

13. The meridamycin synthase complex according to claim 1 comprising a module encoded by a MerB gene, said module having an amino acid sequence selected from the group consisting of amino acids 1 to 1717, amino acids 1718 to 3263, and amino acids 5294 to 7102 of SEQ ID NO:48.

14. The meridamycin synthase complex according to claim 1 comprising a module encoded by a MerC gene, said module having an amino acid sequence selected from the group consisting of amino acids 1 to 1496, amino acids 1497 to 2942, amino acids 2943 to 4470, amino acids 4471 to 5930, amino acids 5931 to 7386, amino acids 7387 to 9509, and amino acids 9510 to 11128 of SEQ ID NO:49.

15. A host cell comprising a heterologous meridamycin synthase complex according to claim 1.

16. A synthetic polyketide synthase comprising a meridamycin module selected from the group consisting of
(a) a MerA module consisting of at least one of amino acids 1-1050, amino acids 1051-2510, amino acids 2511-4183, and amino acids 4184-5172 of SEQ ID NO:47, or a catalytic domain thereof;
(b) a MerB module consisting of at least one of amino acids 1 to 1717, amino acids 1718-3263, amino acids 3264 to 5293, and amino acids 5294 to 7102 of SEQ ID NO: 48, or a catalytic domain thereof;
(c) a MerC module consisting of at least one of amino acids 1 to 1496, amino acids 1497 to 2942, amino acids 2943 to 4470, amino acids 4471 to 5930, amino acids 5931-7386, amino acids 7387 to 9509, and amino acids 9510 to 11128 of SEQ ID NO:49, or a catalytic domain thereof.

17. A nucleic acid sequence encoding a synthetic polyketide synthase according to claim 16.

18. A recombinant vector comprising a nucleic acid sequence according to claim 17.

19. A host cell containing a heterologous meridamycin synthase according to claim 16.

20. An isolated nucleic acid having a nucleotide sequence selected from the group consisting of:
(a) a nucleotide sequence that encodes a polypeptide having the amino acid sequence as set forth in any one of SEQ ID NOs: 31-67;
(b) a nucleic acid sequence that hybridizes to a nucleotide sequence encoding a polypeptide having the amino acid sequence as set forth in any one of SEQ ID NOs: 31-67, said hybridization being performed under stringent conditions;
(c) a nucleic acid sequence that encodes a polypeptide at least 90% homologous to the amino acid sequence set forth in any one of SEQ ID NOs: 31-67; and
(d) an isolated nucleic acid fragment having a nucleotide sequence complementary to the nucleotide sequence of (a), (b) or (c).

21. A chimeric nucleic acid construct comprising a nucleic acid of claim 17 or 20, wherein said nucleic acid is operatively associated with an expression control sequence.

22. The chimeric nucleic acid construct according to claim 21, wherein said construct is an expression vector.

23. A host cell containing a heterologous nucleic acid according to claim 22.

24. The host cell of claim 23, wherein the nucleic acids have nucleotide sequences of SEQ ID NOs: 6-18, nucleotides 3885-4727 of SEQ ID NO:1, nucleotides 9320-10960 of SEQ ID NO:1, SEQ ID NOs 19-30, nucleotides 100527-101036 of SEQ ID NO:1, 104276-105271 of SEQ ID NO:1, 111061-111717 of SEQ ID NO:1, 111846-113225 of SEQ ID NO:1, and 116453-116854 of SEQ ID NO:1.

25. The host cell of claim 23, wherein said host cell contains the nucleic acid of SEQ ID NO: 1.

26. A method for producing a polyketide compound, which method comprises culturing the host cell according to claim 15 under conditions that provide for expression of the amino acids encoded by the nucleic acid.

27. The method of claim 26, wherein the polyketide compound is meridamycin.

28. The method of claim 26, wherein the polyketide compound is modified, and wherein said modified compound comprises addition, removal or substitution of at least one amino acid in the polyketide synthase.

29. The method of claim 26, wherein the MerA nucleic acid sequence (nucleotides 26284-43422 of SEQ ID NO:1) is modified to eliminate the function of the ketoreductase domain to produce C-36 keto-meridamycin.

30. The method of claim 26, wherein the modified polyketide compound results from a modification comprising addition, removal or substitution of at least one amino acid in the group selected from MerP, MerA, MerB and MerC, and MerE (SEQ ID NOs: 46, 47, 48, 49, 50), whereby the modification results in a change of the polyketide compound ring size, a change in the reduction extent of a β-keto group on the polyketide ring, a change in a side chain at an α-carbon of the polyketide compound, a change of the starting unit of the polyketide compound, a change of the pipecolyl moiety with other amino acid residue(s), and a change of the oxidation status of C9.

31. A meridamycin analogue produced from the method claim 30.


SEQUENCE LISTING (71 sequences)

SEQ ID NO: 1 - DNA, 116855 bp, Streptomycetes sp.
ggttttcccagtcacgacggatccccccatcaccgtgagcagcggccactgccgggcgggcgccggagcggcaccgggcgcggcccggccgccgccctccggacgcgcggtgtcccgggtgatcagcgggaagcggcgcgacctgcggatgaaccggccgggccccgcgggcggcgcgggcggctcctgcggctgctccccggccgccggcccgctcgcctcagccgtcatggccggcaccggcgtcggcggccgccttcgcgtcccgctccgcggcctccaccacgttgaccagcagctgggcgcgggtcatcgggccgaccccgcccgggttcggcgagacccagcccgcgacctcggtcacaccggggtgcacatcgccggcgatcttgccgtgctcgtcccggctgacgcccacgtcgagcaccgccgcgcccggcttgacgtcctccggcttgaccaggtgccgcaccccggcggccgccacgatgatgtcggcctggcgcaggatcccgggcaggtcgcgggtgccggtgtggcagagggtgaccgtcgcgttctccgaacggcgggtcagcagcagcccgatcgaccggccgacggtgatgccgcggccgacgaccaccacatgcgcgccgttgatctccacaccgtggtggcgcagcagctggatgacgccctggggcgtgcacggcagcgggccgctctcgttgagcacgaggcggccgaggttcatcgggtgcagtccgtcggcgtccttgaccgggtcgatcagctccagcacccggttggcgtcgatgcccttggggagcggcagctggacgatgtagcccgtgcaggacgggtcctcgttgagctcccggaccgccgcctcgatctcctcctgggtggcggtctcgggcaggtcgcgccggatggaggcgatgccgacctcggcgcagtcgcggtgcttgcccgccacgtaccacttgctgccggggtcctcgcccaccagcacggtcccgaggccgggatggatgcccttggccttcagcgcctccacgcggctgacgagatcggacttgatcgcggctgcggttgccttgccatcgagaatctgcgcggtcatggcttcatcctcccggatgcggggacgtcggatccaatcaggggccggtcggggccggaccggcgaaggccgcaccccgtgatccggtcgggatgcggcctcctgtgctcgctcagcgatcgcccggtgctcgctcgctggtcactcggtactcgctcagtggaagaagtggcgcgtgcccgtgaagtacatggtcacacccgccttctgggcggcctccaccacggcctcgtcacggaccgagccgcccggctgcaccacggccttcacgcccgcctcggccagcacctcgaagccgtccgggaacgggaagaaggcgtcggaggcggcgtacgaaccggcggcccgctcggcaccggcccgctcgacggccagcttcgccgagtccacgcggttgacctggcccatgccgacgccgaccgtggcgccgcccttggcgagcaggatcgcgttggacttcaccgcgcggcaggagcgccaggcgaaggccagctcggcgaggccgtcggcgtccagcgcctcgcccgtggcgagggtccagttggccgggtcgtcgccctcggcctggaggcggtccttgacctggacgagcgtgccgccctcgatggggcgctgctcggcggcctccaccggcgactccgcgcagcgcagcacccggatgttcttcttacgggcgagcgcctcgaccgcgccgtcctcgtacgccggggcgacgatcacctcggtgaagatctcggcgacctgctcggccatggcgaccgacaccgggcggttgacggcgatcaccccgccgaaggccgacagcgggtcgcaggcgtgcgccttacggtgcgcttcggcgacgtccgccccgactgcgatcccgcacgggttggcgtgcttgatgatcgcgacacagggctcggtgtggtcgtacgcggcccggcgggcggcctcggtgtccgtgtagttgttgaacgacatctccttgccgtgcagctgctcggcctccgcgagccccttaccgctgccatcggtgtagagggcggcgggctgatgcgggttctcgccgtagcgcagcacgttcttacgggtgatggtggcgcccaggaagtccgggaaggaggagtcgtccgccgccgcgtagtcggccgcgaaccagttggccaccgccacgtcgtaggcggcggtgtgctggaacgcctcggccgccagccgcttgcgccgctccaggtcgaaaccgccctccgcggcggcctcgaggacgtcgccgtaccgctcggggttgacgaccacggccacggacgggtgattcttggcggcggcccggaccatggaggggccgccgatgtcgatctgctcgacgcactcgtccggcgcggcgcccgaggcgaccgtctcgcggaacggatagaggttgaccacgaccagctcgaaggggtccacgcccagctcccggagctgctcgcggtgcgagtcgagccgctggtcggcgaggatgcccgcgtggacgcgcgggtgcagcgtcttgacgcggccgtcgagacactcggggaagccggtcagctcctcgaccttggtgaccgggaccccggcggcggcgatcttcgcggccgtcgagccggtcgagacgagctggacacccgccgcgtgcagccctcgggccagctcctcgagacccgtcttgtcgtagacgctgaccagcgcgcggcggatgggccgcttggtaccttcggcggtcacgggatccttacctttcgtccctctatgcggtagccgtgacgggccagacgccccacgacctcgacgagcagcgagcgctcgacttccttgatccgctcatggagagcggactcgtcgtcctcgtcccggacctcgaccacgccctgggcgatgatcgggccggtgtcgacgccgtcgtcgacgaagtggacggtgcatccggtcaccttcacaccgtgcgcgagcgcgtcgcgcacgccatgggcgccgggaaagctggggagcagcgcgggatgggtgttgacgcagcggccgccgaaccgggcgaggaactcctggcccaggatcttcatgaacccggccgagacgaccaggtccggctcatgggcggcggtggcctccgccaaggccgcgtcccactcggcgcggccggcgtggtccttgacccggcacacgaacgtggggatcccggcgcgctcggcgcgcgtcaggccctcgatgccgtcacggtcggcgcccacggccaccacctcggcgccgtatcgggccacgccctcggcggcgatggcgtcgagcagcgcctggagattcgtaccggagccggagacgaggacgacgaggcggaccgggcgcccggggcgcgcagggctggcgggggaaggcggggaggccacggcggggctctttctcgcgggcggtgctgccgtttgtgtggtcgtacaaggtcgcggactcatcaattccggggaactctacgaagccgccgaccgtcagcaacgataccggcacacgaagcggcccccaggggacgggggcgggcacgggaggtagcgtctggactgcgaatgcaactgacgccactgacgccgtgggaacgaccgccgcacccgcgcaccgagccgtacggcaccggccgtacccgcgccgtacggcaccaggccgtacccgccccggcgtgcaggacgacgacacaaggggaagacacactccacatgccggaccgacgccgccgcgcggctcatcgcctcactccgccccccgcccaggcccgggggcggcgatgaggcgcgcccgcatgtcgcgcacgacgccgctgctccgggagcagcagtcgtcatcctcctcgtcctcctcgtcctcgtccgcccccggcgggccgaacggggaccagcacgaggacaatccgttcgccccgccgcccgagggcaggccggaccagccctggcggccccgccaccggccggacggctccggcggggagagcggcgagggccgccccggcgcccagggcggccaggacgggccggacggcgaccagagcggtgagcagccgcagcagcccccggcctggggcagccagtggagcagccgccagcccggccgccagaacggcggcttcggcggcaccccgggctccaaccgcccctccggccccggcgggcccggcggcccgcgctgggaccccaacgacccggcccagcggcgcgcccggtacgcgctgctctcgggcatgtgggcgttcttcttcgcgctcttcagcctgccgcagatcgcgctgctgctcggggtgctggccctgtactggggcatcagctcgctgcgggccaagccgcgccgtacggcgccgtccccggccgcggccgcgcccctgaacgccccgcccccgcctccgggcgccgcccgggcagcgctgcccgcgcccggcagcggcccggcgaagtcgcagtcgacggcggcgatcagcggtctggtgacgggcggtctcgcgctcgccatcgtcgcggcgacgttcagcttccaggtcgtctacagcgactactacacctgtgtggacgacgccctcacgcagacctcgcgccacgactgcgaaaccttgctccccgagcagctccgccccctgctgagcacgcaggactgacggcgccgggcccgcacggcggaaggctcgcgcccgcttgccgtccgccttacggttcgcgtttacggttcgcggcgggcgtcgtccggcggcggcgggggctccgtgcgcgaggggtccggggcggtcttggactcggcgggcggctgcggcccgggagcgcgcttccggctcagtgcccagcggcgtgggcgggcggccccgggggcgcccggggtgcccgcgggggtcgcgagtccggccatcgtgcccacaccggtttcgcgtccggtctcccgtcccgtctccgcaccggcgtcgggcgccgccttacggttccgcttgcggtcggcgcccgacgcgcccgggcgcagccactgccaccacggctcggccatcgcctcgcgcacctcggcggactccatgggtgacaccgtgggcagcaccgccgcccgcgcctccgcctcggccgcggcccgcgccgcgcgggcggccttggcctccgtacgggcctccctggctgccgtacgggcctccccggccgccgtacgggcctgcgcgcgggatgtccggcggtccgcccgcgccgccttccactccggccaggaggccctggtggggacgcacagccggtaccagcgcagcaccatcgcgccgggcaccccgatcactcccgtccaggccagcgtgatcacgcccgtgcgccaccagctcgggccgaggtccgcgagcatgccgatgcccagcggtccgcccgagaccccggccaggagcgccatcgcggccgcgcagccgaccgccgccagcgcggcgagcaccagcgtctcggcccagccccagggcggcctgggctcgcccttgccgggccgccgcaccgccgcgatcccgatgagccaggccaccgacgccccggccgcgatccccgtgagccagaccagcggcccgccggagccgtccgtgggcagcgcggcgaccagggggaagtgcggcagctgggggtaggaggtgatgcccagcggcgccaccacactgccgccacccaccgtgaaccccgccccgaccccgtacgccgcgccccagacgatcgcgttcggcagcagcgccaggctcaccaggagcaccgcgaaccgccccgaccacacatcgctgaggttgaggaacgtcacctgcacggcgcccgcgtggctcagcatcgacgtcgccgtaaggagcgcaccgctgccgagcaggacgacgagaccgctggtcccggcccgtagcgcggcggtcagccggcggcgccggcaccagccgcgcgcggccagggacgcggccgtccttacggtccgttcggcgccggggagccgccgcagcctctcgctcacccgtccgggcaggcggagcgggaaccgtccgtcggccgtccacacgccgacggcggcgatgaccccggcgacgaccggcagatgcagcagcgcgctcagcggatcgacacgcagcggcccggtcgaggcgtacaccgcggcagcggtgcccaccagcagatagccgccggtcacccaggcgaaggcggtgcgcggatcgacgacggactcctcgggcacccactgcccgtcgccctcatcgggctccgcctggtagacggcgtgctgggcggcccggtacagcagccagcacggcaccacgctgagcagcagcggggtcagcccgaccggcgcggtgtggccggagagcgtctcggtgcgcacgaggtcggcgccatggccgagcagccataggtcggcggcgacatgcagggctccgtcggggctgctctcgggggaggaagaagtgatccacaacagcagtacgaccacggcgagcgtgccgaggccgagccccgcggcgaccacaccgccgaggaacgcctccctgatggcggaggaacgccggggcgctgagcggtcgcgtgcggacaccgacgggctgcgatcggtcgtttgcgtcacatgaccatgctgccaataacagccgtttcatccctacatcaagggtttggtcgccgtgtcgcggctagtccgcttatgcgtcttttatagagtcgtgggacggatgagtggcttgccgcgttccgaccgggtatccgtcgcccgggaacaccgcgggcaccaccatggggtggtgcgggcccgcggcatcgggccgtggcggcgtcagccggccagcgcggcgcgcgccaggcgcgcggtctcggacggggtcttgccgaccttcacgcccgcggcctcgagggcctccttcttcgcctgggcggtgccggaggagccggagacgatggcacccgcgtggcccatcgtcttgccctcgggggcggtgaagcccgccacatagccgacgaccggcttggtgacgttggccttgatgaagtccgcggcccgctcctcggcgtcgccaccgatctcgccgatcatcacgatcaggtcggtgtcggggtcggcctggaaggcggcgagggcatcgatatgggtggtgccgatgatcgggtcaccgccgatgcccacacaggacgagaagccgatgtcccgcagctcgtacatcatctggtaggtcagcgtgccggacttcgacaccagaccgatccggccgggcttggtgatgtcggccgggatgatgcccgcgttcgactgacccggcgtgatcagacccgggcagttcgggccgatgatgcgcgtcttgttgcccttcttgcccgcgtaggcccagaagttggcggagtcgtggaccgcgatgccctcggtgatcacgacggcgagcggaatctcggcgtcgatcgcctcgatgaccgcactcttggtgaacttctccgggacgaagatgaccgtgacatcggcgccggtggcgtcgatggcctccttgacggagccgaagaccgggatctcggtgccgtcgaagtccacggtggtgccggccttgcgcgggttcacgccgccgacgatgttggtgcccgaggcaagcatccgacgggtgtgcttctgcccttcggacccggtcatcccctggacgatgaccttgctttccttggtgaggaagatagccatggtttctggtgacctcgtcccttacttcgcagccagctcggcggcacgctcggccgcgccgtccatggtgtccacctgctgaacgagcgggtggttggcgtcggtgaggatcttgcgacccagctccgcgttgttgccgtcgaggcgcacgaccagcggcttgctgacgtcctcgcccttggacttcagcagctccagggcctggacgatgccgttggcgaccgcgtcacaggcggtgatgccaccgaagacgttgacgaagaccgacttgacgtccgggtcgccgaggatgatctcgagaccgttggccatcacctcggcggaggcgccaccaccgatgtcgaggaagttggcgggcttgacgttgccgtggttctcgcccgcgtaggcgacgacgtcgagggtggacatgaccagacccgcgccgttgccgatgatgccgacctcgccgtcgagcttgacgtagttgaggcccttggccttggcggccgcctcgagcgggttggccgcggccttgtcctcgagcgcctcgtgctccggctgccggaaggcggcgttctcgtccagggagaccttgccgtccagcgcgatgaccttgccgtcttcggtcttgaccagcgggttgacctcgacgagcagggcgtcttccttgatgaagacggtccacagcttctggagcaccgcgacgacctggtccgcgatctcggccgggaacttcgcggcggcgacgatctcggcggccttctcctcggtcacgccctcgatggcgtccaccgggatcttggcgagcgcctcggggttctgctccgcgacgacctcgatctccacgccgccctcgacggaggccatggcgaggaaggtgcggttggtgcggtccagcaggaaggagacgtagtactcctccttgatgtccgcggtctcggcgagcatcaccttgtggaccgtgtggcccttgatgtccatgcccaggatctggccggccttctcgacggcgtcatccgggtcggaggccagcttgacgccgcccgccttaccgcggccgcccgtcttgacctgcgccttgacgaccgcgcggccgcccagccgctcggccacctcgcgcgccgccccaggcgtgtcgatgacttcaccggccagcaccggtacaccgtgcttggcgaagaggtccttcgcctggtattcgaacaggtccacgcgcgtccgtcccttttccatggatttcgcggtcgttatcagcgcgggcgtgccgcgggggcagcgtgacgacgcgatctgtaacgagggcggcacacgttcgccgggtacgcggcatgtccgtctcgcaggttatctccgtgggccgtgcggccctaaatcgcagatcacactggagcggtgattacggtcacagatcgcgaccgtttccatctatctccgtacgacaaccgggcagccccctccccaacgagggctgcccggtcgatgacgcttgccttacggttttacggtttcgcggcctgcatggccttcacggcctgactgtctgacgcccgccggagcgatcagacggtcggaagcgtgcccggaacggtcggcagctggaccggaacggagggcaggtcggcgggaacctgcgggaggtcggccgggagctgcggcaggtcggccggaacggtgggcagctgcggcaggtcggtgggcagctgcgggaggtcggccggaacggtcggcaggtccgccgggagctgcggcaggtcggccgggagctgcggcaggtcggcgggaacctgcgggaggtcggccgggagctgcggcaggtccgccggaacggtgggcagctgcggcaggtcggtgggcagctgcgggaggtcggccggaacggtcggcaggtccgccgggagctgcgggaggtcggccgggagctgcggcaggtcggccggcagcaccgggagggccgggatgttggcgacgtcggtcaggtcggtgggcaccgccgggagcaggctcagcgcaccgtccttggcgttacccacggtggtctgggcgttcgcggccacgccggtggccaggccgtgggcgaacggcacggtggtgcccgccacgtcctgcgcgaacggaacggcggtggacggcacgctcaccacgaccggcaccaggcggtcgacggtgtcctgggcggccggaaccacgttggaggcggtgcccagcgcgaagccctcggcgctgccggtcacgacgtccacgtacggggtggtgcgggccacggcgtcgtcggccagcgcaccggtgtcacccacggtgcgctcggcgaccgggacgaccttgaccacgaggcggtgcacggcgggcggcagcacgtcggaggccacaccgtcgacgaccggcttggtggtgccgacggcaccctgcaccttgccggtcagctcctccgggcggatgccggcgccggagagggaggcgagcaggtcattggccgaacccggggcggagggcagcttcggggtcatcaccaccggtgaccacggctacgccaaggcggtgctcgactcaccgctccaccgcgacgtgggcgcgctcttcccgcgcggcggcggcatgtcgtgggcctcgaccgcgggcctcggagccctggacctggccaccgtccccaacaagctcaccccgaagcagcgcgccgaggtgcgcgcgatggtgacgaaggccgccgaccgctacgccgcggactccgcgaagtcggcctacggcgtgccgtacgcgccgaaggacggcaagtacgagtggggctccaacagccaggtgctcaacaacatgatcgtgctcgccaccgcacacgacctgacggacaagccccgctacctcgacgcggtgctccgcggcatggactatcttctgggcggcaatccgctcaaccagtcctatgtcaccggccacggcgaacgggactcgcacaaccagcaccaccgtttctgggcccaccagcgcgaccaccggctgccgcatccggcgcccggctcgctggcgggcggcccgaactccgggctgcaggacccggtggccaagaagaagctgaagggctgcgccccggcgatgtgctacaccgacagcctgatggcgttctccaccaacgagatcaccatcaactggaacgccccgctggcctggatcgcgtcgtacgtcgacggtctgggcggcggcgcggcggagcagtccgtgcgctgacccggcccggccggaacacgccgccgcccacccgcgctcgggcgcgggtgggcggcggaccagcggagcggggaggtcagtcggcgccgaactccatcgcggcgcggtcgagcagcttgtcctgtccggacacgtgcccgtccgaggcgatcgcctcggaggcgccctgcggcatcgcgccgatcagcccggtggacgccgcctgcgcggcgccgatcagcgccggatgcgagctgccgaccatgccgagaccggcgtactgctccagcttggcgcgtgagtcggcgatgtcgaggttgcgcatggtcagctggccgatccggtccaccggaccgaaggcggagtcctcggtccgctccatggagagcttgtccgggtggtagctgaacgccggtccggaggtgtccaggatggagtagtcctcaccgcgccgcagccgcagggtcacctcgccggtgatcgccgcgccgacccagcgctgcagcgactcgcgcaccatcagcgcctgcgggtccagccagcggccctcgtacatcagccggccgaggcggcgcccctcggtgtggtaggtggcgacggtgtcctcgttgtggatcgcgttgaccagccgctcgtaggccgcgtgcagcagcgccatgcccggtgcctcgtagatgccccggctcttggcctcgatgacgcggttctcgatctggtcggacatgcccatgccgtgccgaccgccgatggcgttggcctccagcaccagatcgacggcggaggcgaactccttgccgttgatcgttaccgggcggccctgctcgaagccgatcgtgacgtcctcggccgctatctcgaccgacgggtcccagaaccgcacgcccatgatcggctggacgatctcgataccggtgtcgaggtgctcgagcgactttgcctcgtgggtggcgccccagatgttggcatcggtggagtacgccttctccgcgctgtcccggtagggcaggtcatgggcgagcagccactccgacatctccttgcggccgccgagctcgctgacgaagtcggcgtccagccacggcttgtagatccgcagggaggggttggccagcagaccgtagcggtagaaccgctcgatgtcattgcccttgaaggtggagccgtcgccccagatctgcacattgtcctcgagcatggcgcgcaccagcagggtgccggtgaccgcccgcccgagcggggtggtgttgaagtagctgcggccgccggagcggatgtggaacgccccgcaggcgagggccgcgagcccctcctccaccagggccgcccggcagtccaccagacgggcgacctccgcgccgtacgtcgtggcgcgcccggggaccgaggcgatgtcgggctcgtcgtactggccgatgtcggcggtgtaggtgcagggcaccgcgcccttgtcgcgcatccacgcgaccgctaccgaggtgtcgaggccgccggagaaggcgatcccgacgcgttcgccgacagggagggaggtgagaactttggacacagcaggagtatgcagggttacgcatgatcatgcaaggcctcctggtgatcgccatgatccacacctcgttccccgcgcttcgccgggtcggggaccggggcgcccggggcgcttccggacggttttggatgggtccggacggctccaggcgggtccaggcggttccggacggcgaagcgcgggggtgaggatcatgaggtcagatacgcctccacctcactgacctgggccgcgggccagccggtgttgccggtgacgctgagcctcagatagcgcacattcgtgctgtcgggcagggcgacggtgaccttgttgccggacgccgggtcgaagcggtagccctgcgagcccaccaccgtggagtacgaggagccgtcggtgctgcccagcacggacagggtctgggtgcgggcgccccacgccgacgagggcggcagcttcagcaccagcctgcggacggcctggccggcgccgaggtccacggtcagggcctgcggaaaggcgttgttggtcgactcccagtaggtgttcgcgtcgccgtcgaccgccttgccgggggtgtagacgtcccaagagccggtcgcggtggccgggcggcccttggcgaggttgcggcccgggtcgggatccgggttgccccggccgggctggggccagctggcacagtccgaccaggtgctgctccagccggagttgccgccgccgtcgttgaggtcgaaggtgccggagcccgacgggtacgggcagttgtagacgcccgccgcgccgacgctggaggccgtgacatcgccgaacttcaccgccccctgcgcctcggcctggacgacgaccgttccggggttggtcacggtcgcgccggacacattgacgttcttgaccgcgtagcccctgccgccgccggagacgaactcgaaggcgctgtacgggctgtcggtgatggtcgtgttggtgatgttgacggtggcctcgatcgcgctgtcgtaggagtcgacgcgcagggcgcccatcgggtggctccagttggggttcatggcgcccgctcggaccagcgtgttgccgtcgaccgtgatcgtgccggccagcgggtggaacgggtccatgaacttctggttggagatggcgatgccactgcccagggcgttggtgtcggagatcaggttgttcttgaccgtgatgtccgtaccgccgtagatggcgatgccattggcgaggttcggctgcgagatggtgttgctctcgaagctgctgttggtgtccggcgagttcagcgaccacatggcgagcgcgtcgtcgccctggttgcgcaggaagttgttccggacccgtacgttcttggcgctgccgttgaggttgaggccgtcggccgtcatgtccaggaagcggttgttctcgaccacgaggttgtcgttgttgcccatcagccacagaccgaccttcaggtgctgcagccacatgccggacacgctggagcccgggccgagcgagccgttgacgaagttgtcggggttggagtcgacgcgctcggtgacctcgccgatgaccgcgaagtccttgatgtggacgttgccggaggagctggactggtcgatgaaccgcgaggtgtgcaccacggagtgccagctgccggcgccctggagggtgacgttctggacgccgttcagtgaggaggtcagcctgtagtcacccggcgggatccagaccacaccgccctgggctgcggcgatggcgtcccggaacgcctgggtggagtcgccctgcccgctggggtcggcgcccttggaggtgacggacaccgatccggcgggctgggaggcggccgccgcgacctgctcgaagtcggccacgtccacggtgacctgggtgttcgccgcctcgaaggcgatcttgtcaccggcctggacgttctggccgagcagcagccgggcgttgtcgtagaggtggtgggtcttcgaccccgcgatccagccggtgtccacgtacgagtacttggacgtgaccgcgatggtcttggccagcttggtgccgttgacatagacgttcaacgtgcccgactggccgtcgggcacgttgtaggccacgttcaccgcgttcgccgcgcggggcgcggtgaactccacgcgctgcccggcggcgaggcggacggcctggcgcccggatgcctcggaggcgagcgtgccctgggtgaagtcggggccgatcttcgtccccgtggtggtggccgactcggcctcggccgaggcgaagggcagggaggcgcccgcggccgcgtgtgccgccgtgggggtcagggtgacgagcgtgccggccgcgagggcgacggccacgccgatcgccgacaggcgtttggtggatgccgatgcggtactgctgcggtgcatgtgctgatcccttcatggtgggggtggtgggatagcgcggtgcgggggtggcggctcagcggagcagccaggccgccgtgtcctgcggaaggcggccccggtcgtccagcgggccgctgctgagcagaagccgggaggcgccgtccagctcggtgggggtgtccgcgaggttgaccacgcagaccaggccgtccgcacgggcgaaggccaggacaccgtcggccgaggggagccaggtcagcggcccgtcgccgaagccgggggtggtgcggcggatgcggatcgccgcgcggtagaggccgagcatcgagcccggggcctccgtctgcagatcggccgcgtacgccgcccagtgcgcgggctgcggcagccacggctcctcgcgcgagccgaaaccggcgtacggcgcctccgccgcccacggcagcggcacccggcagccgtcccggcccgggtcggtgccgccggagcggaagtgcatcgggtcctggatgcggtcgcgggggatgtcggcctcgggcaggcccagttcctcgccctggtagacgtagaccgcgccgggcagggccagcgacagcagggcggcggcccgtgcccgccgggtgccgagggtgaggtcggtgggggtgccgaagaccttggtggcgaagtcgaaaccggtgtcctcgcgcccgtagcgggtcaccgtgcgggtcacatcgtggttgcacagcacccaggtggccggagctcccaccggagcgtgttcggcgagcgtctcgtcgatcgacgtccgcagccgccgggcgtcccaggggcaggccagaaacgagaagttgaaggcggtgtgcagttcgtcggggcgcagatagcgggcgaagcgctcgctgtccggcagccacacctcaccgacgaagacaccgccgtactcgtcggccacgccgcgccaggagcggtagatgtcatggagctcatcgcggtcgacgtacggatggggatcgcggccctcgacgaagtcgggcagccggggatccttggccagcagggcggccgagtcgatgcgcacccccgcgacaccccgctccaaccagaagcgcaggatgtcctcgtgctcctggcgtacggccggatgggcccagttgaggtccggctgttcgggggcgaacagatgcagataccagtggccgtccggcagccgggtccacgccgggccgccgaactccgacgtccagtcgttgggcggcagttcaccgtgctcgccgcggcccgggcggacgtggaagagctcgcgctcggcgccgcccgcgagggcggcccgccaccaggggtgctggtcggagacgtggttcgggacgatgtccacgatcgtgcggatgcccagctcacgggcctcggcgatgagtttctccgcctcggccagggtgccgaaggccggatcgatggcgcggtagtcggcgacgtcatagccgccgtccttcatgggcgactggtaccaggggctgaaccacagcgcgtcgacgccgagttcggcgagatacggcagcctggcgcggacgcccgcgaggtcgccggtgccatcgccgtccccgtcggcgaagctgcgcacatacacctggtagatgacggcggagcgccaccagtcgttcggcgtccgggcaggggtgggctgggccacggtgggagcctttctgtcgagggggcggtgtcagcccttcgtgctgcccgcgctgatcccggcgatgatgtgccgctggaagacgaggaacagcgcgaccatcgggatgctggcgatgaccatcgcggcgatgagcacggtcagctggatgttctgcgacagctggacgagtgccacgctgatcggctgcttgccggtgtcggagaagaccatcagcggccacaggaagtcctgccacaccgccaccagcgcgaagatcgacacaacgccgagcaccgggcgcgacatgggcagcacgatcgaccacagggtgcgcagcttcccggcgccgtcgatctcggcggcctccaggacatcgcgcgggatctggtcgaagaaccgtttgaggagatagaggttgaaggcgttggcgacggccggcagccagatcgcgagcgggtcgttgagcaggctggtgtggatcagcggcaggtcggcgacggtcaggtacttcggcacgaccagcgcctgggccggaaccatcagcgtggccaggatgccaccgaggatcaccttgccgaaggcgggcttcagcctggacagggcataggcggcggccgtgcagaagaccagctggaacagccaggcgccggctgcctggaccaccgtgttccacaggtgctgcggcagctgcatcaggtcccaggcgtcgctgtagccgctgaggtgccactctttcgggacgatggtgggcggtgtccgcgccacctcgtcgggcgacttcatcgcaccggtcaccatccagtagaccgggaagaggaaggcgatcgcgaacagcaccaccacggtggtgaagaccgtccagtagacggcccggccgcgggggcgggccagggcggcgggggagacgagggtccgggtgctcatgcgtcgtcctccccggagcgggtgagccgcagatagagggcggagaaggcgccgagcagcacgagcagcatcacgctcagcgcacaggcgccaccgaagtcgttgtagaggaaggcgtacttgtagatcaggtagaggaccgtgaccgtggcgttctccgggccaccaccggtgatcacgaacggctcggtgaagacctgcatcgtcgcgatgatctgcagcagcatcagcatgaggatcacgaaccgcgtctgcgggatcgtgacgtggcggacgcgctgcagcaggctcgcgccgtcgagttcggccgcctcgtacagctcaccggggatggactgcagcgccgccaggtagatcaggacggtgccgcccatattggcccaggtggccacggcgacgagggagaccagagcggtgtcggcgccgttggaccagttcgaggtgggcaggtgcaggaagcgcagcgcctcgttggccagcccggcgcccgggtcgtagaaccacttccacagcagggcgctgaccaccggcgggatcatcaccggcagatagaccacgaccctgaagaacgccttggcgtgccgcagttcattgagcacgagggcgagcaggaacgggatcgcgaagccgatgaggagtgccagcagggtgaaggtgagggtgttccgccaggccgcggtgaactccgggtcgtgcaggacgcgggtgaagttggcggtgccgacccattcgggggacgagccgggcgtgtacttctggaaggcgatcacgaccgcgcggatcgccggataccaggagaacagcgcgaagcagatcaggccgccgaggaggaagccataggcccggacctggtcggcgagacggcgccgcccccgaccccccgccgggggcggcgcctgcaccgggtggacggcgatcgcctcggcgggcggccgcgcggcggtcttggtcatcgggtcagccccgggccagaatgttgtcgatcttgtcggaggcttcctccaggagctggtcgacatcggcgtccttcttggtgaggacggcggagacggctccgtcgagcacggagtagatctgctgggcgtgcggcggctcgatcctcatccgcagcttctggttgccgtcgaggaaggtctggtagttgcccacggggacattggcgttggccttcttgacctgctggtccttggcgtcggctgcgccggtgaacagccgtggctcgggcaggcccaccggggcgtttcgcttcttggcgcggacgtagtcgccgaggaagccatcgcccggggtgaggaacatgtggtcgagccacttgagaccggcccggatctgggcgggcgtgtccttcttctggaacatgtagccgtcgccgccgatgagcgtgcccttgccaccgggcatgggggcgatggcgaggtccttgtagttgccgcccttctccttcaccaggatcgggaggttgtcgggcgcggccaggtacatgcccagcttgccggagcccatcagctgctgggcgtcgttgatgaccaggagctgcttgctgcccatcgagtcgtccacccagcgcatgtcgtggaggttccgcaggacggcgcgcgcctcgggggtgtcgatggtggccttcttgccgtccgcgctgacgacatcgccgccctgtgagtacagctcggccgtgaagtgccagccgccctggttctgggcgctgtagtccgcgtagccgaccgtgccatcgcccagcttggcgatcctcttggcgtcggcgcggacctcctcccaggtcatcgggggcttgtcggggtcgagtccggccttctcgaagagcttgcggttgtagatcagacccatcgagtagccggtgcgcgggatgccgtagatcttgccgtcgaccgtgtagatgtcgcgcagctgcttctggagggtggagtagctcttcaactccttgacgtacggcgtgagatcggccgcctggttgatgtcgaccacatgtccggcgtcggtgaagtacgtgtagaagacgttctccatctggcccccggccagcttggcgtcgaacgtcttcgggtcctggcaggggaacgcgtcatgcgcgacgacgtcgatgtccgggttctgcttctcgaaggaggcgatgtcctcctcgaagaacctgcggtcgaccttggcgctcttgggcggcatgcagttgaccgtgatgcgcgtctttccgcccgccgagccgtcgcccgacccgccgcaggcggtgagggcgagggggaacgtgctgagcgcgatgagagtacgacggaacccggtgcttctcatgggtggacccctctgtacaggagcagggaagccccacggccgtgagcggggcgcacacaaccgtgagtgcgccgcacactcaagcaccgacgacatcggcccgcaagatgtcgcgtagattctgtaattattcaactgcgctgcgaatcaggcggattgagcctgtcggtatctctcgaccgccctttcgatcacccctcgacggtcctccgcccgctcactcccgtggcgcctgggcggtggagccgcggaccaccagctccggctcgaacagcagctcctcggacggtacggccaccccgccgatctgcgcgttcagcacctccaccgccgccctgcccatggcctctatgggctggcggacggtggtcagcggcggctcggtgcagttcatgaacgcggagtcgtcgtagccgaccacggacacctgcgacggcacgccgaaccccttgcggcgcgcggctcgtatcgcgcccagggccagcgggtcgctggcgcagatgatgcccgtgacgccccggtcgatcagccgggaggcagcggcgtggccgccctcgatcgagaagatcgcccgggccacgaactcatccggaaggtggcctgcgaccgcccgcgcggcggtcagcttgcgtgccgacggcatgtggtcaccgggcccgagcaccaggccgatccgctcatggccgagggaggccagatgccgccacgcctgctccacggccacggcgtcgtcgcaggagacagccgggaagccgaggtgctcgatggccgcgttgaccagcaccaccgggatgttgcgctcggcgagcagccggtagtggtcatgcggcgcgtcggcctgcgcgtacagcccgcccgcgaacaccaccccggagacctgctgttgcagcagcagcgccacgtaatcggcctcggagaccccgcccttggtctgggtgcacagcaccggggtcagtccaagctgtgccagcgccccaccgatgacctcggcgaacgccgggaagatggggttctgcagctcgggcagcaccagccccaccagccgggcccggtcgccccgcagctgcgtgggccgctcgtagccgaggacgtccagggcggacagcaccgcctgccgggtggctgcggagaccccgggcttaccgttgagtacccggctgaccgtggcctcgctgacaccgaccttcttcgccacttcagcaagtcgtcgcgtcatgcacgcaagcgtagcgcaagcactgcaagtggcttgcgtaagaggggtggagaacgtgtgactccggcggcggctcccggctcggattcaccctatttgccgccctaaaagtgtgggttgacagcggatcagccaacgatctaggttcccttcgaggggctcgctggatgggggacgggggctgtagtgagtgttttggagttgcgtgcgtccgatatcacccggaccgcgcggctggtcggacgtgccgcattatcagaccgaatgatcgaccaattgagctccgggatcgctgccttggaccgcgcggaaaatgaccactcggcgcggcgtggcggagctatcggcgatatcgacgccgagcacaccatcgatgccggtcatacggcccgcgtcgagggcgagcgtcggcggtcggccgagcccaccgtattcgagtccctcgactctcccggctccagcgccgctacgggattcacgctcgaagaaacacttcgcattcgatccctttctcgggtttgccgccgaatgtaatcccggccgtactgccgtttaagaagcgtttacgccatcggttggcaagcgaaagcgacagcaccagcggaataaccgcgaaaagcaatttccatcagtcgtcggggaaggctgtgttgtggggaattcaggcgcacccaaatcccgtggtctcagcgcggccatgagcaatctcttcgagcggaccaggagaaacgaatccaccggcattgtgccggtcgaccggggccgggagctgagggcgtcgttcgcccagcaacggctgtggtttctggaccagttggaacccggcaacgcctcgtacaatctccccttcgcggtgcgggtgcgcggccgcttggacatctctcatctctcccgggccctctcgctcgtggtcgcccggcacgaggcgctgcgcaccaccttcggcgaggccggcggtcagccggtgcagcggatcgagccccccggccccgtcccggtgcgccttgaagcggtgtccggcggctcggaggaggagcggctggccgaggtccggcggctggccggagccgagatcaccgagcccttcgacctgagcaccgggcccctgctgcgcgccaaggcgctgcgactggacgaacaggaccacgtcctgctgctgacggtgcaccatgtggcgacggacgcctggtcacaaggcattgtggtgcgtgagctgtccgtcgcgtacgcgtcgctcgacgccgggcgcgagcccgtgctgcccccgctgcccgtgcagtacgcggactacgcggagtgggagcgcgactggctgtccggcccgaccctgcgccgccagctggactactggacgaagcggctcgacggcatggcgcccgcgctggagctgcccaccgaccggcccaggccctcggtcgccagccaggaaggcgacgcggtgcgctgggagttgccgccggaactgatccgggcggcccgccggctgggcgccggtgagaacgcgaccctctacatgaccctgctggccgctttccagctggtactgggccggtacgtggacagcgacgacatcacggtgggcacccccgtggccaaccggggccgcgccgaggtcgaggggctcatcgggttcttcgtcaacaccgtggtgctgcggaccgacctgtccggcgaccccaccttccgccaactgctgggccgggtccgcgacacggcggcgggtgccttcgcccatggcgacctgcccttcgagtatctggtggagcaggtgcaccccgagcgggacttgtcgcggaacccgctggtccaggtgctcttccagatgatcaacgtaccggcggagcggctcgagctgcccggcgcgcggaccgagccctacgaccacggcggcatcctcacgcgaatggatctggaggtccatctcgtcgagaccggggacggggttctggggcacatcgtcttcagcaaggccctgttcgacacgagcaccatcgaacggctgctgcaccacgtcaccgtcgtcctccggggcgtcctggccgagccggaccggcgcatctccgagatctcgctgctcgacgaggcggagcgggcgaaggtcctggagaagttcaacacgaccacgggccccgtacccgccggatccctgcccgcgctcttcaccgcccaggccgagcgccgccccgatgcggtggccgtgatcagcggtggtgaccgggtgacctacgccgagctggatcagcgggcgaaccagctcgcccatctgctggagggccggggggtcggccccgagaccctggtcgggctctgcgtcgatcgcggcatcgagatgatcgtggcgatcctcgcgatcctcaagctcggagcggcctatgtgccgatcgatccccaccacccccgagaccgcgtccagttcgtccttgccgactccggggtgaccgtcgccgtcacccagcagcgcttcaccggcctgctcgaaaccccggaggcacccgggacgcccgatgcgtccgggacgtccgggatccgcctcatcctgctcgacgccgagcgcgagccgctcgccgggcagccccggaccccgcccacggcacggcccagcgcccagaacctcgcctatgtcatttacacctccggctccaccggagtccccaagggcatcctcatgcccgccacctgtgtgctcaacctggtggcctggcagaagcgggccctgccgatcggtcccgacgccaagacggcacagttcgccacgctgaccttcgatatctcgttgcaggagatcttctccgcgctgctgtacggcgagacgatcgtcgtccccggcgaggaactgcgcatggaccccgccgagttcgccacatgggtccacgccaacgagatcgaccagctcttcgtcccgaatgtgatgctgcgggcgatctccgaggaggtggatccgcacggcaccgagctggccgcactgcgccacctctcacaggccggcgaacccctctccctccaccacgatctgcgcgagctgtgcgcccgccgccccgagttgcggctgcacaaccactacggtcccagcgaagcccatgtggtgacgtcgtactcgctccccgccgaggtggccgagtggccgctcaccgcacccatcggccgcccgatcggcaacacccgggtgtatgtggtcgaccggcggctccggcccgtcccggtgggggtgccaggtgagctgtgcgtggccggagaggggctggccaggggctatctcggccgcccggatctgaccgcttcccggttcgtggcggacccgttccgcggcgacggatcgcgtatgtaccgctccggcgacctggtgcgctggctgcccgacggcaacctggaattcctcggccggatcgatgaccaggtgaagatacgtggcttccggatcgaaccgggcgagatcgaggcgatcctcgcccggcaccaggacgttctgcacacggccgtgatggtgcgcgaggacacccccggcgacaagaggctggtggcctatgtggtggccgatgccaccgccgcggaccggcacggcgggctgaccgagaccctgcgccggcacgtcgagtccgcggtgcccgaatacatggtgccctccgcgttcgtcctgctggacaccatgcccctgacctccggcggcaagatcgaccggaaggcgctgcccgcccccgatctgcgcaccgtgctcgaggtcggctacgtcgccccacgcacccccgaggaagaggccgtctgccgggtttacgcggatctgctcggcgcggccaaggtcggcatcgacgacgacttcttcgcactgggcggccattccctcatcgccaccagggtggtcgccaggctccggtccgccctcggtatcgccgtaccgctgaagaccgtcttccagcagcgcaccccccgagagctggcggccacgctcaccgccgcggcccgctccggtcccgaacccgagctgccgccgctggttcccacgcggcgcgaccagcccgtccccctcaccttcgcacagcagcagacggacctcttcttcgacgatgtcctgaacgccgggcactggaacatccccatggcggtgcgggtgtcgggcgaactggacctcgactgcctgcggcgggcgatggacctgctgatcgaccgccacgaggccctgcgcaccaccttcgtcagggaagccgacggatacgtccaggtgatccggccgagcgcgccggtccaggtggaggtggccgagacgcacgacgagaccgaagcctcggtactggccggccaggaggccgcccgccccttcgacctcacacgcggcccgctggcgagactgcgcgtgctgcggctgtcccagtccgaccatgtgctggtgctcaccctgcaccacctggtcaccgacggctggtcccagggagtgctggtccgagatctgtccatcgtgtacgcggcactgctgcacggcaccgaacccgatctgccacccgcacccgtccagtacgccgatgtcgcgagctgggagcggaagtggttgcgcggtccgctgctgcaacgccaactcgagttctggaagcggcatttcgagggcatgacccccgccgaactgcccaccgaccggccccgcgccgcgtcggcccgctacgagagtgacatcttccactggcgactgccgacggacgccgtcgagaccgcccgacggctgggcgaatcgtgcaacgccaccttgtacatgacgctgctgaccgccctgaaggtggtcatgtccgcccgctcggacaaccaggacgtcctcgtcggcgtgcccacggccaaccgtggccgggacgaactggagaacacggtgggcctcgtctccaagatgctcgcgctgcgcaccgaagtgtccggtgccacggacttcggcacactgctggccacggtgcgcgatgcgatgtccgacgcccatacacaccaggacgtgcccttcgtgtccgttctcaagcacatcggtgaccacaccgccggccccgccggtgacaccgccggcggccgggccgggacgcggctgtcggacgatccgccagtgaaggtgatctttcagatcgtcaacaccccgccgcggccactccggctcaccggactgacggccgagccgttcccgatgacccacccgccggtcacggtcaacgtggacatggagatcgacctgtacgagagcgcggaggacggcggcctcgccggcaccgtgctgttcagcaagtccctcttcgaccgtgccacgatcgagcggttctgcgacgacgtggtggcggtcgtctccgcggccgccgcggatcccggacggccggtctcacaggtgtggcagggccggggccgcgaccagtgaacgatcccgccccgaggaaacgcatggaaccggatgaggccgtcgccgttgtcggaatgtcctgccgctttccgcaggcacccgatcccgaggcgttctggcggctgctgagcgagggcatctcggccatcggtgaggtgcccgcggggcggtggaccgacgaccagcccacgccgtccgggaccgacgagcggtccacgccgcccgccatccgccgcggcggcttcatcgacgacgtcgaccgcttcgaccccgcgttcttcggcatctcaccacgggaagccgcggcgatggacccccagcagcggctgatgctcgagctggcctgggaagggctggaggacgcgggcatcgtgcccgccaccctgcggggcgccaccgtcggagcgttcatcggcgccgggtccgacgactacgcctcgctgatccgcgcccgcggccgttcacaccacacgctgaccggcacccagcggggcatgatcgcgaaccggctctcccatgtgttcggcctgagcggcccgagcgtgaccgtggacgcggcccaggcatcctccctggtcgcggtacacatggccgtggagagcgtgcgccgcggcgagtcacggctcgcgctggcgggcggggtcaacctgaacctctccgcggagaccgccgccgatatcgcggcgttcggcgcactgtccccggacggccgctgcttcaccttcgacgcacgcgccaatggctatgtgcggggcgagggcggcggactcgtcgtcctgaaaccgctctccgacgctctcgccgacggcgacaccgtctactgcgtgatcgagggcagcgcggtcaacaacgacggcggcggtgcatcgctcaccgcacccgacccggacggccagcgacgggtgctccgactcgcccagcggcgggccgcgatctcccccgaggccgttcagtacgtggagctgcacggcaccggaacggcactcggcgacccggcggaagcggcggccctgggcgccgtcttcggccggagcggagcgaggccggtgcagctggggtcggtgaagaccaacatcggccacctcgaagccgccgccggtatcgccggacttctgaagaccgcactggccatccaccaccggcagctgccggccggcctcaattaccgcacgccgaatccccgtatccccatgggcgaactcaacctggagatgcgcctcgcaccgggggagtggccgaagccggacgaccgcctggtcgccggtgtcagctctttcgggatgggcggcaccaactgccatgtcctgctcgccgaaccactcgtcggcgtcccctcccacgcctccgcgcatgcccctgagcccgactccctccccagctcgatcccggccccggtcccggtcccggtcccggtcccggccccggtcccggtcccggccccggccccggccccggccccggtcccggtccccgtcccgcttccgttgtccggggtgtccgctgccgcgcttcgcggccaggcgatgcggctacggccgtatctggagcgatcgccgaacctcaccgacctctccttctccctcgccaccgcacgaacctccttcgaccaccgtgcggtgctgatcaccgggcaggcggccgacgcggcacacggcctggacgcgctcgtcgaaggcggcacggtggcgggtttggtgacgggcacggcgagggcggcgggaaagctcgccttcgccttcgccggccagggctcgcagcgtctcggcatgggacgtgaactcggggccgtcttccccgtctttgcccaggctcttgacgaagtgtgcacggcgctggacgcacacctggaccggccgcttcgggacgtgatccacggtgacgacgccgaaccgctcaaccggacggtgtacgcccaggccggactcttcgcggtggaggtggcgctgttccggctgctggaggacttcggcctcgtaccggacctgctgatcggccactccctcggcgaggtgagcgccgcccatgtcgccggtgtgctgtccttggcggacgccgccaccttcgtcgccgcccgtgggcggctgatgcaggccgtgacggagccgggcgccatggtgtcgctcgaagccaccgaggacgaggtcacccggacgctcatggcgggcggggcatcggacgacggtgcccgggtgtgcgtggcggcggtcaacggccccaccgccacggtgatctcgggggacgagcgcgccgtactcgacctggcggtggagtgggccggtcgcggacgcaagacgaagcggctccggacgagccacgccttccattcgccccatctggaccccgtactggacgagcttcggcacatcgccgagagcctcacgtaccgggcgccccggatcccgctggtgtcgaatgtgaccggccgacgtgccacggcggaagagctgtgttctccggagtactgggtccggcatgtccgccggaccgtacggttcctggacggcgtccgctgtctggaggacgaaggcgtcaccaccatcctggaactgggcccggacaaggcgctcaccaccctggcccgcgactgcctgaccgggcccgggacgctggtgggcacccttcgtcgcgaccggcccgagccgcaggccctggtcaccgcgctggccgagctgtatgtctcgggtgtcgaagtggcatggagcccgctggtgtccggtgggcggcggattccactgcccacgtacgccttccagcggcagcggtactggttctccgctcccgggcccgagagcggaaccacgcctggccatggggtcacatccgggcgcgagcgcacggacaccggcctgagcggcgacgaggcgcccgacaccggcccgagcggcggcgagacgcttggcatggtccgggcgcacgcggccgtcgtgctcggatacgcgtcggcaaccgccatcggcgccgagcacaccttcaagcaactcgggttcgactcgatcaccgccgtcgaactgtgcgaacggctcggtgcggcgaccgcgcttccgctgcccggcaccttgctgttcgactatccgacgcccgccgcgctcgccgagcatctgcaccgcaggctccacggccggacggatgagcaggccgcgcccgcgaccgtgccaacacctgacggcggcgatccggtggtgatcgtggggatgggctgccggttccccggccgggcccactcgccggaggacctgtggcggatcgtggccgacggtgaggacgccatctccggctttccgtccgaccggggctgggacctcgctggtctctaccaccccgaccccgaccaccccggcacgtcatacgcacgcgacggcggattcctctacgacgcggccgagttcgacgcggggttcttcgggatctcaccgcgtgaggccgaggcgatggacccgcagcagcggctgctgctggagacatcgtgggaggcgttggaacgggcgggtatccccgcggaacacatcaagggcagtagcacgggcgtgttcatcggcgcctcgtcggtcggctacgcggcggacgccggagaggcggccgagggctaccagctgaccggcactgccgcgagcgtggcctcgggcagggtgtcctacaccctgggcctcgaaggcccggcggtcaccgtggacacggcatgctcgtcctcgctggtggcattgcacctggccgtacagtcgctgagggcgggcgagtgctcactggcattggcgggcggtgtgaccgtgatggccacaccggcgatgttcgtggagttctcccgtcagcgggggctggccatggacggtcggtgcaaggcgttcgcggcggcggcggacggcacggggtgggccgaaggcgtcggggtgctggtggtcgagcggttgtcggacgccgagcgcaatgggcatcgggtgttggcggtggtgcgtggttctgcggtgaatcaggatggtgcgtcgaatggtttgacggcgccgaatggtccgtcgcagcagcgggtgatccggcaggcgttggcgagtgcgggtcttgtggcgtcggatgtggatgcggtggaggcgcatggtacgggtacgacgctcggtgatccgattgaggcgcaggcgttgttggccacgtacggtcagggtcgggatgcggatcggccgttgtggttggggtcggtgaagtcgaacatcggtcatacgcaggcggccgcgggtgtggctggtgtgatcaagatggtgatggccatgcggcacggggtgctgccgcgaacgctgcacgtggatgagccgtcgacccacgtcgactggtccggcggccgggtagagctgctcaccgggacaacgccatggcccacgacgggtggccttcgccgagcgggcgtctcctcgttcggtgtgagtggcaccaacgctcacgtcatcctggagcaggtcccggagacggcccggccgaccgggcccatcggggaagacgacggcgaagcggcgcccgtcgcctgggtgttgtcgggacagggcgagactgggctgcgggcccaggccgagcggctgtgcgccttcatggcggccgatacccgccccaccccggcggaagtgggatggtcactggcatcgacacgtgcgacgttgtcgcaccgcgcggtggtcgtgggtgctggacgcgacgagttgttgcgtggtgtgaatgcggtggcgaacgggacacccgtgccgggagtggtacggggcaccggagcctccggggacgtggtgttcgtcttcccggggcaggggtcgcagtgggttgggatggcgttggagttggtggagtcgtcgccggtgttcgcgcggcggttgggtgattgtgcggatgcgttggcgccgtttgtggagtggtcgttgttcgatgtgttgggtgatgaggtggcgatcggtcgggttgatgtggtgcagccggtgttgtgggcggtgatggtgtcgttggcggagttgtggcgttcgtttggtgtggtgccgtcggcggtggtggggcattcgcagggtgagatcgcggcggcgtgtgtggcgggtgcgttgactttggaggatggggcgcgtgtggtggccttgcggagcagggcgttgctggctctgtcgggtcggggcggcatggtgtccgtaccggtgtccgccgatcggctccgtgaccgtgtggggttgtcggtggcggcggtgaatggtccggcgtcgacggtcgtgtccggggcggttgaggtgctggaggcggtgctggcggagttcccggaggccaaacggattccggtggattatgcctcgcattcggtgcaggtggaggggatccgggaggggctggcggaggcgttggcgccggttcggccgcgtacgggtcaggtgccgttctattcgacggtgaccggccggctgatggacaccatcgagttggacgcggagtactggtacaggaacctgcgcgagacggtggagttccagagcaccgtcgaacacctcatgcgccagggtcatacggtgtttgtcgaggccagcccgcatccggtgctgaccatcggcgtccaggacaccgccgacaccaccgacactgacatcgtcgtcaccggatcgctgcgccgcgatgatggcactgtccagcggtttctgacctccctggccgagctccacgtgcgcggtgtccggatcgactggggcccgctcttcgccggtgtctcgcccgttgagctgccgacgtacgccttccaacgggaacggttctggcttggggcggacatcgccgagtccgccgtggacacgtggcgataccagatctcctggaagccgctgccggacatggaccccccggccctctccggcacctggctggccgtggtccccgaaggggacgagtgggccatggcgggcgcacgggcgctgatcgagtcgggcacggccagcgtccgtaccctccaggtgacctgcgacgcggaccgccggaccctggccgggccgctgacggatgtggcgggatccgaagacatcgccggtgtcgtctcgttcctggccgccgacgaagttccgcatccggcccaccccgcgctgtcccgggggatggcgcacacggtcgagctgctgtgctcgctcaccactgccgatgtcgaggccccgctgtggtgtgtcacccgggcggccgtcacggcactgcccgcggacccggcgccgagccccgcccaggcggcggtatggggattcggacgggtggccgggctggagcgatccgagcggtggggcggcctgatcgacctgcccgtccactgcgacgcacacgtgctgcggcggttcgtcgccgtactcgcgcaggcagccggtgaggaccaggtggcggtgcggccatcggcggccctgggccgacggttggagccggcgcccaggaccggaccggccggcgcatggcgcccgcacggcacggtgctgatcaccggtggcaccggcgtgctgggcgcacatgtggcacggtggctggcgcggtccggcgcggaacacctggtgctgctcagccgccgtggcccgcaggcccctggggcggccgtgctcgacgacgaactgaccgcgctcggcgtacgagtgaccctgacggcctgcgatgtgaccgaccgggccgctctcgccggggtgctggcatcggtgccggacctcaccgccgtggtccatctcgcggggaccgtgcgattcggcaattccatcgacgcggacctcgacgagtacgccggcgtcttcgacgccaaggtcaccggtgccctgcatctggacgagctcctcgaccactcgtcactggaggcgttcgtcctcttctcctcggcagcggccgtctggggcggtgtcggccaggccggttacgcggcggcgaacgccctgctcgacgcggtggcacagcggcgtcgcgcacgcggtctgccggccacttcgatcggctggggcacctggggcggcagcctcgcgcccgaggacgaggagcggctgagccgcatcggcctgcgcccgatgcggccggaggtggccgtcaccgagctgcgccacgtcgtcggatcggccgagccctgcccggccatcgcggacgtcgactgggagaccttcggcccggccttcacggcaggccggcccagccgcctgctcagcgagttgccgcggctgcgaaacacctccggcgccatggcgatgaccggcgaccacgccgcattgcggaggcgactggccggggtgtccgcggccgaccaggcccggacgctggtggacctggtacgtgaacacgcggcggaactcctggggcaccgcggcccggcggcgatcgaccccacggtgccattccggcaactgggcttcgactcgctgacggcggtcgagctgcggacccggctgaacgcggccacgggactgcgcctcccggccaccttgctgttcgaccacccgagctgccgggcggtcgccgatctgctgcgctcggaactgctcggcgaccggccgggctccctcgcggcgtcgtccgccacggaggctgtgcccgccggcgtggtggcctccgacgagccgatcgccatcgtcgcgatgagctgccgcttcccgggaggcatcggaacccccgaggacttgtggcgggtggtcagcgagggccgggacgtgctctccgacttccccgacgaccgcggctgggacgtggacgcgctgtacgacccggacccggaccggcccggcaccagctatgtgcgtaccggtggattcctccacgacgccgcggagttcgacccggaactcttcgggatctccccgcgtgaggcgctggcgatggatccccagcagcggctgctgctggagtcggcgtggcaggtcctggagcgcgccaggatggcgccgacctccctgcgatccagcaggaccggtgtcttcatcggcggctggggccagggctacccctcggcctcggacgaggggtatgccctgaccggcgccgcgaccagcgtgatgtccggtcgtatcgcctacgcgctggggctggagggccccgccctgaccgtggacacggcatgttcgtcctcgctggtggcgctgcatctggcgagcgaggcgctacggcgcggcgagtgctcgctggcgctcgccggcggcgtgacggtgatggcgacgcccagtacctttgtggagttctcgcgccagcgtgggctggccccggacgggcgctgcaagccgttcgccggggcggcggacggcacggggtggggcgagggcgtgggcatgctgctggtggagcggttgtcggatgctgagcggcttgggcatccggtgctggccgttgtctccggctctgcggtgaatcaagacggtgcgtcgaatggtttgacggcgccgaatggtccgtcgcagcagcgggtgatccgtcaggcgttggcgagtgcgggtcttgtggcgtcggatgtggatgcggtggaggcgcacggtacgggtacgacgctcggtgatccgatcgaggcgcaggcgctgctggccacctacggtcaggaccgggatgcggatcggccgttgtggttggggtccctgaagtcgaacatcggtcatacgcaggcggccgcgggtgtggctggtgtgatcaagatggtgatggccatgcggcacggggtgctgccgcgaacgctgcacgtggatgagccgacaccgaaggtggattggtccgccggcgcggtgggactgctcaccgagtcggccgagtggcggcaggagggccgaccgcgccgagccggggtgtcggctttcggggtgagcggcaccaatgcccatgtgatcctggagcaggccccgaagcacgcaccgggggtggcggccgagggcaggaaggggcgcggggagccgccgacggtgccctgggtgctgtcgggcgcgagcgaggcgggtctgcgggcgcagatcgaaggcttgcgggccttcgctgacgacaaccccacgctcgatccggcggatgtgggctggtcgttggcgtccacacgtgcgcttctgccgtatcgcactgtcgtcgtgggcaccgacctcgacgagttgcggcgtgggttggacgcggcggaggtggtgggcgcggccgagccggaccgtggcgccgtgttggtgttcccggggcaggggtcgcagtgggttgggatggcgttggagttggtggagtcgtcgccggtgttcgcggggcggatgcgtgattgtgcggatgcgttggcgccgttcgccgagtggtcgttgttcggtgtgttgggtgatgaggtggcgcttgggcgggttgatgtggtgcagccggtgttgtgggcggtgatggtgtcgttggcggagttgtggcgttcgtttggtgtggtgccgtcggtggtggtggggcattcgcagggtgagatcgcggcggcgtgtgtggccgggggtctgtcgttggaggacggtgcccgtgtggtggccttgcggagcagggcgttgctggctctgtcgggtcggggtgggatggtgtcggttccggtttctgctgaccggctgcggggtcgtgtggggttgtcggtggcggcggtgaatggtccggtgtcgacggtggtgtcgggggctgttgaggtgctggagggggtgctggcggagttcccgggggccaagcggattccggtggattatgcgtcgcattcggtgcaggtggaggggatccgggaggggttggcggaggcgttggcaccggttcggccgcgtacgggtgaggtgccgttctattcgacggtgaccgggcgattgatggacaccgtggggctggatggggagtactggtatcggaatctgcgtgagacggtggagttccagtccgcgatcgaggggctgctggagcttggtcatacggtgttcgtcgaggccagcccgcatccggtgctgaccgtcggcatccaggacaccgccgagaccacggacaccgacatcctcgtcaccggctcgctgcgccgtgacggcggtggccttgcctctttcctcaccgcgctggcccggctgcatgtccggggtgtcgcggtggagtggcgggaggcgttcgccgggctggacgcccacgccgtggacctgccgacctacgcctttcagcgtcggcgcttctgggcggcctccctgcggcagactcccgggacggccgagttcgaccatcccctcctgggcgcggtgctgcccttgcccgattccggcggcggtctgctcacgggcgtgctcacactggccggacagccgtggctggccgaacactcggtggccggtgtggtgttgttcccggggacggggtttgtggagttggtgttgcaggcggggttgcggtgggggtgtggggtggttgaggagttgactttggaggggccgttggtgcttccggagcggggtgaggttgaggttcaggtttcggtgggtggtgtggatggggcggggtgtcggtcggtgtcggtgttttcgtgtcgtgggggtgagtgggttcggcatgcggtgggtgtgcttggggtgggggatggtgtggtgccgggtgtggaggtgtggccgccggtgggtgcggagcgggttggggtggagggggtttatgaggttttggcggagcgggggtatgtgtatgggccggtgttccaggggttgcgggacgcctggcgccggggcgacgaaatcttcgtggaggcggaggtaccggcggaggcgcggggcgatgcggctcgctgtgccatccatcccgcgctgctcgacgcagggctgcacggcgtcggattgggcggcctgatcagcgacgacggccgggcgtacctgccgttctcctggagcggggtcaggctgcacgcggtcggcgcatccgctgtccggatgacgctgacgcccgccggaccggacgcggtgtcgctgagggtgaccgatgaggcgggcgaggcggtgctgacggcggactcccttgtgctccgcccggtcaccgagggacagctcgccgaagccgagatcggcaaccgcgatgtgcttcatcgggtggagtgggtggatgcgggggcgtgttcggtggggtcgttcgtggagtggggtgaggtggctgctggtggggtggtgccggattgtgtggtgttggccggggctgatgtggcgggtgtgttggaggttttgcggacgtgggtggtggaggagcggtttgagggttcgcggttggtggtggtgacgaggggtgcggtgtcggtcggtggtgagggtttggaggatgtgagtggtggtgcggtgtgggggttggtgcggtcggcgcagtcggagcatccggggcggtttgtgctggtggacgccgatgtagatacggatgtggttccggatgtggtggggctgggggagtggcaggtggcggtgcgtgcgggtcgggtgtgggtgccgcgtctggtggatgtggatgtgagtgtgggtggtgctgtggtgcgtgggggcttgggttcgggtgtggcgttggtgacgggtgggacggggttgctgggtgggttggtggcgcgtcatctggtgtcggcgtatggggtgggtgagttggtgttggtgagtcgtcggggggtggctgcgccgggcgtggaggagttggtgggggagttggaggggttgggcgcgcgggtgcgggtggtggcgtgtgatgtggcggatcggggtgcggtggcggagttggtggggtcgatcgaggggttgcgggtggtggtgcacgcggcgggtgtcgtggatgacggggtgatcggttcgttggacgcggagcggttgtgtggggtgatggggccgaaggcgtggggtgcctggcatctgcatgagctgacgcgtgggttggatctgtcggcgttcgtgttgttctcgtcggcggcgggtgtgttgggcaacgcgggccagggcggctacgcggccgcgaatgggttcctggacgcgctggcggttcaccgtcgggggcggggactccccgcggtgtcgatcgcgtggggcttctgggaggaacgcagcgaactgaccgccgacctggccgaggtgcagctgtcgaggatctcccggtccgtaggggccagcatcagcagcgcacaaggactggatctgttcgacgcggcgcttgccgccgacgagccgatggtgctggccacacccctgaacctgcccgcgttgcgggaccaggccgccgcgggcacgttgccctcgatcctgagcggactggtcaccgctcccgtccgcaggacggccggcaccgggcgcactccggccggactgcggcaccaactcgccggggtgacagaggccgaaaggcagcaccagatcatgcgcctggtgcaggaacatgtggccggcgttctgggacatgcctccgcggagttggtcgacgcctcgcggacgttccaggagatcgggttcgactcgctgaccgccgtggaactgcgcaaccggatcagcgccgccaccggcatacggctgcccgccaccgcggtcttcgaccaccccacgcccaggctgctggccgagcgggtgctggccgaggtagggggctccttgccgaccgccgccccgatcgcgccggtgtcggccgtcgatgacgagccgatcgtgatcgtgggcatgagttgccgcttccccggcggcgtcgagtcccccgaggacctgtggcgcctggtccactcggccaccgacgcggtctccgcgctgcccacggaccggggctgggacctggccaccttgtccggtgccaagggcggcgccggtgcctcgtacgcccgggacggcggattcctttacgacgcggctgagttcgacgccggattcttcgggatctcgccgcgcgaggcgaccgcgatggatccgcagcagcggctgctgctggaggcggcctgggaggtgttcgagcgggccggaatcgccccggacacgctcaaaggcagccggacgggcgtcttcacaggcgtgatgtaccacgactacggctcgtggctcaccgatgtcccggaggacgtcgagggctatctgggcacaggcatcgcgggcagtgtggcgtcggggcgactcgcctatacgttcggccttgaggggcctgccctgacggtggacacggcctgctcctcatcactggtggcgctgcatctggcggccgagtcgctgcggcgcggggagtgctcgctggcactcgcgggcggcgtcaccgtactggcgactccgcaggtcttcgtggagttcacacgccagggcggactcgcaccggatggccggtgcaagcccttcgccgctggtgcggatgggacgggctggtcggagggtgttgggctgctgctggtggagcggttgtcggatgccgagcggaacgggcatccggtgctggccgttgtctccggctccgcggtgaatcaagacggtgcgtcgaatggtttgacggcgccgaatggtccgtcgcagcagcgggtgatccgtcaggcgttggcgaacgccgggctcgccgccagggatgtcgatgcggtggaggcgcatggtacggggacgacgctgggtgatccgatcgaggcgcaggcgttgctggccacgtacggtcagggccgggatgtgggtcagccgttgtggttggggtcggtgaagtcgaacatcggtcatacgcaggcggctgcgggtgtggctggtgtgatcaagatggtgatggctatgcggcacggggtgctgccgcgaacgctgcacgtcgatgagccgtcgccgcatgtggattggtctgctggggcggtggagctcctgggggagcacatgggctggccggaggtcgggcggccccgtcgggcgggtgtctcgtcgttcggggcgagtggcaccaacgcccatgtgattcttgagcaggcccccgacatggcgggtgaacctgagcaaaggccggagcgtaacgaactaccggcgattccctgggtgttctccgctggcgacgaggcgggtttgcgggcacaggccgtacggctacgggccttcgcggaccggaatccggatctggatccggtggatgtggggtggtctttggcgactggtcgtgcggggttgtcgcatcgtgcggtggtggtgggtgcgggtcgtggtgagttgttgggggctttggagggtgtgccggtggtgggtgtgccggtggtgggtgggttgggtgtgttgtttgcgggtcaggggtcgcagcggttggggatgggtcgtgggttgtatgaggggtatccggtgttcgctgcggtgtgggatgaggtgtgcgcgcagctggaccagcatttggataggccggtgggtgaggtggtgtggggtgatgatgccgggttggtcggggagacggtgtatgcgcaggcggggttgttcgcgcttgaggtggcgctgtatcggctgatcgcttcgtggggtgtgaggggggattatctgctgggtcattcgattggtgagttggctgcggcgtatgtggcgggtgtgtggtcgttggaggatgcggggagggtggtggtggcgcggggtcgtttgatgcaggcgttgccgtcgggtggtgcgatggttggggtggcggcgtcggagggtgtggtgcggccgctgctgggcgagggtgtggtggttgcggcggtgaatggtcccgagtcggtggtgctgtcgggtgatgaggatgcggttgaggcggttgtggatgtgttggctgggcgtggggtgcggacgcggcggttgcgggtgagtcatgcgtttcattcggctcgtatggacgggatgctggcggagttcggtgaggtgcttcggggggtggagttccgtgccccgagcgtgcccgtggtgtcgaacgtgtccggtgcggtggcgggtgaggagttgtgttcgccggagtattgggtgcggcatgtgcgggagacggtccggttcgccgatgggctggatactctccgtgagctgggtgtgggttcgttcctggagttggggccggacgggacgttgaccgccttggcggatggcgatggtgtgcctgtcttgcgtcgggatcgtccggagcctctgaccgctatggcggctttgggcgggctgtacgtccggggtgtccagatcgactgggatgcggtgttcccgggtgctcggcgggttgatttgccgacgtatgccttccagcgtgagcggttctggttggagccgtcccctgagcggcccacgacgagcgtggttgacgcggcgttctgggatgcggttgagcgtggggatctcggttcgttcggcatcgatgccgagcagccgctcagcaccgccctgcccgccctctcgtcctggcggagggcgcggcaggagcagtcggtgattgatggctggcgttaccggctcggttggatgccgattccggcggtgtccggggaggtgggcctcaccggtacctggctggttgtggtcgagccgggtgcggacggtactgatgtggctgtcgcgttgcggtcggccggggccggtgtcgaggttgtgacgtcggcggagctgagcgctggtccggttgcgggtgtggtgtcgttggtgtcggtcgaggcgacggtgtcgttgctgcacgtccttgtggcggccggggtcgatgcgccgttgtggtgtgtgactcgtggtgcggtctcggtggtcgacggtgacttggtggatcctggccaggcgggagtctggggtctgggccgtgtgatcggtctggagcatccggatcgttggggcgggctgatcgacttgcctggcgaactggacgatcgcgcggggaatgcgctggtaggcatccttgccgggggcaccggtgaggatcaggtggccatccgtgtcaccggcatatggggtgcccggctggtgcgggcgacgccggtcccgatcggtgacgcgggtggtgaggctgcggccgcgtggcgtgggcgtggtaccgcgctggtcaccggtggtacgggggcgctggggcgccaggtggcgcgctggctggtggacagtggtctggagcgggtcgtgctgacgagccgtcgggggggcgaggcgcccggtgccgtcgagctggtggctgagttggggagccgagtgcgtgtcgtggcctgtgatgtcggcgatcgtgaggagcttgcggctcttttggcgatgctcccggatgtgcggaccatcgtgcatgcggcgggtgtcctcgacgacggggtgctcgaatcgctgacgcccgagcggatccgtgaggtgatgcgggccaaggccgacggcgcgcggcatctccacgagttgacccgtgacatcgacctcgacgccttcgtgttgttctcgtcggctgccgggaccgtgggtaatgcgggtcaggggagctatgcggcggccaacgccgtcctggacgggctggcgtggcgtcgccgggccgagggcttggtggccacatcggtggcctggggagcctgggccgacagcggcatgggggctgggcacgcacgggccatggcaccacggctggcgctggcagcccttcagcgagcgttggacgacgacgagaccgcactcatggtcgcggacgtggattggtcgagcttcggctcccggttcaccgccgtacggccgagcccgctgctgagcgaactgctgccccgctccagcgcgccggtggaaccggtcgaggcactcgccacccggttgcggggcatgtcgcggatcgagcgcgatcgggcggtgctggagctggtccgtgcccaagtggcggccgtgctgggacatgcgaagcccgcttcggtcgacccctcgcggaccttccaggaagtcggcttcgactcgctgaccgcggtggagctgcggaaccggctggccactgccaccggcgtaccgttcccggggtcggtcatcttcgactatccgactcccacggcgctcgccgaccatgtccgggcccggttcgttccggacacggacaacgacgaggacgggggcggcgcgacgtccgtgctcgacgagctgaccaggctggaagccgtgctgtccgacctgtccccgagcgacgtggccggtgccgaggtcgccgcgaagatcaagagcctgctgtcccactggggagcggccaccaacagtgacatcgacatggattccgcgacggacgaggagatgttcgacctcctcggcaaggagttcgggatctcgtgaacctgccgtcgagttcgtctccgagtgagtccagcaccgcgttgagagggccgtcctgtggagaatgaagagaaacttcgtcattacctcaaagaggtcacgaaggatctgcggcagacccgccagcgcttgcaggacgtcgaggcgaagagccgcgagcccatcgcgatcgtcggcatgagctgccgtttccccggtggcatcgcaacgccggaagcgctgtgggacctggtgcgcgagggcggcgacgcggtgtcggagttcccggccgaccgcggatgggacacggagggcctctacgaccccgcgggcggctccgggaagtcggtcacccgctacggcggattcctgcgcggcgtcgccgatttcgacgccgcgctcttcgggatctctccccgtgaggcgatcgcgatggacccgcagcagcggctgatgctggagacctcctgggaagcgttcgagcgggccggtgtcaaccgtgacgcggtgcggggcagccggaccggggtgttcatcggcaccaacggccaggactacgcgacactgctcagcgctgcccgggacgatgtgcaaggccacctcggcacgggcagcgcggccagtgtgctctcgggacgggtcgcctacaccttcggtctcgaagggccgacggtcaccgtggacaccgcgtgctcgtcctcactgatcgccctgcacctggccgtccaggcactgcgcaacggcgagtgcgagctggcgctggcgggcggcgtcacggtgatgacgacgacgaacaccttcgtcgagctgtccaagcagggcgggctggcgccggacggccggtccaaggcgttcgcggcggcggcggacggcaccggctggggtgagggcgccgggatgctgctggtggagcggctgtccgacgccgaacggcacggtcaccccgtgctggcggtggtgcgtggcaccgccgccaaccaggacggcgcgtcgaatgggctgaccgcgccgaacgggccctcccagcgccgggtcatccgcgcggcgctgtccaacgcccagctgtccacgggcgatgtcgacgtggtggaggcacacggcaccggcacccggctcggcgacccgatcgaggcacaggccctgctcgacacctacggtcaggaccgggaccggccgctgtggctcggatcggtcaagtcgaacctgggacacacccaggccgccgcgggtgtcgccggggtcatcaagatggtgctcgccatgcgccacggtgtgctgccgcgcaccctgcacgtggatgaaccgaccccgcatgtggactggtccgccggggcggtgcggctgctcaccgagcggaccccgtggccggaggccgaccggccgcgcagggcgggcgtctccgccttcggagtgagcggcaccaacgcccatgtgatcgtggagcaggcatcggaggccgagcccgtcgagccgccccgggccgaaccggtgacggtgccctgggtgctctcgggccagggcgaggccggtctgcgggccttcgcggcccggctcgccgatgtggccaccgaagcgcaccccggcgacctcggatggaccctggccaccacccgctcggcgctgccgcaccgtgcggtggtgatcggatccacaccagaggaactgcggagcggcctcgcggcggtggccgccggagagccggcctcgaacgtggtggagggagtggccggctccgacaccggcgtggtcttcgtcttcccgggacagggctcgcagtgggccggtatggccgtggaactgctggactcctccccggccttcgcccgccggttcgccgaatgcgcccgtgccctggagacacacctcgactggtccatcgaggacgtggtgcgttccgcgcccggtgcgccctcgctcgacctcatcgaggtcgtccagccggtcctgttcaccatgatggtgtccctcgctgagctgtgggcctcctacgggatcactccatcggccgtggtcggccactcccagggcgagatcgcggcggcctgtgtggccggggcgctgtcgctggaggacgcggccaaggtggtggtgttgcgcagccgcctcttcgccgaaacgctggtgggcaacggcgccatcgcctcggtcgccctgcccgcggaacaactggccacccggatcgagccgtggggcgagcgcctcgtggtggccggggtgaacgggcccgcggccgccacggtggccggcgatccccagagcctcgaggagttcgtcgccgcatgcgcggcggacggcgtacgcgcccgcgtcgtgcccgccaccgtggcctcccacggcccgcaggtggaaccgctgcgggaacggctgctcgccctgctggccgacgtggcgccacgccagtccaccgttccgttctactccacggtgaccggcggactcctggacaccaccgaactcgacgcggactactggttctggaacgcccgtaagccgatcgacttcctcggcgcgctccgggcgctgttcgccgacggccaccgcgtcttcgtggagtcgagcacccaccccgccctgaccatgggggtccaggacaccgcggatgcctccggcgagtccgtggaggtcaccggctcgttgcggcgtggcgagggcgggctcgaccagttccactcggccgtggcgcggctgcatgtgcacggcgtacgggtggactggtccgcggccttcggggcggcgcggcgggtggagctgccgacctaccccttccagcgggagcgttactggctgacgccccggcccggccagggtgacgcctccgccctggggctgggtgcgctcgaccaccccctgctgggggccacggtcgtgctgcccgagtccggcggttgcctgctcaccggtcggctgtccctggccggacagccgtggctggccgatcacgccctctccggtgtggtgttgctgccggggacggggtttgtggagttggtgttgcaggcggggttgcggtgggggtgtggggtggttgaggagttgactttggaggggccgttggttcttccggagcggggtgaggttgaggttcaggtttcggtgggtggtgtggatggggccgggtgtcggtcggtgtcggtgttttcgtgtcgtgggggtgagtgggttcggcatgcggtgggtgtgcttggggtgggggatggtgcggtgccggtggcggaggtgtggccgccggtgggtgcggagcgggttggggtggagggggtttatgaggcgttggcggagcgggggtatgcgtacggcccggtgttccaggggctgcgggacgcctggcgccggggagacgaaatcttcgtcgaggtggcggtggcccaggaggcacgggcggacgcggcgcggtgcgcgatccatcccgcgctgctcgacgccgcgctccacggggtgcgattcggtgacttcgtatccgacgacgaccaggcttatgtgccgttctcctggaccggcgtcacgctgcacgcggtcggtgcgacggtcctgcgcgtcacactgtccccggcaggacgcgacgcgatcgccctccgggccacggacaccaccggtgcgccggtcctgtcggcacgctcactggccctgcgaccggtctccgcccagcagttgaacgacacgcgggggagcaggactgacgccctccatcgggtggagtgggtggacgcgtccggaaccgtggcggtggggggtgaggtggcgccgcggactgaggtggtgcgggtcgtctccgagggtccggatgtggtgggtgaggcgtacgggcatgtgcttgaggttctggagcgggtgcaggcgtgggtggcggatgaggacctggcgggtgagcggttggtggtggtgacgcggggcgctgtcgacacgggtgatggtgtggcggacgtggctggggccgcggtgtggggcctggtgcggtccgcgcagtcggagaacccggggcgtctggtgctggtggacaccgatgacctggacggcgtcgacagtctgcttcccgggatgctggctctggatgaggagcaggtgctggtgcggtcgggtgcggtgcgggtgccgcgtctggctcgggtgccggcgccgggtgaggtatcgggagggtttggttccggtgcggtgttggtgacgggtggcactggtgtgctgggcggtctggtgtcacggcatctggtggcgcggcatggggtgagcaggctggtgctgctgtcgcgtcgcggtgcggaggccgaaggtgcggcggagttgcgggaggagctggaggccgcgggcgccgaggtggtgatcgcggcgtgtgatgccgcggatcgtgaggctctggccggggtgttgtcggggttgtcggcggacttcgccttgagcggtgtggtgcatgcggcgggtgtgctggacgacgggttgctcacgtcgttgacgcgtgagcgggtcgagccggtgttgcgggcgaaggtggacgcggcgtggaacctgcatgagctgaccacgggcatggatctgtcggcgtttgtgctgttctcatcggcggcgggtattctgggcaacgcgggccagggcagttatgcggcggcgaacgggttcctggacgcgctggcggctcatcggcgggcgcggggactgcccgcggtgtcgatcgcgtggggcttctgggaagcacgcagcgagctgacccagcacctgtcggccgacgatctggcgcgtgcccacgcggtgccgatgcccacctcccaggcactggatctgttcgacgcgacgctcgccgccgacgagccgatggtgctggccgcacccctgaacccgcaggcatggtcggacgccggccacctgcctcccgtcctgcgcgatctggtccggccgcggatccggcgcgcggcggagacaaccggcgcccccgaatcggcctccgcgctcggacaccggctggccgccgtcgaccgctccgagtgggaccaggtcgtacgcgaactcgtgcgcaatcacatcgcggcggtgctgcgccatgcctccggggagtcggtggacacctcgcggacgttccaggagatcggcttcgactcgctgaccgccgtggaactgcgcaaccggatcagcgccgccaccggcgtacggctgcccgccaccgccgtgttcgactacccgacaccgcaagcgctggccgagtacctgctcgccgaagtcctcgggaaggacagcgccgccgccgcgacacccgtcggaaccgccctcgtcgccgacgatcccatcgtcatcgtcggaatgagctgccgctaccccggcgggatcacctcgccggaagcgctgtgggacctggtgcgctcggacggcgatgccatatccgtcctgccggccgacagaggatgggacctggacggcctctacgacccggatccggaccgcaccggtacgtcgtacgcccgcagcggtggattcgtctacgacgcggccgagttcgacgccgccttcttcgggatctcgccgcgcgaggccgccgccatggacccgcagcagcggctgctactggaaacctcatgggaggcgttcgaacgcgcgggcatccccgccacctccgtcaagggtgagcggatcggcgtgttcaccggggtgatgcaccacgactacctcacccgcctgtcgaccacaccggacgccgttgagggctatctgggcacgggcgcggcagcgggcgtcgcctcgggccgcgtggcctacaccttcggactcgagggcccggcggtcaccgtggacaccgcctgctcgtcgtcgctggtggccctgcacctcgccgtacaggcgctgcgcctcggcgagtgctcgctcgcgctggccggtggtgtgacggtgatgtccacgcccaccgtcttcgtcgagttctcccgccagcgcgggctcgcgccggacggcaggtgtaaggcgttcgcgggagcggcggacggcaccggcttcgccgaaggcatcggcatgctgctggtcgaacggctctcggacgcacggcgcaacggacaccccgtcctggccgtggtgcggggcagtgcggtgaatcaggatggtgcgtcgaatgggttgacggccccgaatggtccgtcgcagcagcgggtgatccggcaggcgctggcgagcgcggggctgtccacggtggatgtggacgcggtggaggcgcacggtacgggtacgacgctgggtgatccgatcgaggcgcaggcgttgctggccacgtacggtcagggccgggattcggaccggccgttgctgctggggtcgatcaagtcgaacatcggtcacactcaggcggccgccggtgtggctggtgtgatcaagatggtgatggcgatgcgccacggcgtgctgccgcagagcctgcacatcgatgagcccactccccacgtcgactggtccaccggcgcggtggagctcctgagcgaacagacggcatggccggaggccgggcggccccgccgggccggggtgtcgtcgttcggcatcagcgggacgaacgcgcacctgatccttgagcaggctccgctgccgacggcagcggagcggcccggtgacgccgagcccgttccggtcgagcctgccgcggtggtcccgtggatcgtctcggggcgcgaccggcatgccgtgcgcgcgcaggcggaacgactgcgcgcacacgtggtgagccaccctgaccggagggtggcggacatcggtttctcgctgctgaccagccgcgccgtgctggagcaccgagcggtggtactcggcggtgaccatgccgaactgctggccgggctgacggccctggcacgggacgaacccgcaccgggcgtggtggaggccctggacgcggccgagccggggcgcaaggtggtgttcgtcttccccggtcaggggtcgcagtgggccgggatggcgctggaactgatggagtcctcgcccgtgttcgcacggcggatgggcgagtgcgccgatgcgctggctccgctggtggagtggtcgctgccggacgtgctggcggatgagcgagcgctggcccgtgtcgatgtggtgcagccggtgctgtgggcggtgatggtgtcgctggccgagctgtggcgttcgtacggtgtggtgccgtcggcggtggtgggtcactcgcagggtgagatcgcggcggcgtgtgtcgcgggtggcctgtccctggcggacggggcaagggtggtcgtgctgcgcggcaaggcgctgctcgccttgtcgggccggggcggaatggtgtccgttccggtgcccgccgaccggctgcgggaccggcccggggtctccatcgcggcggtgaacggcccatcctcgacagtggtgtccggcggcgacgaggtgctggacgcggtgctggcggagttcccggccgccaagcgcatcccggtggactacgcctcccactcgccccagatcgacgacatccgggacgaactgctgaaggccctggcgccgatcgagccgcgcaccgcggcgatccccttccactccacggtgaccggacggcccatcgacaccgccgacctggacgcggactactggtatcgcaatctgcgcgagaccgtggagctcgagcgggtcatccgtacggcggtcgaggacggccaccacaccttcatcgagatcagcccccacccggtgctgaccacgggcctgcgcgaaacactcgacgacgcggacgcgcacggcggcctcgtactggcctcactgcgccgggacgacggtggccctacccgcttcctcaccgccttggccgaggcgtacgcacacggcgtcgaggtcgactggctgccgctgttcccgggcgcccgccgggtggatctgccgacgtacgccttccagcgcgagcgctactggctggacgcgcccaccgccgaggcccccaccagcgcgatcgacgcggaattctgggccgccgtcgagcgcgaggacctcgagtcgctcgccgcgacgctgcgcgtcgacgggcagccgctgcgcgaagtgctgcccgccctgtcccagtggcggcgcgaacgccgtgacgtctccaccatcgactcatggcgttacacgatccggtggaagccgctcaccccgcccgccacttcaccgaccggcacctggctggtcgtggtctgccatgccgaggccgggcacgagtgggtcgcgggggtgaccgacgcgctgacccgtcacggtgccgagccgctcgtggtcgttctcggcgagcccgaactggaccgtgccgcgctggccgcccggctgggcggcgtactggccgacacccccaggatcagcggtgtggtgtcgctgaccgcgctggacgagagcccgcacccggcgtacccctcggtcccccagggatacgcgatgacgctgctgctctcgcaggcgctcggggacgccagggtggaagctccgctgtggtgcctcacccagcgcggcgtctcgctcggcgatgccggaggcagtggcagtggcagtggcactggcgacggcaggggcaagggcaagggtgatgtggccgtcagccggaagcaggccctgacctggggtctcggcaaggtgatcgctctggaacagcccctgcgctggggcggtttgatcgacctgccggagggcgtggccccgcatacccaggactaccttgccggtgtgctgtccggcacctcggacgaggaccaggtggcgatccgcccgacggggctcttcggccgtaggctggcccacgcgccggcccgcgagcgcggcgggggctggcaaccccgcggcaccgtactggtcaccggtggcaccggagcgctgggcggccatgtcgcccggtggctggccggccagggggctgaacacgtggtgctgaccagtcgccggggcatggccgcgcccggcgccgagcggctggccggggagctggaggcgctcggcgcccgggtgacggtggcggcgtgcgacgtcggtgaccgggacgccctggccgggttgctggccgaggtcggcccgctgaccgctgtggtgcacaccgcggcggtgctcgacgacggcacgctgaactcgctcaccaccgaccagctgcaacgcgtgctgcgcgtcaagaccgacggcgcggtgcatctgcacgaactgacgcgggacatggagctgtccgcgttcgtgctcttctcctcgctgtccggcactctgggcgcacccggtcagggcaactacgcacccggccatgtcttcgtggacacgctggccgagcagcggcgggccgagggcctggtggccacctccatcgcctgggggctgtgggccggtgacggcatgggcgagggcggtgtgggcgacgtggcccgccgccatggcgtaccggagatggcgccggagatggcggtcgccgccatggcacgcgccgtcgagcaggacgacaccgtcgtcacggtggccgagatcgactgggaccggcactacgtcgcgttcaccgcgacccgccccagcccgctgctgtccgacctccccgaggtgcgtgcgctggtcgacgccggagtcggccaggagagcgccgagccgggccacgagcgctcggaattcgcggagcggctcgccgggatggccgagaccgaccggaaccacgcgttgctggacctggtccggcgccatgtcgccgtcgtactcggacacaccggtccggacgcgatcgaccccggccgggccttccacgagatcggcttcgactcggtcaccgcggtcgaactgcgcaaccggctcaaccgggccaccggcctacggctgcccgccaccgtgacgttcgaccagcccaccccgctggcgatggcgcagtacctccgcggcgaactgctgcacgacggccaaggccgatcggcccccgccctcccggtccgcgcgaccggcgcggtggacgacgagcctatcgcgatcgtggggatgagctgccgcttccccggggacgtcgcgtcccccgaggacctgtggcggctgctcgccgacggttccgacgccatcggcgagttccccgagaaccggggctgggacaccgcgcacctcttccacccggaccccgaccaccgaggcacctcctccacccgagcggccgcgttcgtctccggggccggtgagttcgacgccggattcttcgggatctccccgcgggaagcggtggcgatggacccgcaacagcggctgctgctcgaagtgtcatgggaggcgctggagcgggccgggatcgaccccacgaccctgcggggcagcgagaccggcgtgttcacggggacgaacggtcaggactacgcgtcgttgctgaaggcggacgagacgggtgacttcgagggccgggtgggcaccggcaactcggcatcggtcatgtccggccggatctcctacgtcctcggtctcgaaggccccgcgctgaccgtggatacggcgtgctcgtcgtcgctggtggcattgcacctggcggtgcgggccctgcggtcgggcgagtgctcactggccctggcgggaggcgcgagtgtcatgacgaccgccggcatcttcgtggagttctcccgtcagcgcgcgttggcggccgatggacgctgcaaggcgttcgcggcggcggcggacggtaccggctggggtgagggtgccggaatgctggtggtggagcggttgtcggatgctgagcggcttgggcatcgggtgttggcggtggtgcgtggttctgcggtgaatcaggatggtgcgtcgaatggtttgacggcgccgaatggtccgtcgcagcagcgggtgatccggcaggcgctggcgagcgcggggctgtccacggtggatgtggacgcggtggaggcgcacggtacgggtacgacgctgggtgatccgatcgaggcgcaggcgttgctggccacgtacggtcagggccgggattcggaccggccgttgctgctggggtcgatcaagtcgaacatcggtcacactcaggcggccgccggtgtggctggtgtgatcaagatggtgatggcgatgcgccacggtgtgctgccgcagagcctgcacatcgatgagcccactccccacgtcgactggtccaccggcgcggtggagctcctgagcgaacagacggcatggccggagaacacacggccccgccgcgccggggtgtccgccttcggagtgagcggcaccaacgcgcatgtgattctggagcaggcccccgagccgaccgccgcccagcccgaactctcgccggaacgcgacgaaatgagggccgtgccgtgggtggtgacgggtgcgagcgaggccggagtccgcgcacaggccgcgcgcctcatggcctttgtcgacgaccggccggaactccgcccggtgaacatcggctggtcgctggcctcgacccgcgcggccctgtcacaccgtgccgtggtcgtaggtgctgaacgtacggaactgctgcgtgagctggaggccgtggccagtggcagcgtcacggtcggcgaggcccgcacgcattccggggtggtgttcgtcttcccggggcaggggtcgcagtgggttgggatggcgttggagttggtggagtcgtcgccggtgttcgcggggcggatgcgtgattgtgcggatgcgttggccccgtttgtggagtggtcgttgttcgatgtgttgggtgatgaggtggcgcttgggcgggttgatgtggtgcagccggtgttgtgggcggtgatggtgtcgttggcggagttgtggcgttcgtttggtgtggtgccgtcggtggtggtggggcattcgcagggtgagatcgcggcggcgtgtgtggccgggggtctgtcgttggaggacggtgcccgtgtggtggccttgcggagcagggcgttgctggctctgtcgggtcggggcggcatggtgtcggttccggtttctgctgaccggctgcggggtcgtgtggggttgtcggtggcggcggtgaatggtccggtgtcgacggtggtgtcgggggctgttgaggtgctggatggggtgctggcggagttcccggaggcgaggcggattccggtggattatgcgtcgcattcggtgcaggtggaggggatccgggagggtttggcggaggcgttggcgccggttcggccgcgtacgggtgaggtgccgttctattcgacggtgaccggccggctgatggacaccatcgagttggacgcggagtactggtaccggaacctgcgcgagacggtggagttccagagcgcgatcgaggggctgctggagcttggccatacggtgttcgtcgaggccagcccgcatccggtgctgaccattggcatccaggacaccgccgacaccaccgacaccgacatcgtcgtaagcgggtcactgcgccgcgacgacggcggtcctgtccgcttcctcagcaccgtcgggcgactgttcaccgagggcgtgccggtggagtggcagccgctgttcgccgcggccggggcgcgaaaggtcgatctcccgacctatgcgttccagcatgagtggttctggctggatccggtgcgcggcgcgagtgatgtgggcggcgcgggccttgccggtctcgctcaccccttggtgagcgcggtgttgccgctgcccgaatccgatggctgtgtgctgaccggctcgctctcctcggccacccatccttggctgcgtgaccacgccgtgctggacaaggtgttgctgccgggcaccgggttcgtggaactggcccttcaggccgggctgcacctgggctgccggacgctggatgagctgaccctgcaggcgccgctcatgctgcccgcgcacggagacgtacagatccaggtggcggtcggcggaccggacgacagcggccgccggccggtcacggtgtactccaggccgggcaaggaccggacctggatgcggcacgccaccggcagcatcagccccgtcggtgaaacggccaccgtggaccgggcggtgtggcccccggtcggcgccacaccggtcgagctcaccgatgtctacgccgagatgagcacgcacggttacgcgtatgggcccgtcttccaggggctgcgcgccgcatggcgacgtggcgacgaggtgttcgccgaggtggtcctgcccgagacggccgagagcgacgcgggtcgttgcgccatccaccccgccctcctcgacgccgccctgcacggtgccggactgggcacgttcgtgaccgaaccaggccgaccgcaccttccgttcacctggaccggtgtcaccctgcacgccgtcggtgccaccaccttgcgggtcgtcctgtcgcccgccgggccggacgccatctcgctcctggccatggacggcacgggagcgccggtgctgacggcggactctctggccctgcgcccggtgtccgagggcgggctcggcggctcccacgacgactcgctgttccgcgtggactggaccgagctcaccctggacgcctcggacgcctcggacgcaccggaggtgtcggatgaagcggccttcccggtcgtcgagtccgtggcccagctggccggggtggcggcggcccggagcgggcgcggggccgtggtgttcaggctttccaccacggagaccacaggaggcgccgccgaggagagcccggaggacgtctacgcgctcaccagccgtgtcctcaaggtcgcgcaggcgtggttggcggacgaccggttcggggacgcccgcctcgtcgtggtgacgcggggcgcggtcgcgaccacgcccggagagaacccggagagccttgccgccgccgcggtctggggcctcatccgcaccgcgcagaccgagaaccccggccgtttcgtcctcgtggacacggtggacgaggatccgtcggcgttgccgggggtgctcgccaccgatgagccacaggtggcgatccgggcggggaaggcgctggtgcccaggctggtacgggccacctcgtcggcgttgccggtaccagctgagacggacacctggcggctggagaccgacggtcagggcactctggagaacctggtcctctcgccccgcgccgaggcgtccaggccacttgccgcacatgagatccgggtggccgtgcacgcggccggggtcaacttccgcgatgtactgctcgctttggggatgtacccggacaaggccggtctgctgggcagcgaagccgccgggacggtgctggagatcggctccggagtagtgggagtggcaccgggagaccgggtgatgggtctgttctccggtgccttcgcgccggtggcgatcaccgatcaccgactggtggcaccgatcccggaggggtggtccttcccgcaggccgccgccaccccgatcgccttcctcacggcgatgtacgccttgatcgacctggccgaagtgcggagcggcgagtcggtgctggtgcacgcggcggccggtggcgtcgggatggcggcagtgcaggtggcgcgctggctgggcgccgaggtgttcgccaccgcgagcccggccaagtgggatgcggtgcgcgcatgcggggtcgccccgcggcggatcgcttcctcccgctcgccggagttcgcggaccgcttccgctcggacgcaccggacggtgtggatgtcgtactcaactcgctgaccggtgaactcctcaacgcgtcgctcggactgctgcgtcccggtggacggctgatcgagatgggcaggaccgaactccgggacgcacaggaggtgatggcgcgccacggtgtgtcgtaccgggccttcgaactgctcgacgccggtcccgaccgtatcggccgactgctcaccgagctgctcgccctgttccaccagggcgtgttcaccccgctgccactgcgcgtccaggacgtacggcaggcgagtgacgctttccgccacctctcccaggcgcgccacatcggcaagctggccctcaccatcccgcgaccgttgtccggcggcaccgcactgatcaccgggggcaccgggacactgggcggtctggtggctcgccaactggtgcgggagcacggcgtgacggagctggtgctggccagccgtcgtggtgacaccgctccgcaggcggcggagctgctcaccgagctggaggccgccggggcgcgggtgcgggtggccgcatgcgatgtgtcggaccgggacgccatcgccgcactcgtcgcctcgctgccgaacctgcggagcgtggtgcacacggccggtgtcctcgacgacgccgtgatcgggtcgctcaccccggagcggctgcggacggtactgcgtcccaaggcggacgccgcatggcatctgcatgaactgacccgggaccgggaccttgccgagttcgtgttgttctcctcggcggccggagtactcggtggcccagggcagggcaattacgcggcggccaacgccttcctggacgcgctggccgcgcgccgccgggcacagggactgcccgcgacctcgctggcctggggcttctgggagcagcgcagcggactgaccgaacacctgaccaccgatcggctcgcccgggccggcgtcctgccgctgtccaccgacgaggggctggtcctcttcgacgacgcccgcgcgaccggcgacaccctgctggtgccgatgcgttacgaaccgtcctcgccgggccctgagccggtacccgccctgctgcgtggcctcgtacgcgctccgctcgcccgcgcccttccgggcccggccgatggtgtgggcagcggtgtggcggagggcctcacagggctggcggcggacgaacgcctcggcgcactgctcgacctggtccgccgggaggcggcggccgtgctcggccacggcggtccggaatcggtgacaccccagcgtccgttcaaggaactcggcttcgactcgctctccgccgtggaactgcgcaaccggctgcgcgcggcgaccggccgacggctggaggccacccttgtcttcgaccaccccactccggccgtgctcgcacgccacctcgacgccgagctgttcggcgccaccgacgtggcggcgcccgtaccagcaccggcggtcgcgcacccggccgacgagccgatcgccatcgtcggcatgagctgtcggctcccggccggggtggactccccggaggcgctgtggaagctgctggtgagcggcacggacgcgatatcggagctgccccccgaccgcggctgggaccttgacaggctctacgaccaggatccgagccggcccggtacgacatacgccaagaccggtggcttcctgaagaacgcggcggacttcgacgcgggattcttcacgatctccccccgagaggcgctggccgcggatccccagcaacggctgtggctcgaggcgtgctgggaagccttcgaacgcgccggtatcgatccgctcgccctgaagggcacccgaaccggggtgttcgcgggtgccgtttcgacgacgtacggcgcgggtcaggccgccactccggacggctccgaggggtacctgctcaccggcaactccacctccgtgatctccggccgcgtggcctacaccctcggcctcgaaggccccgccgtcaccgtggacaccgcgtgctcgtcctcgctggtcagcgtgcactgggcgtgtgagtccctgcgccggggcgaaagcacactggcgctggcgggcggtgtggcggtgatgacgacaccggacctgctggtcgaattctcccgccagcgcggactcgcaccggacgggcggtgcaagtcgttcgccgccgccgctgacggcacagggttcgccgaaggcgtcggggtgctcgtcctggaacggctgtccgacgccacgcggaacggccaccaggtgctggcggtgatccgcggctccgccgtcaaccaggacggcgcgtccaacggtctgaccgcgccgaacggcccctcgcagcagcgggtgatccggcaggcgctggtgaacgccggactcgcctcccaggatgtcgacgtggtggaggcgcacggtacgggtacgacgctgggcgaccccatcgaggcgcaggctctgctggccacctacggccaggaccgggatccggatcggccgctgctgctgggctccgtgaagtccaacatcgggcacacccaggcggccgcaggtgccgccggactcatcaagatggttctggcgctgcgcaacggcgtactgccgcgcaccctgcacgtcgacgagccctccccgcacgtcgactggtccgccggggccatggagctgctgaccgagcagaccgcgtggcccgaccgggaccacctgcgccgggccggggtgtccgcgttcggagtgagcggcaccaacgcccatgtgatcctcgaacaggccccggagccggatgagaacggcgaaccggacaccgtccggtcgtggttgcccgcggtgccctgggtgctgtcgggcgcgggagcggccgggcttcgggcccaggcccagcggttggcgtccttcgtgcgggagaaccccgggctcgaccccgtggacgtgggctggtccctggtcgcgacccgcgccgccctgtcgcaccgagccgtcgtcgtgggcgcggaccgcacggaactgctgcgcgagctggccgcggtggaatccgtgggcgccgccgaggcggagcgcgacgtggtgttcgtcttcccggggcaggggtcgcagtgggttgggatggcgttggagttggtggagtcgtcgccggtgttcgcggggcggatgcgtgaatgtgccgatgcgctcgccccgtttgtggagtggtcgttgttcggtgtgttgggtgatgaggtggcgctcggtcgggttgatgtggtgcagccggtgttgtgggcggtgatggtgtcgctggcggagttgtggcgttcgtttggtgtggtgccgtcggtggtggtggggcattcgcagggtgagattgcggcggcgtgtgtggcgggtgcgttgactttggaggatggggcgcgtgtggtggccttgcggagcagggcgttgctggctctgtcgggtcggggcggcatggtgtcggttccggtgtccgctgatcggctgcggggtcgtgtggggttgtcggtggcggcggtgaatggtccggtgtcgacggtggtgtcgggggctgttgaggtgctggatggggtgctggcggagttcccggaggcgaggcggattccggtggattatgcgtcgcattcggtgcaggtggaggggatccgggagggtttggcggaggcgttggcgccggttcggccgcgtacgggtgaggtgccgttctattcgacggtgaccggtcggttgatggacaccgtggggctggacggggagtactggtatcggaatctgcgtgagacggtggagttccagagcaccgtcgaagctctgatcggccagggccacacggtgttcgtcgaggccagcccgcatccggtgctgaccgtcggcgtccaggacaccgccgacgcgatggagacccccatagtggccaccggttcgcttcgccgggacgagggaggcgtacgacggttcctgacgtcactggctgaggtatccgtccatggcatcgaggtcaactggcagacggtcttcgacggcaccggcgctcggcgagtcgacttgcccacctacgcgttccagcgtgagcggttctggctggtgccatcgacgggcacgggcgacgcgtccgggctgggcctgggcgccgttgaccatccgctgctgggcgcggcggtgccgcttccggacgcggacggctgtgtgctgaccggtgcgctgtcgctggccgggcagccatggctggccgaccactccgtcctcggcatggtccttctgccgggcaccgcgttcgtggagctcgcgttgcaggcgggggcgcggttcggctgcggcactctggaagagctgacgttgcatgagccgctcgtcctgcccgagcgggagaccgtgcagctccaggtgtcggtcggaggctcggacgacttcggaggccgccccttcacggtgttctcccgctgtgagggtgagtggatacgccacgccgggggcaccctgcgtgtgggcgagcgtggcgatccgcccgcgaacccgtcggtctggccaccggccgatgcccggccggtcgatgtcgccgagttgcacacgacgatggccgagcggggctatcagtacgggcccgccttccagggcctgcggaaggcatggatccgtgacagcgaagtgtttctcgacgtcgcgttgcccgagcaggtgaggggcgacgcggcccgctgcggagtgcatcccgcgttgctggacgcggccctgcaaggcatcggcctcggcgccttcgtcaacgaaccgggccaggcccatctccccttctcctggagcggggtgaccctgcacgcggtgggcgccactgccgtgcgggtgacactcagcccggccggaccggacacggtggccatccggatggcggacaccatcggggcgcccgtgctgtccatcgacgcgctggcgatgcgtccgctcgcggagcagcggctgctcgaggcgggtggcagccgcggcgatgcgctgttccggctggagtggaaggagcttcccgtccccacgggggccaccggcccacgggcgcagtcctggggcctgctgggcggccacgacgagcctcgactgaccgcggcgctgaccgcggccggtgtgtcgccacaacgccatcgggacctcgcctccatcgaccaggtgccggatgtgctggtcctgtcgtgtccgcccgaggcggatggcggcccggccccggaagccacctcgtccgccctccgccgagtgctggaagtggtgcgggagtggctcggggacgcgcggtacaccgatgcccgactgatggtgctcacccgccgcgcggtggccacatccaccggtgacgacgtggaggatctggcggcggccgctgtacggggactcctgcgcaccgcacaacaggagaaccccgaccggctcgtcgtgatcgaccatgacgactcggaccttgaggtgctccccgtggtgctcgggacaggggagcccgaagcggccatccgggccggtaaggtgctggtgcccaggctggtcaaggcggccgtatcggaagggaaggcccctgcctgggacgccggcaccgtgctgatcaccggcgggacggggacactcggcggcctggtcgcccgccatctggtgaccacccatggcgcgcgtgacctggtgctggccagtcgcggaggtgacaccgcgcccggcgccgtggaactggccaccgaactggaggcgctcggtgcccgcatccgcgtcgccgcctgcgatgtggccgatcgtgcccagctgaccgcgctgctcgacaccattccggcgctgcgtgctgtcgtccacaccgcaggtgtggtggacgacggtgtcatcggctcgatgaccgccgaacgcgtggagaccgtcctacggccgaaggcgaacgcggcgtggcacctgcacgcgctgacccgccacctggacctggacgcgttcgtactgttctcctccgccaccggagtgctgggcagcgcgggacagggcaactacgccgcggccaacgccttcctcgacgcgctggccgtgcaccggcgcgcccaggggctccccgcggtgtcggtggcatggggcctgtgggagcggcgcagtggcctgaccgcacatctgtcggagcaggacgtggcccgtatgaccagcacgggcgccgttcccctctccgacgaacgcggtctcgagctgttcgacgccgcgtgccggagtggcgaacccacactcgtggccaccccgttgcaccttcgtgcggtggcggccaccggtacggtgccccacgtgctcagcgcgctggcaccgaccccgccacgccgggccgccgaggccggtgacggtggagtggctctacggcagagccttgccgagatgtcgggcgcggaacagagccagaccgtcctggggctggtacgcgggcaggtcgccgccgtgctgcggcacccggacccgtcggcgatcgacacggcgcggacgttccaggagatcggcttcgactcgctgaccgcggtggagctgcgcaaccggctgggcgccaccaccgggatcaggctggccgcgaccgcgatcttcgactatccgacacctgccacgctggcacagcacctgctcgccgagatcgtgccggagaccgccgacccggtcgcggcccggctcggcgagctggacaaggtggccgccatgatttcggcgatggccgaggacgacaccctgcgcgagcagttgtcctcgcggatggagaccatcgtcgcgatgtgggccgacctgcaccgtccggagcggccgggcacggttgagcgggacctcgaatccgcctcgctcgacgacatgttcggaatcatcgaccaggaactcgatgggtcatgagcagcgagaacgtccgaccggaaatcgaggggactggcacgcggatgtcgaacgacgaaaaggtactcgagtacctcaagaagctcaccgccgatctgcgccagacgcgtcagcgtctccaggacgtcgaggccaagagccgcgagccgatcgcgatcgtcggtatgagctgccgtttcccgggtggggtgagctccccggaagacctgtggcggctgacggagtctgcggtggacgcggtctccggtttccccacggaccgaggctgggacctggacggtctgtacgaccccgacccggatcgcgcgggccggtcgtacgcccgagagggcgcgttcatccccgatgcaggccacttcgaccccggcctcttcgggatctcgccacgtgaggcgctggcgatggatccgcagcagcggctgctgctggaggcatcgtgggaggccctggagcgggcgggtattcccaccgattccctgaagggcagccggaccggggtgttcgccggactgatgtcttccgactatgtctcgcggctgtccgcggtcccggacgaactcgaggggtacgtcggaatcggaagcgcggcgagcgtcgcctccggccgcgtgtcgtacaccctggggcttgagggcccggcggtcaccgtggacacggcgtgttcgtcgtcgttggtggcgttgcatctggcggtgcaggcattgcggtcgggtgagtgctcgctggcgctcgcgggcggtgtcacggtgatggcgacacccggcaccttcgtccagttctcccgccagcgcggcctggccgccgacggccggtgcaaggcgttcgcggcgggggccgacggtaccggctggggcgaaggcgtcggcatgctggtggtggagcggttgtcggatgctgagcggcttgggcatcgggtgttggcggtggtgcgtggttctgcggtgaatcaggatggtgcgtcgaatggtttgacggcgccgaatggtccgtcgcagcagcgggtgatccgtcaggcgttggcgaatgcccgtttgtcggcggtggatgtggatgcggtggaggcgcatggtacgggtacggcgttgggtgatccgattgaggcgcaggcgttgttggccacgtatggtcagggtcgggatgtgggtcggccgttgtggttggggtcggtgaagtcgaacatcggtcatacgcaggcggctgcgggtgtggctggtgtgatcaagatggtgatggcgatgcggcatggggtgttgccgcggacgttgcatgtggatgagccgtcgccgcatgtggattggtctgctggtgcggttgagttgttgacggggcaggtggcgtggccggaggtggatcggccgcgtcgggcgggtgtgtcggcgttcggggtgagtgggacgaatgcgcatgtgattgtggagcaggcgcctgaagtggcggagtctgaggctgaaggtgtggtgttgcctgctgtgccgtgggtggtgtcgggtgtgggtgaggtggcggtgcgggcgcaggtggagcggttgcgggcctttgcggaccggaatccgggtctggatccggtggatgtggggtggtctttggcgactggtcgtgcggggttgtcgcatcgtgcggtggtggtgggtgcgggtcgtggtgagttgttgggggctttggagggtgtgccggtggtgggtgttccggtggtgggtgggttgggtgtgttgtttgcgggtcaggggtcgcagcggttggggatggggcgtgggttgtatgaggggtatccggtgttcgctgcggtgtgggatgaggtgtgcgcgcagctggatcggtatttggataggccggtgggtgaggtggtgtggggtgatgatgccgggttggtcggggagacggtgtatgcgcaggcggggttgttcgcgcttgaggtggcgttgtatcggctgatcgcttcgtggggtgtgagggcggattatctgctgggtcattcgattggtgagttggctgcggcgtatgtggcgggtgtgtggtcgttggaggatgcggtgagggtggtggtggcgcgggggcgtttgatgcaggcgttgccgtcgggtggtgcgatggttgcggtgggggcgtcggagggtgtggtgcggccgctgctgggcgagggtgtggtggttgcggcggtgaatggtcccgagtcggtggtgctgtcgggtgatgaggatgcggttcaggttgtggtggatgtgttggctgggcgtggggtgcggacgcggcggttgcgggtgagtcatgcgtttcattcggctcgtatggacggcatgctggcggagttcggtgaggtgcttcggggcgtggagttccgtgccccgagcgtgcccgtggtgtcgaacgtgtccggtgtggtggcgggcgaggagttgtgttcgccggagtattgggtgcggcatgtgcgggagacggtccggttcgccgatgggctggagacgctgcgcgagctgggtgtgggttcgttcctggagttgggaccggacgggacattgaccgcgctggccgacggcgatggtgtgtcggcgctgcgccgggaccgtccggaaccgactgcggtaatggctgctttgggtgggttgtatgtccggggtgtggaggtcgactgggacgcggtgttcccgggtgctcggcgggtcgatttgccgacgtatgccttccagcgtgagcggttctggctggaaccggccgctgagcagcctgcgacgagcgcggtggacgcggcgttctgggacgcggtcgagcggggcgatgcggagattctcggggttgacgttgagcagccgttgagtgccgcgttgcccgcattggcgtcgtggcgacgggcgcggcaggaagagtcggtcatcgacgcatggcggtatcggctgacctggaccccggtcgcgggtctctcttcgcagctctccggcgtgtggttggtggtggtcgagccggacgaggcggagccggacgtcgtcgccgcgctgcggggcgccggcgccgaggtgcgtgtcgtaacgatcgatgagctggacgcgggcccggtcgcgggcgtggtgtctttgttgtcggtcgagacgacggtgtcattgctccaggcccttgtggcagaggggggcgatgcgccgttgtggtgtgtcactcggggtgcggtctccgtggtggacggggatgtggtggatccgcatgcgtcggccgtctggggtttgggccgtgtgatcggtctggagcatccggaccgttggggcgggctgatcgatctgcccaccgcatggggtgagcgaacctccggcatgttgtgctcggtgctttcgggcgccacgggtgaggaccacacagcgatccgtggcgacgaggtgttgggttgtcgtctgagccgtgcgacgacgtcggcaccggggccgtccactgcctgggaagcgtcggggaccgcgctgatcaccggtggaacgggtgccttggggagccatgtcgcccgatggctcgcggataccggcgtcgaagagatcgtgctgacgagccgacgaggcgcggacgctcccggagcacgggaactggtcgccgaactgtcggccatgggcgtatcggcccgcgtcgtggcgtgtgatgtggccgatcgggacgcggttgcggagctgatcgagaccattccggacctccgcgtggtcgtccacgccgcgggagtaccgagctggggtgcgttgagcacacttaccgcacagggccttcaggatgggatgcgggcgaaggtcgcgggagccatccacctggatgagctgacgcgcgatatgcgcttggacgcctttgtgttgttctcgtcggtggcgggggtgtgggggagcggtagtcagtcggcgtatgcggcggcgaacgcgtttctggatgggttggcgtggcggcgtcgtggtgttgggttggtggcgacgtcggtggcgtgggggatgtggggtggcggtggtatggcggttgggggtgaggagtttctggttgagcgtggtgtgtcggggatggctccggggtcggcggtggctgcgttgcggcgggcgctgtgtgatggtgagacggcgcttgtggtggcggatgtggattgggagcggttcgggccgaggttcaccgcgttgcgtccgagcccactgctgagcgagctgatccccgacaccgtcggctcgggggttccgctgggtgaattcgcggcccgtttccagaccatgtccgagggcgagcgcatgcgcgcggccgtcgagctggtgcgtgtttcggccgcggccgtgctggggcaccagggcccggaggccatcgatcccgtcaggacgttccaggagatcggcttcgactcgctgaccgcggtggaactgcgcaaccggatcgccacggctaccggtatccgcccgccggccacgatggtcttcgactatccgactcctgtggccctcgccgaatatctgagcgtggaattgctcggttcgccgcaggacagtgtgccgccgttgcaggtggccgcgccggacgacggtgatcccattgtcatcgtcggcatgagctgccgcttccccggggacgtcgagtctcccgaggatctgtggcggttgatcgactccgacggcgatgccataacggcctttccgacggaccgtggatgggacctgaccggcctcttcgacacggctgtgggggagtcggggacgtcgtatgcgcgtgttggtggcttcgtccacgacgcgggtgagttcgatccggccttcttcggtatctcgccgcgtgaggcgaccgcgatggatccgcagcagcggctgctgctgcacgcggcatgggaggcgttcgagcgggccggtatcccggccgcctcggtcaggggcagcaggactggagtgttcgtcggagcctcgccgcagggctatggcgccgccgaagcgtcggaaggctatttcctcaccggtagttcgggcagtgtcatttcgggtcgcgtgtcgtacacgctgggtcttgagggcccggcggtcacggtggatacggcgtgttcgtcgtcgttggtggcgttgcatctggcggtgcaggcgttgcggtcgggcgagtgttcgctggcgctcgcgggcggtgtcacggtgatggcgacacccactgctttcgtggagttctcgcgtcagcgtgggctggccgccgatggccgctgcaagtcctttgccgctggtgcggatgggacaggttggtcggagggtgttgggctgctgctggtggagcggttgtcggatgcggagcggcttgggcatcgggtgctggcggtggtgcgtggttctgcggtgaatcaggatggtgcgtcgaatggtttgacggcgccgaatggtccgtcgcagcagcgggtgatccgtcaggcgttggcgaatgcccgtttgtcggcggtggatgtggatgcggtggaggcgcatggtacgggtacggcgttgggtgatccgatcgaggcgcaggccctgttggccacgtatggtcagggtcgggatgtgggtcggccgttgtggttggggtcggtgaagtcgaatattggtcatacgcaggcggctgcgggtgtggctggtgtgatcaagatggtgatggcgctgcggcatggggtgctgccgcgaacgttgcacgtcgatgaaccctccccgcatgtggactggtcgtccggggcggtcgagttgttgagcgagagggctgcttggccggagatgggccgaccgcgtcgggcgggcgtgtcgtcgttcggggtgagcgggacgaacgcgcatgtggtgttggagcaggctcctggggcggtggaggagtctcggggcgagggtgttgcgttgcctgctgtgccgtgggtggtgtcgggtgcgggtgaggtggcggtgcgggcgcaggtggagcggttgcgggccttcgcggaccggaatccgggtctggatccggtggatgtggggtggtctttggtggccactcgttctgggttgtcgcatcgtgcggtggtggtgggtgcggatcgtgaggagttgctgggtgggttgggttcggtggtggtgggtgttccggttgcgggtgggttgggtgtgttgtttgcgggtcaggggtcgcagcggttggggatgggtcgtgggttgtatgaggggtatccggtgttcgctgcggtgtgggatgaggtgtgcggggagctggatcggtatctggataggccggtgggtgaggtggtgtggggtgatgatgccgggttggtcggggagacggtgtatgcgcaggcggggttgttcgcgctggaggtgtcgctgtatcggctgatcgcttcgtggggtgtgaggggggattatctgctgggtcattcgattggtgagttggctgcggcgtatgtggcgggtgtgtggtcgttggaggatgcggggagggtggtggtggcgcgggggcgtttgatgcaggcgttgccgtcgggtggtgcgatggttgcggtggcggcgtcggagggtgaggtgcggccgctgctgggcgagggtgtggtggttgcggcggtgaatggtcccgagtcggtggtggtctcgggggatgaggatgcggttgaggcggttgtggatgtgttggctgggcgtggggtgcggacgcggcggttgcgggtgagtcatgcgtttcattcggctcgtatggacgggatgctcgcggagttcggtgaggtgcttcggggcgtggagttccgtgccccgagcgtgcccgtggtgtcgaacgtgtccggtgcggtggccggtgaagagctctgctcgccggagtattgggtgcgtcatgtgcgggagacggtccgattcgcggatgggctggagacgctccgtgagctgggtgttggttcgttcctggagttggggcctgacgggacgttgaccgccttggcggatggcgatggtgtgcctgtcttgcgtcgggatcgtccggagcctctgaccgttatggcggctttgggtgggctgtacgtccggggtgtccagatcgactgggatgcggtgttcccgggtgctcggcgggttgatttgccgacgtatgccttccagcgtgagcggttctggttggagccgtcccctgagcagcccacgacgagcgcggcggacgcggcgttctgggatgcggttgagcgtggggatctcggttctttcggtatcgatgccgaacagccgctcagcgccgcactgcccgccctctcgtcctggcgccgccgtcaccaggagaggtcactcgtcgagtcctggcggtaccgcctcgactggtccccgatcggcaccgcttccgagcagccgagtctgcgcggcacgtggctggtggtgggcgagggcggagacgacgtggtcgccgtgctgcgggctgcgggggccgatgcgcgagttgtgacaatggcggagctgggcgaggtcgcggctgcgggtgtggtgtcgttgttgccggtcgaggcgacggtgtcactggtgcaggcactggggacggccggggccgatgcgccgttgtggtgtgtgactcggggtgcggtgtcggtggtcgatggtgatgtggtggatccggggcagtcgggggtgtggggtcttggccgggtgatccgtttggagcatccggatcgttggggtggtctgatcgatgtgccggtggtggtggatgaggaggccggggcttggttgtgccgggtgttgggtgggggtacgggggaggaccaggttgcggttcgtggtggtggggcgtggggtgctcggctggtgcgggtgtcgggctcgggttcgggatcgggtggggcggttgtgtggcggggtcgaggggcggcgttggtgacgggcggtacgggtgcgttgggtggtcatgtggcgcggtggttggccggtgctggtgtggagactgttgtgctggcgagtcgtcgggggatggctgcgccggatgcggagcagctggtcgcggagttggaggggttgggtgttgcggtgcgggtggtggcgtgtgatgtggcggatcggggtgcggtggcggagttgttggaggggattggggatttgcgtgtggtggtgcatgcggcgggtgtgctggatgacggtgtgttggagtcgctgacgtctgagcgggttcgtgaggtgatgcgggtcaaggcggagggtgcgcggtatctggatgagttgacgcggggttgggatctggatgcgtttgtgttgttttcttcggctgcggggactgtgggtaatgcgggtcaggggagttatgcggcggcgaatgcggtgttggacgggttggcttggcggcgtcgggcggaggggttggtggccacgtcggtggcttggggagcctgggccgacagcggcatgggggctgggcacgcacgggccatggcaccacggctggcgttggcagcccttcagcgagcgttggacgacgacgagaccgcactgatgatcgcggacgtggattggtcgagcttcggctcccggttcaccgccgtgcggcccagcccgctgctcggtgaattgctgggtggcgccgctcatcccgcgcccgcggtgggcgggttcgtcgaccggctacgggacctccccccggccgagcgggaacggacggtccttgagctcgtacgtggccaggtggccgtcgttctgggacatgccaccccgggggcgatcgacaccgcagcgacattccagtcagccggtttcgactccctgaccgcgatcgaactccgcaatcggctcatggcggccaccggagtgcagacacctgcctcggtcgtcttcgactaccccactccggaacttctcgccggccacctgcgggagcaactgctcggggcagggtcggcagcactctcgacgacggtcgccacggctccggtcgatgacgacccgattgcgatcatcggcatgagctgccgattccctggtggtgtcgactcgcccgaagagctgtggcggctcctggagtcggggacggatgccatttccgcctttccacaagaccgcggctgggacctcgtgggcggagtcgatggcgcgtcggtccgggcgggtggcttcctctacacggcggccgagttcgaccccgcgttcttcgggatctcgccgcgcgaggcgatcgcgatggatccgcagcagcggctgctgctcgaggcctcgtgggaggtcttcgagcgggccgggatcgccgcggacgcgttgcgggacagccccaccggagtgttcgtcggcaccaacggccaggattacgccgccctcgtcggtaacgcgccacagcgtgcggacggccatctggccaccggcagcgcggcgagcgtggcatccggccgactgtcctacaccttcgggctcgagggcccggccatcaccgtggacaccgcgtgttcgtcgtcgctggtggccatgcacctggccgcgcaggcgctgcgctcgggcgaatgccgtatggcccttgcgggcggcgccacggtaatggccacgcccaccgcgttcgccgagttctcccggcaaggcgcgttggccgctgatggccggtgcaaggcgttcgcggcgggcgcggacggcaccggctggggcgaaggcgtaggcattctgctgttggagcggctgtccgacgccgagcggaacggccaccgggtgctggcggtgatgcgtggctccgccgtcaaccaggatggtgcgtcgaatggtttgacggcgccgaatggtccgtcgcagcagcgggtgatccggcaggcgctggcgaacgcacgtctgtccacagtagacgtggacgcggtggaggcgcacggtacgggtacgacgctgggcgaccccatcgaggcgcaggctctgctggccacctacggccaggaccgggatccggatcggccgctgctgctgggctccgtgaagtccaacatcggccatacgcaggccgcggccggtgtggctggtgtgatcaagatggtgatggcgatgcgccacggcgtgctgccgcggagcctacacatcgacgagcccactccccacgtggactggacggccggacggatcgcactgctcaccgaaccgtccccctggcctctgacgggagcgccgcgacgcgccgccgtctcctcgttcggtgtgagtggcaccaacgcgcatgtgatcctcgaacaggcatctgcggtggccgaacccgaggaaaccgacacggcgcgaacacccgaaccgccagctgttccgtgggtgctctcggcacggagcgaggcggggctacgggcgcatgccctcaggcttcggtccttcgtgaacgccgatgctgatctgcgtccagtcgatgtcggctggtcgctggcgtcggctcgctcggtgttgtcacaccgtgcggtggtcgtgggcgcggaccgcgatgaactcctccgtgaactggaggccgtggccagtggcagcgtcacggtcggcgaggcccgcacgcattccggggtggtgtttgtcttcccggggcaggggtcgcagtgggttgggatggcgttggagctcctggagcattcgccggtgttcgcggggcggatgcgtgattgtgcggatgcgttggcgccgtttgtggagtggtcgttgttcgatgtgttgggtgatgaggtggcgctcggtcgggttgatgtggtgcagccggtgttgtgggcggtgatggtgtcgctggccgagttgtggcgttcgtttggtgtggtgccgtcggcggtggtggggcattcgcagggtgagatcgcggcggcgtgtgtggccgggggtctgtcgttggaggacggtgcccgtgtggtggccttgcggagcagggcgttgctggctctgtcgggtaggggcggcatggtgtcggttccggtttctgctgaccggctgcggggtcgtgtggggttgtcggttgcggcggtgaatggtccggtgtcgacggtggtgtcgggggcggttgaggtgctggagggggtgctggcggagttcccggaggccaagcggattccggtggattatgcgtcgcattcggtgcaggtggaggggatccgggagggtttggcggaggcgttggcgccggttcggccgcgtacgggtgaggtgccgttctattcgacggtgaccggccggctgatggacaccatcgagttggacggggagtactggtaccggaatctgcgtgagacggtggagttccagagcaccgtcgaagctctgatcggccagggtcatacggtgttcgtcgaggccagcccgcatccggtgctgaccgtcggcgtccaggacaccgccgacaccaccgacaccgccaccgacatcgtcgtcaccggatcgctgcgccgcgacgacggcggtccggcgcgcttcctcaccgcgctggccgagctgtccgtacgaggggtggcgacggactggcggcaggcgttcgaagggaccggcgcccgacatgtcgacttgccgacctaccccttccagcggcagcgcttttggatcgaacccactgccccggacgtggcccgggaggacgctcgcgtcaccactgcggacggcgagttctgggcggccgtcgagcgcgaagacgccgcatccctggcaacagccctggaggtcgacgacgcctcactgggcaacctgctgcccgccttgtcggcctggcgccgccggcggcacgagtggtccgcattggaggccgtccggtaccaggtcaactggaagcggctcgtcgatgaccgacccgcgatgttgtcaggtgcctggctggtcgtggtttcccaggccgacgccgaccatgagtgggtctccggcgtaagcgagacgctcgccgagtacggggccgagccagtggtgtgcccggtggacgagcgacacctggatcgtgccgtgctggccgaccggctggcgagcatgaccggtacgagcagcacgacgagcacggcgagtatcagcggcgtggtgtcgctggtcgccctggaccagcgcccgcacccggacttcgcctccgtgcccattggtttcgcgatgacggtgctgctgactcaggcgttgggcgacacgggggtggaggccccgctgtggagtctgacccaacacgccgtgtccaccgggcccgctgacaccctcctcgcgtccgcatcggcgcaggcactggtgtggggcgtcggccgagtgatcgcactcgagcagcccctgcgctggggtggtctcatcgacctgccgaccgaggtgaacgcgagggcgcgggaacggctggcaagggtcctgtcaggcgtttcgggcgaggaccaggtcgcgatccggacggtgggggccttcggacgcaggctcgtccatgcacccgcgttgcggaccgacctgccgtcctggcagccgagcgggaccgtactggtcaccggaggcactggagcgctgggcggtcatatcgcgcggtggctggcgcatcagggcgcggagcacctcgtgctgaccagccgacgcggtatggccgcgcccggggcgtccgcactcgtggcggacctggaagcggccggagcggcggtgacggtggccgtgtgcgacgtggccgagcgtgcccaactggccgacctggtggcggatgtcggcccgctgacggctgttgtgcacacggccgccctgctggacgacgcgacggtcgagtccctgaccaccgagcaactgcaccgggtgctccgcgtcaaggtcgacggtgcgacgcatctgcacgagttgacccgtgacatggaactctccgcgttcgtgctcttctcctccttgtccgggacggtcggcacaccggggcagggcaactacgcaccgggcaacgccttcctcgacgcgctggccgagtaccgcaggacccaaggcctggtggcgacatcggtggcctggggcctgtgggccggtgacgggatgggagagggcgaagccggcgaggtggcccggcggcatggtgttcccgcgctgtcgccggagctggcggtggccgctctgcgtgcggccgtcgaacagggcgacgcggtggtcacggttgccgacatcgaatgggaacgccattacgccgccttcaccgcgacgcgccccagccccttgctcgccgaccttccagaggtacggcgactcatcgacgcgggcgccgcttcggccgtcgaggagacggaccgggaccgatccggactcagcgggcgcttggcagggctcgacggggccgaacagcggcgactgctgctcgatttggtacgccgcaatgtcgcggtggtgctcgggcacaccgacccagaagccgtgtcgtcccaccgcgccttccaggagctcggcttcgactccgtgacggcggtcgagttccgcaaccggctgggtgccgcgaccggtctgcggctcccggccactgccgtattcgactacccgaccccgctggccctggcggagtacgcgctgtcggaactgctggggacggtcggggagccccttcgcgtcgagtcgagcggctcccccgtggacgacgatccgatcgtgatcgtgggaatgagctgccgcttccccggcggggtgagctcgccggaggacctgtgggacctcctcaccgagggcggggacgcgatgtcggcgttccccggggaccgtggctgggacctggccgggctcttccacagcgaccccggccacccgggtacctcgtacacccggacaggtggtttcctccatgacgcgaccgcgttcgacgccgacttcttcggcatctcgccacgtgaagcgctggcgatggacccgcagcagcggctgctgctggaggcgtcatgggaggcgttcgagcgggcggggatcgatcctcggtcgctgcggggcagcgagaccggggtgttcgccggcaccaatggtcaggactacgtcagccttttgggcggagatcagccgcaggagttcgagggctatgtcggaacgggcaattcggcatcggtgatgtccggccggatcgcctacgtcctgggccttgagggcccggcgctgacggtggatacggcgtgttcgtcgtcgttggtggcgttgcatctggcggtgcaggcgttgcggtcgggtgagtgttcgctggcgctcgcgggcggtgtcacggtgatggcgacaccgggtctgttcgtggagttctcccgtcagcgtggcctggccgccgatggtcggtgcaaggcgttcgcgggggcggctgatggcaccggtttctccgagggtgtggggatgctggtggtggagcggttgtcggatgctgagcggcttgggcatcgggtgttggcggtggtgcggggcagtgcggtgaatcaggatggtgcgtcgaatggtttgacggcgccgaatggtccgtcgcagcagcgggtgatccgtcaggcgttggcgagcgcgggtcttgtggcggtggatgtggatgcggtggaggcgcatggtacgggtacggcgttgggtgatccgattgaggcgcaggcgttgttggccacgtatggtcagggtcgggatgtgggtcggccgttgtggttgggttcggtgaagtcgaatattggtcatacgcaggcggccgcgggtgtggctggtgtgatcaagatggtgatggcgctgcggcatggggtgttgccgcagagtctgcacatcgatgagccgacaccgcatgtggactggtccaccggcgcggtggagctcctgggggagcacacgggctggccggaggtggatcggccgcgtcgggcgggtgtgtcggcgttcggggtgagtgggacgaatgcgcatgtgattgtggagcaggcgcctgaagtggtggagcctgaggctgaaggtgtggtgttgcctgctgtgccgtgggtggtgtcgggtgtgggtgaggtggcggtgcgggcgcaggtggagcggttgcgggcctttgcggaccggaatccgggtctggatccggtggatgtggggtggtctttggcgactggtcgtgcggggttgtcgcatcgtgcggtggtggtgggtgcggatcgtggtgagttgttgggggctttggagggtgtgccggtggtgggtgttccggtggtgggtgggttgggtgtgttgtttgcggggcaggggtcgcagcggttggggatgggtcgtgggttgtatgaggggtatccggtgttcgctgcggtgtgggatgaggtgtgcgcgcagctggaccagcatttggataggccggtgggtgaggtggtgtggggtgatgatgccgagctaattggcgagacggtgtatgcgcaggcggggttgttcgcgcttgaggtggcgctgtatcggctgatcgcttcgtggggtgtgaggggggattatctgctgggtcattcgattggtgagttggctgcggcgtatgtggcgggtgtgtggtcgttggaggatgcggcgagggtggtggtggcgcggggtcgtttgatgcaggcgttgccgtcgggtggtgcgatggttgcggtggccgtttcggagggtgtggtgcggccgctgctgggcgagggtgtggtggttgcggcggtgaatggtcccgagtcggtggtgctgtcgggtgatgaggatgcggttcaggttgtggtggatgtgttggctgggcgtggggtgcggacgcggcggttgcgggtgagtcatgcgttccattcggctcgtatggacgggatgctggcggagttcggtgaggtgcttgggggcgtggagttccgtgccccgagcgtgcccgtggtgtcgaacgtgtccggtgcggtggcgggtgaggagttgtgttcgccggagtattgggtgcggcatgtgcgggagacggtccggttcgccgatgggctggagacgctgcgcgagctgggtgtgggttcgttcctggagttggggcctgacgggacgttgactgccttggcggatggcgatggtgtgcctgtcttgcgtcgggatcgtccggagcctctgaccgctatggcggctttgggcgggctgtacgtccggggtgtccagatcgactggggggcggtgttcccgggtgctcggcgggtcgatttgccgacgtatgccttccagcgtgagcggttctggttggagccatccgctgagcagcctgcgacgagcgtggtggacgcggcgttctgggacgcggtcgagcggggcgatgcggaggctcttgggggcgatgccgagcagtcgttgagtgccgcgttgcctgctttggcgtcgtggcggcgggcgcagcaggaagagtcggttatcgacgggtggcgttaccggctcggctggacgccgatcccggtggtgctgggggagccatgcctcactggcacttggcgggttgtggtcgaaccgggtgcggacggtaccgatgtggctgccgcgctgcggtcggccggggctgatgccgaggtcgtgacgtcggcggaactgagcgcggggccggtcgcgggtgtggtgtcattgttgtcggtcgaggcgacggtggcgctggtgcaggctctcgggacggtcgggatcgatgcgccgttgtggtgtgtgacgcggggtgcggtctccgtggtggacggggatgtggtggaaccgtacgcgtcggccgtctggggtctgggccgtgtgatcggtctggagcatccggaccgttggggcgggctgatcgacctgcccacggaggcggacgcacgtgtgggtgcgttgttggccggggttctcgccgggcgcaccggggaggatcaggtggcaatccgggccgccggggcgtggggtgcccggctgagccgggcgacaccgattgcggacacgtctggcgggtggcgtggtcggggagctgccttgatcaccgggggtacgggtgcgctgggcggccatgtggcgcgctggctggcggggaccggggtggagcgcatcgtgctgacgagccgccgggggatcgagaccccgggtgcggccgagctggtgaccgagttggaggagttcggagtccaggtgacggtggtcgcgtgcgatgtcgccgatcgggaggcggtcgcgacgctgctggtcaccatccccgatctccgggtcgtcgtacacgccgcaggggtgccgagctggagtgcggtggacagcctgacacccgaggagttcgaggagagcgcgcggtcgaaggttgccggggcggcgaacctggacgcgctcctggcggacgctgagctggacgcctttgtgttgttctcgtcggtggcgggggtgtgggggagcggtagtcagtcggcgtatgcggcggcgaacgcgtttctggatgggttggcgtggcggcgtcgtggtgttgggttggtggcgacgtcggtggcgtgggggatgtggggtggcggtggtatggcggttgggggtgaggagtttctggttgagcgtggtgtgtcggggatggctccggggttggcggtggctgcgttgcggcgggcgctgtgtgatggtgagacggcgcttgtggtggcggatgtggattgggagcggttcgggccgaggttcaccgcgttgcgtccgagcccactgctgagcgagctgatccccgatacgtccgaaccactcgcgtcgacggtgggtgagttcgcggtcgagctgcgcggattgtcgcgcgaggaccgggaccgtgccgtcgtggagctcgtacggacacatgccgccgaggtgttgggccaccagaacccgagcgcgatcgacctggaccggacgttccaggagctgggctttgactcgctgaccgccgtggaattgcgggaccggctcggcacggctactcagctgcgattcccagcgtccgtgatcttcgactacccgactccggcggcactcgccgagcatgtgtgcggggcggccctcggactggccgaagagatacaggtagcgcacacgcccagcgcggtggccgacgatccgatcgtgatcatcggcatgagctgccgattcccgggcggtgtggactctccggaggcgctgtggcggctggtcagcgccggtggcgacgccgtatcgtccttcccgtccgaccgtggctgggacctggccggtgtgtacgacgccgacgccactcgctcgggccggtcgtacgtccgcacgggtggattcctccatgacgcggctgagttcgacgccggattcttcgggatctcgccgcgcgaggcgaccgcgatggatccgcagcagcggctgctgctggaggcgtcctgggaggcgttcgagcgggccggaatcccggcctcgacgctcaagggcagccagaccggcgtcttcgtgggcgcgtccgcacagggctatggcggcggggacgggcaggcgccggaaggatccgaaggataccttctgaccggcaacgcgggcagcgtggtgtccggtcgggtggcctatacgtttgggctggagggcccggcggtcaccgtggacacggcgtgctcgtcctcgttggtggcgctgcactgggcggtgcgggcccttcggtcgggcgagtgctccctcgcgctggccggcggagtgacggtgatggcgacacccgccacctttgtggagttctcacgtcagcgtgggctggccgccgatggccgctgcaagtccttcgccgccggtgcggatgggacgggctggtcggagggtgttgggctgttgctggtggagcggttgtcggatgccgagcggaacgggcatccggtgctggccgttgtctccggctctgcggtgaatcaagacggtgcgtcgaatggtttgacggcgccgaatggtccgtcgcagcagcgggtgatccgtcaggcgttggcgaatgcgggtcttgtggcgtcggatgtggatgcggtggaggcgcacggtacgggtacgacgctgggtgatccgatcgaggcgcaggcgttgttggccacgtacggtcagggtcgggatgcgggtcggccgttgtggttggggtcggtgaagtcgaacatcggtcatacgcaggcggctgcgggtgtggctggtgtgatcaagatggtgatggccatgcggcatggggtgttgccgcggacgttgcatgtggatgagccgtcgccgcatgtggattggtctgctggtgcggtggagttgttgacggggcaggtggcgtggccggaggtggatcggccgcgtcgggcgggtgtgtcggcgttcggggtgagtgggacgaatgcgcatgtgattgtggagcaggcgcctgaagtggtggagcctgaggctgaaggtgtggtgttgcctgctgtgccgtgggtggtgtcgggtgtgggtgaggtggcggtgcgggcgcaggtggagcggttgcgggccttcgcggaccggaatccgggtctggatccggtggatgtggggtggtctttggtggccacccggtctgggttgtcgcatcgtgcggtggtggtggttgcggatggtgaggagttgttgggggctttggagggtgttccggtggtgggtgggttgggtgtgttgtttgcgggtcaggggtcgcagcggttggggatgggtcgtgggttgtatgaggggtatccggtgttcgctgcggcgtgggatgaggtgtgcgcccagctggaccagcatctggataggccggtgggtgaggtggtgtggggtgatgatgccgagctaattggcgagacggtgtatgcgcaggcggggttgttcgcgcttgaggtggcgctgtatcggctggtcgcctcgtggggtgtgagggcggattacctgctgggtcattcgattggtgagttggctgcggcgtatgtggcgggtgtgtggtcgttggaggatgcggcgagggtggtggcggcgcggggacgtttgatgcaggcgttgccgtcgggtggcgcgatggtcgcggtggcggcgtcggagggtgaggtgcggccgctgctgggcgagggtgtggtggttgcggcggtgaacggtcccgagtcggtagtggtctcgggtgatgaggatgcggtgcatgccatcgaggagacgttcgccatgggtggggtgcggacgcggcggttgcgggtgagtcatgcgttccattcggctcgtatggacgggatgctcgcggagttcggtgaggtgcttcggggcgtggagttccgtgccccgagcgtgcctgtcgtgtcgaacgtgtccggtgcggtggccggtgaggagctctgctcgccggagtattgggtgcggcatgtgcgggaaacggtccggttcgccgatgggctggatactctccgtgagctgggtgtgggttcgttcctggagttggggccggacgggacgttgaccgccttggcggatggcgatggtgtgcctgtcttgcgtcgggatcgtccggagcccctgaccgctatggcggctctgggcgggctgtacgtccggggtgtggaggtggactgggacgcggtgttccccggcggtcggcgggtcgatctccccacctacgcgttccaacggcagcggttctggttggagtcggcctcggaccagcctgcgaccagcgcggtggacgcggcgttctgggacgcggtcgagcgcggggatgcgcgggcgctgggcattgacgaggaacagccgttgagtgccgtactgcccgccctctcgtcgtggcggagggcgcggcaggagcagtcggtgattgatggctggcgttatcggctcggttggatgccgattccggcggtgttgggggaggtgggcctcatcggtacctggctggttgtggtcgagccgggtgtggacggtactgatgtggccgcagtgttgcggtcggccggggctggtgtcgaggttgtgacgtcggcggagctgagcgctggtccggttgcgggtgtggtgtcgttggtgtcggtcgaggcgacggtgtcgttgctgcaagtccttgtggcggccggggtcgatgcgccgttgtggtgtgtgactcgtggtgcggtctcggtggtcgacggtgacctggtggatcctggccaggcgggaatctggggtctgggccgtgtgatcggtctggagtgtccggaccgttggggcgggctgatcgacttgcctggcgaactggacgatcgcgcggggaatgcgctggtaggcatccttgccgggggcaccggtgaggatcaggtggccatccgtgtcaccggcatatggggtgcccggctggtgcgggcgacgccggtcccgatcggtgacgcgggtggtgaggctgcggccgcgtggcgtgggcgtggtaccgcgctggtcaccggtggtacgggggcgttggggcgtcaggtggcgcggtggctggtgggcagtggtctggagcgggtcgtgctgacgagccgtcggggggttgaggcgcccggtgccgtcgagctggtggctgagttggggagccgagtgcgtgtcgtggcctgtgatgtcggcgatcgtgaggagcttgcggctcttttggtgacgcttccggatgtgcggaccatcgtgcatgcggcgggtgtcctcgacgacggggtgctcgaatcgctgacgcccgagcggatccgtgaggtgatgcgggccaaggccgacggcgcgcggcatctccacgagttgacccgtgacatcgacctcgacgcctttgtgttgttctcctcggctgccgggaccgtgggtaatgcgggtcaggggagctatgcggcggccaacgccgtcctggacgggctggcgtggcgtcgccgggccgagggcttggtggccacatcggtggcctggggagcctgggccgaatccggtatggccgcggagatggcgcggtcgcagggcatggatccgaggtcggcgctcgccgccctggggctggtgctggccgctgacgagaccacggtgatggtggccgacatcgactgggcgaccttcggggcccggttcaccgcctcacggccgagcccgctgctcagcgagttgctcggcgacggatccgtgtcgaccgaggcagccgacggcgaaccggccgacgcgttcgccacccgcctggaggccatggccgagcgggaacgggcggccaccgtgctggacctcgtccgtacgcatgtggccgctgtcctgggacacacggcatccgaggcgatcgacccggcccggcccttccaggagatcggtttcgactcgctcaccgcggtggagctgcggaaccggctcaccgcggccaccggggtacggttcccggcttccgtgatctacgactacccgaccccggccgcgctcgccgagcacgtgtgccgggaggcgctgggtccgggcggacggacaccggctccggtggtgccacgcccggtggacgacgaaccgatcgccatcatcgggatgagctgccgtttccccggcggggtgagctcgccggaggacctgtgggggctgctggccgagggccgtgacgccgtgtcggacttcccggcggaccgtggctggaacctggccgagctgtacgacccggatcccgaccaccccggctcctcgtacgtccgggcgggcggattccttgatgacgcggccgcgttcgaccccggcttcttcgggatatcgccgcgcgaggcgctcgcgatggacccgcagcagcggctattgctggaggtcgcctgggaggcgttcgagcgcgcccatatgtcccccgccaccctcaagggcagccggaccggggtgttcgtcgggaccaacggccaggattacgccgctctggcgagcggggccccgcggagcgcggaagggtatctgggcacgggcagcgccgccagtgtcgcctcgggccggctggcgtacaccttcggcctcgagggcccggcggtcaccgtggacaccgcctgctcgtcgtcgctggtcgcgctgcacctcgccgcacaggccctgcgctccggtgaatgctccttggccttggccggtggtgcgaccgtcatggccactccggcggccttcctggaattctcccgccagcgtgcgttggcggccgatgggcgctgcaaggcgttcgcggcggcggcggacggcaccggctggggcgagggcgtcggcatgctcctggtggagcggctctccgacgcggagcgcaacggccaccgggtgctggcggtgatgcgtggctccgccgtcaatcaggacggcgcgtccaacgggctcacggcgccgaacggcccgtcgcagcagcgagtgatccgtcaggccctggcgaacgcccggctgtccgccacggacatcgacgtggtggaggcgcacggcaccggcaccagtctcggcgacccgatcgaggcgcaggcactgctcgccacgtacggtcagggccggtcccagaacaagccactgtggctcggctcggtgaagtccaacatcgggcacacccaggcggccgccggcgtggccggtgtgatcaagatggtcatggccatgcgacacggtgtactgccgcggaccctgcatgtcgactcgccctcgccccatgtggactgggcggcggcccgggtcgagttgctcgtcgaagcgagggagtggccgcggaccggcgctcctcgccgggcgggtgtgtcctcgttcggggtcagtggcaccaacgcccatgtcatcgtcgagcaggggccggtggtggcccggcccgatcgggagtcggcgcgcgagccgtcaccctccgtgccgtgggtgctgtcaggtgcgggggaggccgggctgagggcccaggtcgagcgcctggcgtccttcatcgacgcccatccgggcctggatcccgccgatgtcgggtggacgctggtggccggccgttcgtgtcagtcgcaccgcgccgtagtggtgggtgcagacctcgcggagcttcgacgtggactggacgcagtctcgaccggtggcgccgcccggtccggccgcaaggtggtgttcgtcttccccggccaggggtcgcagtgggccggaatggcgttggaactgttggagcattcgccggtgttcgcggagcggatgcgtgcatgcgccgatgcgctcaccccgttcgccgagtggtcgctgttcgatgtgctgggtgatgaggtggcgctcggtcgggttgatgtggtgcagccggtgttgtgggcggtgatggtgtcgctggccgagttgtggcgttcgtttggtgtggtgccgtcggcggtggtggggcattcgcagggtgagatcgcggcggcgtgtgtggccgggggtctgtcgctggaggacggtgcccgtgtggtggccttgcggagcagggcgttgctggctctgtcgggtcggggtgggatggtgtccgtaccggtgtccgccgatcggctccgtgaccgtgcggggttgtcggtggcggcggtgaacggtccggcgtcgacggtggtgtcgggggctgttgaggtgctggatggggtgctggcggagtttccggaggccaaacggattccggtggattacgcctcacactccccgcaggtggccgagatccagcgggagctggcggacgtgctggcgccggtccggccgcgcggtggacagatcgcgttccactcgacggtgaccggacggctcaccgacacctccgaactcgacgccgactactggtaccgcaacctccggcacaccgtggaattccagagcaccgtcgaagccctgatgaaccagggccacaccgtgttcgtcgaggtgagcccgcaccccgtgctgaccatcggcatccaggacaccgccgagaccccaggcacccccgacaccccaggcacccccgacaccgcggacgccaccgacgctcacgaggccaccggcgcccccgacgtcgccaacaccgccgacgtcaccggcgctcccgacgtcaccggcgccgacatcgtcatcaccggatcgctgcgccgcgacgacggtggccccgcccgcttcctcaccgccctcggcgacctccacacccggggcgtggacgtggactggagcccggtcttcaccggagcccggacggtggaccttcccacctacgccttccaacgggaacgcttctggctgaagcccgcgcgggcggtgacccaggcgtccgggctgggcctcggcgatatcgagcaccccctgctgggcgcggtactgcccctgcccggggacgagggcggtgtgctgaccggactgctctccctggacggacagccctggctggcccaccacatggtgcgggacacggttgtcttccccggcacgggattcgtcgaactcgccctgcaggccggtcagcacttcggccactcggtgatcgaggagctgaccctgcatgccccgctggtggtgccggaccagggcggggtccaggtacaggtggccgtatcggcggcggacgaacggggccggaggccggtcacggtgcactcgtgccgtgccggggagtggctgctgcacgcctcgggcactctcggcgccaccggaggcctcgacgtcaccgagccgcgccccgccgacgtggcccggcccctggaggtctggccgcccgagggcgcgcggagcctcgatgtctcggggatgtacgaggcgatggcggagcgcggctacgggtacggtcccgctttccaagggctgcgcgccgcgtggacacgggacgatgagatctacgccgaagtggctctggagccggaggcacaggacgtggcggcgcggtgcggtgcgcatccggccctgctcgacgccgcgctccacggagtggggctcggccgcttcctcaccgaccccggccaggcgtatctgccgttctcctggagcggggtcgcgctgcacgcggtaggcgcctccgccatccgcgtggtgctctccccggccggtacggacgcggtgtcgctggaggtgacggacccgacgggagcgccggtgctgtcggtggcgtcgctctcgttgcgtccgctgtccagcgggcggatcgcggacacccgaggggtggaccaggactcgctgtaccgcgtggactgggtcgagatgccgctgccgactgccccggcaggctcggccccggccgagtacgacgcgccggcgatgttcgacgccctggtattcgacgccccggtcgagtacgacgttctcgcctccgacgcctccgacgcctccgacgcctccgacgcccccggcacccccgacgcctccagtgccccggtgcccgacatgcccgacatggtggtgctgccgtgtgagtcggccggtgacgcggtgtccaccgtcgtgtgccgggcgctggcggcggtacggcgatggctcgccgacgagcgctgtgcccggtcgcggctggccgtgctgacgcgcggcgcgatggccaccgctcccggcgagagcgtcgaagacctcggcgcggcagcggtctggggcctgctccgcagcgcccaggccgagcacccggaccgcttcgtcctcgtcgaccacgacggccaccaggattcccgtgcggtgctcgccgccgcgctggccgccgcggtcgacggtggccatgcgcatctcgcgctgcgccgtggccgtgtcctgacgcctcagctcgctccgctcaccccgtccgcgaccgccctgtccaccaccgcaccgcccgccgccaccccaaccccggaggccggggcaccgtggcggatggacgtcaccagtcagggcacgctggagaacctggccgccgtcccctgcccggaggccgccggtgtcctcggcgccggacaggtgcgggtggcgatgcacgcggccggggtgaacttccgggacgtcgtcgtcgccctcggcatgatccccggtcaggacgtcatcggcagcgagggtgccggagtggtgctcgacatcggccccggtgtgtccggcctggcgcccggtgaccgggtgatgggtctgttctccggggcgttcggccccgtggcggtgaccgatcaccgactgttggcgcggctgccggaaggctggtcgttcgccgacgccgcggccacgccggtggtgttcctgaccgccatgtacgggctgatggacctggccggtctgcgacccggtgaatcggtgctgctgcactcggccgccggcggggtgggcatggccgcgacacaggtggcccgctggctcggcgctgaggtgtacgccaccgcgagcccagggaagtgggacgcgctgcgcgccggaggagtggcggacgaccggatcgcctcgtcccgctccttggagttcgccgaccgcttcggccgggtggacgtggtgctgaactcgctggcgggcgagtacgtggacgcctcgctcggcctgctcgccgacggtggccgtttcctggagatgggcaagaccgacatccgcgacggtgagcgcgtggccgcggagcacggggtgcggtaccaggcgttcgacctcatggacgcggggcccgaccgggtcggggaactgctcaggctgctggtgtcgctcttcgagcgagggatcttcacggcactgccgacccgcgtctgggacgtccggcaggcgggtgacgcgctgcgcttcctctcgcaggcacgccacatcggcaagctggtgctgtccattccgcagccgctgcgggagggggacaccgtgctcatcaccggcggcaccggcacactgggcgggctggtcgcccgtcacctggtcgaacggcacggagtacgggatgtcgtcctggccggccggcgggggccggacgccccggacgcggccgaactcgccgccgccctgcgcgaatacggcgcccgggtgcgggtggtggcctgtgacgtggccgaccgggaccagctggcacggctgctggacaccgtctccggcctgcggatggtggtgcacaccgcgggtgtgctcgacgacggggtgatcgagtcgctcaccccggagcgggtgcgcgaggtcctgaggccgaaagtggacgccgcctggtatctgcacgagctgacggccggtcgtgagctggcggaattcgtggtgttctcctcggccgcgggtgttctgggaagccccgggcagggcgcctacgcggcggcgaacgcctggctggacgcgctgatggcgcatcgccgggccgccgggctgccgggtctctccgtggcctgggggctgtgggccgagcgcagcgggatgaccggccatctgtcggaccgggatctcgcccggatggccagggccggtgccacgcctctcgccaccgatcaggggctccggctcctggacagtgccagggcggccaccgaggcgctcgtgctggccacaccgctggacgccgcggcgctgcgggcacaagccgacgccggggcgctgcccgcgctcttccgcggtctggtccgtgcgccgatccgccgcgcgaccggcgcgggcccggtggaggacgagtcgtcgctgcggggccggatggccgcgatgccggtcgccgagcgcgaacagctggtgctggacctggtccgtacgcaggtggcgaccgtgctggggcacggcaccgccaccgcggtcgacccggcgcgtacgttcgcggagaccggcttcgactcgctcacggccgtcgagctgcgcaaccggctgcgcaccgccaccggggtcaggctgtcggccaccgcgatcttcgactatccgacacccgcggtcctggccggtcatctcctccgggagctggacggcaccgtcggcgaggccgtgacacggcccgccgccccggccgccgccaccgaccgggacccgatcgtgatcgtcggaatggcctgccgctatccgggcggagtggcgtcgcccgaggagttgtgggagctgctcgccaccgggcgcgacgcggtcgcggatctgccggacgaccggggctgggacctggacggcctgtacagcgccgatccggacagctcgggcacctcgtacgtccgctccggtggcttcgtgtacgacgcgggcgagttcgacgccgacttcttcggcatctcgccgcgcgaggcgctcgcgatggatccgcagcagcggttgctgctggaagtggcctgggagacggtggagcgggccggtgtcccggcggcgtcgctgaaggggagccagaccggggtgttcgtcggtgccgcggcacagggctacggcacgggggccgggcaggcggcggagggatccgagggctacttcctgaccggtggcgcgggcagcgtggtctccggccggctctcgtacaccttcggcctggaggggccggcggtcaccgtggacaccgcctgctcgtcgtcgctggtcgcgctgcacctggcggcgcaggccctgcggtccggcgagtgctcgctggcactggccggcggggtgacggtgatggccaccccgggcatcttcgtggagttctcccgacagcgcggactggccgccgacggccgctgcaaggcgttcgccgacgcggcggacggcaccggctggggcgagggcgtcggcatgctgctgctggagcggctgtccgacgcccgccgcaacggccaccgggtcctggcggtcgtacggggctccgccgtcaaccaggacggcgcctcgaacggcctgacggcgccgaacgggccctcgcagcagcgggtgatccgggccgcgctggcgaacgccgggctggccgcgtcggacgtggacgcggtggaggcacacggcaccggcaccagcctgggcgacccgatcgaggcacaggcgctgctggccacctacgggcagcaacgcgaacggccgctgctgctgggctcgatcaagtcgaacatcgggcacacccagtcggccgcgggagtggccggtgtgatcaagatggtgctggcgatgcggcacggggcgctgccccgcaccctgcacgtggaccagccgtcgacccatgtggactggtcggccggtgcggtggagctgctgaccgagcccgccgagtggccggggacctcccgcccccgccgggccggggtgtcctcgttcggggtgagcgggaccaacgcccatgtgatcctcgaacagccacccgcggaggcggagtccgggcccgctccggagtcggcacccgggcccgtcccggcggtggtgcccgggcccgtcccggcggtggtgccatgggtgctctccggccagggcgagcgcggactgcgggcgcaggccgcccggttgcggtccttcctggccgcgcgccccgagtccggcccggccgacgtgggctggtcgctggccgccacccgttcggcgctctcccaccgggccgcggtggtcggggcggaccgggcggaactgctggacggactggccgcgcttgcggccggcgagcccgccccgggcgtggtcttgggcaccgcggacccgggccgggtgggcgtgctgttcgcgggccagggtacgcaacggcccggtatggggcgtgagttgtaccagtcgttcccggttttcgcggcggcgtgggacgaggtgtgcgccgcgctcgacccgcatctggaccgtccgctcggcgaggtggtgaccgatgccaccggcgcgctggacgccaccacgtacacgcaggcgggcctgttcgccctcgaagtgtcgctgttccggctggtgtcctcctggggcgtgcggccggactatctgctgggccactccatcggcgagctggcggccgcgcaggtggccggtctgtggtcgctggaggacgccgccaaggtggtggcggcccggggccggctcatgggcgcgctgccgccgggcggggcgatggtggccctggccgcgccggaggaccaggtacggccgttcctgaccgaccgggtcgccctcgcggccgtgaacgggccgtcgtcggtcgtggtgtccggggacgaggacgcggtgtgcggtgtggccgaggcgttcgccgcccgtggggtgaagacgcggcggctgcgggtcggccacgccttccactcgccgctgatggacgagatgctcatcgcgttcgccgaggtactcgacacggtggacttccgcaccccgcggataccggtggtgtcgaacctctccggtgcggtggcgggggaggagctgtgctcccccgcttactgggtgcggcaggtgcgggagacggtgcggttcgccgccgggcttgagcgtctgcgggagctcggcacgggcaccttcctcgaactcgggccggacggcaccctcaccgccttggcccaggcccagatcaccggggcggacgccgagttcatccccactctgcgcgccgaccggcccgagccggtcacggtcaccaccgccctcgcccagttgcacacacacggtgtggagccggactggtccgcggtcttccccggcgcccaccgggccgagctgccgacctacgccttccagcgctcccgcttctggctggagccctcccgtacacccggtgacgcgggcgacttcgggctcggcgcgctggaccatccgctggtcggcgcgagggtgccgctgcccgacgcggacggcgttctgctcaccggccgcatctccgccgaggcccactcgtggctgatcggtcagcgggcgctgggcgtgcccctgttcccggcgaccggcttcctggaactggtgctccaggcggggctccagtgcgacagccggacggtggacgaactcaccatccatgaaccactcgtcctccccgagcggggcggggtcgaggtgcaggtgtccgtccgtggcgccgacgagtccggccgccgcccggccaccgtgtactgccgccgcgaccagcggtgggtccggcatgccacggccgtcctcggcgcggaccggccgcccgcgccggagccgcgccccgagccctggccgcccaccggcgcccggccgctggagtccggcgggacaccggcgtggcgccgtgacgacgaggtcttcctggacatcgagctgcccgaggtggccggggccgaggccgaacgctggacgctgcatcccgccctgctcgaacaggcgttgcgcggggaggcgctggcagggctggtcacggcggccgaggggacccatctgccgttctcctggacggggatcaccctgcacacgacgggtgccacgagactgcgagccaccctcgcgcccgtcggcccggacacggtctcgctccacgtggccgacgccgccggaacacccgtgctgtcggtggactcgctggcgctccgcccggtgtccggacagcggctgcgccaggccaacgcggcgctgttccggccggtgtgggcggcttgccgcacgcgggccgaaccggacaccggctctgtccgatgggggctcgtcggcgacccggacgcctggaaaccggacacgctcggcgcgccggtcgcgctgtacccggacctgtcggccatcgaggacgtaccggacgtcatcctcctcccgtgcgtatccgagggcggaacggcgtccgaggtggccgtccgcgtatccgagaccgtgcggacgtggttggccggggagcggttcgccgcctcgcgtctggtgctggtgacccggggcgcgctcgccacggcggccggtgaggagctcgaggacctggccgcggccgcggtgtggtcgctggtcgagcccctccaggcggccgtggcgggacggctgacactcgtcgacaccgatacgtccgatctgcgcatgctgcccgccgcggtggccgtgggggaggaccgggtcgcggtccgggcgggagcggtgctggtaccggacctggtcacgccgccggccaccgagcaggatccgcccgcctggggcccggggacggtgctggtcaccggtgggtcggccatggctgtctcccggcatctggtcgccgaacgcggtgtgcgtgacctggtcctggccggggacggcgacatggccgaactggcggccctcggagccacggttcggctcgccccgtgtgatccggcggacggtcaggcgctggcggcgctggtggcggagattcccgggctgcggagcgtggtgcacaccgcggccgacgccccggagcggacccggtccctcttgccggaatccctgcggccacagctgcggtcgggagtggcggcggcctggaacctgcacctggccacgcggggcctggaactggaccgctttgtgctgttcacctccgccgacgggacactgggccccgcgtacgccgacgcgctggccgcacaccggcgggctcgcggactgcccgcggtgtccgtctccaccgatctgggtctcgccctgttcgacgaggcatgcgccgggcccggggaggcgatccgggtcaccaccgccacgccggcccccgcacccaccgaggcggaccggcagccggtggaacaacccccggcggccgaggcctccgcgaccacgttgctggagcggctggccgggcggacggaggacgagcaggacgagatcctgctggagctggtccgtggccaggtcgccatggtgctcggccatcccgacgccaccatggtcgacccggaccgaggcttcgtggaactgggcttcgactcggtggcggccgtgaagctccgcaaccaactggccggagccacccggctcgacctgcccgccagcctcaccttcgaccaccccacggctgtcgatctcgcccgccatctgcgcgccgaaatgctgcccgacgacgcggcggccgccattctcgtgctcgaagagctcaacaagctcgacgattcgatcctcgtgctcgacccggcaagcgcggcacgggtgcggatctcgaccctgctccaggacctggccgcgaaatgggtcgagcggacggatcggccatgaccacacacgatcagttgatgcgcgaacgagggagtcaacagtgagcgagaccttgtcccttcccgggaccgtgaaggccgaacggcgttgtccgtacgacccgccggaggcgcaccgccgactgcgggacaagggcgaactgggcaaactggagctgcccggcggtctggtgatgtggttcctgaccaagcacgacgacatcagggccatgctggccgactcccggttcagcggtgcgagggtgccgtttccggcgatgaacccggagatacccgcgggcttcttcttctccatggacccgccggaccacacccgctaccgccgcacactcaccgccgagttctcggtgcgcggcgcacgcgaactgaccggccggatcgagcggctggccgaccggcacctcgatgcgatggaggcggcgggcacgagcgcggacctcgtggcggcctacgccagtccggtgcccgcgatggtgatctccgaaatcctcggcgtgccgtacacctaccaccagaagttcgaccacgaggtacgcacgctccgggagaccggcggcgacgatcaggccgtcggcgcgatggcgaccgcgtggtgggacgagatgcgcggattcgtgcgtgccaaacgggccgagcccggggacgacatgatcagcaggctgctgcatgatgaggtcgagggcggtgcgctgaccgacgaggaggtggtcggcattgcgatgaccatcattttcgccggtcatgaacccgtggagaacctgatcggcctcggcatgctggcgctgttccaggacggtgagcagctgacccggttgcgggagaaccccgacctcattgacagcgccgtggaggagttccttcgctacttccccgtcaacaacttcggcaccgtgcgcaccgccaccgaggatgcagtgatcaatggtcaccccatcgcgaagggcgagatcgtggccggtctggtgtccaccgccaaccgggaccccgagcggttcgccgatcccgaccgccttgtcctcgaccggtcgcacacctcccacctcgcgttcgggcacggtgtgcaccagtgtctgggccagcagctggcgagggtggaactgaaggtgctcctacagcggctgctcgtcaggttccccgctctgcggctggcggtggccccggaggagatcaggtaccgggagaacacctcgttctacggtgtccacgagctcccggtgacctgggcggccgagtagccgcagccggggccggaagacacggcgcgggcggtggccgcggggtccggcgcgagcggtggccggatacccggccaccggctcagccggcccgggtgacgcccactgccgccctgagatccgcccagaactccggccgaccgcggatctcaagagccgagccggccgccggtcaggcgtgccagcgggcggcggcgacgagccgcgcgcctgcgcaggacgaccgcggcggtggcgcccgccgcggcggcacccgcaccggcgacggctgccaggaccttccgggccttgatcatcatccaggccgaacccgccgccgcggcagccttcttgggcgcctcagcggccacggtacgaccggtggtcacggccttcccggcggccacttgagccttgtcggcggcgacatgagcggtgtgcgccgcggtcgtcgccgcgccggccgcggcggtcttcgccgtggtctccgccttgcccgcggtgtccttggccctttccgcggtctccttggccttggtggcggtggccttcgcgttcacgttggtgctccgggtagtgtgtgcgttcttcttctcggtcatgttcaccgcgttaccactcgacccgacaacaaacccgcgtcctcggggccggccgccacggcgtgacgatccgagggggcggcacaccgggaggcgcgccgcccatcggctcattcgatgtctgagccgcccgaaccacccgagccgcgccgctgctggaacagcacaaaccctccggccagggcgaacagacagacgccgatgatgatgcccacggcgggccaagcggatccgccgtcggatgtcgcggccttcgaaggctccagcgatgtggcatcgggcaactgtcttacgacgaaactgtgttcggcgttctcgccgggtgcgagcgccgggccgccgaccgagtagccgtcatcggtgggcttcagcttccagcccttcggggcctgcttcagcctcacatcgccggggtcgatgcccgtgggcaacacggtccggatctcggtgaaaccggccctcccgtcctccgcctccgactcgaacgtcagcgtgacgtccttcgccagggcgcgggagtcggaggcgctcacctcggtgtgggccagggcgggggtcgccgtggcgaggacgagcgccgaggtggcgacggccagggcgccgatccgtcgcggccgcgggtgggacggcggacgatgtgctttcacggtatctctcctcgtccatggggtggtcacccgagccctcctcaccggtcacccggcgcgctcaccaggtgcgttcacattccccggtacgtacggcccgggcgcgatgttcatcctcgcccagaccggaagcccgcttgccacgcgggggagcaaggagttccggcgtcttcgtggcccgcgcaaggcggaccgagggggtcgccgcgtcccagtgcggtgatgtgcccggtggtcaggtggtccgctggtcccgcagggtccgggacagctcctcctcggtgagcacccggggcgcgggtccggcaccgggcttcgtctcggcgccgttcgccgggctcttgttctcggtcgtcgtcatgagggtgcacctttcgctggtggtggcggaggacgggatcaggcggtggcgaccggtgtggcgggcggattggcgttccgcggcacagcggggggcttgcgcgcaggtcctcgcagtacggtgaacgccaccgcgaaggccgccacgatgagccccgttccgacggcgaaggccaggtggtagccgccggtcagcgcctcggcccgacccttgccccgggagagcagggcatccgtgcgggaggcggccagggtggacagcaccgcgacgcccagcgccatgccgatctgctgggtggtgttgaacagcccggagacgagcccggcctcgtcctccttcgcaccggacattcccaggctggtcagcgcagggagcgccagcccgaaaccggcggcgagcagcatcaccgggaggaggtcggggaggtaccgggcgtgcacggggacgcggacgagcaggccgagaacgccggtcaggagggccagcccggtcagcagcaccgcgcggtcgccgaagcgtgcgctgagccgtgcggagacgccgagggacaccgcgccgatggcgatggcggccgggagcatggccagaccggttccggtggcgtcgtaccccagcacattgcgcagatagagggcgaccaggatctggaacgagaagagcgcggccaccatcaggagctggaccagattggcccccgccaccccgcgcgaccgcaggatccgcaggggcatcagcggggtgcgggcggtggtctggcggaccaggaacagggcgatcaggaggatcgagacggcgccgaggccgagtgtgcgcgccgccgtccagccgtagtccgccaccttgaccacggtgtagatgcccagcatcagcccggtcgtgaccagcagggcgccgaggacatcggcgccggccgcgaggcccggcccgcggtcggcgggcaggacgggtatggcgaccgcgagcgtcagcagcccgatcggcagattgatcaggaagatccagtgccagctgagcgcgtcggtgaggaggccgccgagcacctggccgatcgacgctccggcggcgccggtgaagctgaacacggcgatcgccttcgaccgttcggcgcgttcggtgaagagcgtgacgaggatgcccaggctgaccgccgaggccatcgcgctgccgaccccctggaggaaccgtgcggcgatcagcacagcgggggaggtggccacggccgcgagcaacgaggccgcggtgaacaccgcggtaccggtcaggaacacccgcttgcggccgatgagatcgccgatacggccgccgagcagcagcagaccgccgaacgcgatcaggtaggcgttgacgacccagctgagcccggcgggggagaaccgcagatcgctctggatggcgggcatggccacggtcacgatgctgccgtcgaggatcaccatcagcatgccggtggcgatgaccccgagggccagtcgacgtgtcggggggacacggggagacgtcgggtcggaagaggcggcggacatgcggacactcctgtcagtaggcgcgacaggagtgaccgtagcagatggttttgttgcagactatttgtttcggctctacttgtggcgcatgacgggcggccgcgcggatgtctccgctctacttctcgcgctgccgcgccctccgcgccgggcgggggctctcggcgggcgtggccagatgcccctcggacagccgggtcaacgcctttagcagcgcggcgcgttgggtctcggggagtgtcgccaacgcctcgcgatggacgcggtccacgatctcctggctccgttcggcgatccgcgcgccctcctcggtgaccgcgatgatccgggcccggcgatcgtgggtcgaggcgcgccgctccgcgaggcccgccttctccagggcgtccaccgtcaccaccatcgtggtcttgtccatgtcgccgatctcggcgagctgggcctgggtgcgctcttcctccagggcgtggaccagtacgcagtgcatccgcgccgtcagcccgatttcggcgagcgcggccgacatctgggtgcggaggacgtggctggtgtggtcgaggaggaacgacaggtcgggttcggtcttggtgggcgccatggcggtcatgcggcccagggtaacaattcgatccgtactggattatccggaacagtccatagggaggggtggggtcaggggtcagccgttgcggtagagggcggtgagcagctcgaccgcggtctgggtggcggggtgcgggtcgtcccgccaccaggcgaggcgtaccgcgatgggctcggcgtcgcggaccggccggtaggcgattccgggcctcggatactggttggccgtggactccgccgtcatgccgacgcagcggcccgcggagatcacggtgagccagtcctccacgtcgtgggtctcctccgtggccggccgggagtcgggcggccacagctccgtggtggtggtaccggtcctgcggtcgaccagcagggtgcgcccgctgaggtcggccagccggaccgagcggcgcctggcgagcgggtcgtcggcggccacggcgcacagccgccgctccagtccgacgatggcggagtcgaagcggcgctcgtcgagcggtctgcgcaccacggccaggtcgcaggcgccctccgtcagccccgcggtggcggaattgacgcggacgaggtgcagctccgtctcgggatacgcctgcgcccagcggcgctggaaggcgggggtgtgacggcccagcgcggaccaggcgtagccgatccgcagatgggcgtggcccgatacggcctcccggatcagcccgtccacctcggccagcacccgccgggcgtgtgccaccacccgcagcccggtgcccgtcggggtcacctcgcgggaggtccgccgcaacagccttgtccccagggcgcgttcgagcgctgccagggtgcgggacacggccgcctgggagacgccgagcgcgatggcggcgtcggtgaaggtgccctcgtcgacgatcgcgacgaggcagcgcagttgccgtagctccacatccatacgtccagcgtatagatagaaccccgaacgcattttgcgcatgcatgagccgggcgcacgatcgacgcatgcgactcgcccccgcgtcacgcaccccgtccccacgagcgatggacaccgcacaccggaccgcgccgacccctgccgactacgacctgggacagggcctggagcggggcctggcccctgaccctgatcagcggccgaccggacggcggttcgccggtgtggccacgatgatcggcagtgggctgtccaaccagaccggcgccgcgatcggatcccaggccttccccgtcatcggcccggtcggggtcgtcgccgtccgccagtacgtggccgcgatcgtcctgctggccgtcggcaggccccggttgcggagcttcacctggtggcagtggcggccggtggtggggctcgccgtggtgttcggcaccatgaatctgtccctgtacagcgccatcgaccgcatcggcctcgggctggcggtgaccctggagttcctcggcccgctgtgcatcgcgctcgccggctcacggcgccgcgtggacgcctgctgtgcgctggtcgcggcggccgccgtggtgaccctcatgcgcccgcgcccctcggccgactatctgggtatggggctggggttgctggccgccgtgtgctgggcgtcgtacatcctgctcaaccgcaccgtggggcggcgggtccccggcgcccaggggtcggcggcggccgcggggatctccgcgctgatgttcctgccggtcgggatcgccgtcgccgtccaccagccgccgaccgtgagcgccgcggcgtacgccatcatcgcgggcgtcctctcctcggccgtgccgtacctcgcggacctgttcacgctgcgccgcgtgcccgcccaggcgttcgggctcttcatgagcgtcaaccccgtcctcgccgcactggtcggctgggtcggcctggggcagagcctggggtggacggagtggatcagcgtgggcgccatcgtcgcggccaacgcgctgagcatcctcacccggcgcggctgaaggaccagcgggggtggcccggtgacttggctgacctggacccgggggtggacccggggacggagggccgcgccgcccccaggccaccgctccgcccccgggccaccgctcagcccgcggcctcgaacagcgcctccgcggcggcgatcgcctcggccagggcggtgggctccggccgcagccccgccacgatcgtgtcgatggcgcgcaggtccgcccgctggaggagccgcttctcgttggtgacccactcaccgcgcgccgccagcaccgcgtgcgccgtctggagggcggcggtcgccaccgcccccgcgacctcggtggcctggccacgtcccacgtacgcggccgaggcgtaccgcagggtcaaggccgcccgcccgcgccacgccgggggtgccgcctcacgcagcgccgccgggtactcggggcggggcagggtgccccgcagcacctggttcagggcgagttcggccaccaccagatagctggggatgcccgcgaggtggaacatcagcggctcccagtggaagcggccccgtcgcgactcggcgagttcgtgttccaccacctcgaggtcgcggtagtggacgtccacgcgccgtccgtcgatcgtcagccaggcgcccccgttgaagacaccaccgccccactcgccgagctcggagacctcgccctcccagcccacggcccgcagcgcggccgggtcgaagccgcctcggtagtacagggccaggtcccagtcgctctccggggtgtgggtcccctgcgcacgcgagcccccgagggcgacggcgtgcacggcgggcagggcggcgagtcgctctgcgacatcgtcgaggaacgtgtcgtcggtcatgaggaacatgtcgtcggtcatacggatccgatcgtgtgaagtggatgacgggtgccgcgggcacaccgaacgcacgccggagcacaggctcgaacggcggcgtccatacggatcggtgtgccgggtcatcactccatgacgccgaccttaccgcccccgtcagagggcggcagcggccagcggcggatcagtccttcaccaggcccaggcggaacaggctctccgcggtgtcgaggatggtcgtcaccgggtcgcgcggggtccagccgaacacggaacgcgccttctcggtgcgcaggatcggcacccgctccgtcacgccgaccgcttcccgcgctcgttcgtcgtcgaactcccgcgtgggcacccgggcggcgcgctcgccgaggtgctcggccagcacctgggcgatccacaggaagctgacggtccggtcgccgctggcgaggaagcgctctccggccgcggcggggtgtgccatggcccggaggtggagctcggcgacatcgcgcacgtccaccatgccgaagtgtgcgcgggggacggccgacatcgccccctccagcatcgcccggacgtgttccgtcgaggcggacagccgggggccgagtgccggaccgaagatcccggtcgggttgatcaccgtcagttcgaggccgtccccctccttcgccacgaagtcccaggcggccagctccgcgatggtcttcgagcggatgtagggcgggttgtcgtcctcggggtcggtccagtcgctctcgtcgtactcgtcaccgtccttgtggctgtatcccaccgcggcgaacgaggacgtcatcacgacccgtttcacaccctggtcccgtgcggccctcagcacacgaagggtgccgtcccgcgcggggacgatcagctcgtcggcgttgtccggctggacggcggggaacggtgacgcgacgtggtggacgcgggtgcaccccgccatcgcgtcgtcccagccgtcgtccgtggtcaggtcggcgctgacgatatcgagccgcccgccgggatcgacaccggaggccgcgatggccgaccggacactcgcggcggcgccggtggccgggccgtgtgaacggaccgtggtgcggacccgatggccgctccgcagcaggccgctgatcacatgggtgccgagatagccgctgcctcctgtcaccaggacgagttcgccactaacggtgtcgccacgggcgtcggcgccggcatcagcggacacgggggttgccttgctttccatggggtacttcggatcccttcccaagtgtgtttctcgcagctgtgtctctcacggccgcagcgcgtcgatgacgtccgtcagttcgtcgatcgcccggcgctccgcatcggcgtcggcgcgctcccgcgcgtcccacatgtccgccttgagccgcagatagcgcagccggacctccatgagcgcgatctcccgctccaggcggtccgcgttgcgttggaagaggtcacgcaggggagccgcaccctgatcgccttcgtcgaggtggccgaggtaggcgcgcatgtcctgcatgctcatgccggtggatctcaggcaccccagcgacctgatcgtctccaccacggaggggggatagcgccggtggccactgtcccggtcgcggtccacggcggggatcaagccgatcttctcgtagtagcgcagtgtcggctccgagaggccactcagcctcgacacctgctggatggtcatcggggagccccggacctctgtcctcgtcgttgtcataggacgagcatccgatacttgaagcgcttgaggtcaagcgagccgatcggcctttgcggggacggcggtcccggaagtgggcgcgcccgggcgcttcccggccgtggcgatggagatgtcccgcatgaggagcagcgccccgagggcgaggagcccggcggcgccgccgctccaggagacggtggagccgatggccgtggcggtgggcgcggccgcgccggagtggccgatcagggactgggcgctggccaggccgaccgcgccaccgagctgcttggtgagcgcggaacccgcggtggcggtgcccatgtccgcacgcgggacggcgctctgggtggcgatggtgagcccgcccatggccggtcccgcgccgagcccgacgagcagcagcagaacggacgtcagcgcgagaggggtcgtggcccgcagggcgacgaaggcggcggtaccggcggtgagcagccccgcgccgatcagcaggaccggcttgacgtgcccgctgcgcagcacggtggcggcggtgagccggttgcccagggtcatgccgatgagcagggggagcagcagcagaccggaggcggtggccgaatggccgcggatgtgctggaagtacagcggcaggaagattcccaccggcgccgcggcgacctggaagaagaaaccggcggtcagcagggcggtgtaggtgcggtgccggaacagccgcaggggcaggacggggacggcggcccgccgctcgaccggtatgagcgtggtgagcagcgccagaccgccgagcagacagcccagcacggccgggtccgtccaggagggcgcgtgtccggcggtcgcgttccccttgaggctgaggccggtcagcgcgagggcgagccccgcggcgagcaggaggatccccgccacgtcgagccggccggacggcggggtggcgggacggcggtcgggcagggccaggacgatgacggcgcccgcggccagcccgagcgggaggttgagccagaacgcccagcgccagccgatgtgatcggcgagtaacccgccgaggagcgggccgcccaccatgcccaggatcatcatggcggccatcgccgtctgcatccggatgaggccctgggggcgggacggcgggtggaggtcgcggaccagtgccatgccgagggtcagcagggatccggcacccaggccctggagcgcgcgggagaggatcagggcgggcatcgaggcggacaggccgcaggcgatggagccgatcaggaagacgccgagcccgccgatcagcagccggcggcggccgtggaggtcggagaagcggccgtagaccggcacgctgaccgaggaggtcagcagataggcggtgacgagccagacgtaccaggagtcccctccgccgatctgctcgacgatgcggggcagcgcggtgccgaccacggtgccgtccagcatggccaggaaggcgcagcccagcagggcgatggtgaccagggcccggcggcggtgcgggagcgcttcgtacccgtcgggtccggtcaccgggcctccccgggcgccacgagcgcgccgtgcaggaagaggtccacgacttcctcggtggtcagcggctcgggggcgcccaggcgcccggccgacatcagcgtcagctggaaggcgtcggcgagccgttccggggcgagtcgcaggcggtcccggtcgggctcgaacagcgcggccagcgcggcgcgcggccggaccaggctcgcctcgcggtccgggaggcgcccgtccttgccgggcttgggcgccatgcgctccagccgcccggccgccgcgagcgccccggcgaccgcgccgatgcgcgccatgtgtccgcgcaccacatcggccgcctcggcgagccggtccgcaagcggctggtcaagggcgatcgactccagatgggccacggtgtcatcgggccgcacggcctccgccatacaggccgcgagcagggcgtccttgtcctcgaagacgcggaagatagtgccttccccgatgcccgcggcccgggcgatcttcgcggtcgtcacggtggcgccgtattcgacgacgagggggagcgcggcggcgacgatcatcgcgcggcgctggtcgggatccatggccggagcgcggcggcgggtcggagtggaggggttctctgccttctctgtcatgcgggatacggtacggagtgagtactcactccgtcaatgcacggtgcgcggccacaaggcgagtggcggttcggcttcgacgttgtcggtcagcgcgcggcgagcagggcccggcgcagggcgcggcccgcctcggacgggggatggttgcgcgaggcggcgaggccgagaccgtgcagcggcggtggatcggcgagatcgacctgggtcaggccgggccgcgaggcgatggtctcctcggccacgaaggcgagcccgagacgccgccggaccatggtcagggcggtcgtggtgtcgacgacctccagtgccacggtgcgctgaactccggcggtgccgaagaggctgtcgacgatggtgcggtcgccccaccccgtggggaagtcgatgaagcggcggtcggcgaggtcggcgtaggtcacgccgtgcgcctcggcgagggggtcgtcggtgcggcaggccagcccgaggcgtatccgcgacacatcatcgatgatcagatccgggccgaggacggcggggccgtgcgggggcaccggcagcagcatcaggtcgaacctgccctcgcgcagggcggtggcgtgtccggccagcggaccggtcgagtggcgcagccgcaccacgacatcggggtgctcggcctgaaacgtgctcagcgccccgatcaggtcgaacgagccggtggacaggaccgtcccgagggtgaccgtaccgctgagccccccggtgagacggcccatgtcatcgcgcgcccgctgcgcctccgcgagcaggatccgggcccgggccagcagggtgcgccccgcggtggtcagctccagggtgcggtgcgagcggtcgaagagcgcggtctggaactcctgctccagccgggccacggcggcggaggccgccgactggacgacgtgttcccgctgggcgccgcgggtgaagctgcgctcctcggccaccgcgacgaagtacgccagctgccggagctccaccatcatctccattcgcgatgccacacagcacacatcatcgttggacacgatacctatgggaccgccaccgtggaggggaagcggaacgccccggccggacggcccggttcggcgccacgccccccaacttccccgtgtgccagcacacttcaccacggaaggcatccatcgtcatgagcgtctcagccatccagatcgggctccaccccgatgccatcgactacgaggcgccggagttcgccgccttcgccggtctgagccgggagacgttgcgcgccgccaacgacgacaacctcgccctgctgctcgacgccggatacgaggcggacggctgtcagatcgacttcggggagaccgccctcgacaccatccgcgccatgctcggccgcaagcgctacgacgcggtcctcatcggcgccggggtacggctcaccgcgggcaatacactgctcttcgaatccatcgtcaacctcgtccacaccgcgttgccccacgcgcggttcatcttcaaccactccgccgcggccacccccgacgacatccgccgccactaccccgacccggcctccaccgttcccctcgacgtcccccgcgacctcgaggaggccgcgctgaagaaccccggcaacgccgcccgccccgaagccgcccacggcccgcgggagacgcggtgaccgccccggcccggccccacggtgaggcgaacccggaccttcacaccacggatgtgctcgtcgtcggcggcgggccgaccggaatgaccctggccggggatctggcacgggccggacgcgcggtcaccgtgctggaacgccggccggcgatccatccgtccagccgtgccttcgtcaccatgccccgcaccctggaagtcctcgacagccgtggtctggccgacgacctcctggccggggcgaacaccaccgaagcggtccacctgttcgcgggcgccacgctcgatctgacacatctgccctcccgccaccgatacgggatgatcaccccgcagaccaatgtggaccaggcgctcgaacgctacgcccgcgaccagggcgcccgggtgctgcgcggcaccgaggtcaccggcctcgcccaggacgccgacgcggtcaccgtcaccgcccgcgccgacggcggcggacccgcttccacgtggcgagcccggtacgtcgtgggggcggacggggcgcacagcaccgtccgcggcctcctcggcgccgacttccccggaaggacggttctgacctccgtggtgctggccgatgtccgcctcgccgacggccccaccgggaacgggctcaccctgggcaacacccctgaggtcttcggcttcctcgtgccgtacgggaaggcgcgccccggctggtaccggtcgatgacctgggaccgccgccaccaactgcccgacaaggccgccgtggaggaggcggaggtcacccgcgtactggccgaggccatgggacgtgacgtcggggtccgtgagatcggctggcactcccggttccactgcgatgaacgccaggtccgctcctaccggcacggccgggtcttcctcgccggggacgccgcccacgtgcactccccgatgggcggccagggcatgaacaccggcgtccaggacgcggccaacctcgcctggaagctcgacctcgccctcggcggcgccgacccggccatcctggacacctaccaccgggagcgccaccccgtcggccgccgtgtcctgctccagagcggtgccatgatgcgcgccgtcaccctcgggccgcgcccggcgcggtggctgcgcgaccatctggccccggccctgctgggcgtcggccgggtgcgcgacaccatcgccggaagcttcaccggcgtcaccccgcgctatccgcgcggacggcgacagcacgcactggtgggcacccgcgccaccgaagtcccgctcgccgagggccggttgaccgaactgcagcgggccggtggctttctgctgatccgcgagcggggcgcggcgcgcgtcgacaccacggtggcccaggccgagcgcaccgactccggccccgccctgctggtccgccccgacggctatatcgcctgggccggacccggtgtccgtacggacggccccgacggctggcacaccacatggcgggcctggaccggcccggccaccgatgcggtgcgcgccgggcgctgaacaggagacggggagacggcgccgggcgggcggcccggcgccgacgccctcatccgttccccgtcgcctgcccggcggacagggagtcggggagggcagcggcggccggttcctcgggtcgtcccttgcggaagaaccggagcagcgacgggccgccccggaaacaccacagcgcgatgaccggatcgcagatcgcacagcccagcgacgagccatgcgcgaacgggacgatggggttgcccttgatgtgatgcacgatgtcccacgcggtgtgcagcagccagccgatgccgatgaaggtccacgactccaggccacggtaggccacataggtggcgaccacggtgaaggcgaactcccagccgtccaggccgccgccgctgaggtaggccgcacccgctccgccgaccatgatcgcgttgaagcgccggcggtgcggttcgcgaatcagggacatcaggagcgcgtagaggacaccgatgaagaccggagcgatgtattggatcatgcggaagaacttcctgcgggtgacggaacgttggccgcccggcggggcgacggttcatcacgctagatccgcccccggccgccccacagggccattcccgacacgctccaacggataatcgccggggccggatcatcgccgtggccacggcctccacccggccaccacgctcagggcccgatcacagcagccgccacaggtggtcatcggttccgttgtcgtcgtactgcaccacctgggcgctgttggcggtggacatgccgtcgacacccagcaccttctggctgttcttgttgaggacgcggaaccagccgtcgccgttgtccaccttccgccagaggtgatcggccgtgccgttgtcctcgtactggacgacgatggcgctgttcgcggtggacatccggtcgacgcccaggaccttgccgctgtggccgttgcggatcaggaaccagccgtcgccccggtcgatccactgccaggcgtggtcgcccgtcggtgtgttgtcgtactgcaccacgcgggcgctgttggccgtggacatctcgtcgacggcgagcaccttgccgctgtgcttgttgagcagtcggcggaagggcggctccggggtccacgcccggccgtccggcgcggccgtgggaaagcaggtgatacgcagccgggcggcgcccatggggatgagggtgaccgtctccgccggtgcgtcggcccgggccgggctctgctgaagcggggtgaccacatgctcgtcgtccgagacccactcggcgatacggcgcgcctgggcggtcatgcggaccggggtggtctcgtgggtgaagggattggcggcgagcggaccgtcgtcgcgggtgagcacggggagggctccgggggcgaggccgtagttccacggagtggtggcgtgcacttcgtactcggggaaggtgtcggtgccggcgtagcgcacgaagtcctcgccgatgcgcagggagtacgtcagcgggccgtggtcgacgctgaccgcgccgtgctgcgccgaccaggtccgcagggcggtgcgctgcggcaggcggatcgtcaccacatcgccgtccgtccagctccggtcgaccttgacgaaggccggaccgccgcgcgtggccaccgcccggccgttgacctcgatccgggggttcttgcaccagccggggacccgcagatggagcgggaaggccaccttctcgggggtggacagcgtgagtgtgatggtctcgtcgaacggatagtcggtgtcctcggtgacggtgaccgtcgtaccgcccgccaccttcgcggacacctggcttgcggcgtacagggaggcggcgagccccttgtcgggcgtggccagccacagctcctcgctgaagtacggccagcccatgccgtagttgtgcggacagcagcggtactggtcgacgcccggctggtacgactgcatcgcgaagccgttctggaactgcccctgcgacttcaccgcgttgttcagatcgatgctgttcgcgctggtgatgtagtgggtgccggtgccctgggggtcgagggcggcgggcagcatgttgaacgccaggtcctcgcaccggtcggcccacaccggatcgccggtgatccgggtcagcagctcatggctggccatgaattcgacgatgccgcaggtctcgaagccctgccgggggtctccgaaacccgggcggtagttctcgtccccggcgaagccaccgcccgggaactggccgtatgcgccgagcaccgacgtatagccgcggtaggtcgcctgcctgagctcggcggagccggtcagctgggcgtactgggcgggctcgcggaagccctgggcgatattgacgttgtgcggggtcgggatgttgtcgacccaattggcgccgtacgtgtgcatcttctggacgaggtcgaggaggaacgcctcgccggtgcggcggtggagccacatcgcggtgtcgattccgtcgccccagcggtaggagacccagctggagtcgaaggcgcccgggccctgcgcgttcatgaagcgcaggaagcgggtgaggaaggggacgatgcgctggtcgccggtgaactcctcatgggtgcgcagggccatgaggagggggaggaacggccagaagtcggggccgccgttcagctttgtccgcagggagcgcggcccgaagaagccgtcgctctgctgggtggcgaggatggcgtcgatccatccgcgggcgttggcgagcgccgcctggtcgcgcgtcgccaccgccagcgggacatagccacgcagccagtacggcacctcctcccagccgtcccggtccgggtgggtccacccggtggcgttgatgtcgaggaagtgcgagcgctcctggtaccggccgcagaggccgtggagttggaggcgcagctgctcggccagccagccacgcggggtgatgctcccggacgggagccggtcgaaggcgtcggacagcggggcccttggccgccgggccggggatgccacggcgtccgtggccaggtgcccggcgagtgcgggggcgccgagggtgagggcgctggtgcggaggaagcgacgtcggtcgaggggcatcgtgcggctcctgtcggtggggtgccgaggaagacgggtggcgcctgacatcgttgtcgcgcatcacagcacgccatcggcgcgctgtctataagttcgacaggccgccctgccccggtgggctctatgctgagcgtgatgtccgcacctcagggccagggccccaccttccgtgaactcgtcgtccaggcgctgtcctccgtcgagcgcggctacgatctgctggccccgaagttcgaccacaccgggtaccggacgtcggcctcggtgctggactccgtgaccggcgccctgcgcccgctcgggcccttcgacagcggcctcgacgtgtgctgcggaaccggcgccggcatgggcgtgctgcgccaggtgtgccgggagcggatcaccggcgtcgacttcagcgcgggcatgctggccgtgggccgggagcgtacgcggacggtgccggacgccccgcgcacggactgggtacgcgccgacgcgcgcgccctcccgttcgagccggtcttcgacctggcggtgagcttcggggcgttcg

SEQ ID NO: 2 - DNA, 28 bp, Streptomyces sp.
gagcctgcgggctctgcgactccgctac

SEQ ID NO: 3 - DNA, 27 bp, Streptomyces sp.
gcgagcgaagcagcgcgcgtgtcgcac

SEQ ID NO: 4 - DNA, 32 bp, Streptomyces sp.
  misc_feature (24)..(24): n is inosine
acgctcgcggctacgcaccggccngccgcaag

SEQ ID NO: 5 - DNA, 42 bp, Streptomyces sp.
agctcgcatcgccggctagagccgccggcatccttgcacctg

SEQ ID NO: 6 - DNA, 888 bp, Streptomyces sp.
atgaccgcgcagattctcgatggcaaggcaaccgcagccgcgatcaagtccgatctcgtcagccgcgtggaggcgctgaaggccaagggcatccatcccggcctcgggaccgtgctggtgggcgaggaccccggcagcaagtggtacgtggcgggcaagcaccgcgactgcgccgaggtcggcatcgcctccatccggcgcgacctgcccgagaccgccacccaggaggagatcgaggcggcggtccgggagctcaacgaggacccgtcctgcacgggctacatcgtccagctgccgctccccaagggcatcgacgccaaccgggtgctggagctgatcgacccggtcaaggacgccgacggactgcacccgatgaacctcggccgcctcgtgctcaacgagagcggcccgctgccgtgcacgccccagggcgtcatccagctgctgcgccaccacggtgtggagatcaacggcgcgcatgtggtggtcgtcggccgcggcatcaccgtcggccggtcgatcgggctgctgctgacccgccgttcggagaacgcgacggtcaccctctgccacaccggcacccgcgacctgcccgggatcctgcgccaggccgacatcatcgtggcggccgccggggtgcggcacctggtcaagccggaggacgtcaagccgggcgcggcggtgctcgacgtgggcgtcagccgggacgagcacggcaagatcgccggcgatgtgcaccccggtgtgaccgaggtcgcgggctgggtctcgccgaacccgggcggggtcggcccgatgacccgcgcccagctgctggtcaacgtggtggaggccgcggagcgggacgcgaaggcggccgccgacgccggtgccggccatgacggctga

SEQ ID NO: 7 - DNA, 1566 bp, Streptomyces sp.
gtgaccgccgaaggtaccaagcggcccatccgccgcgcgctggtcagcgtctacgacaagacgggtctcgaggagctggcccgagggctgcacgcggcgggtgtccagctcgtctcgaccggctcgacggccgcgaagatcgccgccgccggggtcccggtcaccaaggtcgaggagctgaccggcttccccgagtgtctcgacggccgcgtcaagacgctgcacccgcgcgtccacgcgggcatcctcgccgaccagcggctcgactcgcaccgcgagcagctccgggagctgggcgtggaccccttcgagctggtcgtggtcaacctctatccgttccgcgagacggtcgcctcgggcgccgcgccggacgagtgcgtcgagcagatcgacatcggcggcccctccatggtccgggccgccgccaagaatcacccgtccgtggccgtggtcgtcaaccccgagcggtacggcgacgtcctcgaggccgccgcggagggcggtttcgacctggagcggcgcaagcggctggcggccgaggcgttccagcacaccgccgcctacgacgtggcggtggccaactggttcgcggccgactacgcggcggcggacgactcctccttcccggacttcctgggcgccaccatcacccgtaagaacgtgctgcgctacggcgagaacccgcatcagcccgccgccctctacaccgatggcagcggtaaggggctcgcggaggccgagcagctgcacggcaaggagatgtcgttcaacaactacacggacaccgaggccgcccgccgggccgcgtacgaccacaccgagccctgtgtcgcgatcatcaagcacgccaacccgtgcgggatcgcagtcggggcggacgtcgccgaagcgcaccgtaaggcgcacgcctgcgacccgctgtcggccttcggcggggtgatcgccgtcaaccgcccggtgtcggtcgccatggccgagcaggtcgccgagatcttcaccgaggtgatcgtcgccccggcgtacgaggacggcgcggtcgaggcgctcgcccgtaagaagaacatccgggtgctgcgctgcgcggagtcgccggtggaggccgccgagcagcgccccatcgagggcggcacgctcgtccaggtcaaggaccgcctccaggccgagggcgacgacccggccaactggaccctcgccacgggcgaggcgctggacgccgacggcctcgccgagctggccttcgcctggcgctcctgccgcgcggtgaagtccaacgcgatcctgctcgccaagggcggcgccacggtcggcgtcggcatgggccaggtcaaccgcgtggactcggcgaagctggccgtcgagcgggccggtgccgagcgggccgccggttcgtacgccgcctccgacgccttcttcccgttcccggacggcttcgaggtgctggccgaggcgggcgtgaaggccgtggtgcagccgggcggctcggtccgtgacgaggccgtggtggaggccgcccagaaggcgggtgtgaccatgtacttcacgggcacgcgccacttcttccactga

SEQ ID NO: 8 - DNA, 657 bp, Streptomyces sp.
gtggcctccccgccttcccccgccagccctgcgcgccccgggcgcccggtccgcctcgtcgtcctcgtctccggctccggtacgaatctccaggcgctgctcgacgccatcgccgccgagggcgtggcccgatacggcgccgaggtggtggccgtgggcgccgaccgtgacggcatcgagggcctgacgcgcgccgagcgcgccgggatccccacgttcgtgtgccgggtcaaggaccacgccggccgcgccgagtgggacgcggccttggcggaggccaccgccgcccatgagccggacctggtcgtctcggccgggttcatgaagatcctgggccaggagttcctcgcccggttcggcggccgctgcgtcaacacccatcccgcgctgctccccagctttcccggcgcccatggcgtgcgcgacgcgctcgcgcacggtgtgaaggtgaccggatgcaccgtccacttcgtcgacgacggcgtcgacaccggcccgatcatcgcccagggcgtggtcgaggtccgggacgaggacgacgagtccgctctccatgagcggatcaaggaagtcgagcgctcgctgctcgtcgaggtcgtggggcgtctggcccgtcacggctaccgcatagagggacgaaaggtaaggatcccgtga

SEQ ID NO: 9 - DNA, 1854 bp, Streptomyces sp.
gtgtccgcacgcgaccgctcagcgccccggcgttcctccgccatcagggaggcgttcctcggcggtgtggtcgccgcggggctcggcctcggcacgctcgccgtggtcgtactgctgttgtggatcacttcttcctcccccgagagcagccccgacggagccctgcatgtcgccgccgacctatggctgctcggccatggcgccgacctcgtgcgcaccgagacgctctccggccacaccgcgccggtcgggctgaccccgctgctgctcagcgtggtgccgtgctggctgctgtaccgggccgcccagcacgccgtctaccaggcggagcccgatgagggcgacgggcagtgggtgcccgaggagtccgtcgtcgatccgcgcaccgccttcgcctgggtgaccggcggctatctgctggtgggcaccgctgccgcggtgtacgcctcgaccgggccgctgcgtgtcgatccgctgagcgcgctgctgcatctgccggtcgtcgccggggtcatcgccgccgtcggcgtgtggacggccgacggacggttcccgctccgcctgcccggacgggtgagcgagaggctgcggcggctccccggcgccgaacggaccgtaaggacggccgcgtccctggccgcgcgcggctggtgccggcgccgccggctgaccgccgcgctacgggccgggaccagcggtctcgtcgtcctgctcggcagcggtgcgctccttacggcgacgtcgatgctgagccacgcgggcgccgtgcaggtgacgttcctcaacctcagcgatgtgtggtcggggcggttcgcggtgctcctggtgagcctggcgctgctgccgaacgcgatcgtctggggcgcggcgtacggggtcggggcggggttcacggtgggtggcggcagtgtggtggcgccgctgggcatcacctcctacccccagctgccgcacttccccctggtcgccgcgctgcccacggacggctccggcgggccgctggtctggctcacggggatcgcggccggggcgtcggtggcctggctcatcgggatcgcggcggtgcggcggcccggcaagggcgagcccaggccgccctggggctgggccgagacgctggtgctcgccgcgctggcggcggtcggctgcgcggccgcgatggcgctcctggccggggtctcgggcggaccgctgggcatcggcatgctcgcggacctcggcccgagctggtggcgcacgggcgtgatcacgctggcctggacgggagtgatcggggtgcccggcgcgatggtgctgcgctggtaccggctgtgcgtccccaccagggcctcctggccggagtggaaggcggcgcgggcggaccgccggacatcccgcgcgcaggcccgtacggcggccggggaggcccgtacggcagccagggaggcccgtacggaggccaaggccgcccgcgcggcgcgggccgcggccgaggcggaggcgcgggcggcggtgctgcccacggtgtcacccatggagtccgccgaggtgcgcgaggcgatggccgagccgtggtggcagtggctgcgcccgggcgcgtcgggcgccgaccgcaagcggaaccgtaaggcggcgcccgacgccggtgcggagacgggacgggagaccggacgcgaaaccggtgtgggcacgatggccggactcgcgacccccgcgggcaccccgggcgcccccggggccgcccgcccacgccgctgggcactgagccggaagcgcgctcccgggccgcagccgcccgccgagtccaagaccgccccggacccctcgcgcacggagcccccgccgccgccggacgacgcccgccgcgaaccgtaa

SEQ ID NO: 10 - DNA, 885 bp, Streptomyces sp.
atggctatcttcctcaccaaggaaagcaaggtcatcgtccaggggatgaccgggtccgaagggcagaagcacacccgtcggatgcttgcctcgggcaccaacatcgtcggcggcgtgaacccgcgcaaggccggcaccaccgtggacttcgacggcaccgagatcccggtcttcggctccgtcaaggaggccatcgacgccaccggcgccgatgtcacggtcatcttcgtcccggagaagttcaccaagagtgcggtcatcgaggcgatcgacgccgagattccgctcgccgtcgtgatcaccgagggcatcgcggtccacgactccgccaacttctgggcctacgcgggcaagaagggcaacaagacgcgcatcatcggcccgaactgcccgggtctgatcacgccgggtcagtcgaacgcgggcatcatcccggccgacatcaccaagcccggccggatcggtctggtgtcgaagtccggcacgctgacctaccagatgatgtacgagctgcgggacatcggcttctcgtcctgtgtgggcatcggcggtgacccgatcatcggcaccacccatatcgatgccctcgccgccttccaggccgaccccgacaccgacctgatcgtgatgatcggcgagatcggtggcgacgccgaggagcgggccgcggacttcatcaaggccaacgtcaccaagccggtcgtcggctatgtggcgggcttcaccgcccccgagggcaagacgatgggccacgcgggtgccatcgtctccggctcctccggcaccgcccaggcgaagaaggaggccctcgaggccgcgggcgtgaaggtcggcaagaccccgtccgagaccgcgcgcctggcgcgcgccgcgctggccggctga

SEQ ID NO: 11 - DNA, 1119 bp, Streptomyces sp.
gtgctggccggtgaagtcatcgacacgcctggggcggcgcgcgaggtggccgagcggctgggcggccgcgcggtcgtcaaggcgcaggtcaagacgggcggccgcggtaaggcgggcggcgtcaagctggcctccgacccggatgacgccgtcgagaaggccggccagatcctgggcatggacatcaagggccacacggtccacaaggtgatgctcgccgagaccgcggacatcaaggaggagtactacgtctccttcctgctggaccgcaccaaccgcaccttcctcgccatggcctccgtcgagggcggcgtggagatcgaggtcgtcgcggagcagaaccccgaggcgctcgccaagatcccggtggacgccatcgagggcgtgaccgaggagaaggccgccgagatcgtcgccgccgcgaagttcccggccgagatcgcggaccaggtcgtcgcggtgctccagaagctgtggaccgtcttcatcaaggaagacgccctgctcgtcgaggtcaacccgctggtcaagaccgaagacggcaaggtcatcgcgctggacggcaaggtctccctggacgagaacgccgccttccggcagccggagcacgaggcgctcgaggacaaggccgcggccaacccgctcgaggcggccgccaaggccaagggcctcaactacgtcaagctcgacggcgaggtcggcatcatcggcaacggcgcgggtctggtcatgtccaccctcgacgtcgtcgcctacgcgggcgagaaccacggcaacgtcaagcccgccaacttcctcgacatcggtggtggcgcctccgccgaggtgatggccaacggtctcgagatcatcctcggcgacccggacgtcaagtcggtcttcgtcaacgtcttcggtggcatcaccgcctgtgacgcggtcgccaacggcatcgtccaggccctggagctgctgaagtccaagggcgaggacgtcagcaagccgctggtcgtgcgcctcgacggcaacaacgcggagctgggtcgcaagatcctcaccgacgccaaccacccgctcgttcagcaggtggacaccatggacggcgcggccgagcgtgccgccgagctggctgcgaagtaa

SEQ ID NO: 12 - DNA, 1341 bp, Streptomyces sp.
gtgccctgcacctacaccgccgacatcggccagtacgacgagcccgacatcgcctcggtccccgggcgcgccacgacgtacggcgcggaggtcgcccgtctggtggactgccgggcggccctggtggaggaggggctcgcggccctcgcctgcggggcgttccacatccgctccggcggccgcagctacttcaacaccaccccgctcgggcgggcggtcaccggcaccctgctggtgcgcgccatgctcgaggacaatgtgcagatctggggcgacggctccaccttcaagggcaatgacatcgagcggttctaccgctacggtctgctggccaacccctccctgcggatctacaagccgtggctggacgccgacttcgtcagcgagctcggcggccgcaaggagatgtcggagtggctgctcgcccatgacctgccctaccgggacagcgcggagaaggcgtactccaccgatgccaacatctggggcgccacccacgaggcaaagtcgctcgagcacctcgacaccggtatcgagatcgtccagccgatcatgggcgtgcggttctgggacccgtcggtcgagatagcggccgaggacgtcacgatcggcttcgagcagggccgcccggtaacgatcaacggcaaggagttcgcctccgccgtcgatctggtgctggaggccaacgccatcggcggtcggcacggcatgggcatgtccgaccagatcgagaaccgcgtcatcgaggccaagagccggggcatctacgaggcaccgggcatggcgctgctgcacgcggcctacgagcggctggtcaacgcgatccacaacgaggacaccgtcgccacctaccacaccgaggggcgccgcctcggccggctgatgtacgagggccgctggctggacccgcaggcgctgatggtgcgcgagtcgctgcagcgctgggtcggcgcggcgatcaccggcgaggtgaccctgcggctgcggcgcggtgaggactactccatcctggacacctccggaccggcgttcagctaccacccggacaagctctccatggagcggaccgaggactccgccttcggtccggtggaccggatcggccagctgaccatgcgcaacctcgacatcgccgactcacgcgccaagctggagcagtacgccggtctcggcatggtcggcagctcgcatccggcgctgatcggcgccgcgcaggcggcgtccaccgggctgatcggcgcgatgccgcagggcgcctccgaggcgatcgcctcggacgggcacgtgtccggacaggacaagctgctcgaccgcgccgcgatggagttcggcgccgactga

SEQ ID NO: 13 - DNA, 2133 bp, Streptomyces sp.
gtggccgtcgccctcgcggccggcacgctcgtcaccctgacccccacggcggcacacgcggccgcgggcgcctccctgcccttcgcctcggccgaggccgagtcggccaccaccacggggacgaagatcggccccgacttcacccagggcacgctcgcctccgaggcatccgggcgccaggccgtccgcctcgccgccgggcagcgcgtggagttcaccgcgccccgcgcggcgaacgcggtgaacgtggcctacaacgtgcccgacggccagtcgggcacgttgaacgtctatgtcaacggcaccaagctggccaagaccatcgcggtcacgtccaagtactcgtacgtggacaccggctggatcgcggggtcgaagacccaccacctctacgacaacgcccggctgctgctcggccagaacgtccaggccggtgacaagatcgccttcgaggcggcgaacacccaggtcaccgtggacgtggccgacttcgagcaggtcgcggcggccgcctcccagcccgccggatcggtgtccgtcacctccaagggcgccgaccccagcgggcagggcgactccacccaggcgttccgggacgccatcgccgcagcccagggcggtgtggtctggatcccgccgggtgactacaggctgacctcctcactgaacggcgtccagaacgtcaccctccagggcgccggcagctggcactccgtggtgcacacctcgcggttcatcgaccagtccagctcctccggcaacgtccacatcaaggacttcgcggtcatcggcgaggtcaccgagcgcgtcgactccaaccccgacaacttcgtcaacggctcgctcggcccgggctccagcgtgtccggcatgtggctgcagcacctgaaggtcggtctgtggctgatgggcaacaacgacaacctcgtggtcgagaacaaccgcttcctggacatgacggccgacggcctcaacctcaacggcagcgccaagaacgtacgggtccggaacaacttcctgcgcaaccagggcgacgacgcgctcgccatgtggtcgctgaactcgccggacaccaacagcagcttcgagagcaacaccatctcgcagccgaacctcgccaatggcatcgccatctacggcggtacggacatcacggtcaagaacaacctgatctccgacaccaacgccctgggcagtggcatcgccatctccaaccagaagttcatggacccgttccacccgctggccggcacgatcacggtcgacggcaacacgctggtccgagcgggcgccatgaaccccaactggagccacccgatgggcgccctgcgcgtcgactcctacgacagcgcgatcgaggccaccgtcaacatcaccaacacgaccatcaccgacagcccgtacagcgccttcgagttcgtctccggcggcggcaggggctacgcggtcaagaacgtcaatgtgtccggcgcgaccgtgaccaaccccggaacggtcgtcgtccaggccgaggcgcagggggcggtgaagttcggcgatgtcacggcctccagcgtcggcgcggcgggcgtctacaactgcccgtacccgtcgggctccggcaccttcgacctcaacgacggcggcggcaactccggctggagcagcacctggtcggactgtgccagctggccccagcccggccggggcaacccggatcccgacccgggccgcaacctcgccaagggccgcccggccaccgcgaccggctcttgggacgtctacacccccggcaaggcggtcgacggcgacgcgaacacctactgggagtcgaccaacaacgcctttccgcaggccctgaccgtggacctcggcgccggccaggccgtccgcaggctggtgctgaagctgccgccctcgtcggcgtggggcgcccgcacccagaccctgtccgtgctgggcagcaccgacggctcctcgtactccacggtggtgggctcgcagggctaccgcttcgacccggcgtccggcaacaaggtcaccgtcgccctgcccgacagcacgaatgtgcgctatctgaggctcagcgtcaccggcaacaccggctggcccgcggcccaggtcagtgaggtggaggcgtatctgacctcatga

SEQ ID NO: 14 - DNA, 1599 bp, Streptomyces sp.
gtggcccagcccacccctgcccggacgccgaacgactggtggcgctccgccgtcatctaccaggtgtatgtgcgcagcttcgccgacggggacggcgatggcaccggcgacctcgcgggcgtccgcgccaggctgccgtatctcgccgaactcggcgtcgacgcgctgtggttcagcccctggtaccagtcgcccatgaaggacggcggctatgacgtcgccgactaccgcgccatcgatccggccttcggcaccctggccgaggcggagaaactcatcgccgaggcccgtgagctgggcatccgcacgatcgtggacatcgtcccgaaccacgtctccgaccagcacccctggtggcgggccgccctcgcgggcggcgccgagcgcgagctcttccacgtccgcccgggccgcggcgagcacggtgaactgccgcccaacgactggacgtcggagttcggcggcccggcgtggacccggctgccggacggccactggtatctgcatctgttcgcccccgaacagccggacctcaactgggcccatccggccgtacgccaggagcacgaggacatcctgcgcttctggttggagcggggtgtcgcgggggtgcgcatcgactcggccgccctgctggccaaggatccccggctgcccgacttcgtcgagggccgcgatccccatccgtacgtcgaccgcgatgagctccatgacatctaccgctcctggcgcggcgtggccgacgagtacggcggtgtcttcgtcggtgaggtgtggctgccggacagcgagcgcttcgcccgctatctgcgccccgacgaactgcacaccgccttcaacttctcgtttctggcctgcccctgggacgcccggcggctgcggacgtcgatcgacgagacgctcgccgaacacgctccggtgggagctccggccacctgggtgctgtgcaaccacgatgtgacccgcacggtgacccgctacgggcgcgaggacaccggtttcgacttcgccaccaaggtcttcggcacccccaccgacctcaccctcggcacccggcgggcacgggccgccgccctgctgtcgctggccctgcccggcgcggtctacgtctaccagggcgaggaactgggcctgcccgaggccgacatcccccgcgaccgcatccaggacccgatgcacttccgctccggcggcaccgacccgggccgggacggctgccgggtgccgctgccgtgggcggcggaggcgccgtacgccggtttcggctcgcgcgaggagccgtggctgccgcagcccgcgcactgggcggcgtacgcggccgatctgcagacggaggccccgggctcgatgctcggcctctaccgcgcggcgatccgcatccgccgcaccacccccggcttcggcgacgggccgctgacctggctcccctcggccgacggtgtcctggccttcgcccgtgcggacggcctggtctgcgtggtcaacctcgcggacacccccaccgagctggacggcgcctcccggcttctgctcagcagcggcccgctggacgaccggggccgccttccgcaggacacggcggcctggctgctccgctga

SEQ ID NO: 15 - DNA, 876 bp, Streptomyces sp.
atgagcacccggaccctcgtctcccccgccgccctggcccgcccccgcggccgggccgtctactggacggtcttcaccaccgtggtggtgctgttcgcgatcgccttcctcttcccggtctactggatggtgaccggtgcgatgaagtcgcccgacgaggtggcgcggacaccgcccaccatcgtcccgaaagagtggcacctcagcggctacagcgacgcctgggacctgatgcagctgccgcagcacctgtggaacacggtggtccaggcagccggcgcctggctgttccagctggtcttctgcacggccgccgcctatgccctgtccaggctgaagcccgccttcggcaaggtgatcctcggtggcatcctggccacgctgatggttccggcccaggcgctggtcgtgccgaagtacctgaccgtcgccgacctgccgctgatccacaccagcctgctcaacgacccgctcgcgatctggctgccggccgtcgccaacgccttcaacctctatctcctcaaacggttcttcgaccagatcccgcgcgatgtcctggaggccgccgagatcgacggcgccgggaagctgcgcaccctgtggtcgatcgtgctgcccatgtcgcgcccggtgctcggcgttgtgtcgatcttcgcgctggtggcggtgtggcaggacttcctgtggccgctgatggtcttctccgacaccggcaagcagccgatcagcgtggcactcgtccagctgtcgcagaacatccagctgaccgtgctcatcgccgcgatggtcatcgccagcatcccgatggtcgcgctgttcctcgtcttccagcggcacatcatcgccgggatcagcgcgggcagcacgaagggctga

SEQ ID NO: 16 - DNA, 1023 bp, Streptomyces sp.
atgtcgaccagctcctggaggaagcctccgacaagatcgacaacattctggcccggggctgacccgatgaccaagaccgccgcgcggccgcccgccgaggcgatcgccgtccacccggtgcaggcgccgcccccggcggggggtcgggggcggcgccgtctcgccgaccaggtccgggcctatggcttcctcctcggcggcctgatctgcttcgcgctgttctcctggtatccggcgatccgcgcggtcgtgatcgccttccagaagtacacgcccggctcgtcccccgaatgggtcggcaccgccaacttcacccgcgtcctgcacgacccggagttcaccgcggcctggcggaacaccctcaccttcaccctgctggcactcctcatcggcttcgcgatcccgttcctgctcgccctcgtgctcaatgaactgcggcacgccaaggcgttcttcagggtcgtggtctatctgccggtgatgatcccgccggtggtcagcgccctgctgtggaagtggttctacgacccgggcgccgggctggccaacgaggcgctgcgcttcctgcacctgcccacctcgaactggtccaacggcgccgacaccgctctggtctccctcgtcgccgtggccacctgggccaatatgggcggcaccgtcctgatctacctggcggcgctgcagtccatccccggtgagctgtacgaggcggccgaactcgacggcgcgagcctgctgcagcgcgtccgccacgtcacgatcccgcagacgcggttcgtgatcctcatgctgatgctgctgcagatcatcgcgacgatgcaggtcttcaccgagccgttcgtgatcaccggtggtggcccggagaacgccacggtcacggtcctctacctgatctacaagtacgccttcctctacaacgacttcggtggcgcctgtgcgctgagcgtgatgctgctcgtgctgctcggcgccttctccgccctctatctgcggctcacccgctccggggaggacgacgcatga

SEQ ID NO: 17 - DNA, 1431 bp, Streptomyces sp.
gtgcttgagtgtgcggcgcactcacggttgtgtgcgccccgctcacggccgtggggcttccctgctcctgtacagaggggtccacccatgagaagcaccgggttccgtcgtactctcatcgcgctcagcacgttccccctcgccctcaccgcctgcggcgggtcgggcgacggctcggcgggcggaaagacgcgcatcacggtcaactgcatgccgcccaagagcgccaaggtcgaccgcaggttcttcgaggaggacatcgcctccttcgagaagcagaacccggacatcgacgtcgtcgcgcatgacgcgttcccctgccaggacccgaagacgttcgacgccaagctggccgggggccagatggagaacgtcttctacacgtacttcaccgacgccggacatgtggtcgacatcaaccaggcggccgatctcacgccgtacgtcaaggagttgaagagctactccaccctccagaagcagctgcgcgacatctacacggtcgacggcaagatctacggcatcccgcgcaccggctactcgatgggtctgatctacaaccgcaagctcttcgagaaggccggactcgaccccgacaagcccccgatgacctgggaggaggtccgcgccgacgccaagaggatcgccaagctgggcgatggcacggtcggctacgcggactacagcgcccagaaccagggcggctggcacttcacggccgagctgtactcacagggcggcgatgtcgtcagcgcggacggcaagaaggccaccatcgacacccccgaggcgcgcgccgtcctgcggaacctccacgacatgcgctgggtggacgactcgatgggcagcaagcagctcctggtcatcaacgacgcccagcagctgatgggctccggcaagctgggcatgtacctggccgcgcccgacaacctcccgatcctggtgaaggagaagggcggcaactacaaggacctcgccatcgcccccatgcccggtggcaagggcacgctcatcggcggcgacggctacatgttccagaagaaggacacgcccgcccagatccgggccggtctcaagtggctcgaccacatgttcctcaccccgggcgatggcttcctcggcgactacgtccgcgccaagaagcgaaacgccccggtgggcctgcccgagccacggctgttcaccggcgcagccgacgccaaggaccagcaggtcaagaaggccaacgccaatgtccccgtgggcaactaccagaccttcctcgacggcaaccagaagctgcggatgaggatcgagccgccgcacgcccagcagatctactccgtgctcgacggagccgtctccgccgtcctcaccaagaaggacgccgatgtcgaccagctcctggaggaagcctccgacaagatcgacaacattctggcccggggctga

SEQ ID NO: 18 - DNA, 978 bp, Streptomyces sp.
gtggcgaagaaggtcggtgtcagcgaggccacggtcagccgggtactcaacggtaagcccggggtctccgcagccacccggcaggcggtgctgtccgccctggacgtcctcggctacgagcggcccacgcagctgcggggcgaccgggcccggctggtggggctggtgctgcccgagctgcagaaccccatcttcccggcgttcgccgaggtcatcggtggggcgctggcacagcttggactgaccccggtgctgtgcacccagaccaagggcggggtctccgaggccgattacgtggcgctgctgctgcaacagcaggtctccggggtggtgttcgcgggcgggctgtacgcgcaggccgacgcgccgcatgaccactaccggctgctcgccgagcgcaacatcccggtggtgctggtcaacgcggccatcgagcacctcggcttcccggctgtctcctgcgacgacgccgtggccgtggagcaggcgtggcggcatctggcctccctcggccatgagcggatcggcctggtgctcgggcccggtgaccacatgccgtcggcacgcaagctgaccgccgcgcgggcggtcgcaggccaccttccggatgagttcgtggcccgggcgatcttctcgatcgagggcggccacgccgctgcctcccggctgatcgaccggggcgtcacgggcatcatctgcgccagcgacccgctggccctgggcgcgatacgagccgcgcgccgcaaggggttcggcgtgccgtcgcaggtgtccgtggtcggctacgacgactccgcgttcatgaactgcaccgagccgccgctgaccaccgtccgccagcccatagaggccatgggcagggcggcggtggaggtgctgaacgcgcagatcggcggggtggccgtaccgtccgaggagctgctgttcgagccggagctggtggtccgcggctccaccgcccaggcgccacgggagtga

SEQ ID NO: 19 - DNA, 519 bp, Streptomyces sp.
gtgtgccgccccctcggatcgtcacgccgtggcggccggccccgaggacgcgggtttgttgtcgggtcgagtggtaacgcggtgaacatgaccgagaagaagaacgcacacactacccggagcaccaacgtgaacgcgaaggccaccgccaccaaggccaaggagaccgcggaaagggccaaggacaccgcgggcaaggcggagaccacggcgaagaccgccgcggccggcgcggcgacgaccgcggcgcacaccgctcatgtcgccgccgacaaggctcaagtggccgccgggaaggccgtgaccaccggtcgtaccgtggccgctgaggcgcccaagaaggctgccgcggcggcgggttcggcctggatgatgatcaaggcccggaaggtcctggcagccgtcgccggtgcgggtgccgccgcggcgggcgccaccgccgcggtcgtcctgcgcaggcgcgcggctcgtcgccgccgcccgctggcacgcctgaccggcggccggctcggctcttga

SEQ ID NO: 20 - DNA, 1485 bp, Streptomyces sp.
atgtccgccgcctcttccgacccgacgtctccccgtgtccccccgacacgtcgactggccctcggggtcatcgccaccggcatgctgatggtgatcctcgacggcagcatcgtgaccgtggccatgcccgccatccagagcgatctgcggttctcccccgccgggctcagctgggtcgtcaacgcctacctgatcgcgttcggcggtctgctgctgctcggcggccgtatcggcgatctcatcggccgcaagcgggtgttcctgaccggtaccgcggtgttcaccgcggcctcgttgctcgcggccgtggccacctcccccgctgtgctgatcgccgcacggttcctccagggggtcggcagcgcgatggcctcggcggtcagcctgggcatcctcgtcacgctcttcaccgaacgcgccgaacggtcgaaggcgatcgccgtgttcagcttcaccggcgccgccggagcgtcgatcggccaggtgctcggcggcctcctcaccgacgcgctcagctggcactggatcttcctgatcaatctgccgatcgggctgctgacgctcgcggtcgccatacccgtcctgcccgccgaccgcgggccgggcctcgcggccggcgccgatgtcctcggcgccctgctggtcacgaccgggctgatgctgggcatctacaccgtggtcaaggtggcggactacggctggacggcggcgcgcacactcggcctcggcgccgtctcgatcctcctgatcgccctgttcctggtccgccagaccaccgcccgcaccccgctgatgcccctgcggatcctgcggtcgcgcggggtggcgggggccaatctggtccagctcctgatggtggccgcgctcttctcgttccagatcctggtcgccctctatctgcgcaatgtgctggggtacgacgccaccggaaccggtctggccatgctcccggccgccatcgccatcggcgcggtgtccctcggcgtctccgcacggctcagcgcacgcttcggcgaccgcgcggtgctgctgaccgggctggccctcctgaccggcgttctcggcctgctcgtccgcgtccccgtgcacgcccggtacctccccgacctcctcccggtgatgctgctcgccgccggtttcgggctggcgctccctgcgctgaccagcctgggaatgtccggtgcgaaggaggacgaggccgggctcgtctccgggctgttcaacaccacccagcagatcggcatggcgctgggcgtcgcggtgctgtccaccctggccgcctcccgcacggatgccctgctctcccggggcaagggtcgggccgaggcgctgaccggcggctaccacctggccttcgccgtcggaacggggctcatcgtggcggccttcgcggtggcgttcaccgtactgcgaggacctgcgcgcaagccccccgctgtgccgcggaacgccaatccgcccgccacaccggtcgccaccgcctga

SEQ ID NO: 21 - DNA, 480 bp, Streptomyces sp.
atggcgcccaccaagaccgaacccgacctgtcgttcctcctcgaccacaccagccacgtcctccgcacccagatgtcggccgcgctcgccgaaatcgggctgacggcgcggatgcactgcgtactggtccacgccctggaggaagagcgcacccaggcccagctcgccgagatcggcgacatggacaagaccacgatggtggtgacggtggacgccctggagaaggcgggcctcgcggagcggcgcgcctcgacccacgatcgccgggcccggatcatcgcggtcaccgaggagggcgcgcggatcgccgaacggagccaggagatcgtggaccgcgtccatcgcgaggcgttggcgacactccccgagacccaacgcgccgcgctgctaaaggcgttgacccggctgtccgaggggcatctggccacgcccgccgagagcccccgcccggcgcggagggcgcggcagcgcgagaagtag

SEQ ID NO: 22 - DNA, 945 bp, Streptomyces sp.
gtgacgcgggggcgagtcgcatgcgtcgatcgtgcgcccggctcatgcatgcgcaaaatgcgttcggggttctatctatacgctggacgtatggatgtggagctacggcaactgcgctgcctcgtcgcgatcgtcgacgagggcaccttcaccgacgccgccatcgcgctcggcgtctcccaggcggccgtgtcccgcaccctggcagcgctcgaacgcgccctggggacaaggctgttgcggcggacctcccgcgaggtgaccccgacgggcaccgggctgcgggtggtggcacacgcccggcgggtgctggccgaggtggacgggctgatccgggaggccgtatcgggccacgcccatctgcggatcggctacgcctggtccgcgctgggccgtcacacccccgccttccagcgccgctgggcgcaggcgtatcccgagacggagctgcacctcgtccgcgtcaattccgccaccgcggggctgacggagggcgcctgcgacctggccgtggtgcgcagaccgctcgacgagcgccgcttcgactccgccatcgtcggactggagcggcggctgtgcgccgtggccgccgacgacccgctcgccaggcgccgctcggtccggctggccgacctcagcgggcgcaccctgctggtcgaccgcaggaccggtaccaccaccacggagctgtggccgcccgactcccggccggccacggaggagacccacgacgtggaggactggctcaccgtgatctccgcgggccgctgcgtcggcatgacggcggagtccacggccaaccagtatccgaggcccggaatcgcctaccggccggtccgcgacgccgagcccatcgcggtacgcctcgcctggtggcgggacgacccgcaccccgccacccagaccgcggtcgagctgctcaccgccctctaccgcaacggctga

SEQ ID NO: 23 - DNA, 825 bp, Streptomyces sp.
gtgcgttcggtgtgcccgcggcacccgtcatccacttcacacgatcggatccgtatgaccgacgacatgttcctcatgaccgacgacacgttcctcgacgatgtcgcagagcgactcgccgccctgcccgccgtgcacgccgtcgccctcgggggctcgcgtgcgcaggggacccacaccccggagagcgactgggacctggccctgtactaccgaggcggcttcgacccggccgcgctgcgggccgtgggctgggagggcgaggtctccgagctcggcgagtggggcggtggtgtcttcaacgggggcgcctggctgacgatcgacggacggcgcgtggacgtccactaccgcgacctcgaggtggtggaacacgaactcgccgagtcgcgacggggccgcttccactgggagccgctgatgttccacctcgcgggcatccccagctatctggtggtggccgaactcgccctgaaccaggtgctgcggggcaccctgccccgccccgagtacccggcggcgctgcgtgaggcggcacccccggcgtggcgcgggcgggcggccttgaccctgcggtacgcctcggccgcgtacgtgggacgtggccaggccaccgaggtcgcgggggcggtggcgaccgccgccctccagacggcgcacgcggtgctggcggcgcgcggtgagtgggtcaccaacgagaagcggctcctccagcgggcggacctgcgcgccatcgacacgatcgtggcggggctgcggccggagcccaccgccctggccgaggcgatcgccgccgcggaggcgctgttcgaggccgcgggctga

SEQ ID NO: 24 - DNA, 1050 bp, Streptomyces sp.
gtgtccgctgatgccggcgccgacgcccgtggcgacaccgttagtggcgaactcgtcctggtgacaggaggcagcggctatctcggcacccatgtgatcagcggcctgctgcggagcggccatcgggtccgcaccacggtccgttcacacggcccggccaccggcgccgccgcgagtgtccggtcggccatcgcggcctccggtgtcgatcccggcgggcggctcgatatcgtcagcgccgacctgaccacggacgacggctgggacgacgcgatggcggggtgcacccgcgtccaccacgtcgcgtcaccgttccccgccgtccagccggacaacgccgacgagctgatcgtccccgcgcgggacggcacccttcgtgtgctgagggccgcacgggaccagggtgtgaaacgggtcgtgatgacgtcctcgttcgccgcggtgggatacagccacaaggacggtgacgagtacgacgagagcgactggaccgaccccgaggacgacaacccgccctacatccgctcgaagaccatcgcggagctggccgcctgggacttcgtggcgaaggagggggacggcctcgaactgacggtgatcaacccgaccgggatcttcggtccggcactcggcccccggctgtccgcctcgacggaacacgtccgggcgatgctggagggggcgatgtcggccgtcccccgcgcacacttcggcatggtggacgtgcgcgatgtcgccgagctccacctccgggccatggcacaccccgccgcggccggagagcgcttcctcgccagcggcgaccggaccgtcagcttcctgtggatcgcccaggtgctggccgagcacctcggcgagcgcgccgcccgggtgcccacgcgggagttcgacgacgaacgagcgcgggaagcggtcggcgtgacggagcgggtgccgatcctgcgcaccgagaaggcgcgttccgtgttcggctggaccccgcgcgacccggtgacgaccatcctcgacaccgcggagagcctgttccgcctgggcctggtgaaggactga

SEQ ID NO: 25 - DNA, 408 bp, Streptomyces sp.
gtgtcgaggctgagtggcctctcggagccgacactgcgctactacgagaagatcggcttgatccccgccgtggaccgcgaccgggacagtggccaccggcgctatcccccctccgtggtggagacgatcaggtcgctggggtgcctgagatccaccggcatgagcatgcaggacatgcgcgcctacctcggccacctcgacgaaggcgatcagggtgcggctcccctgcgtgacctcttccaacgcaacgcggaccgcctggagcgggagatcgcgctcatggaggtccggctgcgctatctgcggctcaaggcggacatgtgggacgcgcgggagcgcgccgacgccgatgcggagcgccgggcgatcgacgaactgacggacgtcatcgacgcgctgcggccgtga

SEQ ID NO: 26 - DNA, 1494 bp, Streptomyces sp.
gtgaccggacccgacgggtacgaagcgctcccgcaccgccgccgggccctggtcaccatcgccctgctgggctgcgccttcctggccatgctggacggcaccgtggtcggcaccgcgctgccccgcatcgtcgagcagatcggcggaggggactcctggtacgtctggctcgtcaccgcctatctgctgacctcctcggtcagcgtgccggtctacggccgcttctccgacctccacggccgccgccggctgctgatcggcgggctcggcgtcttcctgatcggctccatcgcctgcggcctgtccgcctcgatgcccgccctgatcctctcccgcgcgctccagggcctgggtgccggatccctgctgaccctcggcatggcactggtccgcgacctccacccgccgtcccgcccccagggcctcatccggatgcagacggcgatggccgccatgatgatcctgggcatggtgggcggcccgctcctcggcgggttactcgccgatcacatcggctggcgctgggcgttctggctcaacctcccgctcgggctggccgcgggcgccgtcatcgtcctggccctgcccgaccgccgtcccgccaccccgccgtccggccggctcgacgtggcggggatcctcctgctcgccgcggggctcgccctcgcgctgaccggcctcagcctcaaggggaacgcgaccgccggacacgcgccctcctggacggacccggccgtgctgggctgtctgctcggcggtctggcgctgctcaccacgctcataccggtcgagcggcgggccgccgtccccgtcctgcccctgcggctgttccggcaccgcacctacaccgccctgctgaccgccggtttcttcttccaggtcgccgcggcgccggtgggaatcttcctgccgctgtacttccagcacatccgcggccattcggccaccgcctccggtctgctgctgctccccctgctcatcggcatgaccctgggcaaccggctcaccgccgccaccgtgctgcgcagcgggcacgtcaagccggtcctgctgatcggcgcggggctgctcaccgccggtaccgccgccttcgtcgccctgcgggccacgacccctctcgcgctgacgtccgttctgctgctgctcgtcgggctcggcgcgggaccggccatgggcgggctcaccatcgccacccagagcgccgtcccgcgtgcggacatgggcaccgccaccgcgggttccgcgctcaccaagcagctcggtggcgcggtcggcctggccagcgcccagtccctgatcggccactccggcgcggccgcgcccaccgccacggccatcggctccaccgtctcctggagcggcggcgccgccgggctcctcgccctcggggcgctgctcctcatgcgggacatctccatcgccacggccgggaagcgcccgggcgcgcccacttccgggaccgccgtccccgcaaaggccgatcggctcgcttga

SEQ ID NO: 27 - DNA, 642 bp, Streptomyces sp.
atgacagagaaggcagagaacccctccactccgacccgccgccgcgctccggccatggatcccgaccagcgccgcgcgatgatcgtcgccgccgcgctccccctcgtcgtcgaatacggcgccaccgtgacgaccgcgaagatcgcccgggccgcgggcatcggggaaggcactatcttccgcgtcttcgaggacaaggacgccctgctcgcggcctgtatggcggaggccgtgcggcccgatgacaccgtggcccatctggagtcgatcgcccttgaccagccgcttgcggaccggctcgccgaggcggccgatgtggtgcgcggacacatggcgcgcatcggcgcggtcgccggggcgctcgcggcggccgggcggctggagcgcatggcgcccaagcccggcaaggacgggcgcctcccggaccgcgaggcgagcctggtccggccgcgcgccgcgctggccgcgctgttcgagcccgaccgggaccgcctgcgactcgccccggaacggctcgccgacgccttccagctgacgctgatgtcggccgggcgcctgggcgcccccgagccgctgaccaccgaggaagtcgtggacctcttcctgcacggcgcgctcgtggcgcccggggaggcccggtga

SEQ ID NO: 28 - DNA, 1047 bp, Streptomyces sp.
gtgaagtgtgctggcacacggggaagttggggggcgtggcgccgaaccgggccgtccggccggggcgttccgcttcccctccacggtggcggtcccataggtatcgtgtccaacgatgatgtgtgctgtgtggcatcgcgaatggagatgatggtggagctccggcagctggcgtacttcgtcgcggtggccgaggagcgcagcttcacccgcggcgcccagcgggaacacgtcgtccagtcggcggcctccgccgccgtggcccggctggagcaggagttccagaccgcgctcttcgaccgctcgcaccgcaccctggagctgaccaccgcggggcgcaccctgctggcccgggcccggatcctgctcgcggaggcgcagcgggcgcgcgatgacatgggccgtctcaccggggggctcagcggtacggtcaccctcgggacggtcctgtccaccggctcgttcgacctgatcggggcgctgagcacgtttcaggccgagcaccccgatgtcgtggtgcggctgcgccactcgaccggtccgctggccggacacgccaccgccctgcgcgagggcaggttcgacctgatgctgctgccggtgcccccgcacggccccgccgtcctcggcccggatctgatcatcgatgatgtgtcgcggatacgcctcgggctggcctgccgcaccgacgaccccctcgccgaggcgcacggcgtgacctacgccgacctcgccgaccgccgcttcatcgacttccccacggggtggggcgaccgcaccatcgtcgacagcctcttcggcaccgccggagttcagcgcaccgtggcactggaggtcgtcgacaccacgaccgccctgaccatggtccggcggcgtctcgggctcgccttcgtggccgaggagaccatcgcctcgcggcccggcctgacccaggtcgatctcgccgatccaccgccgctgcacggtctcggcctcgccgcctcgcgcaaccatcccccgtccgaggcgggccgcgccctgcgccgggccctgctcgccgcgcgctga

SEQ ID NO: 29 - DNA, 408 bp, Streptomyces sp.
atgtccctgattcgcgaaccgcaccgccggcgcttcaacgcgatcatggtcggcggagcgggtgcggcctacctcagcggcggcggcctggacggctgggagttcgccttcaccgtggtcgccacctatgtggcctaccgtggcctggagtcgtggaccttcatcggcatcggctggctgctgcacaccgcgtgggacatcgtgcatcacatcaagggcaaccccatcgtcccgttcgcgcatggctcgtcgctgggctgtgcgatctgcgatccggtcatcgcgctgtggtgtttccggggcggcccgtcgctgctccggttcttccgcaagggacgacccgaggaaccggccgccgctgccctccccgactccctgtccgccgggcaggcgacggggaacggatga

SEQ ID NO: 30 - DNA, 2451 bp, Streptomyces sp.
atgtcaggcgccacccgtcttcctcggcaccccaccgacaggagccgcacgatgcccctcgaccgacgtcgcttcctccgcaccagcgccctcaccctcggcgcccccgcactcgccgggcacctggccacggacgccgtggcatccccggcccggcggccaagggccccgctgtccgacgccttcgaccggctcccgtccgggagcatcaccccgcgtggctggctggccgagcagctgcgcctccaactccacggcctctgcggccggtaccaggagcgctcgcacttcctcgacatcaacgccaccgggtggacccacccggaccgggacggctgggaggaggtgccgtactggctgcgtggctatgtcccgctggcggtggcgacgcgcgaccaggcggcgctcgccaacgcccgcggatggatcgacgccatcctcgccacccagcagagcgacggcttcttcgggccgcgctccctgcggacaaagctgaacggcggccccgacttctggccgttcctccccctcctcatggccctgcgcacccatgaggagttcaccggcgaccagcgcatcgtccccttcctcacccgcttcctgcgcttcatgaacgcgcagggcccgggcgccttcgactccagctgggtctcctaccgctggggcgacggaatcgacaccgcgatgtggctccaccgccgcaccggcgaggcgttcctcctcgacctcgtccagaagatgcacacgtacggcgccaattgggtcgacaacatcccgaccccgcacaacgtcaatatcgcccagggcttccgcgagcccgcccagtacgcccagctgaccggctccgccgagctcaggcaggcgacctaccgcggctatacgtcggtgctcggcgcatacggccagttcccgggcggtggcttcgccggggacgagaactaccgcccgggtttcggagacccccggcagggcttcgagacctgcggcatcgtcgaattcatggccagccatgagctgctgacccggatcaccggcgatccggtgtgggccgaccggtgcgaggacctggcgttcaacatgctgcccgccgccctcgacccccagggcaccggcacccactacatcaccagcgcgaacagcatcgatctgaacaacgcggtgaagtcgcaggggcagttccagaacggcttcgcgatgcagtcgtaccagccgggcgtcgaccagtaccgctgctgtccgcacaactacggcatgggctggccgtacttcagcgaggagctgtggctggccacgcccgacaaggggctcgccgcctccctgtacgccgcaagccaggtgtccgcgaaggtggcgggcggtacgacggtcaccgtcaccgaggacaccgactatccgttcgacgagaccatcacactcacgctgtccacccccgagaaggtggccttcccgctccatctgcgggtccccggctggtgcaagaacccccggatcgaggtcaacggccgggcggtggccacgcgcggcggtccggccttcgtcaaggtcgaccggagctggacggacggcgatgtggtgacgatccgcctgccgcagcgcaccgccctgcggacctggtcggcgcagcacggcgcggtcagcgtcgaccacggcccgctgacgtactccctgcgcatcggcgaggacttcgtgcgctacgccggcaccgacaccttccccgagtacgaagtgcacgccaccactccgtggaactacggcctcgcccccggagccctccccgtgctcacccgcgacgacggtccgctcgccgccaatcccttcacccacgagaccaccccggtccgcatgaccgcccaggcgcgccgtatcgccgagtgggtctcggacgacgagcatgtggtcaccccgcttcagcagagcccggcccgggccgacgcaccggcggagacggtcaccctcatccccatgggcgccgcccggctgcgtatcacctgctttcccacggccgcgccggacggccgggcgtggaccccggagccgcccttccgccgactgctcaacaagcacagcggcaaggtgctcgccgtcgacgagatgtccacggccaacagcgcccgcgtggtgcagtacgacaacacaccgacgggcgaccacgcctggcagtggatcgaccggggcgacggctggttcctgatccgcaacggccacagcggcaaggtcctgggcgtcgaccggatgtccaccgcgaacagcgccatcgtcgtccagtacgaggacaacggcacggccgatcacctctggcggaaggtggacaacggcgacggctggttccgcgtcctcaacaagaacagccagaaggtgctgggtgtcgacggcatgtccaccgccaacagcgcccaggtggtgcagtacgacgacaacggaaccgatgaccacctgtggcggctgctgtga

SEQ ID NO: 31 - protein, 295 aa, Streptomyces sp.
MTAQILDGKATAAAIKSDLVSRVEALKAKGIHPGLGTVLVGEDPGSKWYVAGKHRDCAEVGIASIRRDLPETATQEEIEAAVRELNEDPSCTGYIVQLPLPKGIDANRVLELIDPVKDADGLHPMNLGRLVLNESGPLPCTPQGVIQLLRHHGVEINGAHVVVVGRGITVGRSIGLLLTRRSENATVTLCHTGTRDLPGILRQADIIVAAAGVRHLVKPEDVKPGAAVLDVGVSRDEHGKIAGDVHPGVTEVAGWVSPNPGGVGPMTRAQLLVNVVEAAERDAKAAADAGAGHDG

SEQ ID NO: 32 - protein, 521 aa, Streptomyces sp.
VTAEGTKRPIRRALVSVYDKTGLEELARGLHAAGVQLVSTGSTAAKIAAAGVPVTKVEELTGFPECLDGRVKTLHPRVHAGILADQRLDSHREQLRELGVDPFELVVVNLYPFRETVASGAAPDECVEQIDIGGPSMVRAAAKNHPSVAVVVNPERYGDVLEAAAEGGFDLERRKRLAAEAFQHTAAYDVAVANWFAADYAAADDSSFPDFLGATITRKNVLRYGENPHQPAALYTDGSGKGLAEAEQLHGKEMSFNNYTDTEAARRAAYDHTEPCVAIIKHANPCGIAVGADVAEAHRKAHACDPLSAFGGVIAVNRPVSVAMAEQVAEIFTEVIVAPAYEDGAVEALARKKNIRVLRCAESPVEAAEQRPIEGGTLVQVKDRLQAEGDDPANWTLATGEALDADGLAELAFAWRSCRAVKSNAILLAKGGATVGVGMGQVNRVDSAKLAVERAGAERAAGSYAASDAFFPFPDGFEVLAEAGVKAVVQPGGSVRDEAVVEAAQKAGVTMYFTGTRHFFH

SEQ ID NO: 33 - protein, 218 aa, Streptomyces sp.
VASPPSPASPARPGRPVRLVVLVSGSGTNLQALLDAIAAEGVARYGAEVVAVGADRDGIEGLTRAERAGIPTFVCRVKDHAGRAEWDAALAEATAAHEPDLVVSAGFMKILGQEFLARFGGRCVNTHPALLPSFPGAHGVRDALAHGVKVTGCTVHFVDDGVDTGPIIAQGVVEVRDEDDESALHERIKEVERSLLVEVVGRLARHGYRIEGRKVRIP

SEQ ID NO: 34 - protein, 280 aa, Streptomyces sp.
MSRTTPLLREQQSSSSSSSSSSSAPGGPNGDQHEDNPFAPPPEGRPDQPWRPRHRPDGSGGESGEGRPGAQGGQDGPDGDQSGEQPQQPPAWGSQWSSRQPGRQNGGFGGTPGSNRPSGPGGPGGPRWDPNDPAQRRARYALLSGMWAFFFALFSLPQIALLLGVLALYWGISSLRAKPRRTAPSPAAAAPLNAPPPPPGAARAALPAPGSGPAKSQSTAAISGLVTGGLALAIVAATFSFQVVYSDYYTCVDDALTQTSRHDCETLLPEQLRPLLSTQD

SEQ ID NO: 35 - protein, 617 aa, Streptomyces sp.
VSARDRSAPRRSSAIREAFLGGVVAAGLGLGTLAVVVLLLWITSSSPESSPDGALHVAADLWLLGHGADLVRTETLSGHTAPVGLTPLLLSVVPCWLLYRAAQHAVYQAEPDEGDGQWVPEESVVDPRTAFAWVTGGYLLVGTAAAVYASTGPLRVDPLSALLHLPVVAGVIAAVGVWTADGRFPLRLPGRVSERLRRLPGAERTVRTAASLAARGWCRRRRLTAALRAGTSGLVVLLGSGALLTATSMLSHAGAVQVTFLNLSDVWSGRFAVLLVSLALLPNAIVWGAAYGVGAGFTVGGGSVVAPLGITSYPQLPHFPLVAALPTDGSGGPLVWLTGIAAGASVAWLIGIAAVRRPGKGEPRPPWGWAETLVLAALAAVGCAAAMALLAGVSGGPLGIGMLADLGPSWWRTGVITLAWTGVIGVPGAMVLRWYRLCVPTRASWPEWKAARADRRTSRAQARTAAGEARTAAREARTEAKAARAARAAAEAEARAAVLPTVSPMESAEVREAMAEPWWQWLRPGASGADRKRNRKAAPDAGAETGRETGRETGVGTMAGLATPAGTPGAPGAARPRRWALSRKRAPGPQPPAESKTAPDPSRTEPPPPPDDARREP

SEQ ID NO: 36 - protein, 294 aa, Streptomyces sp.
MAIFLTKESKVIVQGMTGSEGQKHTRRMLASGTNIVGGVNPRKAGTTVDFDGTEIPVFGSVKEAIDATGADVTVIFVPEKFTKSAVIEAIDAEIPLAVVITEGIAVHDSANFWAYAGKKGNKTRIIGPNCPGLITPGQSNAGIIPADITKPGRIGLVSKSGTLTYQMMYELRDIGFSSCVGIGGDPIIGTTHIDALAAFQADPDTDLIVMIGEIGGDAEERAADFIKANVTKPVVGYVAGFTAPEGKTMGHAGAIVSGSSGTAQAKKEALEAAGVKVGKTPSETARLARAALAG

SEQ ID NO: 37 - protein, 372 aa, Streptomyces sp.
VLAGEVIDTPGAAREVAERLGGRAVVKAQVKTGGRGKAGGVKLASDPDDAVEKAGQILGMDIKGHTVHKVMLAETADIKEEYYVSFLLDRTNRTFLAMASVEGGVEIEVVAEQNPEALAKIPVDAIEGVTEEKAAEIVAAAKFPAEIADQVVAVLQKLWTVFIKEDALLVEVNPLVKTEDGKVIALDGKVSLDENAAFRQPEHEALEDKAAANPLEAAAKAKGLNYVKLDGEVGIIGNGAGLVMSTLDVVAYAGENHGNVKPANFLDIGGGASAEVMANGLEIILGDPDVKSVFVNVFGGITACDAVANGIVQALELLKSKGEDVSKPLVVRLDGNNAELGRKILTDANHPLVQQVDTMDGAAERAAELAAK

SEQ ID NO: 38 - protein, 546 aa, Streptomyces sp.
VPGTVGSWTGTEGRSAGTCGRSAGSCGRSAGTVGSCGRSVGSCGRSAGTVGRSAGSCGRSAGSCGRSAGTCGRSAGSCGRSAGTVGSCGRSVGSCGRSAGTVGRSAGSCGRSAGSCGRSAGSTGRAGMLATSVRSVGTAGSRLSAPSLALPTVVWAFAATPVARPWANGTVVPATSCANGTAVDGTLTTTGTRRSTVSWAAGTTLEAVPSAKPSALPVTTSTYGVVRATASSASAPVSPTVRSATGTTLTTRRCTAGGSTSEATPSTTGLVVPTAPCTLPVSSSGRMPAPEREASRSLAEPGAEGSFGVITTGDHGYAKAVLDSPLHRDVGALFPRGGGMSWASTAGLGALDLATVPNKLTPKQRAEVRAMVTKAADRYAADSAKSAYGVPYAPKDGKYEWGSNSQVLNNMIVLATAHDLTDKPRYLDAVLRGMDYLLGGNPLNQSYVTGHGERDSHNQHHRFWAHQRDHRLPHPAPGSLAGGPNSGLQDPVAKKKLKGCAPAMCYTDSLMAFSTNEITINWNAPLAWIASYVDGLGGGAAEQSVR

SEQ ID NO: 39 - protein, 446 aa, Streptomyces sp.
VPCTYTADIGQYDEPDIASVPGRATTYGAEVARLVDCRAALVEEGLAALACGAFHIRSGGRSYFNTTPLGRAVTGTLLVRAMLEDNVQIWGDGSTFKGNDIERFYRYGLLANPSLRIYKPWLDADFVSELGGRKEMSEWLLAHDLPYRDSAEKAYSTDANIWGATHEAKSLEHLDTGIEIVQPIMGVRFWDPSVEIAAEDVTIGFEQGRPVTINGKEFASAVDLVLEANAIGGRHGMGMSDQIENRVIEAKSRGIYEAPGMALLHAAYERLVNAIHNEDTVATYHTEGRRLGRLMYEGRWLDPQALMVRESLQRWVGAAITGEVTLRLRRGEDYSILDTSGPAFSYHPDKLSMERTEDSAFGPVDRIGQLTMRNLDIADSRAKLEQYAGLGMVGSSHPALIGAAQAASTGLIGAMPQGASEAIASDGHVSGQDKLLDRAAMEFGAD

SEQ ID NO: 40 - protein, 710 aa, Streptomyces sp.
VAVALAAGTLVTLTPTAAHAAAGASLPFASAEAESATTTGTKIGPDFTQGTLASEASGRQAVRLAAGQRVEFTAPRAANAVNVAYNVPDGQSGTLNVYVNGTKLAKTIAVTSKYSYVDTGWIAGSKTHHLYDNARLLLGQNVQAGDKIAFEAANTQVTVDVADFEQVAAAASQPAGSVSVTSKGADPSGQGDSTQAFRDAIAAAQGGVVWIPPGDYRLTSSLNGVQNVTLQGAGSWHSVVHTSRFIDQSSSSGNVHIKDFAVIGEVTERVDSNPDNFVNGSLGPGSSVSGMWLQHLKVGLWLMGNNDNLVVENNRFLDMTADGLNLNGSAKNVRVRNNFLRNQGDDALAMWSLNSPDTNSSFESNTISQPNLANGIAIYGGTDITVKNNLISDTNALGSGIAISNQKFMDPFHPLAGTITVDGNTLVRAGAMNPNWSHPMGALRVDSYDSAIEATVNITNTTITDSPYSAFEFVSGGGRGYAVKNVNVSGATVTNPGTVVVQAEAQGAVKFGDVTASSVGAAGVYNCPYPSGSGTFDLNDGGGNSGWSSTWSDCASWPQPGRGNPDPDPGRNLAKGRPATATGSWDVYTPGKAVDGDANTYWESTNNAFPQALTVDLGAGQAVRRLVLKLPPSSAWGARTQTLSVLGSTDGSSYSTVVGSQGYRFDPASGNKVTVALPDSTNVRYLRLSVTGNTGWPAAQVSEVEAYLTS

SEQ ID NO: 41 - protein, 532 aa, Streptomyces sp.
VAQPTPARTPNDWWRSAVIYQVYVRSFADGDGDGTGDLAGVRARLPYLAELGVDALWFSPWYQSPMKDGGYDVADYRAIDPAFGTLAEAEKLIAEARELGIRTIVDIVPNHVSDQHPWWRAALAGGAERELFHVRPGRGEHGELPPNDWTSEFGGPAWTRLPDGHWYLHLFAPEQPDLNWAHPAVRQEHEDILRFWLERGVAGVRIDSAALLAKDPRLPDFVEGRDPHPYVDRDELHDIYRSWRGVADEYGGVFVGEVWLPDSERFARYLRPDELHTAFNFSFLACPWDARRLRTSIDETLAEHAPVGAPATWVLCNHDVTRTVTRYGREDTGFDFATKVFGTPTDLTLGTRRARAAALLSLALPGAVYVYQGEELGLPEADIPRDRIQDPMHFRSGGTDPGRDGCRVPLPWAAEAPYAGFGSREEPWLPQPAHWAAYAADLQTEAPGSMLGLYRAAIRIRRTTPGFGDGPLTWLPSADGVLAFARADGLVCVVNLADTPTELDGASRLLLSSGPLDDRGRLPQDTAAWLLR

SEQ ID NO: 42 - protein, 291 aa, Streptomyces sp.
MSTRTLVSPAALARPRGRAVYWTVFTTVVVLFAIAFLFPVYWMVTGAMKSPDEVARTPPTIVPKEWHLSGYSDAWDLMQLPQHLWNTVVQAAGAWLFQLVFCTAAAYALSRLKPAFGKVILGGILATLMVPAQALVVPKYLTVADLPLIHTSLLNDPLAIWLPAVANAFNLYLLKRFFDQIPRDVLEAAEIDGAGKLRTLWSIVLPMSRPVLGVVSIFALVAVWQDFLWPLMVFSDTGKQPISVALVQLSQNIQLTVLIAAMVIASIPMVALFLVFQRHIIAGISAGSTKG

SEQ ID NO: 43 - protein, 340 aa, Streptomyces sp.
MSTSSWRKPPTRSTTFWPGADPMTKTAARPPAEAIAVHPVQAPPPAGGRGRRRLADQVRAYGFLLGGLICFALFSWYPAIRAVVIAFQKYTPGSSPEWVGTANFTRVLHDPEFTAAWRNTLTFTLLALLIGFAIPFLLALVLNELRHAKAFFRVVVYLPVMIPPVVSALLWKWFYDPGAGLANEALRFLHLPTSNWSNGADTALVSLVAVATWANMGGTVLIYLAALQSIPGELYEAAELDGASLLQRVRHVTIPQTRFVILMLMLLQIIATMQVFTEPFVITGGGPENATVTVLYLIYKYAFLYNDFGGACALSVMLLVLLGAFSALYLRLTRSGEDDA

SEQ ID NO: 44 - protein, 476 aa, Streptomyces sp.
VLECAAHSRLCAPRSRPWGFPAPVQRGPPMRSTGFRRTLIALSTFPLALTACGGSGDGSAGGKTRITVNCMPPKSAKVDRRFFEEDIASFEKQNPDIDVVAHDAFPCQDPKTFDAKLAGGQMENVFYTYFTDAGHVVDINQAADLTPYVKELKSYSTLQKQLRDIYTVDGKIYGIPRTGYSMGLIYNRKLFEKAGLDPDKPPMTWEEVRADAKRIAKLGDGTVGYADYSAQNQGGWHFTAELYSQGGDVVSADGKKATIDTPEARAVLRNLHDMRWVDDSMGSKQLLVINDAQQLMGSGKLGMYLAAPDNLPILVKEKGGNYKDLAIAPMPGGKGTLIGGDGYMFQKKDTPAQIRAGLKWLDHMFLTPGDGFLGDYVRAKKRNAPVGLPEPRLFTGAADAKDQQVKKANANVPVGNYQTFLDGNQKLRMRIEPPHAQQIYSVLDGAVSAVLTKKDADVDQLLEEASDKIDNILARG

SEQ ID NO: 45 - protein, 325 aa, Streptomyces sp.
VAKKVGVSEATVSRVLNGKPGVSAATRQAVLSALDVLGYERPTQLRGDRARLVGLVLPELQNPIFPAFAEVIGGALAQLGLTPVLCTQTKGGVSEADYVALLLQQQVSGVVFAGGLYAQADAPHDHYRLLAERNIPVVLVNAAIEHLGFPAVSCDDAVAVEQAWRHLASLGHERIGLVLGPGDHMPSARKLTAARAVAGHLPDEFVARAIFSIEGGHAAASRLIDRGVTGIICASDPLALGAIRAARRKGFGVPSQVSVVGYDDSAFMNCTEPPLTTVRQPIEAMGRAAVEVLNAQIGGVAVPSEELLFEPELVVRGSTAQAPRE

SEQ ID NO: 46 - protein, 1572 aa, Streptomyces sp.
VGNSGAPKSRGLSAAMSNLFERTRRNESTGIVPVDRGRELRASFAQQRLWFLDQLEPGNASYNLPFAVRVRGRLDISHLSRALSLVVARHEALRTTFGEAGGQPVQRIEPPGPVPVRLEAVSGGSEEERLAEVRRLAGAEITEPFDLSTGPLLRAKALRLDEQDHVLLLTVHHVATDAWSQGIVVRELSVAYASLDAGREPVLPPLPVQYADYAEWERDWLSGPTLRRQLDYWTKRLDGMAPALELPTDRPRPSVASQEGDAVRWELPPELIRAARRLGAGENATLYMTLLAAFQLVLGRYVDSDDITVGTPVANRGRAEVEGLIGFFVNTVVLRTDLSGDPTFRQLLGRVRDTAAGAFAHGDLPFEYLVEQVHPERDLSRNPLVQVLFQMINVPAERLELPGARTEPYDHGGILTRMDLEVHLVETGDGVLGHIVFSKALFDTSTIERLLHHVTVVLRGVLAEPDRRISEISLLDEAERAKVLEKFNTTTGPVPAGSLPALFTAQAERRPDAVAVISGGDRVTYAELDQRANQLAHLLEGRGVGPETLVGLCVDRGIEMIVAILAILKLGAAYVPIDPHHPRDRVQFVLADSGVTVAVTQQRFTGLLETPEAPGTPDASGTSGIRLILLDAEREPLAGQPRTPPTARPSAQNLAYVIYTSGSTGVPKGILMPATCVLNLVAWQKRALPIGPDAKTAQFATLTFDISLQEIFSALLYGETIVVPGEELRMDPAEFATWVHANEIDQLFVPNVMLRAISEEVDPHGTELAALRHLSQAGEPLSLHHDLRELCARRPELRLHNHYGPSEAHVVTSYSLPAEVAEWPLTAPIGRPIGNTRVYVVDRRLRPVPVGVPGELCVAGEGLARGYLGRPDLTASRFVADPFRGDGSRMYRSGDLVRWLPDGNLEFLGRIDDQVKIRGFRIEPGEIEAILARHQDVLHTAVMVREDTPGDKRLVAYVVADATAADRHGGLTETLRRHVESAVPEYMVPSAFVLLDTMPLTSGGKIDRKALPAPDLRTVLEVGYVAPRTPEEEAVCRVYADLLGAAKVGIDDDFFALGGHSLIATRVVARLRSALGIAVPLKTVFQQRTPRELAATLTAAARSGPEPELPPLVPTRRDQPVPLTFAQQQTDLFFDDVLNAGHWNIPMAVRVSGELDLDCLRRAMDLLIDRHEALRTTFVREADGYVQVIRPSAPVQVEVAETHDETEASVLAGQEAARPFDLTRGPLARLRVLRLSQSDHVLVLTLHHLVTDGWSQGVLVRDLSIVYAALLHGTEPDLPPAPVQYADVASWERKWLRGPLLQRQLEFWKRHFEGMTPAELPTDRPRAASARYESDIFHWRLPTDAVETARRLGESCNATLYMTLLTALKVVMSARSDNQDVLVGVPTANRGRDELENTVGLVSKMLALRTEVSGATDFGTLLATVRDAMSDAHTHQDVPFVSVLKHIGDHTAGPAGDTAGGRAGTRLSDDPPVKVIFQIVNTPPRPLRLTGLTAEPFPMTHPPVTVNVDMEIDLYESAEDGGLAGTVLFSKSLFDRATIERFCDDVVAVVSAAAADPGRPVSQVWQGRGRDQ

SEQ ID NO: 47 - protein, 5712 aa, Streptomyces sp.
VAGPGPRPVNDPAPRKRMEPDEAVAVVGMSCRFPQAPDPEAFWRLLSEGISAIGEVPAGRWTDDQPTPSGTDERSTPPAIRRGGFIDDVDRFDPAFFGISPREAAAMDPQQRLMLELAWEGLEDAGIVPATLRGATVGAFIGAGSDDYASLIRARGRSHHTLTGTQRGMIANRLSHVFGLSGPSVTVDAAQASSLVAVHMAVESVRRGESRLALAGGVNLNLSAETAADIAAFGALSPDGRCFTFDARANGYVRGEGGGLVVLKPLSDALADGDTVYCVIEGSAVNNDGGGASLTAPDPDGQRRVLRLAQRRAAISPEAVQYVELHGTGTALGDPAEAAALGAVFGRSGARPVQLGSVKTNIGHLEAAAGIAGLLKTALAIHHRQLPAGLNYRTPNPRIPMGELNLEMRLAPGEWPKPDDRLVAGVSSFGMGGTNCHVLLAEPLVGVPSHASAHAPEPDSLPSSIPAPVPVPVPVPAPVPVPAPAPAPAPVPVPVPLPLSGVSAAALRGQAMRLRPYLERSPNLTDLSFSLATARTSFDHRAVLITGQAADAAHGLDALVEGGTVAGLVTGTARAAGKLAFAFAGQGSQRLGMGRELGAVFPVFAQALDEVCTALDAHLDRPLRDVIHGDDAEPLNRTVYAQAGLFAVEVALFRLLEDFGLVPDLLIGHSLGEVSAAHVAGVLSLADAATFVAARGRLMQAVTEPGAMVSLEATEDEVTRTLMAGGASDDGARVCVAAVNGPTATVISGDERAVLDLAVEWAGRGRKTKRLRTSHAFHSPHLDPVLDELRHIAESLTYRAPRIPLVSNVTGRRATAEELCSPEYWVRHVRRTVRFLDGVRCLEDEGVTTILELGPDKALTTLARDCLTGPGTLVGTLRRDRPEPQALVTALAELYVSGVEVAWSPLVSGGRRIPLPTYAFQRQRYWFSAPGPESGTTPGHGVTSGRERTDTGLSGDEAPDTGPSGGETLGMVRAHAAVVLGYASATAIGAEHTFKQLGFDSITAVELCERLGAATALPLPGTLLFDYPTPAALAEHLHRRLHGRTDEQAAPATVPTPDGGDPVVIVGMGCRFPGRAHSPEDLWRIVADGEDAISGFPSDRGWDLAGLYHPDPDHPGTSYARDGGFLYDAAEFDAGFFGISPREAEAMDPQQRLLLETSWEALERAGIPAEHIKGSSTGVFIGASSVGYAADAGEAAEGYQLTGTAASVASGRVSYTLGLEGPAVTVDTACSSSLVALHLAVQSLRAGECSLALAGGVTVMATPAMFVEFSRQRGLAMDGRCKAFAAAADGTGWAEGVGVLVVERLSDAERNGHRVLAVVRGSAVNQDGASNGLTAPNGPSQQRVIRQALASAGLVASDVDAVEAHGTGTTLGDPIEAQALLATYGQGRDADRPLWLGSVKSNIGHTQAAAGVAGVIKMVMAMRHGVLPRTLHVDEPSTHVDWSGGRVELLTGTTPWPTTGGLRRAGVSSFGVSGTNAHVILEQVPETARPTGPIGEDDGEAAPVAWVLSGQGETGLRAQAERLCAFMAADTRPTPAEVGWSLASTRATLSHRAVVVGAGRDELLRGVNAVANGTPVPGVVRGTGASGDVVFVFPGQGSQWVGMALELVESSPVFARRLGDCADALAPFVEWSLFDVLGDEVAIGRVDVVQPVLWAVMVSLAELWRSFGVVPSAVVGHSQGEIAAACVAGALTLEDGARVVALRSRALLALSGRGGMVSVPVSADRLRDRVGLSVAAVNGPASTVVSGAVEVLEAVLAEFPEAKRIPVDYASHSVQVEGIREGLAEALAPVRPRTGQVPFYSTVTGRLMDTIELDAEYWYRNLRETVEFQSTVEHLMRQGHTVFVEASPHPVLTIGVQDTADTTDTDIVVTGSLRRDDGTVQRFLTSLAELHVRGVRIDWGPLFAGVSPVELPTYAFQRERFWLGADIAESAVDTWRYQISWKPLPDMDPPALSGTWLAVVPEGDEWAMAGARALIESGTASVRTLQVTCDADRRTLAGPLTDVAGSEDIAGVVSFLAADEVPHPAHPALSRGMAHTVELLCSLTTADVEAPLWCVTRAAVTALPADPAPSPAQAAVWGFGRVAGLERSERWGGLIDLPVHCDAHVLRRFVAVLAQAAGEDQVAVRPSAALGRRLEPAPRTGPAGAWRPHGTVLITGGTGVLGAHVARWLARSGAEHLVLLSRRGPQAPGAAVLDDELTALGVRVTLTACDVTDRAALAGVLASVPDLTAVVHLAGTVRFGNSIDADLDEYAGVFDAKVTGALHLDELLDHSSLEAFVLFSSAAAVWGGVGQAGYAAANALLDAVAQRRRARGLPATSIGWGTWGGSLAPEDEERLSRIGLRPMRPEVAVTELRHVVGSAEPCPAIADVDWETFGPAFTAGRPSRLLSELPRLRNTSGAMAMTGDHAALRRRLAGVSAADQARTLVDLVREHAAELLGHRGPAAIDPTVPFRQLGFDSLTAVELRTRLNAATGLRLPATLLFDHPSCRAVADLLRSELLGDRPGSLAASSATEAVPAGVVASDEPIAIVAMSCRFPGGIGTPEDLWRVVSEGRDVLSDFPDDRGWDVDALYDPDPDRPGTSYVRTGGFLHDAAEFDPELFGISPREALAMDPQQRLLLESAWQVLERARMAPTSLRSSRTGVFIGGWGQGYPSASDEGYALTGAATSVMSGRIAYALGLEGPALTVDTACSSSLVALHLASEALRRGECSLALAGGVTVMATPSTFVEFSRQRGLAPDGRCKPFAGAADGTGWGEGVGMLLVERLSDAERLGHPVLAVVSGSAVNQDGASNGLTAPNGPSQQRVIRQALASAGLVASDVDAVEAHGTGTTLGDPIEAQALLATYGQDRDADRPLWLGSLKSNIGHTQAAAGVAGVIKMVMAMRHGVLPRTLHVDEPTPKVDWSAGAVGLLTESAEWRQEGRPRRAGVSAFGVSGTNAHVILEQAPKHAPGVAAEGRKGRGEPPTVPWVLSGASEAGLRAQIEGLRAFADDNPTLDPADVGWSLASTRALLPYRTVVVGTDLDELRRGLDAAEVVGAAEPDRGAVLVFPGQGSQWVGMALELVESSPVFAGRMRDCADALAPFAEWSLFGVLGDEVALGRVDVVQPVLWAVMVSLAELWRSFGVVPSVVVGHSQGEIAAACVAGGLSLEDGARVVALRSRALLALSGRGGMVSVPVSADRLRGRVGLSVAAVNGPVSTVVSGAVEVLEGVLAEFPGAKRIPVDYASHSVQVEGIREGLAEALAPVRPRTGEVPFYSTVTGRLMDTVGLDGEYWYRNLRETVEFQSAIEGLLELGHTVFVEASPHPVLTVGIQDTAETTDTDILVTGSLRRDGGGLASFLTALARLHVRGVAVEWREAFAGLDAHAVDLPTYAFQRRRFWAASLRQTPGTAEFDHPLLGAVLPLPDSGGGLLTGVLTLAGQPWLAEHSVAGVVLFPGTGFVELVLQAGLRWGCGVVEELTLEGPLVLPERGEVEVQVSVGGVDGAGCRSVSVFSCRGGEWVRHAVGVLGVGDGVVPGVEVWPPVGAERVGVEGVYEVLAERGYVYGPVFQGLRDAWRRGDEIFVEAEVPAEARGDAARCAIHPALLDAGLHGVGLGGLISDDGRAYLPFSWSGVRLHAVGASAVRMTLTPAGPDAVSLRVTDEAGEAVLTADSLVLRPVTEGQLAEAEIGNRDVLHRVEWVDAGACSVGSFVEWGEVAAGGVVPDCVVLAGADVAGVLEVLRTWVVEERFEGSRLVVVTRGAVSVGGEGLEDVSGGAVWGLVRSAQSEHPGRFVLVDADVDTDVVPDVVGLGEWQVAVRAGRVWVPRLVDVDVSVGGAVVRGGLGSGVALVTGGTGLLGGLVARHLVSAYGVGELVLVSRRGVAAPGVEELVGELEGLGARVRVVACDVADRGAVAELVGSIEGLRVVVHAAGVVDDGVIGSLDAERLCGVMGPKAWGAWHLHELTRGLDLSAFVLFSSAAGVLGNAGQGGYAAANGFLDALAVHRRGRGLPAVSIAWGFWEERSELTADLAEVQLSRISRSVGASISSAQGLDLFDAALAADEPMVLATPLNLPALRDQAAAGTLPSILSGLVTAPVRRTAGTGRTPAGLRHQLAGVTEAERQHQIMRLVQEHVAGVLGHASAELVDASRTFQEIGFDSLTAVELRNRISAATGIRLPATAVFDHPTPRLLAERVLAEVGGSLPTAAPIAPVSAVDDEPIVIVGMSCRFPGGVESPEDLWRLVHSATDAVSALPTDRGWDLATLSGAKGGAGASYARDGGFLYDAAEFDAGFFGISPREATAMDPQQRLLLEAAWEVFERAGIAPDTLKGSRTGVFTGVMYHDYGSWLTDVPEDVEGYLGTGIAGSVASGRLAYTFGLEGPALTVDTACSSSLVALHLAAESLRRGECSLALAGGVTVLATPQVFVEFTRQGGLAPDGRCKPFAAGADGTGWSEGVGLLLVERLSDAERNGHPVLAVVSGSAVNQDGASNGLTAPNGPSQQRVIRQALANAGLAARDVDAVEAHGTGTTLGDPIEAQALLATYGQGRDVGQPLWLGSVKSNIGHTQAAAGVAGVIKMVMAMRHGVLPRTLHVDEPSPHVDWSAGAVELLGEHMGWPEVGRPRRAGVSSFGASGTNAHVILEQAPDMAGEPEQRPERNELPAIPWVFSAGDEAGLRAQAVRLRAFADRNPDLDPVDVGWSLATGRAGLSHRAVVVGAGRGELLGALEGVPVVGVPVVGGLGVLFAGQGSQRLGMGRGLYEGYPVFAAVWDEVCAQLDQHLDRPVGEVVWGDDAGLVGETVYAQAGLFALEVALYRLIASWGVRGDYLLGHSIGELAAAYVAGVWSLEDAGRVVVARGRLMQALPSGGAMVGVAASEGVVRPLLGEGVVVAAVNGPESVVLSGDEDAVEAVVDVLAGRGVRTRRLRVSHAFHSARMDGMLAEFGEVLRGVEFRAPSVPVVSNVSGAVAGEELCSPEYWVRHVRETVRFADGLDTLRELGVGSFLELGPDGTLTALADGDGVPVLRRDRPEPLTAMAALGGLYVRGVQIDWDAVFPGARRVDLPTYAFQRERFWLEPSPERPTTSVVDAAFWDAVERGDLGSFGIDAEQPLSTALPALSSWRRARQEQSVIDGWRYRLGWMPIPAVSGEVGLTGTWLVVVEPGADGTDVAVALRSAGAGVEVVTSAELSAGPVAGVVSLVSVEATVSLLHVLVAAGVDAPLWCVTRGAVSVVDGDLVDPGQAGVWGLGRVIGLEHPDRWGGLIDLPGELDDRAGNALVGILAGGTGEDQVAIRVTGIWGARLVRATPVPIGDAGGEAAAAWRGRGTALVTGGTGALGRQVARWLVDSGLERVVLTSRRGGEAPGAVELVAELGSRVRVVACDVGDREELAALLAMLPDVRTIVHAAGVLDDGVLESLTPERIREVMRAKADGARHLHELTRDIDLDAFVLFSSAAGTVGNAGQGSYAAANAVLDGLAWRRRAEGLVATSVAWGAWADSGMGAGHARAMAPRLALAALQRALDDDETALMVADVDWSSFGSRFTAVRPSPLLSELLPRSSAPVEPVEALATRLRGMSRIERDRAVLELVRAQVAAVLGHAKPASVDPSRTFQEVGFDSLTAVELRNRLATATGVPFPGSVIFDYPTPTALADHVRARFVPDTDNDEDGGGATSVLDELTRLEAVLSDLSPSDVAGAEVAAKIKSLLSHWGAATNSDIDMDSATDEEMFDLLGKEFGIS

SEQ ID NO: 48 - protein, 7102 aa, Streptomyces sp.
VENEEKLRHYLKEVTKDLRQTRQRLQDVEAKSREPIAIVGMSCRFPGGIATPEALWDLVREGGDAVSEFPADRGWDTEGLYDPAGGSGKSVTRYGGFLRGVADFDAALFGISPREAIAMDPQQRLMLETSWEAFERAGVNRDAVRGSRTGVFIGTNGQDYATLLSAARDDVQGHLGTGSAASVLSGRVAYTFGLEGPTVTVDTACSSSLIALHLAVQALRNGECELALAGGVTVMTTTNTFVELSKQGGLAPDGRSKAFAAAADGTGWGEGAGMLLVERLSDAERHGHPVLAVVRGTAANQDGASNGLTAPNGPSQRRVIRAALSNAQLSTGDVDVVEAHGTGTRLGDPIEAQALLDTYGQDRDRPLWLGSVKSNLGHTQAAAGVAGVIKMVLAMRHGVLPRTLHVDEPTPHVDWSAGAVRLLTERTPWPEADRPRRAGVSAFGVSGTNAHVIVEQASEAEPVEPPRAEPVTVPWVLSGQGEAGLRAFAARLADVATEAHPGDLGWTLATTRSALPHRAVVIGSTPEELRSGLAAVAAGEPASNVVEGVAGSDTGVVFVFPGQGSQWAGMAVELLDSSPAFARRFAECARALETHLDWSIEDVVRSAPGAPSLDLIEVVQPVLFTMMVSLAELWASYGITPSAVVGHSQGEIAAACVAGALSLEDAAKVVVLRSRLFAETLVGNGAIASVALPAEQLATRIEPWGERLVVAGVNGPAAATVAGDPQSLEEFVAACAADGVRARVVPATVASHGPQVEPLRERLLALLADVAPRQSTVPFYSTVTGGLLDTTELDADYWFWNARKPIDFLGALRALFADGHRVFVESSTHPALTMGVQDTADASGESVEVTGSLRRGEGGLDQFHSAVARLHVHGVRVDWSAAFGAARRVELPTYPFQRERYWLTPRPGQGDASALGLGALDHPLLGATVVLPESGGCLLTGRLSLAGQPWLADHALSGVVLLPGTGFVELVLQAGLRWGCGVVEELTLEGPLVLPERGEVEVQVSVGGVDGAGCRSVSVFSCRGGEWVRHAVGVLGVGDGAVPVAEVWPPVGAERVGVEGVYEALAERGYAYGPVFQGLRDAWRRGDEIFVEVAVAQEARADAARCAIHPALLDAALHGVRFGDFVSDDDQAYVPFSWTGVTLHAVGATVLRVTLSPAGRDAIALRATDTTGAPVLSARSLALRPVSAQQLNDTRGSRTDALHRVEWVDASGTVAVGGEVAPRTEVVRVVSEGPDVVGEAYGHVLEVLERVQAWVADEDLAGERLVVVTRGAVDTGDGVADVAGAAVWGLVRSAQSENPGRLVLVDTDDLDGVDSLLPGMLALDEEQVLVRSGAVRVPRLARVPAPGEVSGGFGSGAVLVTGGTGVLGGLVSRHLVARHGVSRLVLLSRRGAEAEGAAELREELEAAGAEVVIAACDAADREALAGVLSGLSADFALSGVVHAAGVLDDGLLTSLTRERVEPVLRAKVDAAWNLHELTTGMDLSAFVLFSSAAGILGNAGQGSYAAANGFLDALAAHRRARGLPAVSIAWGFWEARSELTQHLSADDLARAHAVPMPTSQALDLFDATLAADEPMVLAAPLNPQAWSDAGHLPPVLRDLVRPRIRRAAETTGAPESASALGHRLAAVDRSEWDQVVRELVRNHIAAVLRHASGESVDTSRTFQEIGFDSLTAVELRNRISAATGVRLPATAVFDYPTPQALAEYLLAEVLGKDSAAAATPVGTALVADDPIVIVGMSCRYPGGITSPEALWDLVRSDGDAISVLPADRGWDLDGLYDPDPDRTGTSYARSGGFVYDAAEFDAAFFGISPREAAAMDPQQRLLLETSWEAFERAGIPATSVKGERIGVFTGVMHHDYLTRLSTTPDAVEGYLGTGAAAGVASGRVAYTFGLEGPAVTVDTACSSSLVALHLAVQALRLGECSLALAGGVTVMSTPTVFVEFSRQRGLAPDGRCKAFAGAADGTGFAEGIGMLLVERLSDARRNGHPVLAVVRGSAVNQDGASNGLTAPNGPSQQRVIRQALASAGLSTVDVDAVEAHGTGTTLGDPIEAQALLATYGQGRDSDRPLLLGSIKSNIGHTQAAAGVAGVIKMVMAMRHGVLPQSLHIDEPTPHVDWSTGAVELLSEQTAWPEAGRPRRAGVSSFGISGTNAHLILEQAPLPTAAERPGDAEPVPVEPAAVVPWIVSGRDRHAVRAQAERLRAHVVSHPDRRVADIGFSLLTSRAVLEHRAVVLGGDHAELLAGLTALARDEPAPGVVEALDAAEPGRKVVFVFPGQGSQWAGMALELMESSPVFARRMGECADALAPLVEWSLPDVLADERALARVDVVQPVLWAVMVSLAELWRSYGVVPSAVVGHSQGEIAAACVAGGLSLADGARVVVLRGKALLALSGRGGMVSVPVPADRLRDRPGVSIAAVNGPSSTVVSGGDEVLDAVLAEFPAAKRIPVDYASHSPQIDDIRDELLKALAPIEPRTAAIPFHSTVTGRPIDTADLDADYWYRNLRETVELERVIRTAVEDGHHTFIEISPHPVLTTGLRETLDDADAHGGLVLASLRRDDGGPTRFLTALAEAYAHGVEVDWLPLFPGARRVDLPTYAFQRERYWLDAPTAEAPTSAIDAEFWAAVEREDLESLAATLRVDGQPLREVLPALSQWRRERRDVSTIDSWRYTIRWKPLTPPATSPTGTWLVVVCHAEAGHEWVAGVTDALTRHGAEPLVVVLGEPELDRAALAARLGGVLADTPRISGVVSLTALDESPHPAYPSVPQGYAMTLLLSQALGDARVEAPLWCLTQRGVSLGDAGGSGSGSGTGDGRGKGKGDVAVSRKQALTWGLGKVIALEQPLRWGGLIDLPEGVAPHTQDYLAGVLSGTSDEDQVAIRPTGLFGRRLAHAPARERGGGWQPRGTVLVTGGTGALGGHVARWLAGQGAEHVVLTSRRGMAAPGAERLAGELEALGARVTVAACDVGDRDALAGLLAEVGPLTAVVHTAAVLDDGTLNSLTTDQLQRVLRVKTDGAVHLHELTRDMELSAFVLFSSLSGTLGAPGQGNYAPGHVFVDTLAEQRRAEGLVATSIAWGLWAGDGMGEGGVGDVARRHGVPEMAPEMAVAAMARAVEQDDTVVTVAEIDWDRHYVAFTATRPSPLLSDLPEVRALVDAGVGQESAEPGHERSEFAERLAGMAETDRNHALLDLVRRHVAVVLGHTGPDAIDPGRAFHEIGFDSVTAVELRNRLNRATGLRLPATVTFDQPTPLAMAQYLRGELLHDGQGRSAPALPVRATGAVDDEPIAIVGMSCRFPGDVASPEDLWRLLADGSDAIGEFPENRGWDTAHLFHPDPDHRGTSSTRAAAFVSGAGEFDAGFFGISPREAVAMDPQQRLLLEVSWEALERAGIDPTTLRGSETGVFTGTNGQDYASLLKADETGDFEGRVGTGNSASVMSGRISYVLGLEGPALTVDTACSSSLVALHLAVRALRSGECSLALAGGASVMTTAGIFVEFSRQRALAADGRCKAFAAAADGTGWGEGAGMLVVERLSDAERLGHRVLAVVRGSAVNQDGASNGLTAPNGPSQQRVIRQALASAGLSTVDVDAVEAHGTGTTLGDPIEAQALLATYGQGRDSDRPLLLGSIKSNIGHTQAAAGVAGVIKMVMAMRHGVLPQSLHIDEPTPHVDWSTGAVELLSEQTAWPENTRPRRAGVSAFGVSGTNAHVILEQAPEPTAAQPELSPERDEMRAVPWVVTGASEAGVRAQAARLMAFVDDRPELRPVNIGWSLASTRAALSHRAVVVGAERTELLRELEAVASGSVTVGEARTHSGVVFVFPGQGSQWVGMALELVESSPVFAGRMRDCADALAPFVEWSLFDVLGDEVALGRVDVVQPVLWAVMVSLAELWRSFGVVPSVVVGHSQGEIAAACVAGGLSLEDGARVVALRSRALLALSGRGGMVSVPVSADRLRGRVGLSVAAVNGPVSTVVSGAVEVLDGVLAEFPEARRIPVDYASHSVQVEGIREGLAEALAPVRPRTGEVPFYSTVTGRLMDTIELDAEYWYRNLRETVEFQSAIEGLLELGHTVFVEASPHPVLTIGIQDTADTTDTDIVVSGSLRRDDGGPVRFLSTVGRLFTEGVPVEWQPLFAAAGARKVDLPTYAFQHEWFWLDPVRGASDVGGAGLAGLAHPLVSAVLPLPESDGCVLTGSLSSATHPWLRDHAVLDKVLLPGTGFVELALQAGLHLGCRTLDELTLQAPLMLPAHGDVQIQVAVGGPDDSGRRPVTVYSRPGKDRTWMRHATGSISPVGETATVDRAVWPPVGATPVELTDVYAEMSTHGYAYGPVFQGLRAAWRRGDEVFAEVVLPETAESDAGRCAIHPALLDAALHGAGLGTFVTEPGRPHLPFTWTGVTLHAVGATTLRVVLSPAGPDAISLLAMDGTGAPVLTADSLALRPVSEGGLGGSHDDSLFRVDWTELTLDASDASDAPEVSDEAAFPVVESVAQLAGVAAARSGRGAVVFRLSTTETTGGAAEESPEDVYALTSRVLKVAQAWLADDRFGDARLVVVTRGAVATTPGENPESLAAAAVWGLIRTAQTENPGRFVLVDTVDEDPSALPGVLATDEPQVAIRAGKALVPRLVRATSSALPVPAETDTWRLETDGQGTLENLVLSPRAEASRPLAAHEIRVAVHAAGVNFRDVLLALGMYPDKAGLLGSEAAGTVLEIGSGVVGVAPGDRVMGLFSGAFAPVAITDHRLVAPIPEGWSFPQAAATPIAFLTAMYALIDLAEVRSGESVLVHAAAGGVGMAAVQVARWLGAEVFATASPAKWDAVRACGVAPRRIASSRSPEFADRFRSDAPDGVDVVLNSLTGELLNASLGLLRPGGRLIEMGRTELRDAQEVMARHGVSYRAFELLDAGPDRIGRLLTELLALFHQGVFTPLPLRVQDVRQASDAFRHLSQARHIGKLALTIPRPLSGGTALITGGTGTLGGLVARQLVREHGVTELVLASRRGDTAPQAAELLTELEAAGARVRVAACDVSDRDAIAALVASLPNLRSVVHTAGVLDDAVIGSLTPERLRTVLRPKADAAWHLHELTRDRDLAEFVLFSSAAGVLGGPGQGNYAAANAFLDALAARRRAQGLPATSLAWGFWEQRSGLTEHLTTDRLARAGVLPLSTDEGLVLFDDARATGDTLLVPMRYEPSSPGPEPVPALLRGLVRAPLARALPGPADGVGSGVAEGLTGLAADERLGALLDLVRREAAAVLGHGGPESVTPQRPFKELGFDSLSAVELRNRLRAATGRRLEATLVFDHPTPAVLARHLDAELFGATDVAAPVPAPAVAHPADEPIAIVGMSCRLPAGVDSPEALWKLLVSGTDAISELPPDRGWDLDRLYDQDPSRPGTTYAKTGGFLKNAADFDAGFFTISPREALAADPQQRLWLEACWEAFERAGIDPLALKGTRTGVFAGAVSTTYGAGQAATPDGSEGYLLTGNSTSVISGRVAYTLGLEGPAVTVDTACSSSLVSVHWACESLRRGESTLALAGGVAVMTTPDLLVEFSRQRGLAPDGRCKSFAAAADGTGFAEGVGVLVLERLSDATRNGHQVLAVIRGSAVNQDGASNGLTAPNGPSQQRVIRQALVNAGLASQDVDVVEAHGTGTTLGDPIEAQALLATYGQDRDPDRPLLLGSVKSNIGHTQAAAGAAGLIKMVLALRNGVLPRTLHVDEPSPHVDWSAGAMELLTEQTAWPDRDHLRRAGVSAFGVSGTNAHVILEQAPEPDENGEPDTVRSWLPAVPWVLSGAGAAGLRAQAQRLASFVRENPGLDPVDVGWSLVATRAALSHRAVVVGADRTELLRELAAVESVGAAEAERDVVFVFPGQGSQWVGMALELVESSPVFAGRMRECADALAPFVEWSLFGVLGDEVALGRVDVVQPVLWAVMVSLAELWRSFGVVPSVVVGHSQGEIAAACVAGALTLEDGARVVALRSRALLALSGRGGMVSVPVSADRLRGRVGLSVAAVNGPVSTVVSGAVEVLDGVLAEFPEARRIPVDYASHSVQVEGIREGLAEALAPVRPRTGEVPFYSTVTGRLMDTVGLDGEYWYRNLRETVEFQSTVEALIGQGHTVFVEASPHPVLTVGVQDTADAMETPIVATGSLRRDEGGVRRFLTSLAEVSVHGIEVNWQTVFDGTGARRVDLPTYAFQRERFWLVPSTGTGDASGLGLGAVDHPLLGAAVPLPDADGCVLTGALSLAGQPWLADHSVLGMVLLPGTAFVELALQAGARFGCGTLEELTLHEPLVLPERETVQLQVSVGGSDDFGGRPFTVFSRCEGEWIRHAGGTLRVGERGDPPANPSVWPPADARPVDVAELHTTMAERGYQYGPAFQGLRKAWIRDSEVFLDVALPEQVRGDAARCGVHPALLDAALQGIGLGAFVNEPGQAHLPFSWSGVTLHAVGATAVRVTLSPAGPDTVAIRMADTIGAPVLSIDALAMRPLAEQRLLEAGGSRGDALFRLEWKELPVPTGATGPRAQSWGLLGGHDEPRLTAALTAAGVSPQRHRDLASIDQVPDVLVLSCPPEADGGPAPEATSSALRRVLEVVREWLGDARYTDARLMVLTRRAVATSTGDDVEDLAAAAVRGLLRTAQQENPDRLVVIDHDDSDLEVLPVVLGTGEPEAAIRAGKVLVPRLVKAAVSEGKAPAWDAGTVLITGGTGTLGGLVARHLVTTHGARDLVLASRGGDTAPGAVELATELEALGARIRVAACDVADRAQLTALLDTIPALRAVVHTAGVVDDGVIGSMTAERVETVLRPKANAAWHLHALTRHLDLDAFVLFSSATGVLGSAGQGNYAAANAFLDALAVHRRAQGLPAVSVAWGLWERRSGLTAHLSEQDVARMTSTGAVPLSDERGLELFDAACRSGEPTLVATPLHLRAVAATGTVPHVLSALAPTPPRRAAEAGDGGVALRQSLAEMSGAEQSQTVLGLVRGQVAAVLRHPDPSAIDTARTFQEIGFDSLTAVELRNRLGATTGIRLAATAIFDYPTPATLAQHLLAEIVPETADPVAARLGELDKVAAMISAMAEDDTLREQLSSRMETIVAMWADLHRPERPGTVERDLESASLDDMFGIIDQELDGS

SEQ ID NO: 49 - protein, 11188 aa, Streptomycetes sp.
MSSENVRPEIEGTGTRMSNDEKVLEYLKKLTADLRQTRQRLQDVEAKSREPIAIVGMSCRFPGGVSSPEDLWRLTESAVDAVSGFPTDRGWDLDGLYDPDPDRAGRSYAREGAFIPDAGHFDPGLFGISPREALAMDPQQRLLLEASWEALERAGIPTDSLKGSRTGVFAGLMSSDYVSRLSAVPDELEGYVGIGSAASVASGRVSYTLGLEGPAVTVDTACSSSLVALHLAVQALRSGECSLALAGGVTVMATPGTFVQFSRQRGLAADGRCKAFAAGADGTGWGEGVGMLVVERLSDAERLGHRVLAVVRGSAVNQDGASNGLTAPNGPSQQRVIRQALANARLSAVDVDAVEAHGTGTALGDPIEAQALLATYGQGRDVGRPLWLGSVKSNIGHTQAAAGVAGVIKMVMAMRHGVLPRTLHVDEPSPHVDWSAGAVELLTGQVAWPEVDRPRRAGVSAFGVSGTNAHVIVEQAPEVAESEAEGVVLPAVPWVVSGVGEVAVRAQVERLRAFADRNPGLDPVDVGWSLATGRAGLSHRAVVVGAGRGELLGALEGVPVVGVPVVGGLGVLFAGQGSQRLGMGRGLYEGYPVFAAVWDEVCAQLDRYLDRPVGEVVWGDDAGLVGETVYAQAGLFALEVALYRLIASWGVRADYLLGHSIGELAAAYVAGVWSLEDAVRVVVARGRLMQALPSGGAMVAVGASEGVVRPLLGEGVVVAAVNGPESVVLSGDEDAVQVVVDVLAGRGVRTRRLRVSHAFHSARMDGMLAEFGEVLRGVEFRAPSVPVVSNVSGVVAGEELCSPEYWVRHVRETVRFADGLETLRELGVGSFLELGPDGTLTALADGDGVSALRRDRPEPTAVMAALGGLYVRGVEVDWDAVFPGARRVDLPTYAFQRERFWLEPAAEQPATSAVDAAFWDAVERGDAEILGVDVEQPLSAALPALASWRRARQEESVIDAWRYRLTWTPVAGLSSQLSGVWLVVVEPDEAEPDVVAALRGAGAEVRVVTIDELDAGPVAGVVSLLSVETTVSLLQALVAEGGDAPLWCVTRGAVSVVDGDVVDPHASAVWGLGRVIGLEHPDRWGGLIDLPTAWGERTSGMLCSVLSGATGEDHTAIRGDEVLGCRLSRATTSAPGPSTAWEASGTALITGGTGALGSHVARWLADTGVEEIVLTSRRGADAPGARELVAELSAMGVSARVVACDVADRDAVAELIETIPDLRVVVHAAGVPSWGALSTLTAQGLQDGMRAKVAGAIHLDELTRDMRLDAFVLFSSVAGVWGSGSQSAYAAANAFLDGLAWRRRGVGLVATSVAWGMWGGGGMAVGGEEFLVERGVSGMAPGSAVAALRRALCDGETALVVADVDWERFGPRFTALRPSPLLSELIPDTVGSGVPLGEFAARFQTMSEGERMRAAVELVRVSAAAVLGHQGPEAIDPVRTFQEIGFDSLTAVELRNRIATATGIRPPATMVFDYPTPVALAEYLSVELLGSPQDSVPPLQVAAPDDGDPIVIVGMSCRFPGDVESPEDLWRLIDSDGDAITAFPTDRGWDLTGLFDTAVGESGTSYARVGGFVHDAGEFDPAFFGISPREATAMDPQQRLLLHAAWEAFERAGIPAASVRGSRTGVFVGASPQGYGAAEASEGYFLTGSSGSVISGRVSYTLGLEGPAVTVDTACSSSLVALHLAVQALRSGECSLALAGGVTVMATPTAFVEFSRQRGLAADGRCKSFAAGADGTGWSEGVGLLLVERLSDAERLGHRVLAVVRGSAVNQDGASNGLTAPNGPSQQRVIRQALANARLSAVDVDAVEAHGTGTALGDPIEAQALLATYGQGRDVGRPLWLGSVKSNIGHTQAAAGVAGVIKMVMALRHGVLPRTLHVDEPSPHVDWSSGAVELLSERAAWPEMGRPRRAGVSSFGVSGTNAHVVLEQAPGAVEESRGEGVALPAVPWVVSGAGEVAVRAQVERLRAFADRNPGLDPVDVGWSLVATRSGLSHRAVVVGADREELLGGLGSVVVGVPVAGGLGVLFAGQGSQRLGMGRGLYEGYPVFAAVWDEVCGELDRYLDRPVGEVVWGDDAGLVGETVYAQAGLFALEVSLYRLIASWGVRGDYLLGHSIGELAAAYVAGVWSLEDAGRVVVARGRLMQALPSGGAMVAVAASEGEVRPLLGEGVVVAAVNGPESVVVSGDEDAVEAVVDVLAGRGVRTRRLRVSHAFHSARMDGMLAEFGEVLRGVEFRAPSVPVVSNVSGAVAGEELCSPEYWVRHVRETVRFADGLETLRELGVGSFLELGPDGTLTALADGDGVPVLRRDRPEPLTVMAALGGLYVRGVQIDWDAVFPGARRVDLPTYAFQRERFWLEPSPEQPTTSAADAAFWDAVERGDLGSFGIDAEQPLSAALPALSSWRRRHQERSLVESWRYRLDWSPIGTASEQPSLRGTWLVVGEGGDDVVAVLRAAGADARVVTMAELGEVAAAGVVSLLPVEATVSLVQALGTAGADAPLWCVTRGAVSVVDGDVVDPGQSGVWGLGRVIRLEHPDRWGGLIDVPVVVDEEAGAWLCRVLGGGTGEDQVAVRGGGAWGARLVRVSGSGSGSGGAVVWRGRGAALVTGGTGALGGHVARWLAGAGVETVVLASRRGMAAPDAEQLVAELEGLGVAVRVVACDVADRGAVAELLEGIGDLRVVVHAAGVLDDGVLESLTSERVREVMRVKAEGARYLDELTRGWDLDAFVLFSSAAGTVGNAGQGSYAAANAVLDGLAWRRRAEGLVATSVAWGAWADSGMGAGHARAMAPRLALAALQRALDDDETALMIADVDWSSFGSRFTAVRPSPLLGELLGGAAHPAPAVGGFVDRLRDLPPAERERTVLELVRGQVAVVLGHATPGAIDTAATFQSAGFDSLTAIELRNRLMAATGVQTPASVVFDYPTPELLAGHLREQLLGAGSAALSTTVATAPVDDDPIAIIGMSCRFPGGVDSPEELWRLLESGTDAISAFPQDRGWDLVGGVDGASVRAGGFLYTAAEFDPAFFGISPREAIAMDPQQRLLLEASWEVFERAGIAADALRDSPTGVFVGTNGQDYAALVGNAPQRADGHLATGSAASVASGRLSYTFGLEGPAITVDTACSSSLVAMHLAAQALRSGECRMALAGGATVMATPTAFAEFSRQGALAADGRCKAFAAGADGTGWGEGVGILLLERLSDAERNGHRVLAVMRGSAVNQDGASNGLTAPNGPSQQRVIRQALANARLSTVDVDAVEAHGTGTTLGDPIEAQALLATYGQDRDPDRPLLLGSVKSNIGHTQAAAGVAGVIKMVMAMRHGVLPRSLHIDEPTPHVDWTAGRIALLTEPSPWPLTGAPRRAAVSSFGVSGTNAHVILEQASAVAEPEETDTARTPEPPAVPWVLSARSEAGLRAHALRLRSFVNADADLRPVDVGWSLASARSVLSHRAVVVGADRDELLRELEAVASGSVTVGEARTHSGVVFVFPGQGSQWVGMALELLEHSPVFAGRMRDCADALAPFVEWSLFDVLGDEVALGRVDVVQPVLWAVMVSLAELWRSFGVVPSAVVGHSQGEIAAACVAGGLSLEDGARVVALRSRALLALSGRGGMVSVPVSADRLRGRVGLSVAAVNGPVSTVVSGAVEVLEGVLAEFPEAKRIPVDYASHSVQVEGIREGLAEALAPVRPRTGEVPFYSTVTGRLMDTIELDGEYWYRNLRETVEFQSTVEALIGQGHTVFVEASPHPVLTVGVQDTADTTDTATDIVVTGSLRRDDGGPARFLTALAELSVRGVATDWRQAFEGTGARHVDLPTYPFQRQRFWIEPTAPDVAREDARVTTADGEFWAAVEREDAASLATALEVDDASLGNLLPALSAWRRRRHEWSALEAVRYQVNWKRLVDDRPAMLSGAWLVVVSQADADHEWVSGVSETLAEYGAEPVVCPVDERHLDRAVLADRLASMTGTSSTTSTASISGVVSLVALDQRPHPDFASVPIGFAMTVLLTQALGDTGVEAPLWSLTQHAVSTGPADTLLASASAQALVWGVGRVIALEQPLRWGGLIDLPTEVNARARERLARVLSGVSGEDQVAIRTVGAFGRRLVHAPALRTDLPSWQPSGTVLVTGGTGALGGHIARWLAHQGAEHLVLTSRRGMAAPGASALVADLEAAGAAVTVAVCDVAERAQLADLVADVGPLTAVVHTAALLDDATVESLTTEQLHRVLRVKVDGATHLHELTRDMELSAFVLFSSLSGTVGTPGQGNYAPGNAFLDALAEYRRTQGLVATSVAWGLWAGDGMGEGEAGEVARRHGVPALSPELAVAALRAAVEQGDAVVTVADIEWERHYAAFTATRPSPLLADLPEVRRLIDAGAASAVEETDRDRSGLSGRLAGLDGAEQRRLLLDLVRRNVAVVLGHTDPEAVSSHRAFQELGFDSVTAVEFRNRLGAATGLRLPATAVFDYPTPLALAEYALSELLGTVGEPLRVESSGSPVDDDPIVIVGMSCRFPGGVSSPEDLWDLLTEGGDAMSAFPGDRGWDLAGLFHSDPGHPGTSYTRTGGFLHDATAFDADFFGISPREALAMDPQQRLLLEASWEAFERAGIDPRSLRGSETGVFAGTNGQDYVSLLGGDQPQEFEGYVGTGNSASVMSGRIAYVLGLEGPALTVDTACSSSLVALHLAVQALRSGECSLALAGGVTVMATPGLFVEFSRQRGLAADGRCKAFAGAADGTGFSEGVGMLVVERLSDAERLGHRVLAVVRGSAVNQDGASNGLTAPNGPSQQRVIRQALASAGLVAVDVDAVEAHGTGTALGDPIEAQALLATYGQGRDVGRPLWLGSVKSNIGHTQAAAGVAGVIKMVMALRHGVLPQSLHIDEPTPHVDWSTGAVELLGEHTGWPEVDRPRRAGVSAFGVSGTNAHVIVEQAPEVVEPEAEGVVLPAVPWVVSGVGEVAVRAQVERLRAFADRNPGLDPVDVGWSLATGRAGLSHRAVVVGADRGELLGALEGVPVVGVPVVGGLGVLFAGQGSQRLGMGRGLYEGYPVFAAVWDEVCAQLDQHLDRPVGEVVWGDDAELIGETVYAQAGLFALEVALYRLIASWGVRGDYLLGHSIGELAAAYVAGVWSLEDAARVVVARGRLMQALPSGGAMVAVAVSEGVVRPLLGEGVVVAAVNGPESVVLSGDEDAVQVVVDVLAGRGVRTRRLRVSHAFHSARMDGMLAEFGEVLGGVEFRAPSVPVVSNVSGAVAGEELCSPEYWVRHVRETVRFADGLETLRELGVGSFLELGPDGTLTALADGDGVPVLRRDRPEPLTAMAALGGLYVRGVQIDWGAVFPGARRVDLPTYAFQRERFWLEPSAEQPATSVVDAAFWDAVERGDAEALGGDAEQSLSAALPALASWRRAQQEESVIDGWRYRLGWTPIPVVLGEPCLTGTWRVVVEPGADGTDVAAALRSAGADAEVVTSAELSAGPVAGVVSLLSVEATVALVQALGTVGIDAPLWCVTRGAVSVVDGDVVEPYASAVWGLGRVIGLEHPDRWGGLIDLPTEADARVGALLAGVLAGRTGEDQVAIRAAGAWGARLSRATPIADTSGGWRGRGAALITGGTGALGGHVARWLAGTGVERIVLTSRRGIETPGAAELVTELEEFGVQVTVVACDVADREAVATLLVTIPDLRVVVHAAGVPSWSAVDSLTPEEFEESARSKVAGAANLDALLADAELDAFVLFSSVAGVWGSGSQSAYAAANAFLDGLAWRRRGVGLVATSVAWGMWGGGGMAVGGEEFLVERGVSGMAPGLAVAALRRALCDGETALVVADVDWERFGPRFTALRPSPLLSELIPDTSEPLASTVGEFAVELRGLSREDRDRAVVELVRTHAAEVLGHQNPSAIDLDRTFQELGFDSLTAVELRDRLGTATQLRFPASVIFDYPTPAALAEHVCGAALGLAEEIQVAHTPSAVADDPIVIIGMSCRFPGGVDSPEALWRLVSAGGDAVSSFPSDRGWDLAGVYDADATRSGRSYVRTGGFLHDAAEFDAGFFGISPREATAMDPQQRLLLEASWEAFERAGIPASTLKGSQTGVFVGASAQGYGGGDGQAPEGSEGYLLTGNAGSVVSGRVAYTFGLEGPAVTVDTACSSSLVALHWAVRALRSGECSLALAGGVTVMATPATFVEFSRQRGLAADGRCKSFAAGADGTGWSEGVGLLLVERLSDAERNGHPVLAVVSGSAVNQDGASNGLTAPNGPSQQRVIRQALANAGLVASDVDAVEAHGTGTTLGDPIEAQALLATYGQGRDAGRPLWLGSVKSNIGHTQAAAGVAGVIKMVMAMRHGVLPRTLHVDEPSPHVDWSAGAVELLTGQVAWPEVDRPRRAGVSAFGVSGTNAHVIVEQAPEVVEPEAEGVVLPAVPWVVSGVGEVAVRAQVERLRAFADRNPGLDPVDVGWSLVATRSGLSHRAVVVVADGEELLGALEGVPVVGGLGVLFAGQGSQRLGMGRGLYEGYPVFAAAWDEVCAQLDQHLDRPVGEVVWGDDAELIGETVYAQAGLFALEVALYRLVASWGVRADYLLGHSIGELAAAYVAGVWSLEDAARVVAARGRLMQALPSGGAMVAVAASEGEVRPLLGEGVVVAAVNGPESVVVSGDEDAVHAIEETFAMGGVRTRRLRVSHAFHSARMDGMLAEFGEVLRGVEFRAPSVPVVSNVSGAVAGEELCSPEYWVRHVRETVRFADGLDTLRELGVGSFLELGPDGTLTALADGDGVPVLRRDRPEPLTAMAALGGLYVRGVEVDWDAVFPGGRRVDLPTYAFQRQRFWLESASDQPATSAVDAAFWDAVERGDARALGIDEEQPLSAVLPALSSWRRARQEQSVIDGWRYRLGWMPIPAVLGEVGLIGTWLVVVEPGVDGTDVAAVLRSAGAGVEVVTSAELSAGPVAGVVSLVSVEATVSLLQVLVAAGVDAPLWCVTRGAVSVVDGDLVDPGQAGIWGLGRVIGLECPDRWGGLIDLPGELDDRAGNALVGILAGGTGEDQVAIRVTGIWGARLVRATPVPIGDAGGEAAAAWRGRGTALVTGGTGALGRQVARWLVGSGLERVVLTSRRGVEAPGAVELVAELGSRVRVVACDVGDREELAALLVTLPDVRTIVHAAGVLDDGVLESLTPERIREVMRAKADGARHLHELTRDIDLDAFVLFSSAAGTVGNAGQGSYAAANAVLDGLAWRRRAEGLVATSVAWGAWAESGMAAEMARSQGMDPRSALAALGLVLAADETTVMVADIDWATFGARFTASRPSPLLSELLGDGSVSTEAADGEPADAFATRLEAMAERERAATVLDLVRTHVAAVLGHTASEAIDPARPFQEIGFDSLTAVELRNRLTAATGVRFPASVIYDYPTPAALAEHVCREALGPGGRTPAPVVPRPVDDEPIAIIGMSCRFPGGVSSPEDLWGLLAEGRDAVSDFPADRGWNLAELYDPDPDHPGSSYVRAGGFLDDAAAFDPGFFGISPREALAMDPQQRLLLEVAWEAFERAHMSPATLKGSRTGVFVGTNGQDYAALASGAPRSAEGYLGTGSAASVASGRLAYTFGLEGPAVTVDTACSSSLVALHLAAQALRSGECSLALAGGATVMATPAAFLEFSRQRALAADGRCKAFAAAADGTGWGEGVGMLLVERLSDAERNGHRVLAVMRGSAVNQDGASNGLTAPNGPSQQRVIRQALANARLSATDIDVVEAHGTGTSLGDPIEAQALLATYGQGRSQNKPLWLGSVKSNIGHTQAAAGVAGVIKMVMAMRHGVLPRTLHVDSPSPHVDWAAARVELLVEAREWPRTGAPRRAGVSSFGVSGTNAHVIVEQGPVVARPDRESAREPSPSVPWVLSGAGEAGLRAQVERLASFIDAHPGLDPADVGWTLVAGRSCQSHRAVVVGADLAELRRGLDAVSTGGAARSGRKVVFVFPGQGSQWAGMALELLEHSPVFAERMRACADALTPFAEWSLFDVLGDEVALGRVDVVQPVLWAVMVSLAELWRSFGVVPSAVVGHSQGEIAAACVAGGLSLEDGARVVALRSRALLALSGRGGMVSVPVSADRLRDRAGLSVAAVNGPASTVVSGAVEVLDGVLAEFPEAKRIPVDYASHSPQVAEIQRELADVLAPVRPRGGQIAFHSTVTGRLTDTSELDADYWYRNLRHTVEFQSTVEALMNQGHTVFVEVSPHPVLTIGIQDTAETPGTPDTPGTPDTADATDAHEATGAPDVANTADVTGAPDVTGADIVITGSLRRDDGGPARFLTALGDLHTRGVDVDWSPVFTGARTVDLPTYAFQRERFWLKPARAVTQASGLGLGDIEHPLLGAVLPLPGDEGGVLTGLLSLDGQPWLAHHMVRDTVVFPGTGFVELALQAGQHFGHSVIEELTLHAPLVVPDQGGVQVQVAVSAADERGRRPVTVHSCRAGEWLLHASGTLGATGGLDVTEPRPADVARPLEVWPPEGARSLDVSGMYEAMAERGYGYGPAFQGLRAAWTRDDEIYAEVALEPEAQDVAARCGAHPALLDAALHGVGLGRFLTDPGQAYLPFSWSGVALHAVGASAIRVVLSPAGTDAVSLEVTDPTGAPVLSVASLSLRPLSSGRIADTRGVDQDSLYRVDWVEMPLPTAPAGSAPAEYDAPAMFDALVFDAPVEYDVLASDASDASDASDAPGTPDASSAPVPDMPDMVVLPCESAGDAVSTVVCRALAAVRRWLADERCARSRLAVLTRGAMATAPGESVEDLGAAAVWGLLRSAQAEHPDRFVLVDHDGHQDSRAVLAAALAAAVDGGHAHLALRRGRVLTPQLAPLTPSATALSTTAPPAATPTPEAGAPWRMDVTSQGTLENLAAVPCPEAAGVLGAGQVRVAMHAAGVNFRDVVVALGMIPGQDVIGSEGAGVVLDIGPGVSGLAPGDRVMGLFSGAFGPVAVTDHRLLARLPEGWSFADAAATPVVFLTAMYGLMDLAGLRPGESVLLHSAAGGVGMAATQVARWLGAEVYATASPGKWDALRAGGVADDRIASSRSLEFADRFGRVDVVLNSLAGEYVDASLGLLADGGRFLEMGKTDIRDGERVAAEHGVRYQAFDLMDAGPDRVGELLRLLVSLFERGIFTALPTRVWDVRQAGDALRFLSQARHIGKLVLSIPQPLREGDTVLITGGTGTLGGLVARHLVERHGVRDVVLAGRRGPDAPDAAELAAALREYGARVRVVACDVADRDQLARLLDTVSGLRMVVHTAGVLDDGVIESLTPERVREVLRPKVDAAWYLHELTAGRELAEFVVFSSAAGVLGSPGQGAYAAANAWLDALMAHRRAAGLPGLSVAWGLWAERSGMTGHLSDRDLARMARAGATPLATDQGLRLLDSARAATEALVLATPLDAAALRAQADAGALPALFRGLVRAPIRRATGAGPVEDESSLRGRMAAMPVAEREQLVLDLVRTQVATVLGHGTATAVDPARTFAETGFDSLTAVELRNRLRTATGVRLSATAIFDYPTPAVLAGHLLRELDGTVGEAVTRPAAPAAATDRDPIVIVGMACRYPGGVASPEELWELLATGRDAVADLPDDRGWDLDGLYSADPDSSGTSYVRSGGFVYDAGEFDADFFGISPREALAMDPQQRLLLEVAWETVERAGVPAASLKGSQTGVFVGAAAQGYGTGAGQAAEGSEGYFLTGGAGSVVSGRLSYTFGLEGPAVTVDTACSSSLVALHLAAQALRSGECSLALAGGVTVMATPGIFVEFSRQRGLAADGRCKAFADAADGTGWGEGVGMLLLERLSDARRNGHRVLAVVRGSAVNQDGASNGLTAPNGPSQQRVIRAALANAGLAASDVDAVEAHGTGTSLGDPIEAQALLATYGQQRERPLLLGSIKSNIGHTQSAAGVAGVIKMVLAMRHGALPRTLHVDQPSTHVDWSAGAVELLTEPAEWPGTSRPRRAGVSSFGVSGTNAHVILEQPPAEAESGPAPESAPGPVPAVVPGPVPAVVPWVLSGQGERGLRAQAARLRSFLAARPESGPADVGWSLAATRSALSHRAAVVGADRAELLDGLAALAAGEPAPGVVLGTADPGRVGVLFAGQGTQRPGMGRELYQSFPVFAAAWDEVCAALDPHLDRPLGEVVTDATGALDATTYTQAGLFALEVSLFRLVSSWGVRPDYLLGHSIGELAAAQVAGLWSLEDAAKVVAARGRLMGALPPGGAMVALAAPEDQVRPFLTDRVALAAVNGPSSVVVSGDEDAVCGVAEAFAARGVKTRRLRVGHAFHSPLMDEMLIAFAEVLDTVDFRTPRIPVVSNLSGAVAGEELCSPAYWVRQVRETVRFAAGLERLRELGTGTFLELGPDGTLTALAQAQITGADAEFIPTLRADRPEPVTVTTALAQLHTHGVEPDWSAVFPGAHRAELPTYAFQRSRFWLEPSRTPGDAGDFGLGALDHPLVGARVPLPDADGVLLTGRISAEAHSWLIGQRALGVPLFPATGFLELVLQAGLQCDSRTVDELTIHEPLVLPERGGVEVQVSVRGADESGRRPATVYCRRDQRWVRHATAVLGADRPPAPEPRPEPWPPTGARPLESGGTPAWRRDDEVFLDIELPEVAGAEAERWTLHPALLEQALRGEALAGLVTAAEGTHLPFSWTGITLHTTGATRLRATLAPVGPDTVSLHVADAAGTPVLSVDSLALRPVSGQRLRQANAALFRPVWAACRTRAEPDTGSVRWGLVGDPDAWKPDTLGAPVALYPDLSAIEDVPDVILLPCVSEGGTASEVAVRVSETVRTWLAGERFAASRLVLVTRGALATAAGEELEDLAAAAVWSLVEPLQAAVAGRLTLVDTDTSDLRMLPAAVAVGEDRVAVRAGAVLVPDLVTPPATEQDPPAWGPGTVLVTGGSAMAVSRHLVAERGVRDLVLAGDGDMAELAALGATVRLAPCDPADGQALAALVAEIPGLRSVVHTAADAPERTRSLLPESLRPQLRSGVAAAWNLHLATRGLELDRFVLFTSADGTLGPAYADALAAHRRARGLPAVSVSTDLGLALFDEACAGPGEAIRVTTATPAPAPTEADRQPVEQPPAAEASATTLLERLAGRTEDEQDEILLELVRGQVAMVLGHPDATMVDPDRGFVELGFDSVAAVKLRNQLAGATRLDLPASLTFDHPTAVDLARHLRAEMLPDDAAAAILVLEELNKLDDSILVLDPASAARVRISTLLQDLAAKWVERTDRP

SEQ ID NO: 50 - protein, 397 aa, Streptomyces sp.
VSETLSLPGTVKAERRCPYDPPEAHRRLRDKGELGKLELPGGLVMWFLTKHDDIRAMLADSRFSGARVPFPAMNPEIPAGFFFSMDPPDHTRYRRTLTAEFSVRGARELTGRIERLADRHLDAMEAAGTSADLVAAYASPVPAMVISEILGVPYTYHQKFDHEVRTLRETGGDDQAVGAMATAWWDEMRGFVRAKRAEPGDDMISRLLHDEVEGGALTDEEVVGIAMTIIFAGHEPVENLIGLGMLALFQDGEQLTRLRENPDLIDSAVEEFLRYFPVNNFGTVRTATEDAVINGHPIAKGEIVAGLVSTANRDPERFADPDRLVLDRSHTSHLAFGHGVHQCLGQQLARVELKVLLQRLLVRFPALRLAVAPEEIRYRENTSFYGVHELPVTWAAE

SEQ ID NO: 51 - protein, 172 aa, Streptomyces sp.
VCRPLGSSRRGGRPRGRGFVVGSSGNAVNMTEKKNAHTTRSTNVNAKATATKAKETAERAKDTAGKAETTAKTAAAGAATTAAHTAHVAADKAQVAAGKAVTTGRTVAAEAPKKAAAAAGSAWMMIKARKVLAAVAGAGAAAAGATAAVVLRRRAARRRRPLARLTGGRLGS

SEQ ID NO: 52 - protein, 169 aa, Streptomyces sp.
VGFSFQPFGACFSLTSPGSMPVGNTVRISVKPALPSSASDSNVSVTSFARARESEALTSVWARAGVAVARTSAEVATARAPIRRGRGWDGGRCAFTVSLLVHGVVTRALLTGHPARSPGAFTFPGTYGPGAMFILAQTGSPLATRGSKEFRRLRGPRKADRGGRRVPVR

SEQ ID NO: 53 - protein, 494 aa, Streptomyces sp.
MSAASSDPTSPRVPPTRRLALGVIATGMLMVILDGSIVTVAMPAIQSDLRFSPAGLSWVVNAYLIAFGGLLLLGGRIGDLIGRKRVFLTGTAVFTAASLLAAVATSPAVLIAARFLQGVGSAMASAVSLGILVTLFTERAERSKAIAVFSFTGAAGASIGQVLGGLLTDALSWHWIFLINLPIGLLTLAVAIPVLPADRGPGLAAGADVLGALLVTTGLMLGIYTVVKVADYGWTAARTLGLGAVSILLIALFLVRQTTARTPLMPLRILRSRGVAGANLVQLLMVAALFSFQILVALYLRNVLGYDATGTGLAMLPAAIAIGAVSLGVSARLSARFGDRAVLLTGLALLTGVLGLLVRVPVHARYLPDLLPVMLLAAGFGLALPALTSLGMSGAKEDEAGLVSGLFNTTQQIGMALGVAVLSTLAASRTDALLSRGKGRAEALTGGYHLAFAVGTGLIVAAFAVAFTVLRGPARKPPAVPRNANPPATPVATA

SEQ ID NO: 54 - protein, 159 aa, Streptomyces sp.
MAPTKTEPDLSFLLDHTSHVLRTQMSAALAEIGLTARMHCVLVHALEEERTQAQLAEIGDMDKTTMVVTVDALEKAGLAERRASTHDRRARIIAVTEEGARIAERSQEIVDRVHREALATLPETQRAALLKALTRLSEGHLATPAESPRPARRARQREK

SEQ ID NO: 55 - protein, 314 aa, Streptomyces sp.
VTRGRVACVDRAPGSCMRKMRSGFYLYAGRMDVELRQLRCLVAIVDEGTFTDAAIALGVSQAAVSRTLAALERALGTRLLRRTSREVTPTGTGLRVVAHARRVLAEVDGLIREAVSGHAHLRIGYAWSALGRHTPAFQRRWAQAYPETELHLVRVNSATAGLTEGACDLAVVRRPLDERRFDSAIVGLERRLCAVAADDPLARRRSVRLADLSGRTLLVDRRTGTTTTELWPPDSRPATEETHDVEDWLTVISAGRCVGMTAESTANQYPRPGIAYRPVRDAEPIAVRLAWWRDDPHPATQTAVELLTALYRNG

SEQ ID NO: 56 - protein, 331 aa, Streptomyces sp.
MSRAHDRRMRLAPASRTPSPRAMDTAHRTAPTPADYDLGQGLERGLAPDPDQRPTGRRFAGVATMIGSGLSNQTGAAIGSQAFPVIGPVGVVAVRQYVAAIVLLAVGRPRLRSFTWWQWRPVVGLAVVFGTMNLSLYSAIDRIGLGLAVTLEFLGPLCIALAGSRRRVDACCALVAAAAVVTLMRPRPSADYLGMGLGLLAAVCWASYILLNRTVGRRVPGAQGSAAAAGISALMFLPVGIAVAVHQPPTVSAAAYAIIAGVLSSAVPYLADLFTLRRVPAQAFGLFMSVNPVLAALVGWVGLGQSLGWTEWISVGAIVAANALSILTRRG

SEQ ID NO: 57 - protein, 274 aa, Streptomyces sp.
VRSVCPRHPSSTSHDRIRMTDDMFLMTDDTFLDDVAERLAALPAVHAVALGGSRAQGTHTPESDWDLALYYRGGFDPAALRAVGWEGEVSELGEWGGGVFNGGAWLTIDGRRVDVHYRDLEVVEHELAESRRGRFHWEPLMFHLAGIPSYLVVAELALNQVLRGTLPRPEYPAALREAAPPAWRGRAALTLRYASAAYVGRGQATEVAGAVATAALQTAHAVLAARGEWVTNEKRLLQRADLRAIDTIVAGLRPEPTALAEAIAAAEALFEAAG

SEQ ID NO: 58 - protein, 349 aa, Streptomyces sp.
VSADAGADARGDTVSGELVLVTGGSGYLGTHVISGLLRSGHRVRTTVRSHGPATGAAASVRSAIAASGVDPGGRLDIVSADLTTDDGWDDAMAGCTRVHHVASPFPAVQPDNADELIVPARDGTLRVLRAARDQGVKRVVMTSSFAAVGYSHKDGDEYDESDWTDPEDDNPPYIRSKTIAELAAWDFVAKEGDGLELTVINPTGIFGPALGPRLSASTEHVRAMLEGAMSAVPRAHFGMVDVRDVAELHLRAMAHPAAAGERFLASGDRTVSFLWIAQVLAEHLGERAARVPTREFDDERAREAVGVTERVPILRTEKARSVFGWTPRDPVTTILDTAESLFRLGLVKD

SEQ ID NO: 59 - protein, 135 aa, Streptomyces sp.
VSRLSGLSEPTLRYYEKIGLIPAVDRDRDSGHRRYPPSVVETIRSLGCLRSTGMSMQDMRAYLGHLDEGDQGAAPLRDLFQRNADRLEREIALMEVRLRYLRLKADMWDARERADADAERRAIDELTDVIDALRP

SEQ ID NO: 60 - protein, 497 aa, Streptomyces sp.
VTGPDGYEALPHRRRALVTIALLGCAFLAMLDGTVVGTALPRIVEQIGGGDSWYVWLVTAYLLTSSVSVPVYGRFSDLHGRRRLLIGGLGVFLIGSIACGLSASMPALILSRALQGLGAGSLLTLGMALVRDLHPPSRPQGLIRMQTAMAAMMILGMVGGPLLGGLLADHIGWRWAFWLNLPLGLAAGAVIVLALPDRRPATPPSGRLDVAGILLLAAGLALALTGLSLKGNATAGHAPSWTDPAVLGCLLGGLALLTTLIPVERRAAVPVLPLRLFRHRTYTALLTAGFFFQVAAAPVGIFLPLYFQHIRGHSATASGLLLLPLLIGMTLGNRLTAATVLRSGHVKPVLLIGAGLLTAGTAAFVALRATTPLALTSVLLLLVGLGAGPAMGGLTIATQSAVPRADMGTATAGSALTKQLGGAVGLASAQSLIGHSGAAAPTATAIGSTVSWSGGAAGLLALGALLLMRDISIATAGKRPGAPTSGTAVPAKADRLA

SEQ ID NO: 61 - protein, 213 aa, Streptomyces sp.
MTEKAENPSTPTRRRAPAMDPDQRRAMIVAAALPLVVEYGATVTTAKIARAAGIGEGTIFRVFEDKDALLAACMAEAVRPDDTVAHLESIALDQPLADRLAEAADVVRGHMARIGAVAGALAAAGRLERMAPKPGKDGRLPDREASLVRPRAALAALFEPDRDRLRLAPERLADAFQLTLMSAGRLGAPEPLTTEEVVDLFLHGALVAPGEAR

SEQ ID NO: 62 - protein, 348 aa, Streptomyces sp.
VKCAGTRGSWGAWRRTGPSGRGVPLPLHGGGPIGIVSNDDVCCVASRMEMMVELRQLAYFVAVAEERSFTRGAQREHVVQSAASAAVARLEQEFQTALFDRSHRTLELTTAGRTLLARARILLAEAQRARDDMGRLTGGLSGTVTLGTVLSTGSFDLIGALSTFQAEHPDVVVRLRHSTGPLAGHATALREGRFDLMLLPVPPHGPAVLGPDLIIDDVSRIRLGLACRTDDPLAEAHGVTYADLADRRFIDFPTGWGDRTIVDSLFGTAGVQRTVALEVVDTTTALTMVRRRLGLAFVAEETIASRPGLTQVDLADPPPLHGLGLAASRNHPPSEAGRALRRALLAAR

SEQ ID NO: 63 - protein, 218 aa, Streptomyces sp.
MPHSTHHRWTRYLWDRHRGGEAERPGRTARFGATPPNFPVCQHTSPRKASIVMSVSAIQIGLHPDAIDYEAPEFAAFAGLSRETLRAANDDNLALLLDAGYEADGCQIDFGETALDTIRAMLGRKRYDAVLIGAGVRLTAGNTLLFESIVNLVHTALPHARFIFNHSAAATPDDIRRHYPDPASTVPLDVPRDLEEAALKNPGNAARPEAAHGPRETR

SEQ ID NO: 64 - protein, 459 aa, Streptomyces sp.
VLERRPAIHPSSRAFVTMPRTLEVLDSRGLADDLLAGANTTEAVHLFAGATLDLTHLPSRHRYGMITPQTNVDQALERYARDQGARVLRGTEVTGLAQDADAVTVTARADGGGPASTWRARYVVGADGAHSTVRGLLGADFPGRTVLTSVVLADVRLADGPTGNGLTLGNTPEVFGFLVPYGKARPGWYRSMTWDRRHQLPDKAAVEEAEVTRVLAEAMGRDVGVREIGWHSRFHCDERQVRSYRHGRVFLAGDAAHVHSPMGGQGMNTGVQDAANLAWKLDLALGGADPAILDTYHRERHPVGRRVLLQSGAMMRAVTLGPRPARWLRDHLAPALLGVGRVRDTIAGSFTGVTPRYPRGRRQHALVGTRATEVPLAEGRLTELQRAGGFLLIRERGAARVDTTVAQAERTDSGPALLVRPDGYIAWAGPGVRTDGPDGWHTTWRAWTGPATDAVRAGR

SEQ ID NO: 65 - protein, 135 aa, Streptomyces sp.
MSLIREPHRRRFNAIMVGGAGAAYLSGGGLDGWEFAFTVVATYVAYRGLESWTFIGIGWLLHTAWDIVHHIKGNPIVPFAHGSSLGCAICDPVIALWCFRGGPSLLRFFRKGRPEEPAAAALPDSLSAGQATGNG

SEQ ID NO: 66 - protein, 816 aa, Streptomyces sp.
MSGATRLPRHPTDRSRTMPLDRRRFLRTSALTLGAPALAGHLATDAVASPARRPRAPLSDAFDRLPSGSITPRGWLAEQLRLQLHGLCGRYQERSHFLDINATGWTHPDRDGWEEVPYWLRGYVPLAVATRDQAALANARGWIDAILATQQSDGFFGPRSLRTKLNGGPDFWPFLPLLMALRTHEEFTGDQRIVPFLTRFLRFMNAQGPGAFDSSWVSYRWGDGIDTAMWLHRRTGEAFLLDLVQKMHTYGANWVDNIPTPHNVNIAQGFREPAQYAQLTGSAELRQATYRGYTSVLGAYGQFPGGGFAGDENYRPGFGDPRQGFETCGIVEFMASHELLTRITGDPVWADRCEDLAFNMLPAALDPQGTGTHYITSANSIDLNNAVKSQGQFQNGFAMQSYQPGVDQYRCCPHNYGMGWPYFSEELWLATPDKGLAASLYAASQVSAKVAGGTTVTVTEDTDYPFDETITLTLSTPEKVAFPLHLRVPGWCKNPRIEVNGRAVATRGGPAFVKVDRSWTDGDVVTIRLPQRTALRTWSAQHGAVSVDHGPLTYSLRIGEDFVRYAGTDTFPEYEVHATTPWNYGLAPGALPVLTRDDGPLAANPFTHETTPVRMTAQARRIAEWVSDDEHVVTPLQQSPARADAPAETVTLIPMGAARLRITCFPTAAPDGRAWTPEPPFRRLLNKHSGKVLAVDEMSTANSARVVQYDNTPTGDHAWQWIDRGDGWFLIRNGHSGKVLGVDRMSTANSAIVVQYEDNGTADHLWRKVDNGDGWFRVLNKNSQKVLGVDGMSTANSAQVVQYDDNGTDDHLWRLL

SEQ ID NO: 67 - protein, 134 aa, Streptomyces sp.
MSAPQGQGPTFRELVVQALSSVERGYDLLAPKFDHTGYRTSASVLDSVTGALRPLGPFDSGLDVCCGTGAGMGVLRQVCRERITGVDFSAGMLAVGRERTRTVPDAPRTDWVRADARALPFEPVFDLAVSFGAF

SEQ ID NO: 68 - DNA, 28 bp, Streptomyces sp.
tgcaagcttctcgcgtctggtgctggtg

SEQ ID NO: 69 - DNA, 23 bp, Streptomyces sp.
atcttcgcccttgtcccgcagtc

SEQ ID NO: 70 - DNA, 21 bp, Streptomyces sp.
atcgctctgcggctggcggtg

SEQ ID NO: 71 - DNA, 28 bp, Streptomyces sp.
tgctctagagccacgaagacgccggaac